(19) United States
(12) Patent Application Publication
(10) Pub. No.: US 2021/0040118 A1
(43) Pub. Date: Feb. 11, 2021

(54) IMMUNOMODULATORS, COMPOSITIONS AND METHODS THEREOF

(71) Applicant: BETTA PHARMACEUTICALS CO., LTD, Hangzhou (CN)

(72) Inventors: Yiqian WANG, Beijing (CN); Bang FU, Beijing (CN); Yao ZHANG, Beijing (CN); Xiangyong LIU, Beijing (CN); Jiabing WANG, Beijing (CN); Lieming DING, Hangzhou (US)

(21) Appl. No.: 17/041,455

(22) PCT Filed: Apr. 3, 2019
(86) PCT No.: PCT/CN2019/081192

§ 371 Date: Sep. 25, 2020

(30) Foreign Application Priority Data
Apr. 3, 2018 (CN) PCT/CN2018/081776
Apr. 19, 2018 (CN) PCT/CN2018/083808
May 2, 2018 (CN) PCT/CN2018/085256
Jul. 12, 2018 (CN) PCT/CN2018/095508

(51) Int. Cl. C07D 513/04; C07D 417/14; C07D 495/04; C07D 471/04; C07D 277/56; C07D 487/04; C07D 271/10; C07D 519/00; C07D 498/04; C07D 285/12

(52) U.S. Cl. CPC C07D 513/04; C07D 417/14; C07D 495/04; C07D 471/04; C07D 285/12; C07D 487/04; C07D 271/10; C07D 519/00; C07D 498/04; C07D 277/56

(57) ABSTRACT

The present invention relates to compounds of Formula I, methods of using the compounds as immunomodulators, and pharmaceutical compositions comprising such compounds. The compounds are useful in treating, preventing or ameliorating diseases or disorders such as cancer or infections.

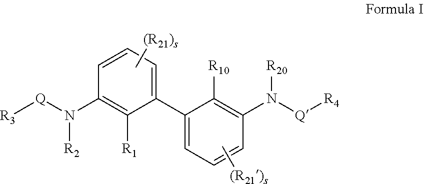

### CROSS REFERENCE TO RELATED APPLICATIONS

This application is a 371 of international PCT patent application PCT/CN2019/081192 filed on Apr. 3, 2019, which claims all benefits to PCT/CN2018/081776, filed Apr. 3, 2018, PCT/CN2018/083808, filed Apr. 19, 2018, PCT/CN2018/085256, filed May 2, 2018, and PCT/CN2018/095508, filed Jul. 12, 2018, the contents of which are hereby incorporated by reference.

### FIELD OF THE INVENTION

The present application is concerned with pharmaceutically active compounds. The disclosure provides compounds as well as their compositions and methods of use. The compounds modulate PD-1/PD-L1 protein/protein interaction and are useful in the treatment of various diseases including infectious diseases and cancer.

### BACKGROUND OF THE INVENTION

The immune system plays an important role in controlling and eradicating diseases such as cancer. However, cancer cells often develop strategies to evade or to suppress the immune system in order to favor their growth. One such mechanism is altering the expression of co-stimulatory and co-inhibitory molecules expressed on immune cells (Postow et al, J. Clinical Oncology 2015, 1-9). Blocking the signaling of an inhibitory immune checkpoint, such as PD-1, has proven to be a promising and effective treatment modality.

The interaction between PD-1 and PD-L1 results in a decrease in tumor infiltrating lymphocytes, a decrease in T-cell receptor mediated proliferation, and immune evasion by the cancerous cells (Dong et al, J. Mol Med., 81:281-287 (2003); Blank et al, Cancer Immunol Immunother., 54:307-314 (2005); Konishi et al, Clin. Cancer Res., 10:5094-5100 (2004)). Immune suppression can be reversed by inhibiting the local interaction of PD-1 with PD-L1, and the effect is additive when the interaction of PD-1 with PD-L2 is blocked as well (Iwai et al., Proc. Natl. Acad. Sci. USA, 99: 12293-12297 (2002); Brown et al, J. Immunol, 170: 1257-1266 (2003)).

Programmed cell death-1 (PD-1), also known as CD279, is a cell surface receptor expressed on activated T cells, natural killer T cells, B cells, and macrophages (Greenwald et al, Annu. Rev. Immunol 2005, 23:515-548; Okazaki and Honjo, Trends Immunol 2006, (4): 195-201). It functions as an intrinsic negative feedback system to prevent the activation of T-cells, which in turn reduces autoimmunity and promotes self-tolerance. In addition, PD-1 is also known to play a critical role in the suppression of antigen-specific T cell response in diseases like cancer and viral infection (Sharpe et al, Nat Immunol 2007 8, 239-245; Postow et al, J. Clinical Oncol 2015, 1-9).

The structure of PD-1 consists of an extracellular immunoglobulin variable-like domain followed by a transmembrane region and an intracellular domain (Parry et al, Mol Cell Biol 2005, 9543-9553). The intracellular domain contains two phosphorylation sites located in an immunoreceptor tyrosine-based inhibitory motif and an immunoreceptor tyrosine-based switch motif, which suggests that PD-1 negatively regulates T cell receptor-mediated signals. PD-1 has two ligands, PD-L1 and PD-L2 (Parry et al, Mol Cell Biol 2005, 9543-9553; Latchman et al, Nat Immunol 2001, 2, 261-268), and they differ in their expression patterns. PD-L1 protein is upregulated on macrophages and dendritic cells in response to lipopolysaccharide and GM-CSF treatment, and on T cells and B cells upon T cell receptor and B cell receptor signaling. PD-L1 is also highly expressed on almost all tumor cells, and the expression is further increased after IFN-γ treatment (Iwai et al, PNAS2002, 99(19): 12293-7; Blank et al, Cancer Res 2004, 64(3): 1140-5). In fact, tumor PD-L1 expression status has been shown to be prognostic in multiple tumor types (Wang et al, Eur J Surg Oncol 2015; Huang et al, Oncol Rep 2015; Sabatier et al, Oncotarget 2015, 6(7): 5449-5464). PD-L2 expression, in contrast, is more restricted and is expressed mainly by dendritic cells (Nakae et al, J Immunol 2006, 177:566-73). Ligation of PD-1 with its ligands PD-L1 and PD-L2 on T cells delivers a signal that inhibits IL-2 and IFN-γ production, as well as cell proliferation induced upon T cell receptor activation (Carter et al, Eur J Immunol 2002, 32(3):634-43; Freeman et al, J Exp Med 2000, 192(7): 1027-34). The mechanism involves recruitment of SHP-2 or SHP-1 phosphatases to inhibit T cell receptor signaling such as Syk and Lck phosphorylation (Sharpe et al, Nat Immunol 2007, 8, 239-245). Activation of the PD-1 signaling axis also attenuates PKC-θ activation loop phosphorylation, which is necessary for the activation of NF-κB and API pathways, and for cytokine production such as IL-2, IFN-γ and TNF (Sharpe et al, Nat Immunol 2007, 8, 239-245; Carter et al, Eur J Immunol 2002, 32(3):634-43; Freeman et al, J Exp Med 2000,192(7): 1027-34).

Several lines of evidence from preclinical animal studies indicate that PD-1 and its ligands negatively regulate immune responses. PD-1-deficient mice have been shown to develop lupus-like glomerulonephritis and dilated cardiomyopathy (Nishimura et al, Immunity 1999, 11:41-151; Nishimura et al, Science 2001, 291:319-322). Using an LCMV model of chronic infection, it has been shown that PD-1/PD-L1 interaction inhibits activation, expansion and acquisition of effector functions of virus-specific CD8 T cells (Barber et al, Nature 2006, 439, 682-7).

Together, these data support the development of a therapeutic approach to block the PD-1 mediated inhibitory signaling cascade in order to augment or “rescue” T cell response. Most of the currently approved medicines in immunotherapy are monoclonal antibodies. However, small molecule inhibitors that directly target PD-1 or PD-L1 are still not approved, there is only CA170 have been evaluated clinically.

Accordingly, there is still great demand for more potent, and more easily administered therapeutics against PD-1/PD-L1 protein/protein interactions. In this invention, applicant discovered potent small molecules that can have activity as inhibitors of the interaction of PD-L1 with PD-1, and thus may be useful for therapeutic administration to enhance immunity against cancer and/or infectious diseases. These small molecules are expected to be useful as pharmaceuticals with desirable stability, solubility, bioavailability, therapeutic index and toxicity values that are crucial to become efficient medicines to promote human health.

### SUMMARY OF INVENTION

The present invention relates to compounds that are used as inhibitors of the functional interaction between PD-L1 and PD-1. Inhibitors of the interaction between PD-L1 and PD-1 are useful in the treatment of cancers and infectious diseases.

The compounds of the invention have the general structures as Formula I. A compound of Formula I, or a stereoisomer, tautomer, pharmaceutically acceptable salt, prodrug, chelate, non-covalent complex, or solvate thereof,

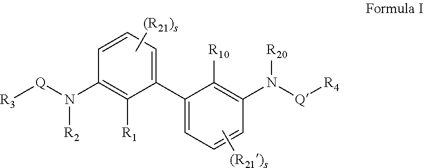

wherein,

Q and Q′ are each independently selected from absent, C(O) or C(R₂)₂;

R₁ and R₁₀ are each independently selected from H, halogen, CN, or C₁₋₈alkyl;

R₂ and R₂₀ are each independently selected from H, or C₁₋₈alkyl; or

R₁ and R₂ together with the atoms to which they are attached form a 5- to 6-member heterocyclic ring; or

R₁₀ and R₂₀ together with the atoms to which they are attached form a 5- to 6-member heterocyclic ring;

R₃ and R₄ are each independently selected from heterocyclic ring or heteroaryl ring optionally comprising 1, 2 or 3 hetero atoms independently selected from N, S, or O, wherein the heterocyclic ring or heteroaryl ring is monocyclic or bicyclic, optionally substituted with C₁₋₈alkyl, —(CH₂)ₚ—COOH, —(CH₂)ₚ—CONR₃₀R₄₀, —(CH₂)ₚ—OH, —(CH₂)ₚ-heterocyclyl, —C₃₋₇cycloalkyl, —C₃₋₇heterocyclyl, or —(CH₂)ₚ—NR₃₀R₄₀, wherein C₁₋₈alkyl, —(CH₂)ₚ—COOH, —(CH₂)ₚ—CONR₃₀R₄₀, —(CH₂)ₚ—OH, —(CH₂)ₚ-heterocyclyl, —C₃₋₇cycloalkyl, —C₃₋₇heterocyclyl, or —(CH₂)ₚ—NR₃₀R₄₀ is optionally substituted with C₁₋₈alkyl, halogen, OH, CN, COOH, or NR₃₀R₄₀;

R₃₀ and R₄₀ are each independently selected from H, C₁₋₈alkyl, —C₃₋₇cycloalkyl, or —C₃₋₇heterocyclyl; or

R₃₀ and R₄₀ together with the atoms to which they are attached form a 5- to 6-member heterocyclic ring;

R₂₁ and R₂₁′ are each independently selected from halogen, CN, OH, COOH, or C₁₋₈alkyl;

s and p are each independently selected from 0, 1, 2 or 3.

In some embodiments of Formula I, Q and Q′ are each independently selected from absent, C(O) or CH₂.

In some embodiments of Formula I, s and p are each independently selected from 0 or 1.

In some embodiments of Formula I, R₁ is independently selected from methyl, F or Cl.

In some embodiments of Formula I, R₂ is independently selected from H or methyl.

In other embodiments, R₁ and R₁₀ together with the atoms to which they are attached form a 5-member heterocyclic ring.

In other embodiments, R₂ and R₂₀ together with the atoms to which they are attached form a 5-member heterocyclic ring.

In some embodiments of Formula I, R₂₁ and R₂₁′ are each independently selected from —CH₃, F, Cl or CN.

In some embodiments of Formula I, R₃ and R₄ are each independently selected from

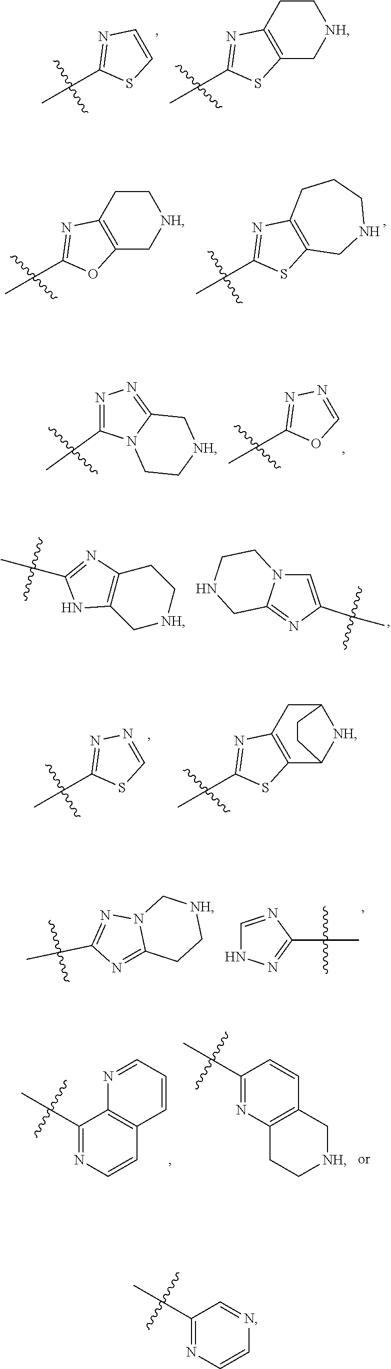

which is each unsubstituted or substituted with at least one substituent selected from C₁₋₆alkyl, —(CH₂)ₚ—COOH, —(CH₂)ₚ—CONR₃₀R₄₀, —(CH₂)ₚ—OH, —(CH₂)ₚ-heterocyclyl, —C₃₋₇cycloalkyl, —C₃₋₇heterocyclyl, or —(CH₂)ₚ—NR₃₀R₄₀, wherein C₁₋₆alkyl, —(CH₂)ₚ—COOH, —(CH₂)ₚ—NH₂, —(CH₂)ₚ—CONR₃₀R₄₀, —(CH₂)ₚ—OH, —(CH₂)ₚ-heterocyclyl, —C₃₋₇cycloalkyl, —C₃₋₇heterocyclyl, or —(CH₂)ₚ—NR₃₀R₄₀ is optionally substituted with C₁₋₈alkyl, halogen, OH, CN, COOH, or NR₃₀R₄₀;

R₃₀ and R₄₀ are each independently selected from H, C₁₋₈alkyl, —C₃₋₇cycloalkyl, or —C₃₋₇heterocyclyl; or

R₃₀ and R₄₀ together with the atoms to which they are attached form a 5- to 6-member heterocyclic ring.

In some embodiments of Formula I, R₃₀ and R₄₀ are each independently selected from H, or C₁₋₃alkyl.

In some embodiments of Formula I, wherein R₃ and R₄ are each independently selected from

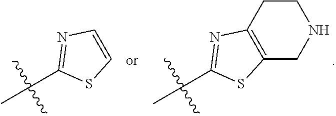 [SMILES: *c1nccs1 ; *c1nc2c(s1)CNCC2]

In some embodiments of Formula I, wherein, R₃ and R₄ are each independently selected from

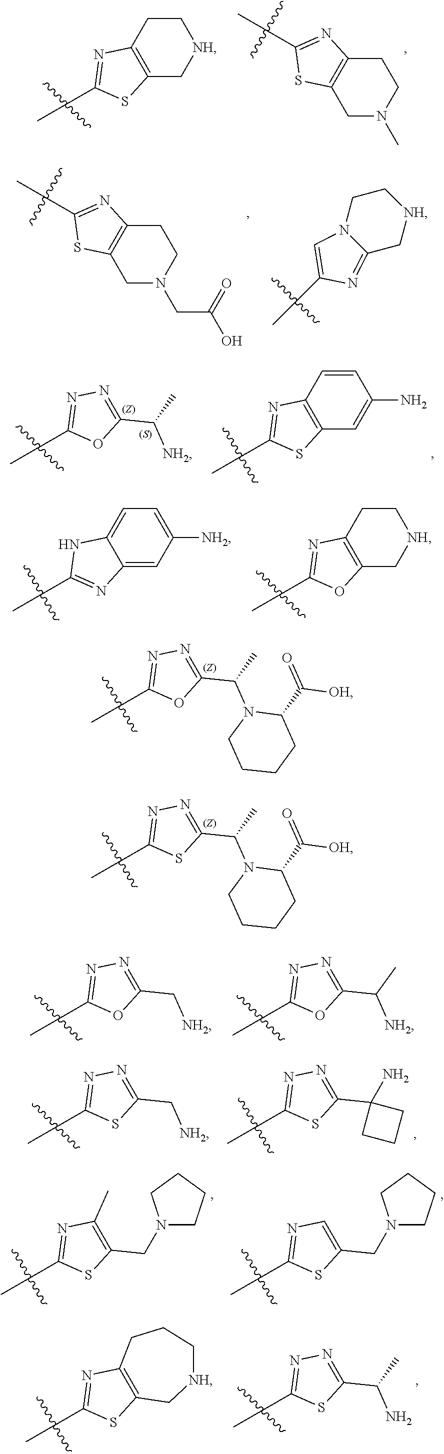

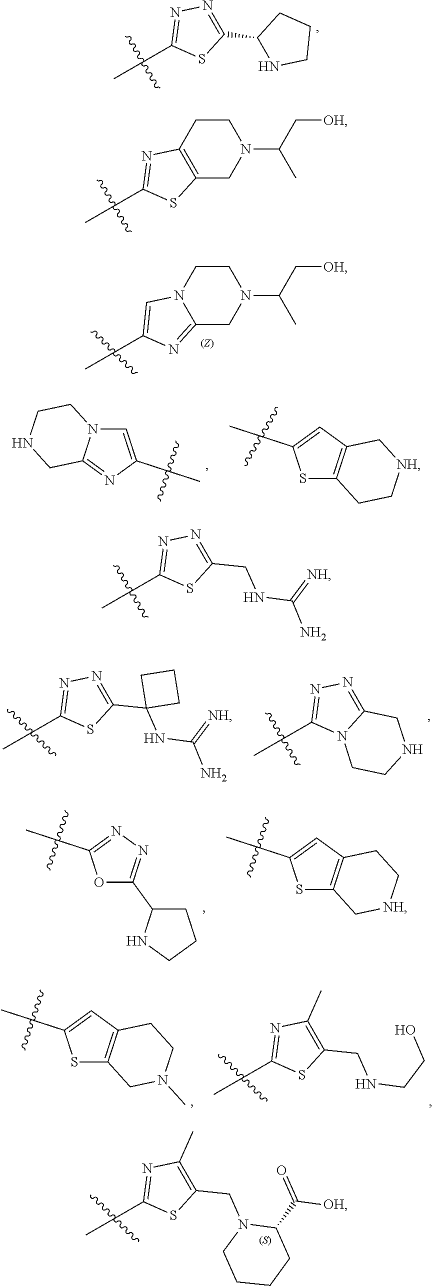

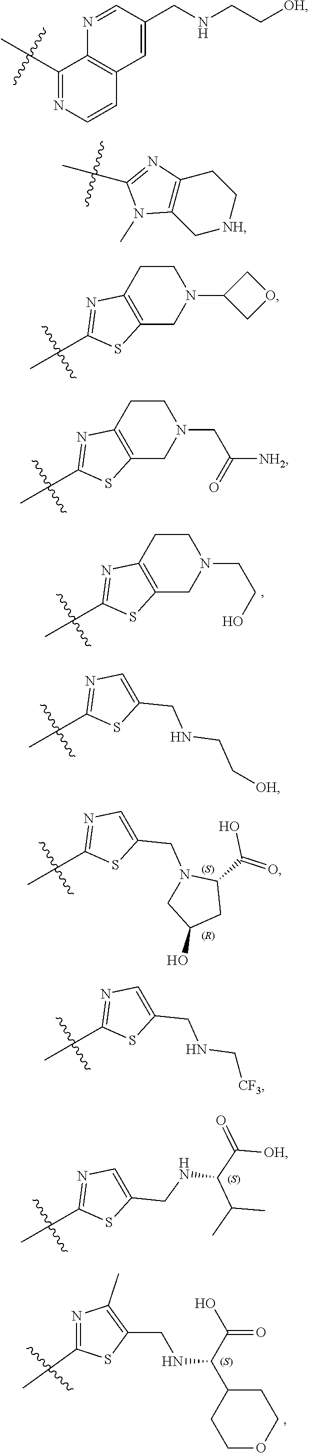

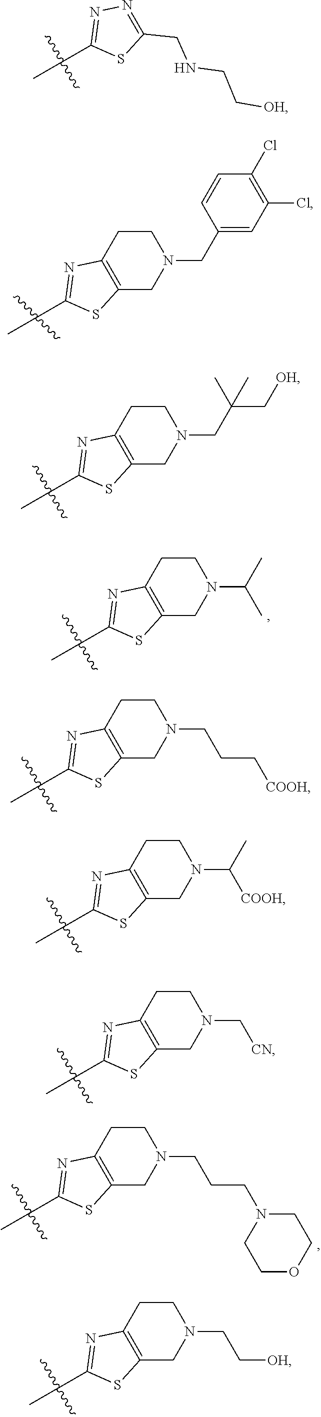

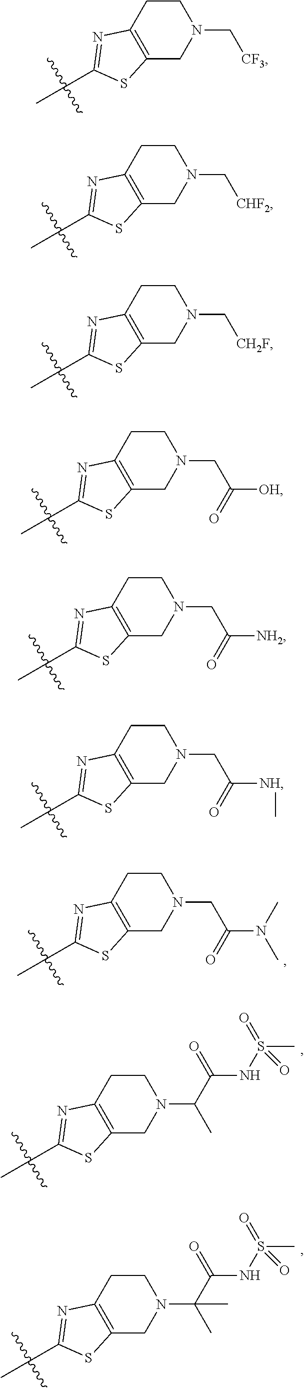

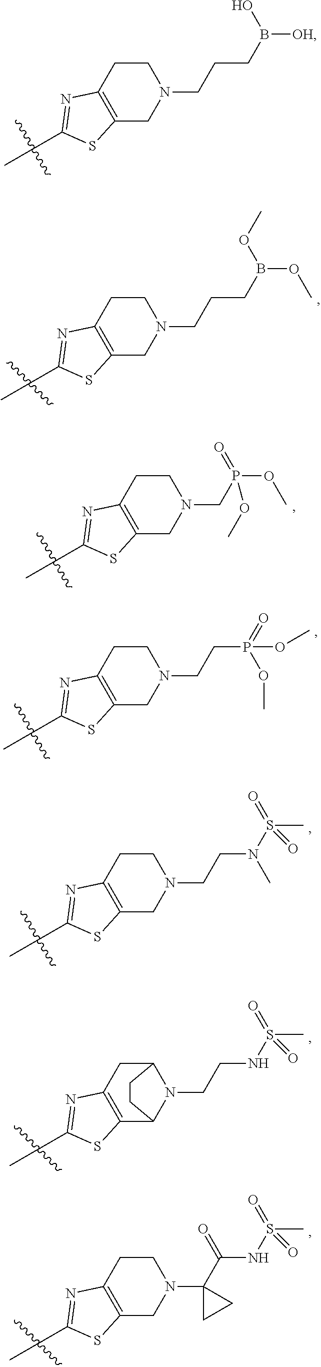

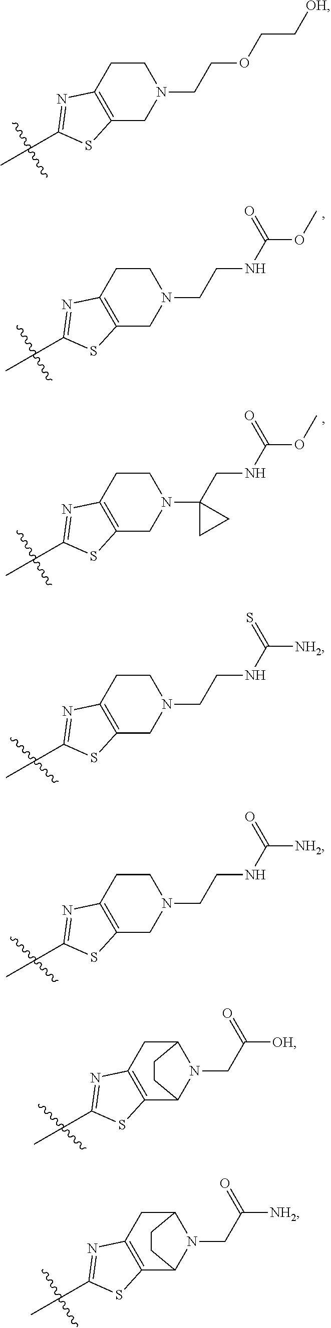

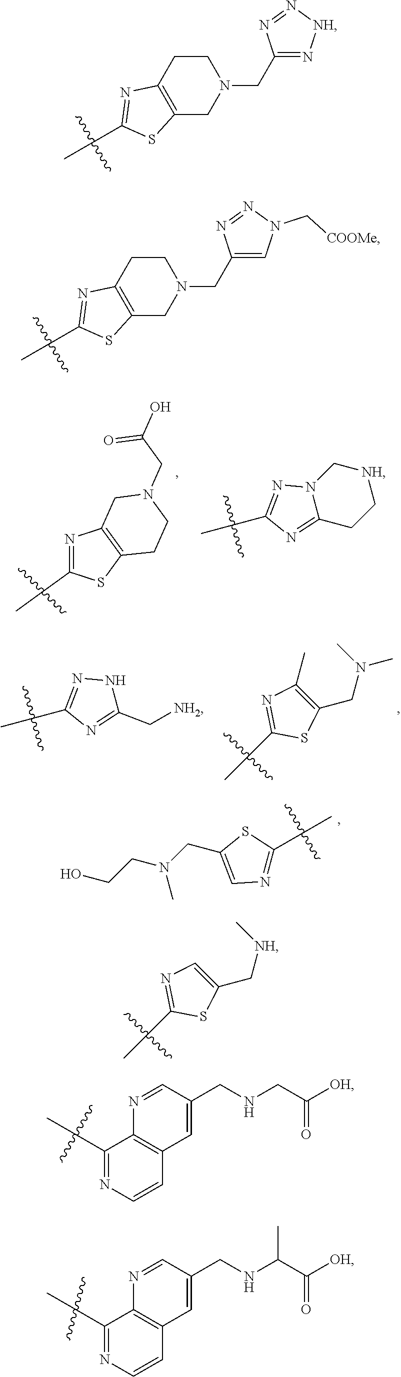

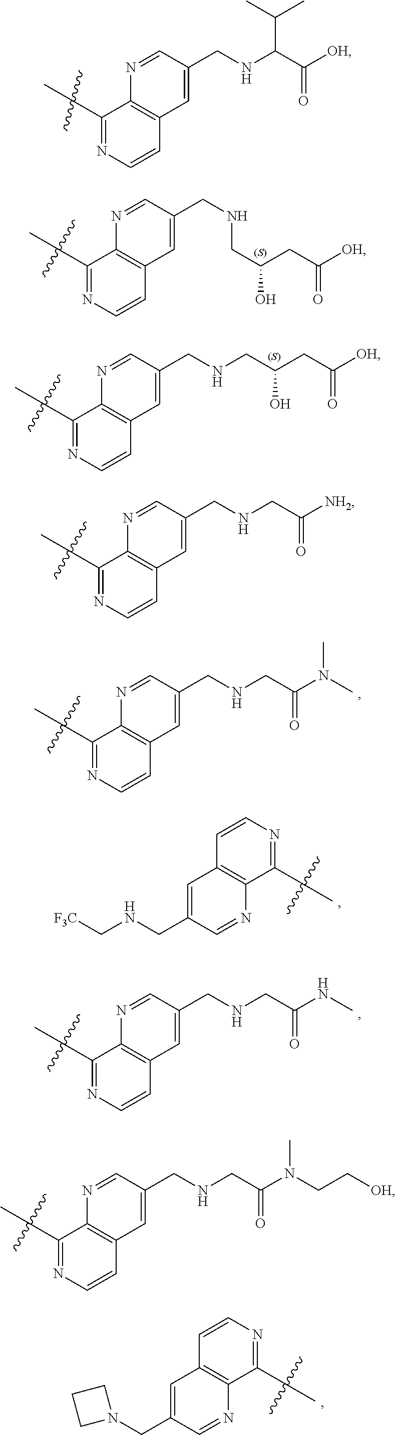

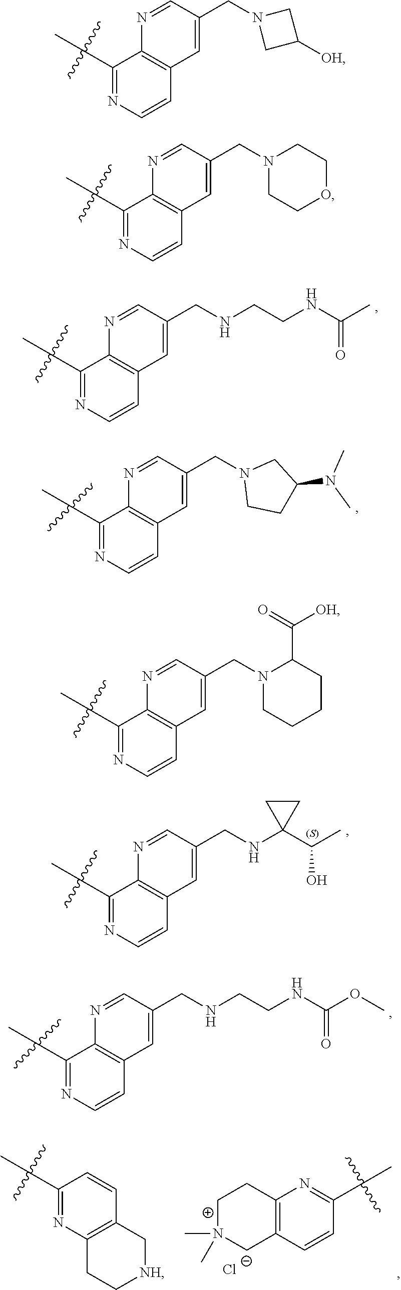

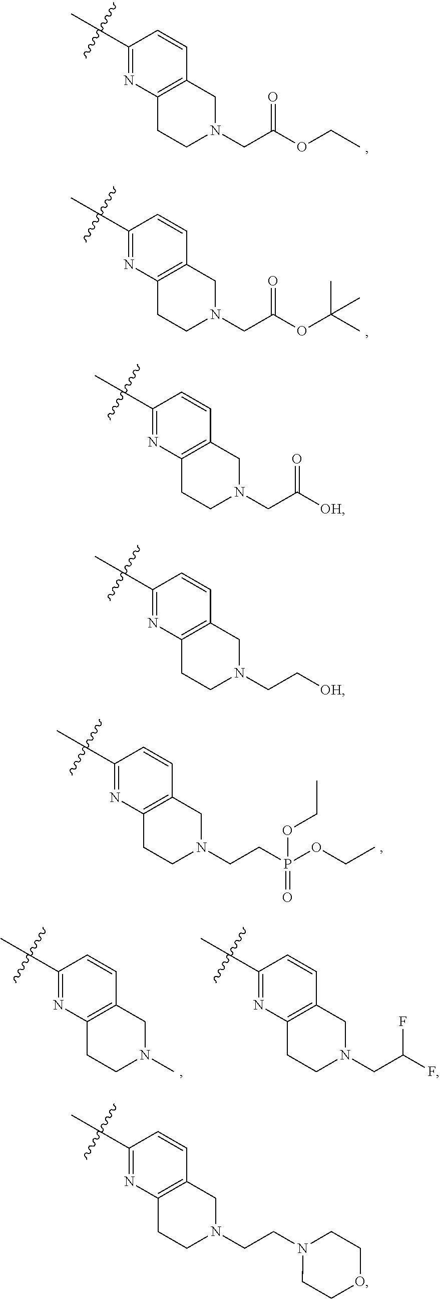

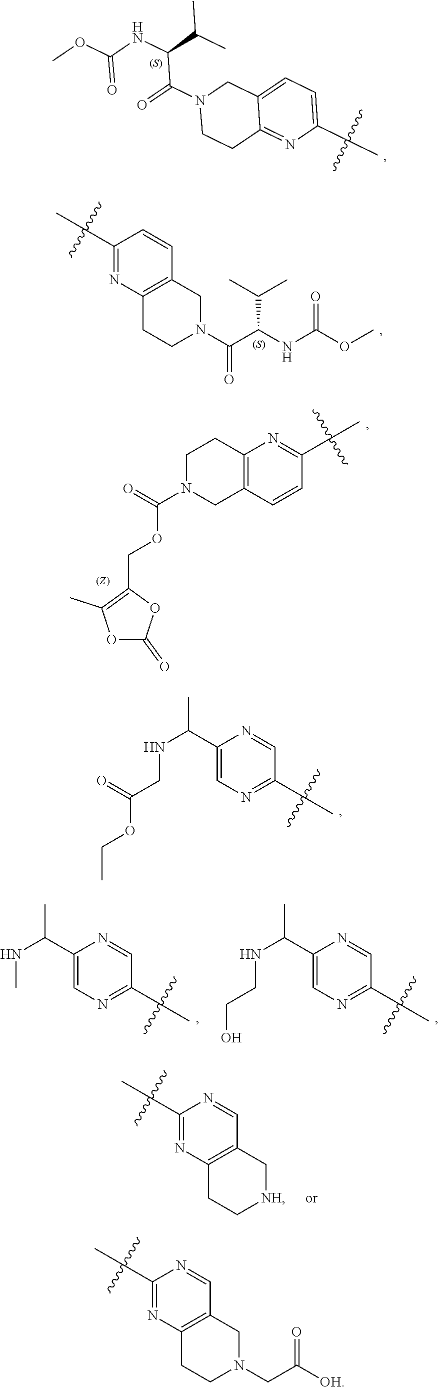

In some embodiments of Formula I, the compound is of Formula II,

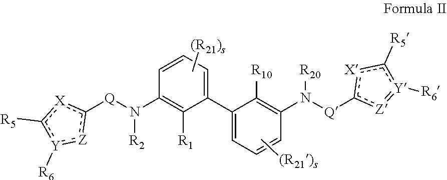

wherein,

R₁ and R₁₀ are each independently selected from H, halogen, CN, or C₁₋₈alkyl;

R₂ and R₂₀ are each independently selected from H, or C₁₋₈alkyl; or

R₁ and R₂ together with the atoms to which they are attached form a 5- to 6-member heterocyclic ring; or

R₁₀ and R₂₀ together with the atoms to which they are attached form a 5- to 6-member heterocyclic ring;

Q and Q′ are each independently selected from absent, C(O) or C(R₂)₂;

X, Y, Z, X′, Y′ or Z′ is independently selected from N, S, O or C;

R₅, R₆, R₅′ and R₆′ are each independently selected from (CH₂)ₚ—NR₃₀R₄₀, (CH₂)ₘ-cycloalkyl, (CH₂)ₘ-heterocyclyl, C₁₋₈alkyl, wherein (CH₂)ₚ—NR₃₀R₄₀, (CH₂)ₘ-heterocyclyl, C₁₋₈alkyl are each unsubstituted or substituted with at least one substituent selected from C₁₋₈alkyl, —COOH, —NH₂, (CH₂)ₘ-hydroxyl, or CN; or

R₅ and R₆ together with the atoms to which they are attached form a 6- to 7-member heterocyclic ring comprising 1, 2 or 3 hetero atoms independently selected from N, O or S, the heterocyclic ring being unsubstituted or substituted with at least one substituent selected from C₁₋₈alkyl, (CH₂)ₘ-carboxyl, (CH₂)ₘ-hydroxyl, (CH₂)ₘ-heterocyclyl, (CH₂)ₘ-aryl, (CH₂)ₘ-amido, (CH₂)ₘ—CN, (CH₂)ₘ—CF₃, (CH₂)ₘ—CHF₂, (CH₂)ₘCH₂F, or (CH₂)ₘ—NH₂; or

R₅′ and R₆′ together with the atoms to which they are attached form a 6- to 7-member heterocyclic ring comprising 1, 2 or 3 hetero atoms independently selected from N, O or S, the heterocyclic ring being unsubstituted or substituted with at least one substituent selected from C₁₋₈alkyl, (CH₂)ₘ-carboxyl, (CH₂)ₘ-hydroxyl, (CH₂)ₘ-heterocyclyl, (CH₂)ₘ-aryl, (CH₂)ₘ-amido, (CH₂)ₘ—CN, (CH₂)ₘ—CF₃, (CH₂)ₘ—CHF₂, (CH₂)ₘ—CH₂F, or (CH₂)ₘ—NH₂;

R₃₀ and R₄₀ are each independently selected from H, C₁₋₈alkyl, —C₃₋₇cycloalkyl, or —C₃₋₇heterocyclyl; or

R₃₀ and R₄₀ together with the atoms to which they are attached form a 5- to 6-member heterocyclic ring;

R₂₁ and R₂₁′ are each independently selected from halogen, CN, OH, COOH, or C₁₋₈alkyl;

p, s, and m are each independently selected from 0, 1, 2 or 3;

[symbol] is a single bond or a double bond.

In some embodiments of Formula II, wherein, R₁ and R₁₀ are independently selected from H, methyl, F or Cl.

In some embodiments of Formula II, wherein, R₂ and R₂₀ are independently selected from H or methyl.

In some embodiments of Formula II, R₁ and R₁₀ together with the atoms to which they are attached form a 5-member heterocyclic ring.

In some embodiments of Formula II, R₂ and R₂₀ together with the atoms to which they are attached form a 5-member heterocyclic ring.

In some embodiments of Formula II, R₂₁ and R₂₁′ are each independently selected from —CH₃, F, Cl or CN.

In some embodiments of Formula II, wherein, X and X′ are each independently selected from O, S or N.

In some embodiments of Formula II, wherein, Y and Y′ are each independently selected from C, S or N.

In some embodiments of Formula II, wherein, Z and Z′ are each independently selected from C, or N.

In some embodiments of Formula II, wherein, R₅ and R₅′ are each independently selected from —CH₃,

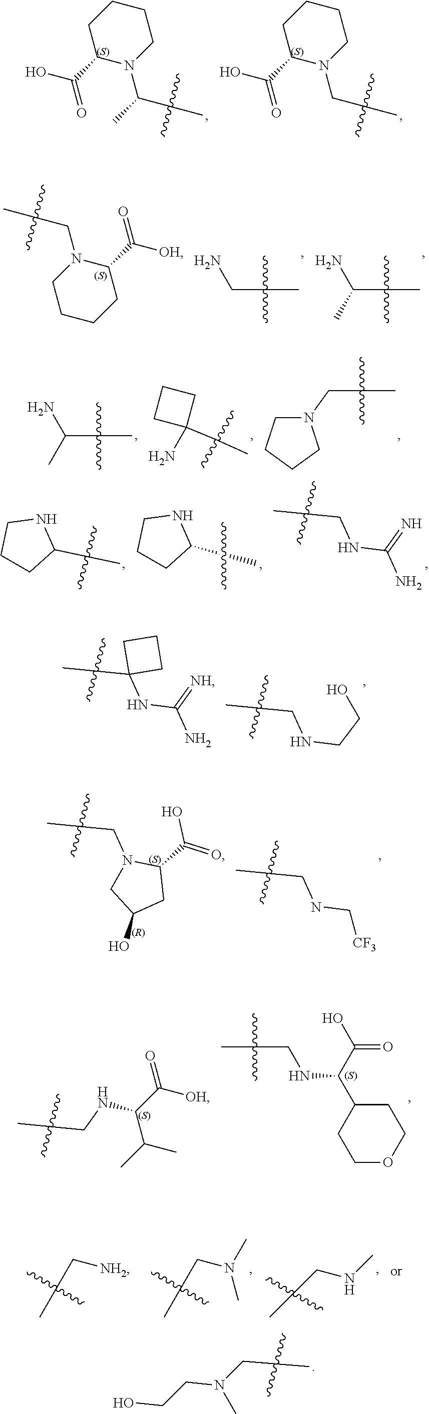

In some embodiments of Formula II, wherein, R₆ and R₆′ are each independently selected from absent, H or methyl.

In some embodiments of Formula I, the compound is of Formula III:

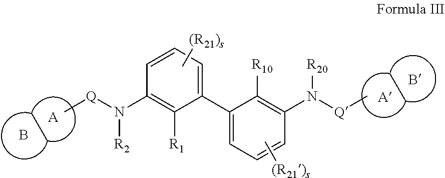

wherein,

R₁ and R₁₀ are each independently selected from H, halogen, CN, or C₁₋₈alkyl;

R₂ and R₂₀ are each independently selected from H, or C₁₋₈alkyl; or

R₁ and R₂ together with the atoms to which they are attached form a 5- to 6-member heterocyclic ring; or

R₁₀ and R₂₀ together with the atoms to which they are attached form a 5- to 6-member heterocyclic ring;

Q and Q′ are each independently selected from absent, C(O) or C(R₂)₂;

Ring A and Ring A′ are independently a 5-6 membered aromatic heterocyclic ring, which is each unsubstituted or substituted with at least one substituent selected from C₁₋₈alkyl, halogen, OH, or CN;

Ring B and Ring B′ are independently a 5-7 membered heterocyclic ring, which is each unsubstituted or substituted with at least one substituent selected from C₁₋₈alkyl, —(CH₂)ₚ—COOH, —(CH₂)ₚ—CONR₃₀R₄₀, —(CH₂)ₚ—OH, —(CH₂)ₚ-heterocyclyl, —C₃₋₇cycloalkyl, —C₃₋₇heterocyclyl, or —(CH₂)ₚ—NR₃₀R₄₀, wherein C₁₋₈alkyl, —(CH₂)ₚ—COOH, —(CH₂)ₚ—CONR₃₀R₄₀, —(CH₂)ₚ—OH, —(CH₂)ₚ-heterocyclyl, —C₃₋₇cycloalkyl, —C₃₋₇heterocyclyl, or —(CH₂)ₚ—NR₃₀R₄₀ is optionally substituted with C₁₋₈alkyl halogen, OH, CN, COOH, or NR₃₀R₄₀;

R₃₀ and R₄₀ are each independently selected from H, C₁₋₈alkyl, —C₃₋₇cycloalkyl, or —C₃₋₇heterocyclyl; or

R₃₀ and R₄₀ together with the atoms to which they are attached form a 5- to 6-member heterocyclic ring;

R₂₁ and R₂₁′ are each independently selected from halogen, CN, OH, COOH, or C₁₋₈alkyl;

p and s are each independently selected from 0, 1, 2 or 3.

In some embodiments of Formula III, wherein R₁ and R₁₀ are independently selected from H, methyl F or Cl.

In some embodiments of Formula III, wherein, R₂ and R₂₀ are independently selected from H or methyl.

In some embodiments of Formula III, wherein R₁ and R₂ together with the atoms to which they are attached form a 5-membered heterocyclic ring.

In some embodiments of Formula III, wherein R₁₀ and R₂₀ together with the atoms to which they are attached form a 5-membered heterocyclic ring.

In some embodiments of Formula III, R₂₁ and R₂₁′ are each independently selected from —CH₃, F, Cl or CN.

In some embodiments of Formula III, s and p are each independently selected from 0 or 1.

In some embodiments of Formula III,

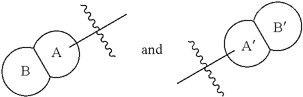

are each independently selected from

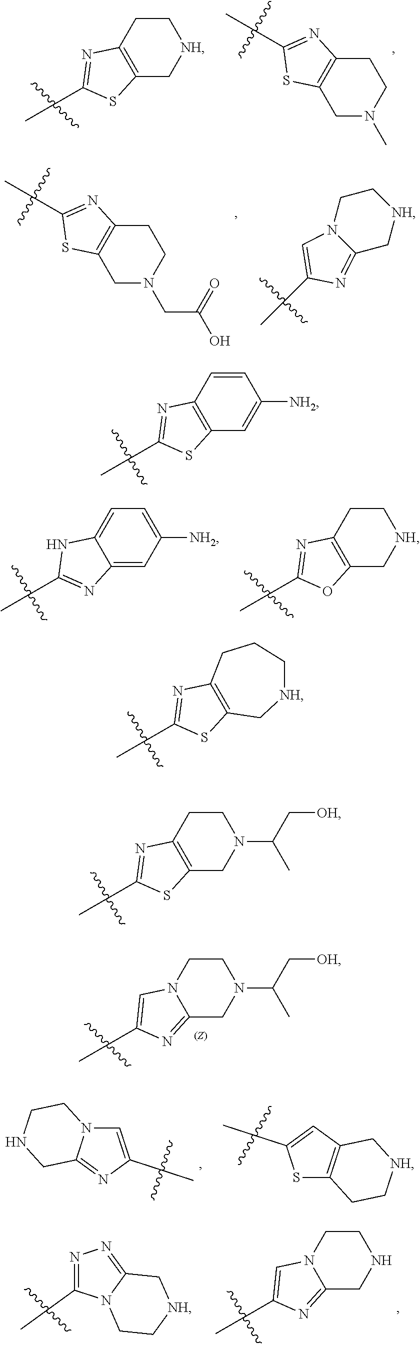

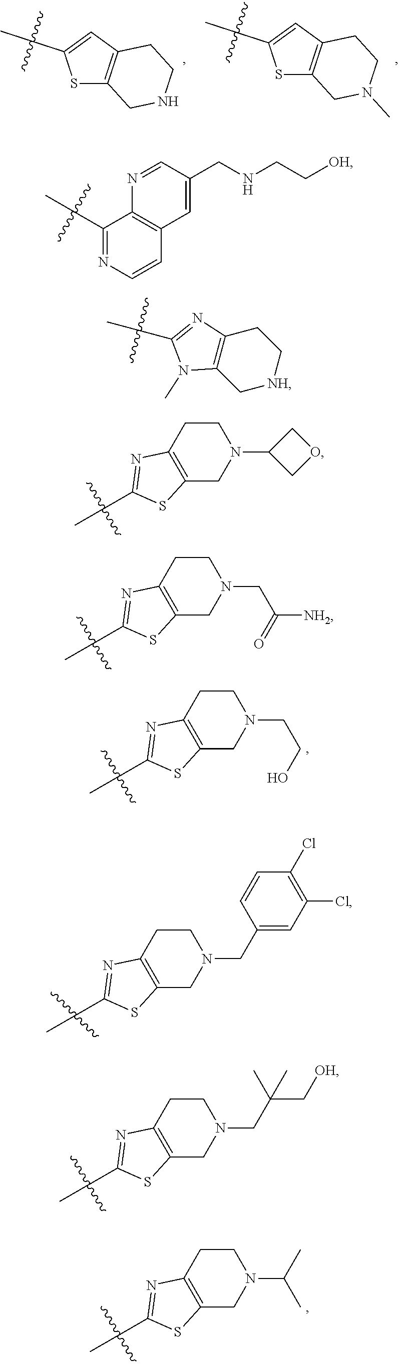

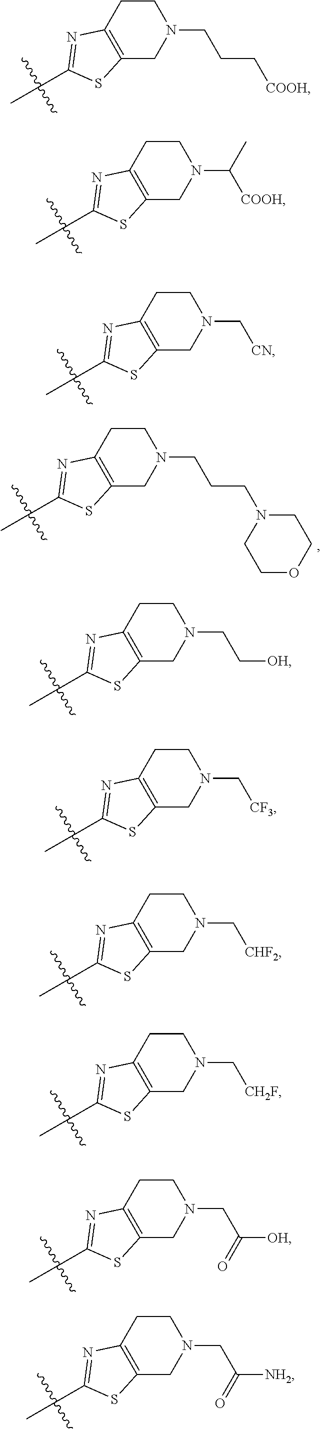

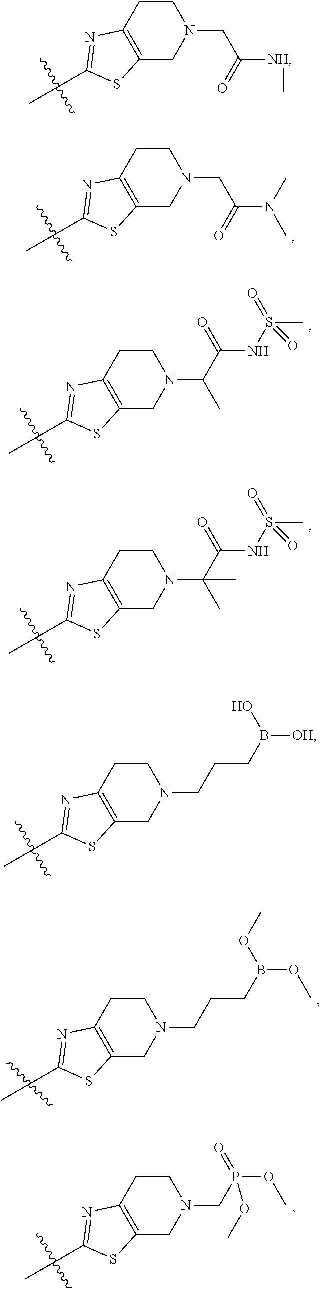

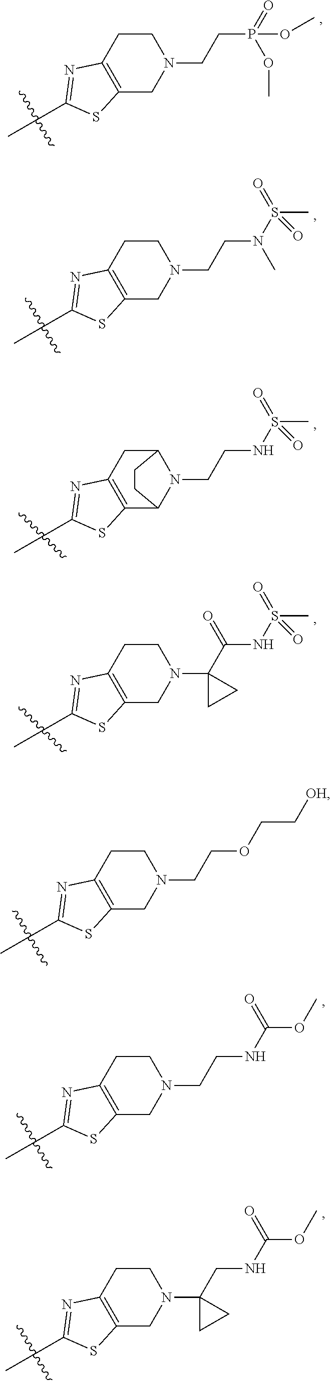

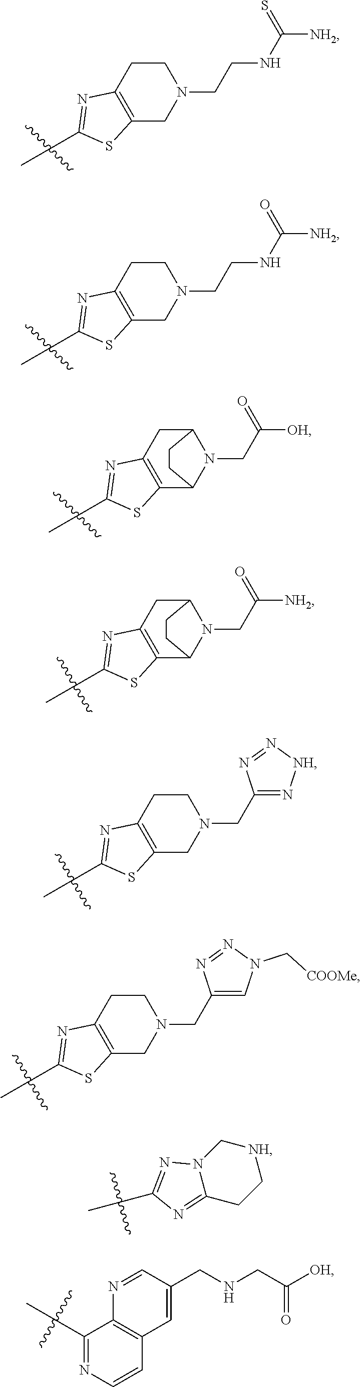

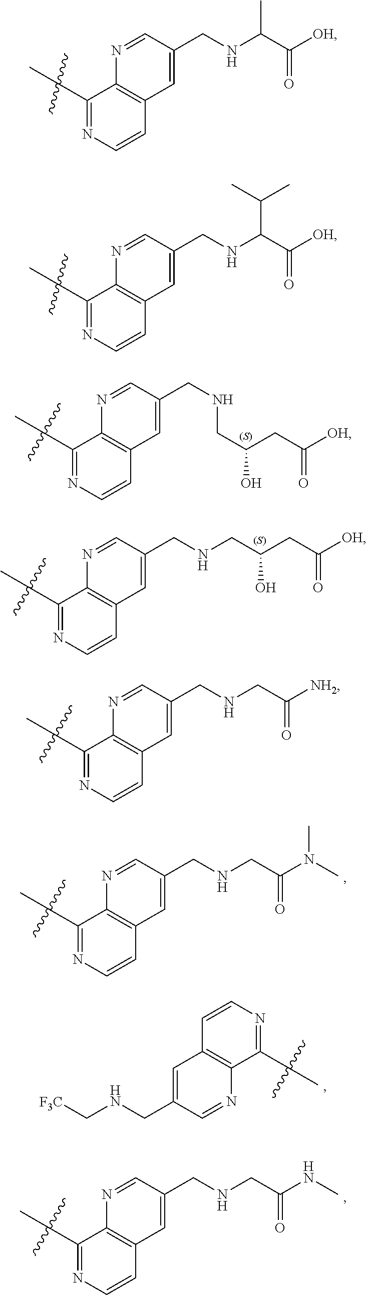

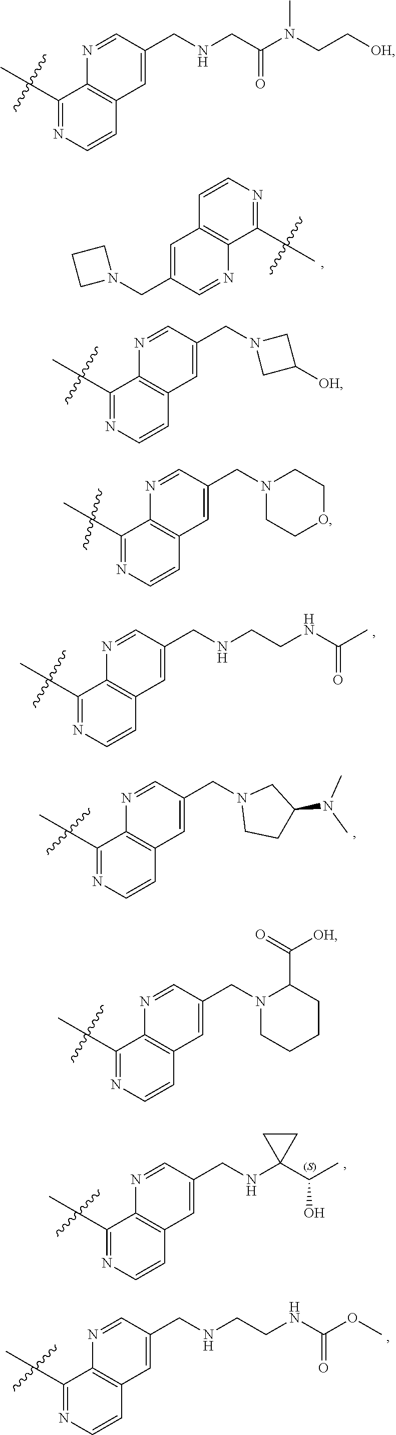

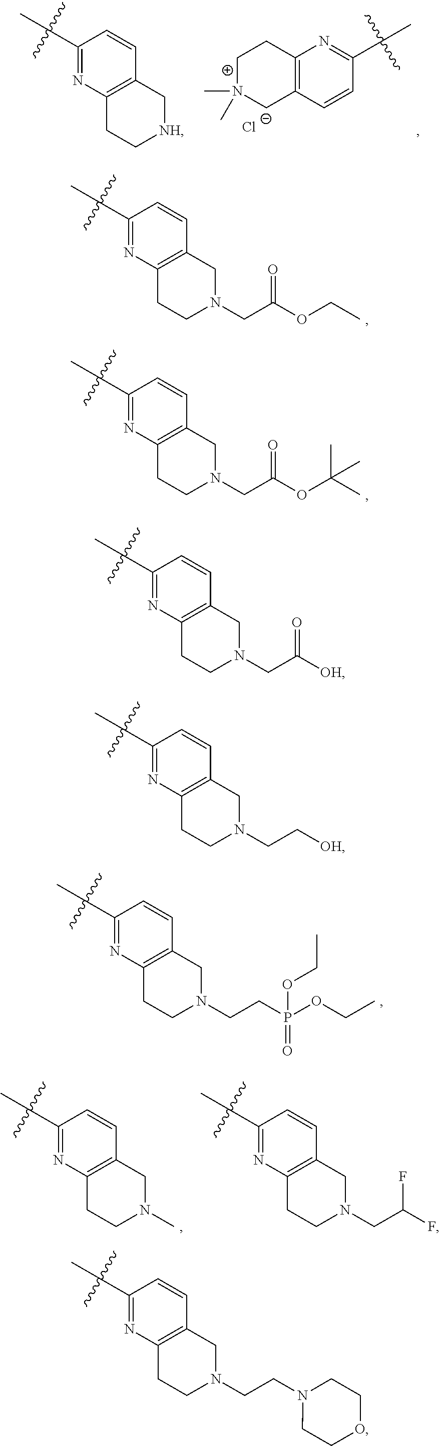

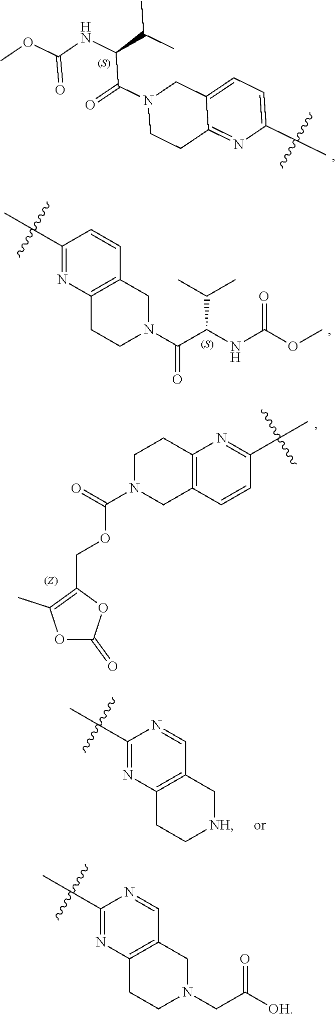

In some embodiments, Q and Q′ are the same, R₁ and R₁₀ are the same, R₂ and R₂₀ are the same, R₃ and R₄ are the same, R₂₁ and R₂₁′ are the same.

The present invention further provides some preferred technical solutions with regard to compound of Formula (I), Formula (II) or Formula (III), wherein the compound is:
1) N,N′-(2,2′-dimethyl-[1,1′-biphenyl]-3,3′-diyl)bis(4,5,6,7-tetrahydrothiazolo[5,4-c]pyridine-2-carboxamide);

2) N,N′-(2,2′-dimethyl-[1,1′-biphenyl]-3,3′-diyl)bis(5-methyl-4,5,6,7-tetrahydrothiazolo[5,4-c]pyridine-2-carboxamide);
3) N,N′-(2,2′-dimethyl-[1,1′-biphenyl]-3,3′-diyl)bis(5-(pyrrolidin-2-yl)-1,3,4-thiadiazole-2-carboxamide);
4) 2,2′-((((2,2′-dimethyl-[1,1′-biphenyl]-3,3′-diyl)bis(azanediyl))bis(carbonyl))bis(6,7-dihydrothiazolo[5,4-c]pyridine-2,5(4H)-diyl))diacetic acid;
5) N-(2,2′-dimethyl-3′-(N-methyl-5,6,7,8-tetrahydroimidazo[1,2-a]pyrazine-2-carboxamido)-[1,1′-biphenyl]-3-yl)-N-methyl-4,5,6,7-tetrahydrothieno[2,3-c]pyridine-2-carboxamide;
6) 2-((8-((3′-((3-(((2-hydroxyethyl)amino)methyl)-1,7-naphthyridin-8-yl)amino)-2,2′-dimethyl-[1,1′-biphenyl]-3-yl)amino)quinolin-3-yl)amino)ethan-1-ol;
7) N,N′-(2,2′-dimethyl-[1,1′-biphenyl]-3,3′-diyl)bis(5-(((2-hydroxyethyl)amino)methyl)thiazole-2-carboxamide);
8) N,N′-(2,2′-dimethyl-[1,1′-biphenyl]-3,3′-diyl)bis(5,6,7,8-tetrahydroimidazo[1,2-a]pyrazine-2-carboxamide);
9) N,N′-(2,2′-dimethyl-[1,1′-biphenyl]-3,3′-diyl)bis(5-((S)-1-aminoethyl)-1,3,4-oxadiazole-2-carboxamide);
10) N-(2,2′-dimethyl-3′-(5,6,7,8-tetrahydroimidazo[1,2-a]pyrazine-2-carboxamido)-[1,1′-biphenyl]-3-yl)-4,5,6,7-tetrahydrothiazolo[5,4-c]pyridine-2-carboxamide;
11) N-(3′-(6-aminobenzo[d]thiazole-2-carboxamido)-2,2′-dimethyl-[1,1′-biphenyl]-3-yl)-4,5,6,7-tetrahydrothiazolo[5,4-c]pyridine-2-carboxamide;
12) N-(3′-(5-amino-H-benzo[d]imidazole-2-carboxamido)-2,2′-dimethyl-[1,1′-biphenyl]-3-yl)-4,5,6,7-tetrahydrothiazolo[5,4-c]pyridine-2-carboxamide;
13) N,N′-(2,2′-dimethyl-[1,1′-biphenyl]-3,3′-diyl)bis(4,5,6,7-tetrahydrooxazolo[5,4-c]pyridine-2-carboxamide);
14) N-(2,2′-dimethyl-3′-(4,5,6,7-tetrahydrothiazolo[5,4-c]pyridine-2-carboxamido)-[1,1′-biphenyl]-3-yl)-4,5,6,7-tetrahydrooxazolo[5,4-c]pyridine-2-carboxamide;
15) N-(2,2′-dimethyl-3′-(4,5,6,7-tetrahydrothieno[3,2-c]pyridine-2-carboxamido)-[1,1′-biphenyl]-3-yl)-4,5,6,7-tetrahydrothiazolo[5,4-c]pyridine-2-carboxamide;
16) (2S,2'S)-1,1′-((1S,1'S)-((((2,2′-dimethyl-[1,1′-biphenyl]-3,3′-diyl)bis(azanediyl))bis(carbonyl))bis(1,3,4-oxadiazole-5,2-diyl))bis(ethane-1,1-diyl))bis(piperidine-2-carboxylic acid);
17) (2S,2'S)-1,1′-((1S,1'S)-((((2,2′-dimethyl-[1,1′-biphenyl]-3,3′-diyl)bis(azanediyl))bis(carbonyl))bis(1,3,4-thiadiazole-5,2-diyl))bis(ethane-1,1-diyl))bis(piperidine-2-carboxylic acid);
18) N,N′-(2,2′-dimethyl-[1,1′-biphenyl]-3,3′-diyl)bis(5-(aminomethyl)-1,3,4-oxadiazole-2-carboxamide);
19) N,N′-(2,2′-dimethyl-[1,1′-biphenyl]-3,3′-diyl)bis(5-(1-aminoethyl)-1,3,4-oxadiazole-2-carboxamide);
20) N,N′-(2,2′-dimethyl-[1,1′-biphenyl]-3,3′-diyl)bis(5-(aminomethyl)-1,3,4-thiadiazole-2-carboxamide);
21) N-(3′-(6-amino-N-methylbenzo[d]thiazole-2-carboxamido)-2,2′-dimethyl-[1,1′-biphenyl]-3-yl)-N-methyl-4,5,6,7-tetrahydrothiazolo[5,4-c]pyridine-2-carboxamide;
22) N3-((6-aminobenzo[d]thiazol-2-yl)methyl)-N3,N3′,2,2′-tetramethyl-N3′-((4,5,6,7-tetrahydrothiazolo[5,4-c]pyridin-2-yl)methyl)-[1,1′-biphenyl]-3,3′-diamine;
23) N,N′-(2,2′-dimethyl-[1,1′-biphenyl]-3,3′-diyl)bis(N-methyl-4,5,6,7-tetrahydrothiazolo[5,4-c]pyridine-2-carboxamide);
24) N3,N3′,2,2′-tetramethyl-N3,N3′-bis((4,5,6,7-tetrahydrothiazolo[5,4-c]pyridin-2-yl)methyl)-[1,1′-biphenyl]-3,3′-diamine;
25)2,2′-dimethyl-N3,N3′-bis((4,5,6,7-tetrahydrothiazolo[5,4-c]pyridin-2-yl)methyl)-[1,1′-biphenyl]-3,3′-diamine;
26) N3,N3′-bis((5-(1-aminocyclobutyl)-1,3,4-thiadiazol-2-yl)methyl)-2,2′-dimethyl-[1,1′-biphenyl]-3,3′-diamine;
27) N,N′-(2,2′-dimethyl-[1,1′-biphenyl]-3,3′-diyl)bis(5-(1-aminocyclobutyl)-1,3,4-thiadiazole-2-carboxamide);
28) N,N′-(2,2′-dimethyl-[1,1′-biphenyl]-3,3′-diyl)bis(4-methyl-5-(pyrrolidin-1-ylmethyl)thiazole-2-carboxamide);
29) N,N′-(2,2′-dimethyl-[1,1′-biphenyl]-3,3′-diyl)bis(5-(pyrrolidin-1-ylmethyl)thiazole-2-carboxamide);
30) N,N′-(2,2′-dimethyl-[1,1′-biphenyl]-3,3′-diyl)bis(5,6,7,8-tetrahydro-4H-thiazolo[5,4-c]azepine-2-carboxamide);
31) N,N′-(2,2′-dimethyl-[1,1′-biphenyl]-3,3′-diyl)bis(5-((S)-1-aminoethyl)-1,3,4-thiadiazole-2-carboxamide);
32) N,N′-(2,2′-dimethyl-[1,1′-biphenyl]-3,3′-diyl)bis(4,5,6,7-tetrahydrothieno[2,3-c]pyridine-2-carboxamide);
33) [4,4′-biindoline]-1,1′-diylbis((6,7-dihydro-4H-512-thiazolo[5,4-c]pyridin-2-yl)methanone);
34) N-(2,2′-dimethyl-3′-(N-methyl-5,6,7,8-tetrahydroimidazo[1,2-a]pyrazine-2-carboxamido)-[1,1′-biphenyl]-3-yl)-N-methyl-4,5,6,7-tetrahydrothieno[3,2-c]pyridine-2-carboxamide;
35) N-(2,2′-dimethyl-3′-(5,6,7,8-tetrahydroimidazo[1,2-a]pyrazine-2-carboxamido)-[1,1′-biphenyl]-3-yl)-4,5,6,7-tetrahydrothieno[3,2-c]pyridine-2-carboxamide;
36) N,N′-(2,2′-dimethyl-[1,1′-biphenyl]-3,3′-diyl)bis(7-(1-hydroxypropan-2-yl)-5,6,7,8-tetrahydroimidazo[1,2-a]pyrazine-2-carboxamide);
37) N,N′-(2,2′-dimethyl-[1,1′-biphenyl]-3,3′-diyl)bis(5-(guanidinomethyl)-1,3,4-thiadiazole-2-carboxamide);
38) N,N′-(2,2′-dimethyl-[1,1′-biphenyl]-3,3′-diyl)bis(5-(1-guanidinocyclobutyl)-1,3,4-thiadiazole-2-carboxamide);
39) N,N′-(2,2′-dimethyl-[1,1′-biphenyl]-3,3′-diyl)bis(5,6,7,8-tetrahydro-[1,2,4]triazolo[4,3-a]pyrazine-3-carboxamide);
40) N,N′-(2,2′-dicyano-[1,1′-biphenyl]-3,3′-diyl)bis(5,6,7,8-tetrahydroimidazo[1,2-a]pyrazine-2-carboxamide);
41) N,N′-(2,2′-dimethyl-[1,1′-biphenyl]-3,3′-diyl)bis(5-(pyrrolidin-2-yl)-1,3,4-oxadiazole-2-carboxamide);
42) N-(2,2′-dimethyl-3′-(5,6,7,8-tetrahydroimidazo[1,2-a]pyrazine-2-carboxamido)-[1,1′-biphenyl]-3-yl)-4,5,6,7-tetrahydrothieno[2,3-c]pyridine-2-carboxamide;
43) N,N′-(2,2′-dichloro-[1,1′-biphenyl]-3,3′-diyl)bis(4,5,6,7-tetrahydrothiazolo[5,4-c]pyridine-2-carboxamide);
44) N,N′-(2,2′-dichloro-[1,1′-biphenyl]-3,3′-diyl)bis(5-methyl-4,5,6,7-tetrahydrothiazolo[5,4-c]pyridine-2-carboxamide);
45) N,N′-(2,2′-dimethyl-[1,1′-biphenyl]-3,3′-diyl)bis(5-(1-hydroxypropan-2-yl)-4,5,6,7-tetrahydrothiazolo[5,4-c]pyridine-2-carboxamide);
46) 1,1′-bis((6,7-dihydro-4H-512-thiazolo[5,4-c]pyridin-2-yl)methyl)-4,4′-biindoline;
47) 1,1′-bis(1-(5-methyl-4,5,6,7-tetrahydrothiazolo[5,4-c]pyridin-2-yl)ethyl)-4,4′-biindoline;
48) N,N′-(2-chloro-2′-methylbiphenyl-3,3′-diyl)bis(4,5,6,7-tetrahydrothiazolo[5,4-c]pyridine-2-carboxamide);
49) N,N′-(2-chloro-2′-methylbiphenyl-3,3′-diyl)bis(5-methyl-4,5,6,7-tetrahydrothiazolo[5,4-c]pyridine-2-carboxamide);
50) N-(2-chloro-2′-methyl-3′-(4,5,6,7-tetrahydrothiazolo[5,4-c]pyridine-2-carboxamido)biphenyl-3-yl)-5-methyl-4,5,6,7-tetrahydrothiazolo[5,4-c]pyridine-2-carboxamide;
51) N,N′-(2,2′-dimethylbiphenyl-3,3′-diyl)bis(5-((2-hydroxyethylamino)methyl)-4-methylthiazole-2-carboxamide);
52) (2S,2'S)-1,1′-(2,2′-(2,2′-dimethylbiphenyl-3,3′-diyl)bis(azanediyl)bis(oxomethylene)bis(4-methylthiazole-5,2-diyl))bis(methylene)dipiperidine-2-carboxylic acid;
53) (2S,2'S)-2,2′-(2,2′-(2,2′-dimethylbiphenyl-3,3′-diyl)bis(azanediyl)bis(oxomethylene)bis(4-methylthiazole-5,2-diyl))bis(methylene)bis(azanediyl)bis(2-(tetrahydro-2H-pyran-4-yl)acetic acid);
54) (2S,2'S)-1,1′-(2,2′-(2-chloro-2′-methylbiphenyl-3,3′-diyl)bis(azanediyl)bis(oxomethylene)bis(4-methylthiazole-5,2-diyl))bis(methylene)dipiperidine-2-carboxylic acid;
55) (2S,2'S)-2,2′-(2,2′-(2-chloro-2′-methylbiphenyl-3,3′-diyl)bis(azanediyl)bis(oxomethylene)bis(4-methylthiazole-5,2-diyl))bis(methylene)bis(azanediyl)bis(2-(tetrahydro-2H-pyran-4-yl)acetic acid);
56) (2S,2'S)-1,1′-(2,2′-(4,4′-biindoline-1,1′-diylbis(oxomethylene))bis(4-methylthiazole-5,2-diyl))bis(methylene)dipiperidine-2-carboxylic acid;
57) (2S,2'S)-2,2′-(2,2′-(4,4′-biindoline-1,1′-diylbis(oxomethylene))bis(4-methylthiazole-5,2-diyl))bis(methylene)bis(azanediyl)bis(2-(tetrahydro-2H-pyran-4-yl)acetic acid);
58) (S)-1-((2-(3-(1-(5-(((S)-2-carboxypiperidin-1-yl)methyl)-4-methylthiazole-2-carbonyl)indolin-4-yl)-2-methylphenylcarbamoyl)-4-methylthiazol-5-yl)methyl)piperidine-2-carboxylic acid;
59) (S)-2-((2-(3-(1-(5-(((S)-carboxy(tetrahydro-2H-pyran-4-yl)methylamino)methyl)-4-methylthiazole-2-carbonyl)indolin-4-yl)-2-methylphenylcarbamoyl)-4-methylthiazol-5-yl)methylamino)-2-(tetrahydro-2H-pyran-4-yl)acetic acid;
60) (S)-1-((2-(3-(1-(5-(((S)-2-carboxypiperidin-1-yl)methyl)-4-methylthiazole-2-carbonyl)indolin-4-yl)-2-chlorophenylcarbamoyl)-4-methylthiazol-5-yl)methyl)piperidine-2-carboxylic acid;
61) (S)-2-((2-(3-(1-(5-(((S)-carboxy(tetrahydro-2H-pyran-4-yl)methylamino)methyl)-4-methylthiazole-2-carbonyl)indolin-4-yl)-2-chlorophenylcarbamoyl)-4-methylthiazol-5-yl)methylamino)-2-(tetrahydro-2H-pyran-4-yl)acetic acid;
62) (S)-1-((2-(3-(1-(5-(((S)-2-carboxypiperidin-1-yl)methyl)-4-methylthiazole-2-carbonyl)indolin-4-yl)phenylcarbamoyl)-4-methylthiazol-5-yl)methyl)piperidine-2-carboxylic acid;
63) (S)-2-((2-(3-(1-(5-(((S)-carboxy(tetrahydro-2H-pyran-4-yl)methylamino)methyl)-4-methylthiazole-2-carbonyl)indolin-4-yl)phenylcarbamoyl)-4-methylthiazol-5-yl)methylamino)-2-(tetrahydro-2H-pyran-4-yl)acetic acid;
64) N,N′-(2,2′-dimethylbiphenyl-3,3′-diyl)bis(3-methyl-4,5,6,7-tetrahydro-3H-imidazo[4,5-c]pyridine-2-carboxamide);
65) 2,2′-((((2,2′-dichloro-[1,1′-biphenyl]-3,3′-diyl)bis(azanediyl))bis(carbonyl))bis(6,7-dihydrothiazolo[5,4-c]pyridine-2,5(4H)-diyl))diacetic acid;
66) N,N′-(2,2′-dichloro-[1,1′-biphenyl]-3,3′-diyl)bis(5-(((2-hydroxyethyl)amino)methyl)thiazole-2-carboxamide);
67) (2S,2'S,4R,4′R)-1,1′-(((((2,2′-dichloro-[1,1′-biphenyl]-3,3′-diyl)bis(azanediyl))bis(carbonyl))bis(thiazole-2,5-diyl))bis(methylene))bis(4-hydroxypyrrolidine-2-carboxylic acid);
68) N,N′-(2,2′-dichloro-[1,1′-biphenyl]-3,3′-diyl)bis(5-(((2,2,2-trifluoroethyl)amino)methyl)thiazole-2-carboxamide);
69) ((2-((3′-(5-((((S)-1-carboxy-2-methylpropyl)amino)methyl)thiazole-2-carboxamido)-2,2′-dichloro-[1,1′-biphenyl]-3-yl)carbamoyl)thiazol-5-yl)methyl)-L-valine;
70) [4,4′-biindoline]-1,1′-diylbis((5-(((2-hydroxyethyl)amino)methyl)thiazol-2-yl)methanone);
71) (2S,2'S)-2,2′-((((((2,2′-dichloro-[1,1′-biphenyl]-3,3′-diyl)bis(azanediyl))bis(carbonyl))bis(thiazole-2,5-diyl))bis(methylene))bis(azanediyl))bis(2-(tetrahydro-2H-pyran-4-yl)acetic acid);
72) (S)-1-((2-((3-(1-(5-(((S)-2-carboxypiperidin-1-yl)methyl)thiazole-2-carbonyl)indolin-4-yl)-2-methylphenyl)carbamoyl)thiazol-5-yl)methyl)piperidine-2-carboxylic acid;
73) N,N′-(2,2′-dimethyl-[1,1′-biphenyl]-3,3′-diyl)bis(5-(((2-hydroxyethyl)amino)methyl)-1,3,4-thiadiazole-2-carboxamide);
74) N,N′-(2,2′-dicyano-[1,1′-biphenyl]-3,3′-diyl)bis(4,5,6,7-tetrahydrothiazolo[5,4-c]pyridine-2-carboxamide);
75) N,N′-(2,2′-dimethyl-[1,1′-biphenyl]-3,3′-diyl)bis(5-(3,4-dichlorobenzyl)-4,5,6,7-tetrahydrothiazolo[5,4-c]pyridine-2-carboxamide);
76) N,N′-(2,2′-dimethyl-[1,1′-biphenyl]-3,3′-diyl)bis(5-(3-hydroxy-2,2-dimethylpropyl)-4,5,6,7-tetrahydrothiazolo[5,4-c]pyridine-2-carboxamide);
77) N,N′-(2,2′-dimethyl-[1,1′-biphenyl]-3,3′-diyl)bis(5-isopropyl-4,5,6,7-tetrahydrothiazolo[5,4-c]pyridine-2-carboxamide);
78) 4,4′-((((2,2′-dimethyl-[1,1′-biphenyl]-3,3′-diyl)bis(azanediyl))bis(carbonyl))bis(6,7-dihydrothiazolo[5,4-c]pyridine-2,5(4H)-diyl))dibutyric acid;
79) 2,2′-((((2,2′-dimethyl-[1,1′-biphenyl]-3,3′-diyl)bis(azanediyl))bis(carbonyl))bis(6,7-dihydrothiazolo[5,4-c]pyridine-2,5(4H)-diyl))dipropionic acid;
80) N,N′-(2,2′-dimethyl-[1,1′-biphenyl]-3,3′-diyl)bis(5-(cyanomethyl)-4,5,6,7-tetrahydrothiazolo[5,4-c]pyridine-2-carboxamide);
81) N,N′-(2,2′-dimethyl-[1,1′-biphenyl]-3,3′-diyl)bis(5-(3-morpholinopropyl)-4,5,6,7-tetrahydrothiazolo[5,4-c]pyridine-2-carboxamide);
82) N,N′-(2,2′-dimethyl-[1,1′-biphenyl]-3,3′-diyl)bis(5-(2-hydroxyethyl)-4,5,6,7-tetrahydrothiazolo[5,4-c]pyridine-2-carboxamide);
83) [4,4′-biindoline]-1,1′-diylbis((5-methyl-4,5,6,7-tetrahydrothiazolo[5,4-c]pyridin-2-yl)methanone);
84) [4,4′-biindoline]-1,1′-diylbis((5-(2,2,2-trifluoroethyl)-4,5,6,7-tetrahydrothiazolo[5,4-c]pyridin-2-yl)methanone);
85) [4,4′-biindoline]-1,1′-diylbis((5-(2,2-difluoroethyl)-4,5,6,7-tetrahydrothiazolo[5,4-c]pyridin-2-yl)methanone);
86) 2,2′-(([4,4′-biindoline]-1,1′-dicarbonyl)bis(6,7-dihydrothiazolo[5,4-c]pyridine-2,5(4H)-diyl))diacetamide;
87) [4,4′-biindoline]-1,1′-diylbis((5-(2-fluoroethyl)-4,5,6,7-tetrahydrothiazolo[5,4-c]pyridin-2-yl)methanone);
88) [4,4′-biindoline]-1,1′-diylbis((5-(oxetan-3-yl)-4,5,6,7-tetrahydrothiazolo[5,4-c]pyridin-2-yl)methanone);
89) N,N′-(2,2′-dichloro-[1,1′-biphenyl]-3,3′-diyl)bis(4,5,6,7-tetrahydrothiazolo[5,4-c]pyridine-2-carboxamide);
90) N,N′-(2,2′-dichloro-[1,1′-biphenyl]-3,3′-diyl)bis(5-(2-amino-2-oxoethyl)-4,5,6,7-tetrahydrothiazolo[5,4-c]pyridine-2-carboxamide);
91) N,N′-(2,2′-dichloro-[1,1′-biphenyl]-3,3′-diyl)bis(5-(2-hydroxyethyl)-4,5,6,7-tetrahydrothiazolo[5,4-c]pyridine-2-carboxamide);
92) N,N′-(2,2′-dichloro-[1,1′-biphenyl]-3,3′-diyl)bis(5-(oxetan-3-yl)-4,5,6,7-tetrahydrothiazolo[5,4-c]pyridine-2-carboxamide);
93) N,N′-(5,5′-dichloro-2,2′-dimethyl-[1,1′-biphenyl]-3,3′-diyl)bis(4,5,6,7-tetrahydrothiazolo[5,4-c]pyridine-2-carboxamide);
94) N,N′-(2,2′,4,4′-tetramethyl-[1,1′-biphenyl]-3,3′-diyl)bis(4,5,6,7-tetrahydrothiazolo[5,4-c]pyridine-2-carboxamide);
95) N-(5-fluoro-2-methyl-3-(1-(4,5,6,7-tetrahydrothiazolo[5,4-c]pyridine-2-carbonyl)indolin-4-yl)phenyl)-4,5,6,7-tetrahydrothiazolo[5,4-c]pyridine-2-carboxamide;
96) N,N′-(5,5′-difluoro-2,2′-dimethyl-[1,1′-biphenyl]-3,3′-diyl)bis(4,5,6,7-tetrahydrothiazolo[5,4-c]pyridine-2-carboxamide);
97) N-(2,2′-dichloro-3′-(5-(((2-hydroxyethyl)amino)methyl)thiazole-2-carboxamido)-[1,1′-biphenyl]-3-yl)-4,5,6,7-tetrahydrothiazolo[5,4-c]pyridine-2-carboxamide;
98) N-(2,2′-dichloro-3′-((3-(((2-hydroxyethyl)amino)methyl)-1,7-naphthyridin-8-yl)amino)-[1,1′-biphenyl]-3-yl)-5-(((2-hydroxyethyl)amino)methyl)thiazole-2-carboxamide;
99) (5-(((2-hydroxyethyl)amino)methyl)thiazol-2-yl)(1′-(4,5,6,7-tetrahydrothiazolo[5,4-c]pyridine-2-carbonyl)-[4,4′-biindolin]-1-yl)methanone;
100) (1′-(3-(((2-hydroxyethyl)amino)methyl)-1,7-naphthyridin-8-yl)-[4,4′-biindolin]-1-yl)(4,5,6,7-tetrahydrothiazolo[5,4-c]pyridin-2-yl)methanone;
101) N-(2-chloro-3-(1-(4,5,6,7-tetrahydrothiazolo[5,4-c]pyridine-2-carbonyl)indolin-4-yl)phenyl)-5-(((2-hydroxyethyl)amino)methyl)thiazole-2-carboxamide;
102) dimethyl 2,2′-(([4,4′-biindoline]-1,1′-dicarbonyl)bis(6,7-dihydrothiazolo[5,4-c]pyridine-2,5(4H)-diyl))diacetate;
103) 2,2′-(([4,4′-biindoline]-1,1′-dicarbonyl)bis(6,7-dihydrothiazolo[5,4-c]pyridine-2,5(4H)-diyl))diacetic acid;
104) 2,2′-(([4,4′-biindoline]-1,1′-dicarbonyl)bis(6,7-dihydrothiazolo[5,4-c]pyridine-2,5(4H)-diyl))diacetamide;
105) 2,2′-(([4,4′-biindoline]-1,1′-dicarbonyl)bis(6,7-dihydrothiazolo[5,4-c]pyridine-2,5(4H)-diyl))bis(N-methylacetamide);
106) 2,2′-(([4,4′-biindoline]-1,1′-dicarbonyl)bis(6,7-dihydrothiazolo[5,4-c]pyridine-2,5(4H)-diyl))bis(N,N-dimethylacetamide);
107) 2-(2-(1′-(5-(2-amino-2-oxoethyl)-4,5,6,7-tetrahydrothiazolo[5,4-c]pyridine-2-carbonyl)-[4,4′-biindoline]-1-carbonyl)-6,7-dihydrothiazolo[5,4-c]pyridin-5(4H)-yl)acetic acid;
108) 2-(2-(1′-(5-(2-(methylamino)-2-oxoethyl)-4,5,6,7-tetrahydrothiazolo[5,4-c]pyridine-2-carbonyl)-[4,4′-biindoline]-1-carbonyl)-6,7-dihydrothiazolo[5,4-c]pyridin-5(4H)-yl)acetic acid;
109) 2-(2-(1′-(5-(2-(dimethylamino)-2-oxoethyl)-4,5,6,7-tetrahydrothiazolo[5,4-c]pyridine-2-carbonyl)-[4,4′-biindoline]-1-carbonyl)-6,7-dihydrothiazolo[5,4-c]pyridin-5(4H)-yl)acetic acid;
110) 2,2′-(([4,4′-biindoline]-1,1′-dicarbonyl)bis(5,6,7,8-tetrahydro-4H-5,8-epiminocyclohepta[d]thiazole-2,9-diyl))diacetic acid;
111) 2-(2-(1′-(9-(2-amino-2-oxoethyl)-5,6,7,8-tetrahydro-4H-5,8-epiminocyclohepta[d]thiazole-2-carbonyl)-[4,4′-biindoline]-1-carbonyl)-5,6,7,8-tetrahydro-4H-5,8-epiminocyclohepta[d]thiazol-9-yl)acetic acid;
112) [4,4′-biindoline]-1,1′-diylbis((5-((dimethylamino)methyl)thiazol-2-yl)methanone);
113) [4,4′-biindoline]-1,1′-diylbis((5-((methylamino)methyl)thiazol-2-yl)methanone);
114) N-(3-(1-(5-((dimethylamino)methyl)-4-methylthiazole-2-carbonyl)indolin-4-yl)phenyl)-5-(((2-hydroxyethyl)(methyl)amino)methyl)thiazole-2-carboxamide;
115) (5-((dimethylamino)methyl)-4-methylthiazol-2-yl)(1′-(5-(((2-hydroxyethyl)(methyl)amino)methyl)thiazole-2-carbonyl)-[4,4′-biindolin]-1-yl)methanone;
116) [4,4′-biindoline]-1,1′-diylbis((5-(((2-hydroxyethyl)(methyl)amino)methyl)thiazol-2-yl)methanone);
117) N,N′-((([4,4′-biindoline]-1,1′-dicarbonyl)bis(6,7-dihydrothiazolo[5,4-c]pyridine-2,5(4H)-diyl))bis(ethane-2,1-diyl))dimethanesulfonamide;
118) N,N′-((([4,4′-biindoline]-1,1′-dicarbonyl)bis(6,7-dihydrothiazolo[5,4-c]pyridine-2,5(4H)-diyl))bis(ethane-2,1-diyl))bis(N-methylmethanesulfonamide);
119) 2,2′-(([4,4′-biindoline]-1,1′-dicarbonyl)bis(6,7-dihydrothiazolo[4,5-c]pyridine-2,5(4H)-diyl))diacetic acid;
120) [4,4′-biindoline]-1,1′-diylbis((5-(2-(2-hydroxyethoxy)ethyl)-4,5,6,7-tetrahydrothiazolo[5,4-c]pyridin-2-yl)methanone);
121) ((([4,4′-biindoline]-1,1′-dicarbonyl)bis(6,7-dihydrothiazolo[5,4-c]pyridine-2,5(4H)-diyl))bis(propane-3,1-diyl))diboronic acid;
122) tetramethyl((([4,4′-biindoline]-1,1′-dicarbonyl)bis(6,7-dihydrothiazolo[5,4-c]pyridine-2,5(4H)-diyl))bis(propane-3,1-diyl))diboronate;
123) dimethyl ((2-(1′-(5-((dimethoxyphosphoryl)methyl)-4,5,6,7-tetrahydrothiazolo[5,4-c]pyridine-2-carbonyl)-[4,4′-biindoline]-1-carbonyl)-6,7-dihydrothiazolo[5,4-c]pyridin-5(4H)-yl)methyl)phosphonate;
124) dimethyl (2-(2-(1′-(5-(2-(dimethoxyphosphoryl)ethyl)-4,5,6,7-tetrahydrothiazolo[5,4-c]pyridine-2-carbonyl)-[4,4′-biindoline]-1-carbonyl)-6,7-dihydrothiazolo[5,4-c]pyridin-5(4H)-yl)ethyl)phosphonate;
125) [4,4′-biindoline]-1,1′-diylbis((5-((2H-tetrazol-5-yl)methyl)-4,5,6,7-tetrahydrothiazolo[5,4-c]pyridin-2-yl)methanone);
126) N,N′-((([4,4′-biindoline]-1,1′-dicarbonyl)bis(5,6,7,8-tetrahydro-4H-5,8-epiminocyclohepta[d]thiazole-2,9-diyl))bis(ethane-2,1-diyl))dimethanesulfonamide;
127) 2,2′-(([4,4′-biindoline]-1,1′-dicarbonyl)bis(6,7-dihydrothiazolo[5,4-c]pyridine-2,5(4H)-diyl))bis(N-(methylsulfonyl)propanamide);
128) 2,2′-(([4,4′-biindoline]-1,1′-dicarbonyl)bis(6,7-dihydrothiazolo[5,4-c]pyridine-2,5(4H)-diyl))bis(2-methyl-N-(methylsulfonyl)propanamide);
129) 1,1′-(([4,4′-biindoline]-1,1′-dicarbonyl)bis(6,7-dihydrothiazolo[5,4-c]pyridine-2,5(4H)-diyl))bis(N-(methylsulfonyl)cyclopropane-1-carboxamide);
130) dimethyl ((([4,4′-biindoline]-1,1′-dicarbonyl)bis(6,7-dihydrothiazolo[5,4-c]pyridine-2,5(4H)-diyl))bis(ethane-2,1-diyl))dicarbamate;
131) dimethyl (((([4,4′-biindoline]-1,1′-dicarbonyl)bis(6,7-dihydrothiazolo[5,4-c]pyridine-2,5(4H)-diyl))bis(cyclopropane-1,1-diyl))bis(methylene))dicarbamate;
132) dimethyl (((([4,4′-biindoline]-1,1′-dicarbonyl)bis(6,7-dihydrothiazolo[5,4-c]pyridine-2,5(4H)-diyl))bis(methylene))bis(cyclopropane-1,1-diyl))dicarbamate;
133) 1,1′-((([4,4′-biindoline]-1,1′-dicarbonyl)bis(6,7-dihydrothiazolo[5,4-c]pyridine2,5(4H)-diyl))bis(ethane-2,1-diyl))bis(thiourea);
134) 1,1′-((([4,4′-biindoline]-1,1′-dicarbonyl)bis(6,7-dihydrothiazolo[5,4-c]pyridine-2,5(4H)-diyl))bis(ethane-2,1-diyl))diurea;
135) dimethyl 2,2′-(((([4,4′-biindoline]-1,1′-dicarbonyl)bis(6,7-dihydrothiazolo[5,4-c]pyridine-2,5(4H)-diyl))bis(methylene))bis(1H-1,2,3-triazole-4,1-diyl))diacetate;
136) [4,4′-biindoline]-1,1′-diylbis((5,6,7,8-tetrahydro-[1,2,4]triazolo[1,5-a]pyrazin-2-yl)methanone);
137) (5-((12-azanyl)methyl)-1H-1,2,4-triazol-3-yl)(1′-(5-(aminomethyl)-1H-1,2,4-triazole-3-carbonyl)-[4,4′-biindolin]-1-yl)methanone;
138) 2,2′-(((((2,2′-dimethyl-[1,1′-biphenyl]-3,3′-diyl)bis(azanediyl))bis(1,7-naphthyridine-8,3-diyl))bis(methylene))bis(azanediyl))diacetic acid;
139) ((8-((3′-((3-(((2-hydroxyethyl)amino)methyl)-1,7-naphthyridin-8-yl)amino)-2,2′-dimethyl-[1,1′-biphenyl]-3-yl)amino)-1,7-naphthyridin-3-yl)methyl)glycine;
140) 2,2′-(((((2,2′-dimethyl-[1,1′-biphenyl]-3,3′-diyl)bis(azanediyl))bis(1,7-naphthyridine-8,3-diyl))bis(methylene))bis(azanediyl))dipropionic acid;
141) (S)-4-(((8-((3′-((3-(((carboxymethyl)amino)methyl)-1,7-naphthyridin-8-yl)amino)-2,2′-dimethyl-[1,1′-biphenyl]-3-yl)amino)-1,7-naphthyridin-3-yl)methyl)amino)-3-hydroxybutanoic acid;
142) 2,2′-(((((2,2′-dimethyl-[1,1′-biphenyl]-3,3′-diyl)bis(azanediyl))bis(1,7-naphthyridine-8,3-diyl))bis(methylene))bis(azanediyl))bis(3-methylbutanoic acid);
143) (3S,3'S)-4,4′-(((((2,2′-dimethyl-[1,1′-biphenyl]-3,3′-diyl)bis(azanediyl))bis(1,7-naphthyridine-8,3-diyl))bis(methylene))bis(azanediyl))bis(3-hydroxybutanoic acid);
144) 2,2′-(((((2,2′-dimethyl-[1,1′-biphenyl]-3,3′-diyl)bis(azanediyl))bis(1,7-naphthyridine-8,3-diyl))bis(methylene))bis(azanediyl))diacetamide;
145) 2,2′-(((((2,2′-dimethyl-[1,1′-biphenyl]-3,3′-diyl)bis(azanediyl))bis(1,7-naphthyridine-8,3-diyl))bis(methylene))bis(azanediyl))bis(N,N-dimethylacetamide);
146) 2-(((8-((2,2′-dimethyl-3′-((3-(((2,2,2-trifluoroethyl)amino)methyl)-1,7-naphthyridin-8-yl)amino)-[1,1′-biphenyl]-3-yl)amino)-1,7-naphthyridin-3-yl)methyl)amino)-N,N-dimethylacetamide;
147) 2-(((8-((2,2′-dimethyl-3′-((3-(((2-(methylamino)-2-oxoethyl)amino)methyl)-1,7-naphthyridin-8-yl)amino)-[1,1′-biphenyl]-3-yl)amino)-1,7-naphthyridin-3-yl)methyl)amino)-N-(2-hydroxyethyl)-N-methylacetamide;
148) ((8-((3′-((3-(((2-(dimethylamino)-2-oxoethyl)amino)methyl)-1,7-naphthyridin-8-yl)amino)-2,2′-dimethyl-[1,1′-biphenyl]-3-yl)amino)-1,7-naphthyridin-3-yl)methyl)glycine;
149) ((8-((3′-((3-(azetidin-1-ylmethyl)-1,7-naphthyridin-8-yl)amino)-2,2′-dimethyl-[1,1′-biphenyl]-3-yl)amino)-1,7-naphthyridin-3-yl)methyl)glycine;
150) 1,1′-((((2,2′-dimethyl-[1,1′-biphenyl]-3,3′-diyl)bis(azanediyl))bis(1,7-naphthyridine-8,3-diyl))bis(methylene))bis(azetidin-3-ol);
151) 2,2′-dimethyl-N3,N3′-bis(3-(morpholinomethyl)-1,7-naphthyridin-8-yl)-[1,1′-biphenyl]-3,3′-diamine;
152) N,N′-((((((2,2′-dimethyl-[1,1′-biphenyl]-3,3′-diyl)bis(azanediyl))bis(1,7-naphthyridine-8,3-diyl))bis(methylene))bis(azanediyl))bis(ethane-2,1-diyl))diacetamide;
153) N3,N3′-bis(3-(((S)-3-(dimethylamino)pyrrolidin-1-yl)methyl)-1,7-naphthyridin-8-yl)-2,2′-dimethyl-[1,1′-biphenyl]-3,3′-diamine;
154) (S)-((8-((3′-((3-((3-(dimethylamino)pyrrolidin-1-yl)methyl)-1,7-naphthyridin-8-yl)amino)-2,2′-dimethyl-[1,1′-biphenyl]-3-yl)amino)-1,7-naphthyridin-3-yl)methyl)glycine;
155) 1,1′-((((2,2′-dimethyl-[1,1′-biphenyl]-3,3′-diyl)bis(azanediyl))bis(1,7-naphthyridine-8,3-diyl))bis(methylene))bis(piperidine-2-carboxylic acid);
156) 2-(((8-((2,2′-dimethyl-3′-((3-(((2,2,2-trifluoroethyl)amino)methyl)-1,7-naphthyridin-8-yl)amino)-[1,1′-biphenyl]-3-yl)amino)-1,7-naphthyridin-3-yl)methyl)amino)ethan-1-ol;
157) (1S,1'S)-1,1′-((((((2,2′-dimethyl-[1,1′-biphenyl]-3,3′-diyl)bis(azanediyl))bis(1,7-naphthyridine-8,3-diyl))bis(methylene))bis(azanediyl))bis(cyclopropane-1,1-diyl))bis(ethan-1-ol);
158) dimethyl ((((((2,2′-dimethyl-[1,1′-biphenyl]-3,3′-diyl)bis(azanediyl))bis(1,7-naphthyridine-8,3-diyl))bis(methylene))bis(azanediyl))bis(ethane-2,1-diyl))dicarbamate;
159) 2-(2-(4-(3-((3-(((2-((methoxycarbonyl)amino)ethyl)amino)methyl)-1,7-naphthyridin-8-yl)amino)-2-methylphenyl)indoline-1-carbonyl)-3a,6,7,7a-tetrahydrothiazolo[5,4-c]pyridin-5(4H)-yl)acetic acid;
160) ((8-((3-(1-(5-(carboxymethyl)-3a,4,5,6,7,7a-hexahydrothiazolo[5,4-c]pyridine-2-carbonyl)indolin-4-yl)-2-methylphenyl)amino)-1,7-naphthyridin-3-yl)methyl)glycine;
161) 3-(2-(4-(3-((3-(((carboxymethyl)amino)methyl)-1,7-naphthyridin-8-yl)amino)-2-methylphenyl)indoline-1-carbonyl)-3a,6,7,7a-tetrahydrothiazolo[5,4-c]pyridin-5(4H)-yl)propanoic acid;
162) 3-(2-(4-(3-((3-(((2-hydroxyethyl)amino)methyl)-1,7-naphthyridin-8-yl)amino)-2-methylphenyl)indoline-1-carbonyl)-3a,6,7,7a-tetrahydrothiazolo[5,4-c]pyridin-5(4H)-yl)propanoic acid;
163) ((8-((3-(1-(5-(carboxymethyl)-3a,4,5,6,7,7a-hexahydrothiazolo[5,4-c]pyridine-2-carbonyl)indolin-4-yl)-2-methylphenyl)amino)-1,7-naphthyridin-3-yl)methyl)alanine;
164) 2-(2-(4-(3-((3-(((2-amino-2-oxoethyl)amino)methyl)-1,7-naphthyridin-8-yl)amino)-2-methylphenyl)indoline-1-carbonyl)-3a,6,7,7a-tetrahydrothiazolo[5,4-c]pyridin-5(4H)-yl)acetic acid;
165) 2-(2-(4-(3-((3-(((2-(dimethylamino)-2-oxoethyl)amino)methyl)-1,7-naphthyridin-8-yl)amino)-2-methylphenyl)indoline-1-carbonyl)-3a,6,7,7a-tetrahydrothiazolo[5,4-c]pyridin-5(4H)-yl)acetic acid;
166) 2-(2-(4-(3-((3-(((2-((2-hydroxyethyl)(methyl)amino)-2-oxoethyl)amino)methyl)-1,7-naphthyridin-8-yl)amino)-2-methylphenyl)indoline-1-carbonyl)-3a,6,7,7a-tetrahydrothiazolo[5,4-c]pyridin-5(4H)-yl)acetic acid;
167) N,N′-(2,2′-dimethyl-[1,1′-biphenyl]-3,3′-diyl)bis(5,6,7,8-tetrahydro-1,6-naphthyridine-2-carboxamide);
168) dimethyl ((2S,2'S)-(([4,4′-biindoline]-1,1′-dicarbonyl)bis(7,8-dihydro-1,6-naphthyridine-2,6(5H)-diyl))bis(3-methyl-1-oxobutane-1,2-diyl))dicarbamate;
169) dimethyl ((2S,2'S)-((((2,2′-dimethyl-[1,1′-biphenyl]-3,3′-diyl)bis(azanediyl))bis(carbonyl))bis(7,8-dihydro-1,6-naphthyridine-2,6(5H)-diyl))bis(3-methyl-1-oxobutane-1,2-diyl))dicarbamate;
170) ethyl 2-(2-((2,2′-dimethyl-3′-(5,6,7,8-tetrahydro-1,6-naphthyridine-2-carboxamido)-[1,1′-biphenyl]-3-yl)carbamoyl)-7,8-dihydro-1,6-naphthyridin-6(5H)-yl)acetate;
171) 2-((3′-(6,6-dimethyl-5,6,7,8-tetrahydro-614-1,6-naphthyridin-7-ylium-2-carboxamido)-2,2′-dimethyl-[1,1′-biphenyl]-3-yl)carbamoyl)-6,6-dimethyl-5,6,7,8-tetrahydro-1,6-naphthyridin-6-ium chloride;
172) di-tert-butyl 2,2′-((((2,2′-dimethyl-[1,1′-biphenyl]-3,3′-diyl)bis(azanediyl))bis(carbonyl))bis(7,8-dihydro-1,6-naphthyridine-2,6(5H)-diyl))diacetate;
173) 2,2′-((((2,2′-dimethyl-[1,1′-biphenyl]-3,3′-diyl)bis(azanediyl))bis(carbonyl))bis(7,8-dihydro-1,6-naphthyridine-2,6(5H)-diyl))diacetic acid;
174) N,N′-(2,2′-dimethyl-[1,1′-biphenyl]-3,3′-diyl)bis(6-(2-hydroxyethyl)-5,6,7,8-tetrahydro-1,6-naphthyridine-2-carboxamide);
175) N,N′-(2,2′-dimethyl-[1,1′-biphenyl]-3,3′-diyl)bis(6-methyl-5,6,7,8-tetrahydro-1,6-naphthyridine-2-carboxamide);
176) N,N′-(2,2′-dimethyl-[1,1′-biphenyl]-3,3′-diyl)bis(6-(2-morpholinoethyl)-5,6,7,8-tetrahydro-1,6-naphthyridine-2-carboxamide);
177) diethyl (2-(2-((3′-(((6-(2-(diethoxyphosphoryl)ethyl)-5,6,7,8-tetrahydro-1,6-naphthyridin-2-yl)methyl)amino)-2,2′-dimethyl-[1,1′-biphenyl]-3-yl)carbamoyl)-7,8-dihydro-1,6-naphthyridin-6(5H)-yl)ethyl)phosphonate;
178) 2-(2-((2,2′-dimethyl-3′-(5,6,7,8-tetrahydro-1,6-naphthyridine-2-carboxamido)-[1,1′-biphenyl]-3-yl)carbamoyl)-7,8-dihydro-1,6-naphthyridin-6(5H)-yl)acetic acid;
179) diethyl 2,2′-((((((2,2′-dimethyl-[1,1′-biphenyl]-3,3′-diyl)bis(azanediyl))bis(carbonyl))bis(pyrazine-5,2-diyl))bis(ethane-1,1-diyl))bis(azanediyl))diacetate;
180) N,N′-(2,2′-dimethyl-[1,1′-biphenyl]-3,3′-diyl)bis(5-(1-(methylamino)ethyl)pyrazine-2-carboxamide);
181) N,N′-(2,2′-dimethyl-[1,1′-biphenyl]-3,3′-diyl)bis(5-(1-((2-hydroxyethyl)amino)ethyl)pyrazine-2-carboxamide);
182) 6-acetyl-N-(2,2′-dimethyl-3′-(5,6,7,8-tetrahydro-1,6-naphthyridine-2-carboxamido)-[1,1′-biphenyl]-3-yl)-5,6,7,8-tetrahydro-1,6-naphthyridine-2-carboxamide;
183) N-(3′-(5-acetyl-1-methyl-4,5,6,7-tetrahydro-1H-imidazo[4,5-c]pyridine-2-carboxamido)-2,2′-dimethyl-[1,1′-biphenyl]-3-yl)-5,6,7,8-tetrahydro-1,6-naphthyridine-2-carboxamide;
184) bis((5-methyl-2-oxo-1,3-dioxol-4-yl)methyl) 2,2′-(((2,2′-dimethyl-[1,1′-biphenyl]-3,3′-diyl)bis(azanediyl))bis(carbonyl))bis(7,8-dihydro-1,6-naphthyridine-6(5H)-carboxylate);
185) N-(2,2′-dimethyl-3′-(5,6,7,8-tetrahydro-1,6-naphthyridine-2-carboxamido)-[1,1′-biphenyl]-3-yl)-6-methyl-5,6,7,8-tetrahydro-1,6-naphthyridine-2-carboxamide;
186) N-(2,2′-dimethyl-3′-(5,6,7,8-tetrahydro-1,6-naphthyridine-2-carboxamido)-[1,1′-biphenyl]-3-yl)-6-(2-hydroxyethyl)-5,6,7,8-tetrahydro-1,6-naphthyridine-2-carboxamide;
187) N,N′-(2,2′-dimethyl-[1,1′-biphenyl]-3,3′-diyl)bis(6-(2,2-difluoroethyl)-5,6,7,8-tetrahydro-1,6-naphthyridine-2-carboxamide);
188) N,N′-(2,2′-dimethyl-[1,1′-biphenyl]-3,3′-diyl)bis(5,6,7,8-tetrahydropyrido[4,3-d]pyrimidine-2-carboxamide);
189) [4,4′-biindoline]-1,1′-diylbis((5,6,7,8-tetrahydropyrido[4,3-d]pyrimidin-2-yl)methanone); or
190) 2,2′-((((2,2′-dimethyl-[1,1′-biphenyl]-3,3′-diyl)bis(azanediyl))bis(carbonyl))bis(7,8-dihydropyrido[4,3-d]pyrimidine-2,6(5H)-diyl))diacetic acid.

The present invention also provides a pharmaceutical composition comprising a compound of any of the present invention and a pharmaceutically acceptable excipient. Such as hydroxypropyl methyl cellulose. In the composition, the said compound in a weight ratio to the said excipient within the range from about 0.0001 to about 10.

The present invention additionally provided a use of a pharmaceutical composition of Formula (I) or Formula (II) or Formula (III) for the preparation of a medicament for treating a disease in a subject.

The present invention further provides some preferred technical solutions with regard to above-mentioned uses.

In some embodiments, a medicament thus prepared can be used for the treatment or prevention of, or for delaying or preventing onset or progression in, cancer, cancer metastasis, an immunological disorder. The cancer is colon cancer, gastric cancer, thyroid cancer, lung cancer, leukemia, pancreatic cancer, melanoma, multiple melanoma, brain cancer, renal cancer, prostate cancer, ovarian cancer or breast cancer.

The present invention provided a method of inhibiting PD-1/PD-L1 interaction, said method comprising administering to a patient a compound of any one of claims 1-12, or a pharmaceutically acceptable salt or a stereoisomer thereof.

The present invention provided a method of treating a disease associated with inhibition of PD-1/PD-L1 interaction, said method comprising administering to a patient in need thereof a therapeutically effective amount of a compound of the present invention, or a pharmaceutically acceptable salt or a stereoisomer thereof. Wherein the disease is colon cancer, gastric cancer, thyroid cancer, lung cancer, leukemia, pancreatic cancer, melanoma, multiple melanoma, brain cancer, renal cancer, prostate cancer, ovarian cancer or breast cancer.

The present invention provided a method of enhancing, stimulating and/or increasing the immune response in a patient, said method comprising administering to the patient in need thereof a therapeutically effective amount of a compound of the present invention, or a pharmaceutically acceptable salt or a stereoisomer thereof.

The present invention also provides a use of the present compound or its pharmaceutical composition for the preparation of a medicament.

In some embodiments, the medicament is used for the treatment or prevention of cancer.

In some embodiments, the cancer is colon cancer, gastric cancer, thyroid cancer, lung cancer, leukemia, pancreatic cancer, melanoma, multiple melanoma, brain cancer, renal cancer, prostate cancer, ovarian cancer or breast cancer.

In some embodiments, the medicament is used as an inhibitor of PD-1/PD-L1 interaction.

The general chemical terms used in the formula above have their usual meanings. For example, the term “halogen”, as used herein, unless otherwise indicated, means fluoro, chloro, bromo or iodo. The preferred halogen groups include F, Cl and Br.

As used herein, unless otherwise indicated, alkyl includes saturated monovalent hydrocarbon radicals having straight, branched or cyclic moieties. For example, alkyl radicals include methyl, ethyl, propyl, isopropyl, cyclcopropyl, n-butyl, isobutyl, sec-butyl, t-butyl, cyclcobutyl, n-pentyl, 3-(2-methyl) butyl, 2-pentyl, 2-methylbutyl, neopentyl, cyclcopentyl, n-hexyl, 2-hexyl, 2-methylpentyl and cyclohexyl. Similarly, C₁₋₈, as in C₁₋₈alkyl is defined to identify the group as having 1, 2, 3, 4, 5, 6, 7 or 8 carbon atoms in a linear or branched arrangement.

Alkenyl and alkynyl groups include straight, branched chain or cyclic alkenes and alkynes. Likewise, “C₂₋₈ alkenyl” and “C₂₋₈ alkynyl” means an alkenyl or alkynyl radicals having 2, 3, 4, 5, 6, 7 or 8 carbon atoms in a linear or branched arrangement.

Alkoxy radicals are oxygen ethers formed from the previously described straight, branched chain or cyclic alkyl groups.

The term “aryl”, as used herein, unless otherwise indicated, refers to an unsubstituted or substituted mono- or polycyclic ring system containing carbon ring atoms. The preferred aryls are mono cyclic or bicyclic 6-10 membered aromatic ring systems. Phenyl and naphthyl are preferred aryls. The most preferred aryl is phenyl.

The term “heterocyclyl”, as used herein, unless otherwise indicated, represents an unsubstituted or substituted stable three to eight membered monocyclic saturated ring system which consists of carbon atoms and from one to three heteroatoms selected from N, O or S, and wherein the nitrogen or sulfur heteroatoms may optionally be oxidized, and the nitrogen heteroatom may optionally be quaternized. The heterocyclyl group may be attached at any heteroatom or carbon atom which results in the creation of a stable structure. Examples of such heterocyclyl groups include, but are not limited to azetidinyl, pyrrolidinyl, piperidinyl, piperazinyl, oxopiperazinyl, oxopiperidinyl, oxoazepinyl, azepinyl, tetrahydrofuranyl, dioxolanyl, tetrahydroimidazolyl, tetrahydrothiazolyl, tetrahydrooxazolyl, tetrahydropyranyl, morpholinyl, thiomorpholinyl, thiamorpholinyl sulfoxide, thiamorpholinyl sulfone and oxadiazolyl.

The term “heteroaryl”, as used herein, unless otherwise indicated, represents an unsubstituted or substituted stable five or six membered monocyclic aromatic ring system or an unsubstituted or substituted nine or ten membered benzo-fused heteroaromatic ring system or bicyclic heteroaromatic ring system which consists of carbon atoms and from one to four heteroatoms selected from N, O or S, and wherein the nitrogen or sulfur heteroatoms may optionally be oxidized, and the nitrogen heteroatom may optionally be quaternized. The heteroaryl group may be attached at any heteroatom or carbon atom which results in the creation of a stable structure. Examples of heteroaryl groups include, but are not limited to thienyl, furanyl, imidazolyl, isoxazolyl, oxazolyl, pyrazolyl, pyrrolyl, thiazolyl, thiadiazolyl, triazolyl, pyridyl, pyridazinyl, indolyl, azaindolyl, indazolyl, benzimidazolyl, benzofuranyl, benzothienyl, benzisoxazolyl, benzoxazolyl, benzopyrazolyl, benzothiazolyl, benzothiadiazolyl, benzotriazolyl adeninyl, quinolinyl or isoquinolinyl.

The term “alkenyloxy” refers to the group —O-alkenyl, where alkenyl is defined as above.

The term “alknyloxy” refers to the group —O-alknyl, where alknyl is defined as above.

The term “cycloalkyl” to a cyclic saturated alkyl chain having from 3 to 12 carbon atoms, for example, cyclopropyl, cyclobutyl, cyclobutyl, cyclobutyl.

The term “substituted” refers to a group in which one or more hydrogen atoms are each independently replaced with the same or different substituent(s). Typical substituents include, but are not limited to, halogen (F, Cl, Br or I), C₁₋₈ alkyl, C₃₋₁₂ cycloalkyl, —OR¹, SR¹, ═O, ═S, —C(O)R¹, —C(S)R¹, ═NR¹, —C(O)OR¹, —C(S)OR¹, —NR¹R², —C(O)NR¹R², cyano, nitro, —S(O)₂R¹, —OS(O₂)OR¹, —OS(O)₂R¹, —OP(O)(OR)(OR²); wherein R¹ and R² is independently selected from —H, lower alkyl, lower haloalkyl. In some embodiments, the substituent(s) is independently selected from the group consisting of —F, —Cl, —Br, —I, —OH, trifluromethoxy, ethoxy, propyloxy, iso-propyloxy, n-butyloxy, isobutyloxy, t-butyloxy, —SCH₃, —SC₂H₅, formaldehyde group, —C(OCH₃), cyano, nitro, CF₃,—OCF₃, amino, dimethylamino, methyl thio, sulfonyl and acetyl.

The term “composition”, as used herein, is intended to encompass a product comprising the specified ingredients in the specified amounts, as well as any product which results, directly or indirectly, from combinations of the specified ingredients in the specified amounts. Accordingly, pharmaceutical compositions containing the compounds of the present invention as the active ingredient as well as methods of preparing the instant compounds are also part of the present invention. Furthermore, some of the crystalline forms for the compounds may exist as polymorphs and as such are intended to be included in the present invention. In addition, some of the compounds may form solvates with water (i.e., hydrates) or common organic solvents and such solvates are also intended to be encompassed within the scope of this invention.

Examples of substituted alkyl group include, but not limited to, 2-aminoethyl, 2-hydroxyethyl, pentachloroethyl, trifluoromethyl, methoxymethyl, pentafluoroethyl and piperazinylmethyl.

Examples of substituted alkoxy groups include, but not limited to, aminomethoxy, thrifluoromethoxy, 2-diethylaminoethoxy, 2-ethoxycarbonylethoxy, 3-hydroxypropoxy.

The compounds of the present invention may also be present in the form of pharmaceutically acceptable salts. For use in medicine, the salts of the compounds of this invention refer to non-toxic “pharmaceutically acceptable salts”. The pharmaceutically acceptable salt forms include pharmaceutically acceptable acidic/anionic or basic/cationic salts. The pharmaceutically acceptable acidic/anionic salt generally takes a form in which the basic nitrogen is protonated with an inorganic or organic acid. Representative organic or inorganic acids include hydrochloric, hydrobromic, hydriodic, perchloric, sulfuric, nitric, phosphoric, acetic, propionic, glycolic, lactic, succinic, maleic, fumaric, malic, tartaric, citric, benzoic, mandelic, methanesulfonic, hydroxyethanesulfonic, benzenesulfonic, oxalic, pamoic, 2-naphthalenesulfonic, p-toluenesulfonic, cyclohexanesulfamic, salicylic, saccharinic or trifluoroacetic. Pharmaceutically acceptable basic/cationic salts include, and are not limited to aluminum, calcium, chloroprocaine, choline, diethanolamine, ethylenediamine, lithium, magnesium, potassium, sodium and zinc.

The present invention includes within its scope the prodrugs of the compounds of this invention. In general, such prodrugs will be functional derivatives of the compounds that are readily converted in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the subject. Conventional procedures for the selection and preparation of suitable prodrug derivatives are described, for example, in “Design of Prodrugs”, ed. H. Bundgaard, Elsevier, 1985.

It is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques know in the art as well as those methods set forth herein.

The present invention includes compounds described herein can contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof.

The above Formula I and II are shown without a definitive stereochemistry at certain positions. The present invention includes all stereoisomers of Formula I and II and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

When a tautomer of the compound of Formula I and II exists, the present invention includes any possible tautomers and pharmaceutically acceptable salts thereof, and mixtures thereof, except where specifically stated otherwise.

When the compound of Formula I and II and pharmaceutically acceptable salts thereof exist in the form of solvates or polymorphic forms, the present invention includes any possible solvates and polymorphic forms. A type of a solvent that forms the solvate is not particularly limited so long as the solvent is pharmacologically acceptable. For example, water, ethanol, propanol, acetone or the like can be used.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N′,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, formic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, formic, hydrochloric, maleic, phosphoric, sulfuric and tartaric acids, particularly preferred are formic and hydrochloric acid. Since the compounds of Formula I are intended for pharmaceutical use they are preferably provided in substantially pure form, for example at least 60% pure, more suitably at least 75% pure, especially at least 98% pure (% are on a weight for weight basis).

The pharmaceutical compositions of the present invention comprise a compound represented by Formula I (or a pharmaceutically acceptable salt thereof) as an active ingredient, a pharmaceutically acceptable carrier and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds represented by Formula I, or a prodrug, or a metabolite, or pharmaceutically acceptable salts thereof, of this invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the compound represented by Formula I, or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound, or a pharmaceutically acceptable salt, of Formula I. The compounds of Formula I, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include such as lactose, terra alba, sucrose, talc, gelatin, agar, pectin, acacia, magnesium stearate, and stearic acid. Examples of liquid carriers include such as sugar syrup, peanut oil, olive oil, and water. Examples of gaseous carriers include such as carbon dioxide and nitrogen. In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient. For example, a formulation intended for the oral administration to humans may contain from about 0.5 mg to about 5 g of active agent, compounded with an appropriate and convenient amount of carrier material which may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, utilizing a compound represented by Formula I of this invention, or a pharmaceutically acceptable salt thereof, via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including antioxidants) and the like. Furthermore, other adjuvants can be included to render the formulation isotonic with the blood of the intended recipient. Compositions containing a compound described by Formula I, or pharmaceutically acceptable salts thereof, may also be prepared in powder or liquid concentrate form.

Generally, dosage levels on the order of from about 0.01 mg/kg to about 150 mg/kg of body weight per day are useful in the treatment of the above-indicated conditions, or alternatively about 0.5 mg to about 7 g per patient per day. For example, colon cancer, rectal cancer, mantle cell lymphoma, multiple myeloma, breast cancer, prostate cancer, glioblastoma, squamous cell esophageal cancer, liposarcoma, T-cell lymphoma melanoma, pancreatic cancer, glioblastoma or lung cancer, may be effectively treated by the administration of from about 0.01 to 50 mg of the compound per kilogram of body weight per day, or alternatively about 0.5 mg to about 3.5 g per patient per day.

It is understood, however, that lower or higher doses than those recited above may be required. Specific dose level and treatment regimens for any particular subject will depend upon a variety of factors, including the activity of the specific compound employed, the age, body weight, general health, sex, diet, time of administration, route of administration, rate of excretion, drug combination, the severity and course of the particular disease undergoing therapy, the subject disposition to the disease, and the judgment of the treating physician.

These and other aspects will become apparent from the following written description of the invention.

The following Examples are provided to better illustrate the present invention. All parts and percentages are by weight and all temperatures are degrees Celsius, unless explicitly stated otherwise.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results. The compounds of the Examples have been found to inhibit the activity of PD-1/PD-L1 protein/protein interaction according to at least one assay described herein.

### EXAMPLES

Experimental procedures for compounds of the invention are provided below. Open Access Preparative LCMS Purification of some of the compounds prepared was performed on Waters mass directed fractionation systems. The basic equipment setup, protocols and control software for the operation of these systems have been described in detail in literature. See, e.g., Blom, “Two-Pump At Column Dilution Configuration for Preparative LC-MS”, K. Blom, J. Combi. Chem., 2002, 4, 295-301; Blom et al, “Optimizing Preparative LC-MS Configurations and Methods for Parallel Synthesis Purification”, J. Combi. Chem., 2003, 5, 670-83; and Blom et al., “Preparative LC-MS Purification: Improved Compound Specific Method Optimization”, J. Combi. Chem., 2004, 6, 874-883.

The following abbreviations have been used in the examples:
Boc: t-butyloxycarbonyl;
BSA: Bovine serum album;
DCM: Dichloromethane;
DIEA: Diisopropylethylamine;
DMF: N, N-Dimethylformarmide;
DMSO: Dimethyl sulfoxide;
Et₂O: Ethyl ether;
EtOAc: Ethyl acetate;
h or hrs: hour or hours;
HATU:O-(7-azabenzotrizol-1-yl)-N,N,N,N-tetramethyluronium hexafluorophosphate;
HTRF: Homogeneous Time Resolved Fluorescence
MeCN: Methyl cyanide;
min: minute;
Pd(dppf)Cl.CH₂Cl₂:1,1′-Bis(diphenylphosphino)ferrocene-palladium(II) dichloride dichloromethane complex
t or r.t.: room temperature;
TFA: trifluoroacetic acid;
THF: Tetrahydrofuran.

### Example 1 Synthesis of Compound 1

### N,N′-(2,2′-dimethyl-[1,1′-biphenyl]-3,3′-diyl)bis(4,5,6,7-tetrahydrothiazolo[5,4-c]pyridine-2-carboxamide)

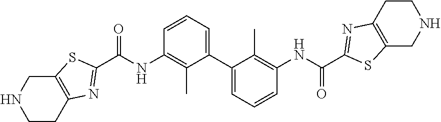 [SMILES: Cc1c(NC(=O)c2nc3c(s2)CNCC3)cccc1-c1cccc(NC(=O)c2nc3c(s2)CNCC3)c1C]

### Step 1: Preparation of 2-methyl-3-(4,4,5,5-tetramethyl-1,3,2-dioxaborolan-2-yl)aniline (M1)

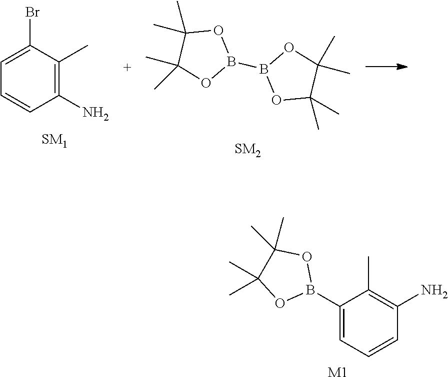

A mixture of 3-bromo-2-methylaniline (4.000 g), 4,4,4′,4′,5,5,5′,5′-octamethyl-2,2′-bi(1,3,2-dioxaborolane) (6.550 g) and potassium acetate (4.220 g) in 1,4-dioxane (44.8 mL) and DMSO (9.0 mL) was purged with nitrogen for 10 min. [1,1′-Bis(diphenylphosphino)ferrocene]-dichloropalladium DCM adduct (0.527 g) was added, the mixture was purged for another 5 min then was heated at reflux for 2 h. The mixture was cooled and filtered through Celite. The solids were washed with EtOAc, and the combined filtrates were washed with water and brine, and dried and concentrated. The residue was purified by column chromatography (eluting with hexane-EtOAc using a gradient from 20:1 to 85:15). 2-methyl-3-(4,4,5,5-tetramethyl-1,3,2-dioxaborolan-2-yl)aniline (compound M) as a light yellow waxy solid (4.400 g, 88%).

¹H NMR (400 MHz, chloroform-d) δ 7.21 (1H, dd, J=7.3, 1.0 Hz), 7.02 (1H, t, J=7.7 Hz), 6.75 (1H, dd, J=7.8, 1.0 Hz), 3.54 (2H, br. s.), 2.37 (3H, s), 1.34 (12H, s). Mass spectrum m/z 233.3, 234.3, 235.3 (M+H)+.

### Step 2: Preparation of 2,2′-dimethyl-[1,1′-biphenyl]-3,3′-diamine (M2)

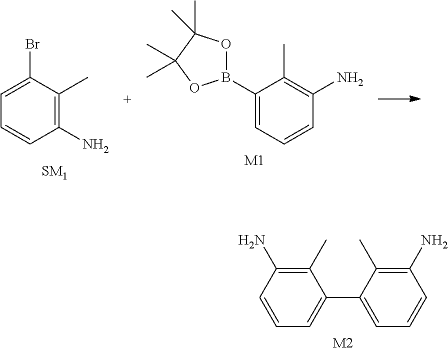

A mixture of 3-bromo-2-methylaniline (1.000 g), 2-methyl-3-(4,4,5,5-tetramethyl-1,3,2-dioxaborolan-2-yl)aniline (1.270 g) and potassium acetate (850 mg) in 1,4-dioxane (15 mL) and water (2.0 mL) was purged with nitrogen for 10 min. [1,1′-Bis(diphenylphosphino)ferrocene]-dichloropalladium DCM adduct (20 mg) was added, the mixture was purged for another 5 min then was heated at reflux for 2.7 h. The mixture was cooled and filtered through Celite. The solids were washed with EtOAc, and the combined filtrates were washed with water and brine, and dried and concentrated. The residue was purified by column chromatography (eluting with hexane-EtOAc using a gradient from 10:1 to 85:15). 2,2′-dimethyl-[1,1′-biphenyl]-3,3′-diamine (compound M2) as a light yellow waxy solid (900 mg, 79%).

### Step 3: Preparation of di-tert-butyl 2,2′-(((2,2′-dimethyl-[1,1′-biphenyl]-3,3′-diyl)bis(azanediyl))bis(carbonyl))bis(6,7-dihydrothiazolo[5,4-c]pyridine-5(4H)-carboxylate) (M3)

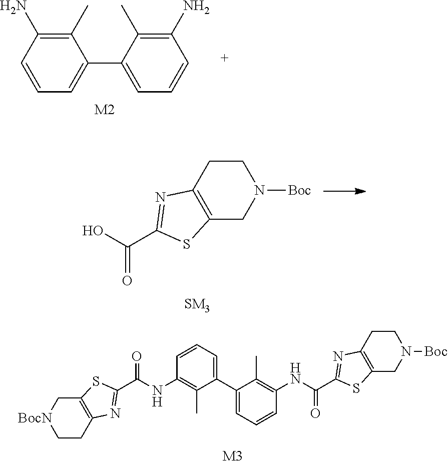 [SMILES: Cc1c(N)cccc1-c1cccc(N)c1C ; "M2" ; CC(C)(C)OC(=O)N1CCc2nc(C(=O)O)sc2C1 ; Cc1c(NC(=O)c2nc3c(s2)CN(C(=O)OC(C)(C)C)CC3)cccc1-c1cccc(NC(=O)c2nc3c(s2)CN(C(=O)OC(C)(C)C)CC3)c1C ; "M3"]

To a solution of 2,2′-dimethyl-[1,1′-biphenyl]-3,3′-diamine (100 mg) in dry dicloromethane was added HATU (300 mg) and DIEA (232 mg), A solution of 5-(tert-butoxycarbonyl)-4,5,6,7-tetrahydrothiazolo[5,4-c]pyridine-2-carboxylic acid (282 mg) in dicloromethane was added slowly and stirred at 40° C. for 4 hrs and then at room temperature overnight. Reaction mass was then concentrated and purified by column chromatography to afford the di-tert-butyl 2,2′-(((2,2′-dimethyl-[1,1′-biphenyl]-3,3′-diyl)bis(azanediyl))bis(carbonyl))bis(6,7-dihydrothiazolo[5,4-c]pyridine-5(4H)-carboxylate) (compound M3) as a light yellow solid (250 mg).

### Step 4: Preparation of N,N′-(2,2′-dimethyl-[1,1′-biphenyl]-3,3′-diyl)bis(4,5,6,7-tetrahydrothiazolo[5,4-c]pyridine-2-carboxamide) (Compound 1)

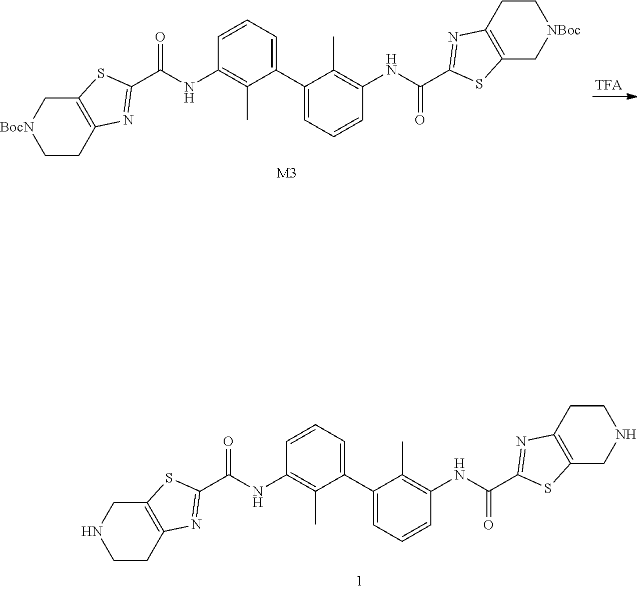 [SMILES: Cc1c(NC(=O)c2nc3c(s2)CN(C(=O)OC(C)(C)C)CC3)cccc1-c1cccc(NC(=O)c2nc3c(s2)CN(C(=O)OC(C)(C)C)CC3)c1C ; "M3" ; Cc1c(NC(=O)c2nc3c(s2)CNCC3)cccc1-c1cccc(NC(=O)c2nc3c(s2)CNCC3)c1C]

To a solution of 2,2′-(((2,2′-dimethyl-[1,1′-biphenyl]-3,3′-diyl)bis(azanediyl))bis (carbonyl))-bis(6,7-dihydrothiazolo[5,4-c]pyridine-5(4H)-carboxylate) (200 mg) in dicloromethane was added TFA (5 mL), and stirred at 40° C. for hrs. Reaction mass was then concentrated and washed by n-hexane to afford the N,N′-(2,2′-dimethyl-[1,1′-biphenyl]-3,3′-diyl)bis(4,5,6,7-tetrahydrothiazolo[5,4-c]pyridine-2-carboxamide) (compound 1) as a light yellow solid (125 mg).

### Example 2 Synthesis of Compound 2

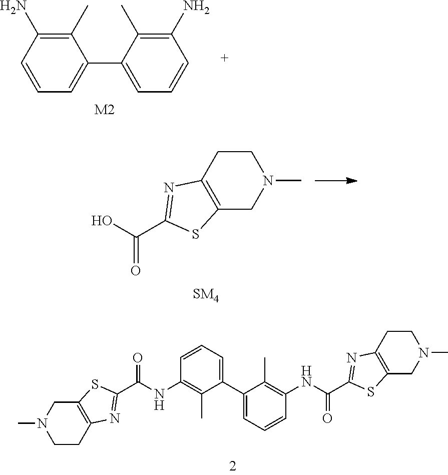

Prepare the compound M2 as described for Example 1.

Then, to a solution of 2,2′-dimethyl-[1,1′-biphenyl]-3,3′-diamine (100 mg, 0.45 mmol) in dry dichloromethane was added HATU (300 mg) and DIEA (232 mg). A solution of 5-methyl-4,5,6,7-tetrahydrothiazolo[5,4-c]pyridine-2-carboxylic acid (232 mg) in dicloromethane was added slowly and stirred at 40° C. for 4 hrs and then at room temperature overnight. Reaction mass was then concentrated and purified by column chromatography to afford the N,N′-(2,2′-dimethyl-[1,1′-biphenyl]-3,3′-diyl)bis(5-methyl-4,5,6,7-tetrahydrothiazolo[5,4-c]pyridine-2-carboxamide) (Compound 2) as a light yellow solid (180 mg).

### Example 3 Synthesis of Compound 3

### Step 1: Preparation of tert-butyl (S)-2-(2-(2-ethoxy-2-oxoacetyl)hydrazine-1-carbonyl)pyrrolidine-1-carboxylate (M3)

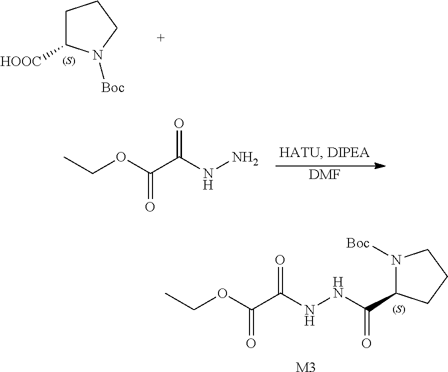

To a solution of Boc-L-proline (2.150 g) and ethyl 2-hydrazinyl-2-oxoacetate (1.980 g) in dry DMF was added DIPEA (2.600 g). HATU (5.700 g) was added in small portions at room temperature. The mixture was stirred for 2 h at the same temperature. DMF was evaporated under reduced pressure. The residue was purified directly by RP-column (mobile phase: MeCN:water=30:70) to afford tert-butyl (S)-2-(2-(2-ethoxy-2-oxoacetyl)hydrazine-2-carbonyl)pyrrolidine-1-carboxylate as a white solid (2.420 g).

### Step 2: Preparation of ethyl (S)-5-(1-(tert-butoxycarbonyl)pyrrolidin-2-yl)-1,3,4-thiadiazole-2-carboxylate (M33)

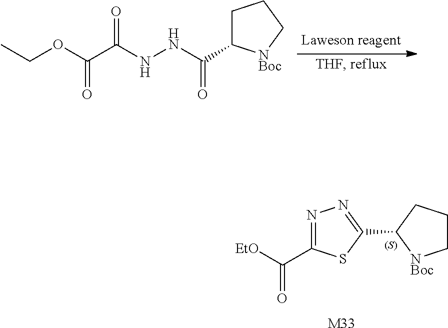

To a solution of tert-butyl (S)-2-(2-(2-ethoxy-2-oxoacetyl)hydrazine-1-carbonyl)pyrrolidine-1-carboxylate (2.310 g) in THF was added Lawesson reagent (3.400 g). The resulting mixture was heated to reflux for 2 h. The reaction was quenched by saturate Na₂CO₃ solution and extracted by EtOAc for 3 times. The combined organic phase was washed with water and brine then dried over Na₂SO₄. The resulting solution was concentrated and purified by silicagel (eluting with hexane-EtOAc using a gradient from 10:1 to 7:1) to afford ethyl (S)-5-(1-(tert-butoxycarbonyl)pyrrolidin-2-yl)-1,3,4-thiadiazole-2-carboxylate as a light yellow solid (1.610 g).

### Step 3: Preparation of (S)-5(1-((tert-butoxycarbonyl)pyrrolidin-2-yl)-1,3,4-thiadiazole-2-carboxylic acid (M333)

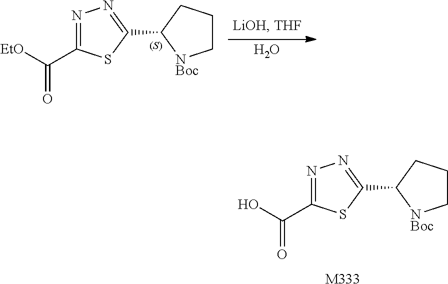

To a solution of ethyl (S)-5-(1-(tert-butoxycarbonyl)pyrrolidin-2-yl)-1,3,4-thiadiazole-2-carboxylate (1.610 g) in THF/water=1:1 (20 mL) was added LiOH (0.860 g). The resulting mixture was stirred for 3 h at room temperature. The reaction was quenched by 2M HCl and the PH value was adjusted to 4-5. Water and THF was evaporated out. The resulted solid was purified by RP-column (mobile phase: MeCN:water using a gradient from 10:90 to 30:70) to afford (S)-5-(1-(tert-butoxycarbonyl)pyrrolidin-2-yl)-1,3,4-thiadiazole-2-carboxylic acid as a white solid (0.900 g).

### Step 4: Preparation of Compound 3

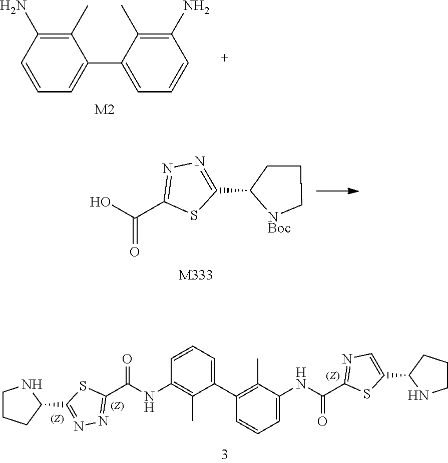

Prepare the compound M2 as described for Example 1.

Then, to a solution of 2,2′-dimethyl-[,1′-biphenyl]-3,3′-diamine (100 mg, 0.45 mmol) in dry dichloromethane was added HATU (300 mg) and DIEA (232 mg), A solution of (S)-5-(1-(tert-butoxycarbonyl)pyrrolidin-2-yl)-1,3,4-thiadiazole-2-carboxylic acid (312 mg) in dicloromethane was added slowly and stirred at 40° C. for 4 hrs and then at room temperature overnight. Reaction mass was then concentrated and purified by column chromatography to afford the Boc-protected intermediate (180 mg).

The above Boc-protected intermediate was dissolved in DCM 5 mL. TFA (1 mL) was added slowly at room temperature. The mixture could be stirred at room temperature for 2 h. The reaction was quenched by 10% Na₂CO₃ solution and extracted by DCM for 3 times. The combined organic layers was dried over Na₂SO₄. Na₂SO₄ was filtered out. The resulting solution was concentrated under reduced pressure to afford N,N′-(2,2′-dimethyl-[,1′-biphenyl]-3,3′-diyl)bis(5-((S)-pyrrolidin-2-yl)-1,3,4-thiadiazole-2-carboxamide) (compound 3) as a white solid (89 mg).

Prepare the following examples (shown in Table 1) essentially as described for Example 1, 2, or 3 using the corresponding starting materials.

TABLE 1

| | | | Physical |
|---|---|---|---|
| | | | Data |
| EX | | | (MS) |
| No. | Chemical Name | Structure | (M + H)⁺ |
| 1 | N,N′-(2,2′-dimethyl-[1,1′- biphenyl]- 3,3′-diyl)bis(4,5,6,7- tetrahydrothiazolo[5,4- c]pyridine-2- carboxamide) | 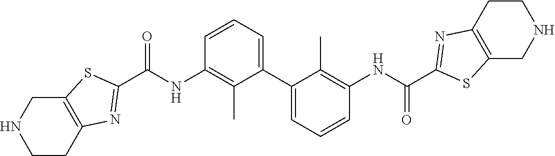 [SMILES: Cc1c(NC(=O)c2nc3c(s2)CNCC3)cccc1-c1cccc(NC(=O)c2nc3c(s2)CNCC3)c1C] | 545.2 |
| 2 | N,N′-(2,2′-dimethyl-[1,1′- biphenyl]-3,3′-diyl)bis(5- methyl-4,5,6,7- tetrahydrothiazolo[5,4- c]pyridine-2- carboxamide) | 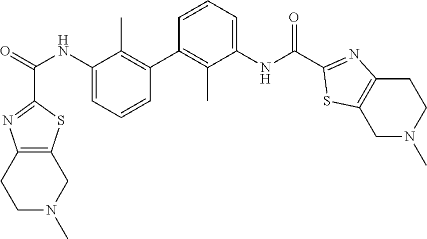 [SMILES: Cc1c(NC(=O)c2nc3c(s2)CN(C)CC3)cccc1-c1cccc(NC(=O)c2nc3c(s2)CN(C)CC3)c1C] | 573.2 |
| 3 | N,N′-(2,2′-dimethyl-[1,1′- biphenyl]-3,3′-diyl)bis(5- ((S)-pyrrolidin-2-yl)- 1,3,4-thiadiazole-2- carboxamide) | 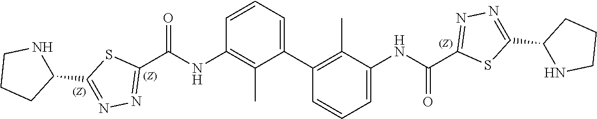 [SMILES: Cc1c(NC(=O)c2nnc([C@@H]3CCCN3)s2)cccc1-c1cccc(NC(=O)c2nnc([C@@H]3CCCN3)s2)c1C] | 575.2 |
| 4 | 2,2′-((((2,2′-dimethyl- [1,1′-biphenyl]- 3,3′-diyl)bis(azanediyl)) bis(carbonyl))bis(6,7- dihydrothiazolo[5,4- c]pyridine-2,5(4H)-diyl)) diacetic acid | 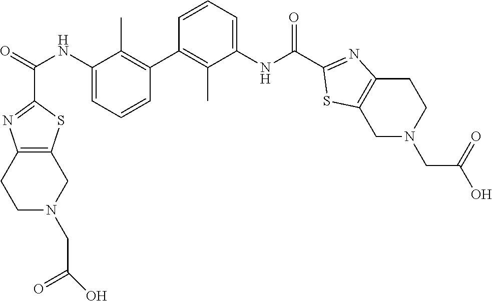 [SMILES: Cc1c(NC(=O)c2nc3c(s2)CN(CC(=O)O)CC3)cccc1-c1cccc(NC(=O)c2nc3c(s2)CN(CC(=O)O)CC3)c1C] | 661.2 |
| 5 | N-(2,2′-dimethyl-3′-(N- methyl-5,6,7,8-tetrahydro- imidazo[1,2-a]pyrazine- 2-carboxamido)-[1,1′- biphenyl]-3-yl)-N-methyl- 4,5,6,7-tetrahydro- thieno[2,3-c]pyridine- 2-carboxamide | 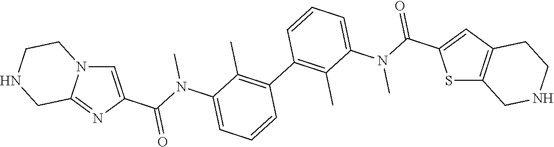 [SMILES: Cc1c(-c2cccc(N(C)C(=O)c3cc4c(s3)CNCC4)c2C)cccc1N(C)C(=O)c1cn2c(n1)CNCC2] | 555.3 |
| 6 | 2-(((8-((3′-((3-((2- hydroxyethyl)amino)-1,7- naphthyridin-8-yl)amino)- 2,2′-dimethyl-[1,1′- biphenyl]-3-yl)amino)- 1,7-naphthyridin-3- yl)methyl)amino)ethan- 1-ol | 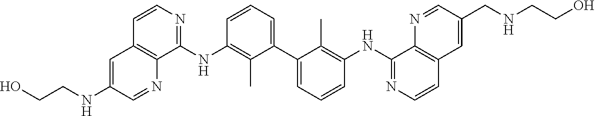 [SMILES: Cc1c(Nc2nccc3cc(CNCCO)cnc23)cccc1-c1cccc(Nc2nccc3cc(NCCO)cnc23)c1C] | 601.3 |
| 7 | N,N′-(2,2′-dimethyl-[1,1′- biphenyl]-3,3′-diyl)bis(5- (((2-hydroxyethyl)amino) methyl)thiazole-2- carboxamide) | 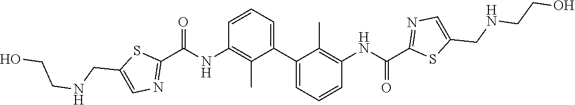 [SMILES: Cc1c(NC(=O)c2ncc(CNCCO)s2)cccc1-c1cccc(NC(=O)c2ncc(CNCCO)s2)c1C] | 581.2 |
| 8 | N,N′-(2,2′-dimethyl-[1,1′- biphenyl]- 3,3′-diyl)bis(5,6,7,8- tetrahydroimidazo[1,2- a]pyrazine-2- carboxamide) | 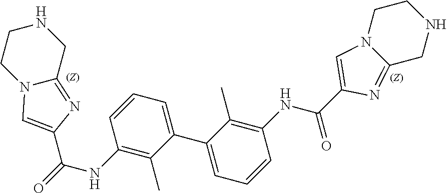 [SMILES: Cc1c(NC(=O)c2cn3c(n2)CNCC3)cccc1-c1cccc(NC(=O)c2cn3c(n2)CNCC3)c1C] | 511.3 |
| 9 | N,N′-(2,2′-dimethyl-[1,1′- biphenyl]-3,3′- diyl)bis(5-((S)-1- aminoethyl)- 1,3,4-oxadiazole-2- carboxamide) | 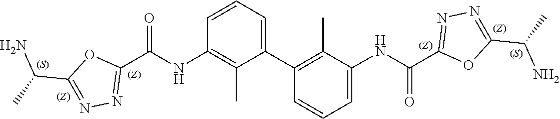 | 491.2 |
| 10 | N-(2,2′-dimethyl-3′- (5,6,7,8-tetrahydro- imidazo[1,2-a]pyrazine-2- carboxamido)-[1,1′- biphenyl]-3-yl)-4,5,6,7- tetrahydrothiazolo[5,4- c]pyridine-2-carboxamide | 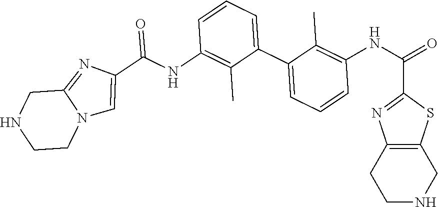 [SMILES: Cc1c(NC(=O)c2cn3c(n2)CNCC3)cccc1-c1cccc(NC(=O)c2nc3c(s2)CNCC3)c1C] | 528.2 |
| 11 | N-(3′-(6- aminobenzo[d]thiazole-2- carboxamido)-2,2′- dimethyl-[1,1′- biphenyl]-3-yl)-4,5,6,7- tetrahydrothiazolo[5,4- c]pyridine-2-carboxamide | 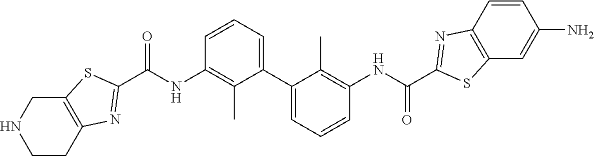 [SMILES: Cc1c(NC(=O)c2nc3c(s2)CNCC3)cccc1-c1cccc(NC(=O)c2nc3ccc(N)cc3s2)c1C] | 555.2 |
| 12 | N-(3′-(5-amino-1H- benzo[d]imidazole-2- carboxamido)-2,2′- dimethyl-[1,1′-biphenyl]- 3-yl)-4,5,6,7-tetrahydro- thiazolo[5,4-c]pyridine- 2-carboxamide | 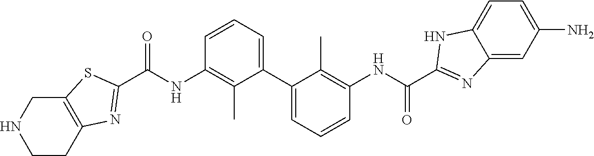 [SMILES: Cc1c(NC(=O)c2nc3cc(N)ccc3[nH]2)cccc1-c1cccc(NC(=O)c2nc3c(s2)CNCC3)c1C] | 538.2 |
| 13 | N,N′-(2,2′-dimethyl- [1,1′-biphenyl]- 3,3′-diyl)bis(4,5,6,7- tetrahydrooxazolo[5,4- c]pyridine-2- carboxamide) | 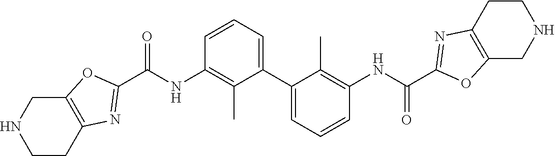 [SMILES: Cc1c(NC(=O)c2nc3c(o2)CNCC3)cccc1-c1cccc(NC(=O)c2nc3c(o2)CNCC3)c1C] | 513.2 |
| 14 | N-(2,2′-dimethyl-3′- (4,5,6,7-tetrahydro- thiazolo[5,4-c]pyridine-2- carboxamido)-[1,1′- biphenyl]- 3-yl)-4,5,6,7- tetrahydrooxazolo[5,4- c]pyridine-2- carboxamide | 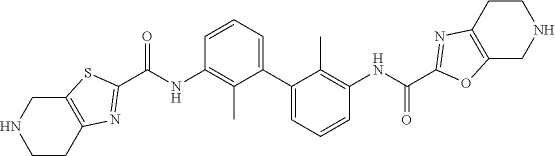 [SMILES: Cc1c(NC(=O)c2nc3c(o2)CNCC3)cccc1-c1cccc(NC(=O)c2nc3c(s2)CNCC3)c1C] | 529.2 |
| 15 | N-(2,2′-dimethyl-3′- (4,5,6,7-tetrahydro- thieno[3,2-c]pyridine-2- carboxamido)-[1,1′- biphenyl]- 3-yl)-4,5,6,7- tetrahydrothiazolo[5,4- c]pyridine-2- carboxamide | 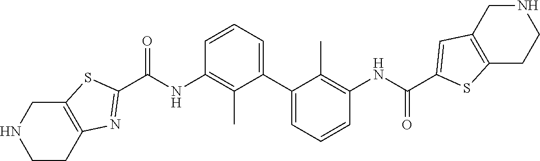 [SMILES: Cc1c(NC(=O)c2cc3c(s2)CCNC3)cccc1-c1cccc(NC(=O)c2nc3c(s2)CNCC3)c1C] | 544.2 |
| 16 | (2S,2′S)-1,1′-((1S,1′S)- ((((2,2′-dimethyl-[1,1′- biphenyl]-3,3′- diyl)bis(azanediyl)) bis(carbonyl)) bis(1,3,4-oxadiazole-5,2- diyl))bis(ethane-1,1- diyl))bis(piperidine-2- | 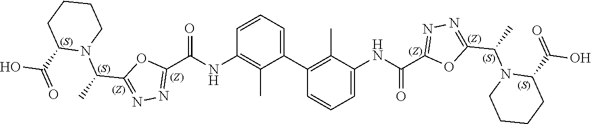 | 715.3 |
| | carboxylic acid) | | |
| 17 | (2S,2′S)-1,1′-((1S,1′S)- ((((2,2′-dimethyl-[1,1′- biphenyl]-3,3′- diyl)bis(azanediyl)) bis(carbonyl))bis(1,3,4- thiadiazole-5,2-diyl)) bis(ethane-1,1- diyl))bis(piperidine-2- | 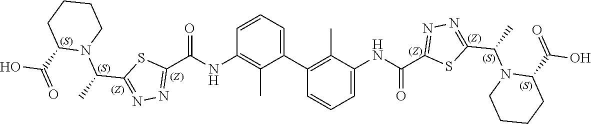 | 747.3 |
| | carboxylic acid) | | |
| 18 | N,N′-(2,2′-dimethyl- [1,1′-biphenyl]- 3,3′-diyl)bis(5- (aminomethyl)-1,3,4- oxadiazole-2- carboxamide) | 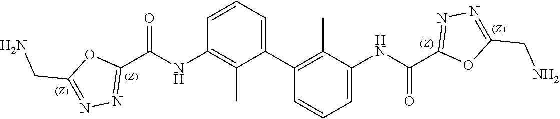 [SMILES: Cc1c(NC(=O)c2nnc(CN)o2)cccc1-c1cccc(NC(=O)c2nnc(CN)o2)c1C] | 463.2 |
| 19 | N,N′-(2,2′-dimethyl- [1,1′-biphenyl]- 3,3′-diyl)bis(5-(1- aminoethyl)-1,3,4- oxadiazole-2- carboxamide) | 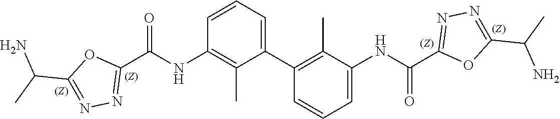 [SMILES: Cc1c(NC(=O)c2nnc(C(C)N)o2)cccc1-c1cccc(NC(=O)c2nnc(C(C)N)o2)c1C] | 491.2 |
| 20 | N,N′-(2,2′-dimethyl- [1,1′-biphenyl]- 3,3′-diyl)bis(5- (aminomethyl)-1,3,4- thiadiazole-2- carboxamide) | 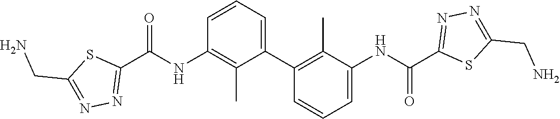 [SMILES: Cc1c(NC(=O)c2nnc(CN)s2)cccc1-c1cccc(NC(=O)c2nnc(CN)s2)c1C] | 495.1 |
| 21 | N-(3′-(6-amino-N- methylbenzo[d]thiazole-2- carboxamido)-2,2′- dimethyl-[1,1′- biphenyl]-3-yl)-N- methyl-4,5,6,7- tetrahydrothiazolo[5,4- c]pyridine-2-carboxamide | 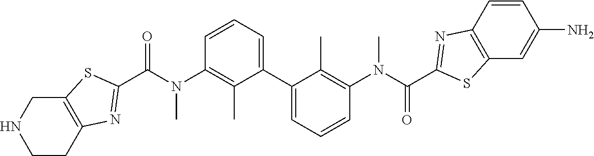 [SMILES: Cc1c(-c2cccc(N(C)C(=O)c3nc4ccc(N)cc4s3)c2C)cccc1N(C)C(=O)c1nc2c(s1)CNCC2] | 583.2 |
| 22 | N3-((6-amino- benzo[d]thiazol-2- yl)methyl)-N3,N3′,2,2′- tetramethyl-N3′-((4,5,6,7- tetrahydrothiazolo[5,4- c]pyridin-2-yl)methyl)- [1,1′-biphenyl]-3,3′- diamine | 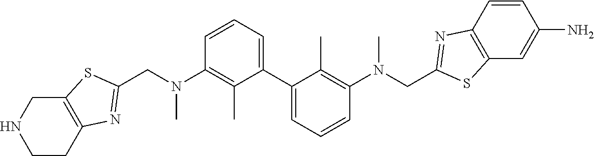 [SMILES: Cc1c(-c2cccc(N(C)Cc3nc4ccc(N)cc4s3)c2C)cccc1N(C)Cc1nc2c(s1)CNCC2] | 555.2 |
| 23 | N,N′-(2,2′-dimethyl- [1,1′-biphenyl]- 3,3′-diyl)bis(N-methyl- 4,5,6,7-tetrahydro- thiazolo[5,4-c]pyridine-2- carboxamide) | 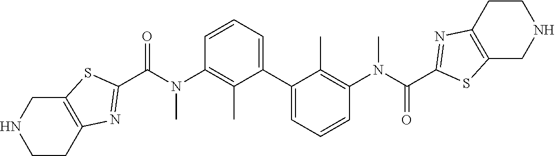 [SMILES: Cc1c(-c2cccc(N(C)C(=O)c3nc4c(s3)CNCC4)c2C)cccc1N(C)C(=O)c1nc2c(s1)CNCC2] | 573.2 |
| 24 | N3,N3′,2,2′-tetramethyl- N3,N3′-bis((4,5,6,7- tetrahydrothiazolo[5,4- c]pyridin-2-yl)methyl)- [1,1′-biphenyl]-3,3′- diamine | 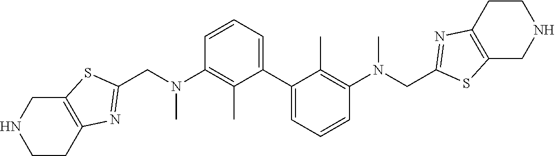 [SMILES: Cc1c(-c2cccc(N(C)Cc3nc4c(s3)CNCC4)c2C)cccc1N(C)Cc1nc2c(s1)CNCC2] | 545.3 |
| 25 | 2,2′-dimethyl-N3,N3′- bis((4,5,6,7- tetrahydrothiazolo[5,4- c]pyridin-2-yl)methyl)- [1,1′-biphenyl]-3,3′- diamine | 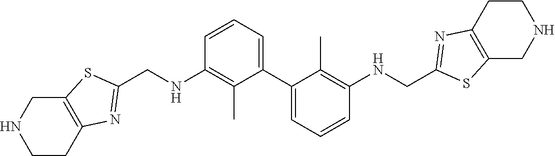 [SMILES: Cc1c(NCc2nc3c(s2)CNCC3)cccc1-c1cccc(NCc2nc3c(s2)CNCC3)c1C] | 517.2 |
| 26 | N3,N3′-bis((5-(1-amino- cyclobutyl)-1,3,4- thiadiazol-2-yl)methyl)- 2,2′-dimethyl- [1,1′-biphenyl]- 3,3′-diamine | 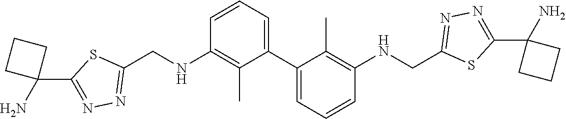 [SMILES: Cc1c(NCc2nnc(C3(N)CCC3)s2)cccc1-c1cccc(NCc2nnc(C3(N)CCC3)s2)c1C] | 547.2 |
| 27 | N,N′-(2,2′-dimethyl- [1,1′-biphenyl]- 3,3′-diyl)bis(5-(1- aminocyclobutyl)- 1,3,4-thiadiazole-2- carboxamide) | 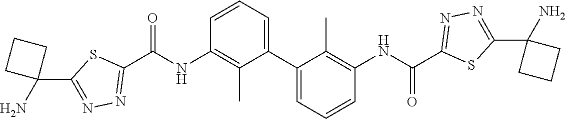 [SMILES: Cc1c(NC(=O)c2nnc(C3(N)CCC3)s2)cccc1-c1cccc(NC(=O)c2nnc(C3(N)CCC3)s2)c1C] | 575.2 |
| 28 | N,N′-(2,2′-dimethyl- [1,1′-biphenyl]- 3,3′-diyl)bis(4-methyl- 5-(pyrrolidin- 1-ylmethyl)thiazole- 2-carboxamide) | 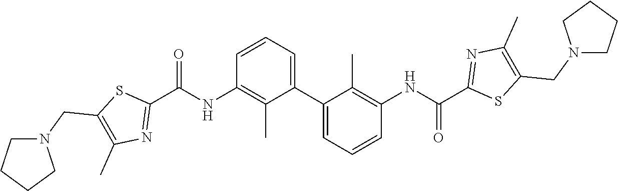 [SMILES: Cc1nc(C(=O)Nc2cccc(-c3cccc(NC(=O)c4nc(C)c(CN5CCCC5)s4)c3C)c2C)sc1CN1CCCC1] | 629.3 |
| 29 | N,N′-(2,2′-dimethyl- [1,1′-biphenyl]-3,3′- diyl)bis(5-(pyrrolidin- 1-ylmethyl)thiazole- 2-carboxamide) | 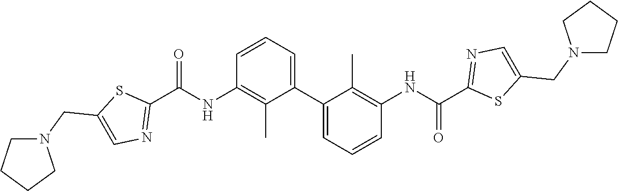 [SMILES: Cc1c(NC(=O)c2ncc(CN3CCCC3)s2)cccc1-c1cccc(NC(=O)c2ncc(CN3CCCC3)s2)c1C] | 601.2 |
| 30 | N,N′-(2,2′-dimethyl- [1,1′-biphenyl]- 3,3′-diyl)bis(5,6,7,8- tetrahydro-4H- thiazolo[5,4-c]azepine- 2-carboxamide) | 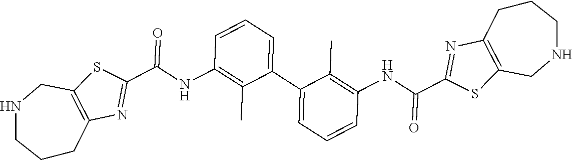 [SMILES: Cc1c(NC(=O)c2nc3c(s2)CNCCC3)cccc1-c1cccc(NC(=O)c2nc3c(s2)CNCCC3)c1C] | 573.2 |
| 31 | N,N′-(2,2′-dimethyl- [1,1′-biphenyl]- 3,3′-diyl)bis(5-((S)- 1-aminoethyl)- 1,3,4-thiadiazole-2- carboxamide) | 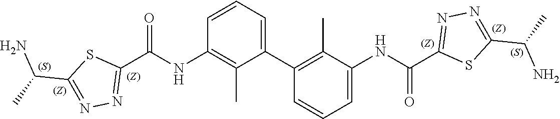 | 523.2 |
| 32 | N,N′-(2,2′-dimethyl- [1,1′-biphenyl]- 3,3′-diyl)bis(4,5,6,7- tetrahydrothieno[2,3- c]pyridine-2- carboxamide) | 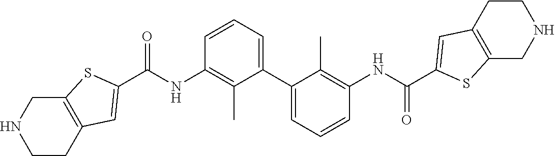 [SMILES: Cc1c(NC(=O)c2cc3c(s2)CNCC3)cccc1-c1cccc(NC(=O)c2cc3c(s2)CNCC3)c1C] | 543.2 |

### Example 33 Synthesis of Compound 33

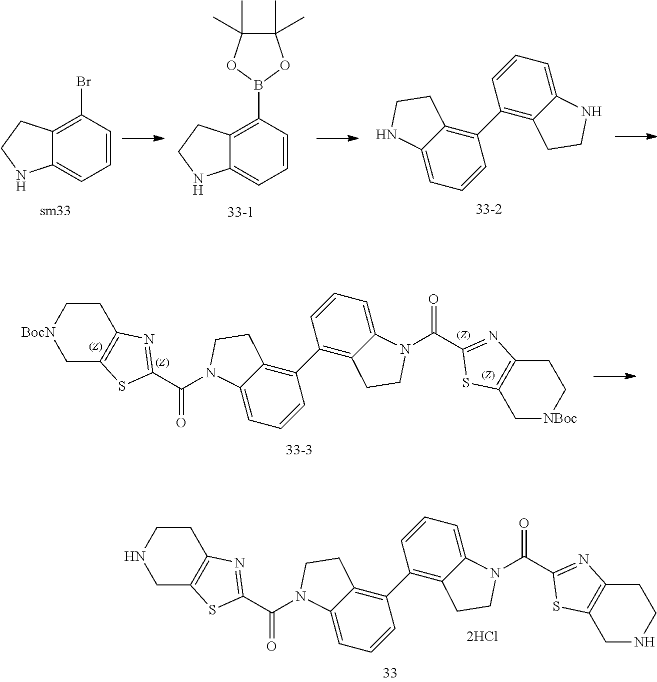 [SMILES: Brc1cccc2c1CCN2 ; CC1(C)OB(c2cccc3c2CCN3)OC1(C)C ; c1cc2c(c(-c3cccc4c3CCN4)c1)CCN2 ; CC(C)(C)OC(=O)N1CCc2nc(C(=O)N3CCc4c(-c5cccc6c5CCN6C(=O)c5nc6c(s5)CN(C(=O)OC(C)(C)C)CC6)cccc43)sc2C1 ; O=C(c1nc2c(s1)CNCC2)N1CCc2c(-c3cccc4c3CCN4C(=O)c3nc4c(s3)CNCC4)cccc21]

### Step 1: Preparation of 4-(4,4,5,5-tetramethyl-1,3,2-dioxaborolan-2-yl)indoline (33-1)

A mixture of 4-bromoindoline (1.000 g.), 4,4,4′,4′, 5,5,5′,5′-octamethyl-2,2′-bi(1,3,2-dioxaborolane) (2.550 g) and potassium acetate (2.220 g) in 1,4-dioxane (14.8 mL) and DMSO (1.0 mL) was purged with nitrogen for 10 min. [1,1′-Bis(diphenylphosphino)ferrocene]-dichloropalladium DCM adduct (0.127 g) was added, the mixture was purged for another 5 min then was heated at reflux for 2 h. The mixture was cooled and filtered through Celite. The solids were washed with EtOAc, and the combined filtrates were washed with water and brine, and dried and concentrated. The residue was purified by column chromatography (eluting with hexane-EtOAc using a gradient from 20:1 to 85:15). 4-(4,4,5,5-tetramethyl-1,3,2-dioxaborolan-2-yl)indoline (compound 2-1) as a light yellow waxy solid (1.400 g).

### Step 2: Preparation of 4,4′-biindoline (33-2)

A mixture of 4-(4,4,5,5-tetramethyl-1,3,2-dioxaborolan-2-yl)indoline (1.000 g), 4-bromoindoline (0.700 g) and potassium acetate (350 mg) in 1,4-dioxane (15 mL) and water (2.0 mL) was purged with nitrogen for 10 min. [1,1′-Bis(diphenylphosphino)ferrocene]-dichloropalladium DCM (20 mg) was added, the mixture was purged for another 5 min then was heated at reflux for 2.7 h. The mixture was cooled and filtered through Celite. The solids were washed with EtOAc, and the combined filtrates were washed with water and brine, and dried and concentrated. The residue was purified by column chromatography (eluting with hexane-EtOAc using a gradient from 10:1 to 85:15). 4,4′-biindoline (700 mg).

### Step 3: Preparation of 2-4 di-tert-butyl 2,2′-([4,4′-biindoline]-1,1′-dicarbonyl)bis(6,7-dihydrothiazolo[5,4-c]pyridine-5(4H)-carboxylate) (33-3)

To a solution of 4,4′-biindoline (100 mg) in dry dicloromethane was added HATU (300 mg) and DTEA (240 mg), A solution of 5-(tert-butoxycarbonyl)-4,5,6,7-tetrahydrothiazolo[5,4-c]pyridine-2-carboxylic acid (293 mg) in dicloromethane was added slowly and stirred at 40° C. for 4 hrs and then at room temperature overnight. Reaction mass was then concentrated and purified by column chromatography to afford the di-tert-butyl 2,2′-([4,4′-biindoline]-1,1′-dicarbonyl)bis(6,7-dihydrothiazolo[5,4-c]pyridine-5(4H)-carboxylate) (compound 33-3) as a light yellow solid (130 mg).

### Step 4: Preparation of [4,4′-biindoline]-1,1′-diylbis((4,5,6,7-tetrahydrothiazolo[5,4-c]pyridin-2-yl)methanone) (33)

To a solution of di-tert-butyl 2,2′-([4,4′-biindoline]-1,1′-dicarbonyl)bis(6,7-dihydrothiazolo[5,4-c]pyridine-5(4H)-carboxylate) (compound 33-3) (200 mg) in dicloromethane was added TFA (5 mL), and stirred at 40° C. for 5 hrs. Reaction mass was then concentrated and washed by n-hexane to afford the [4,4′-biindoline]-1,1′-diylbis((4,5,6,7-tetrahydrothiazolo[5,4-c]pyridin-2-yl)methanone) (compound 33) as a light yellow solid (125 mg).

### Example 34 Synthesis of Compound 34

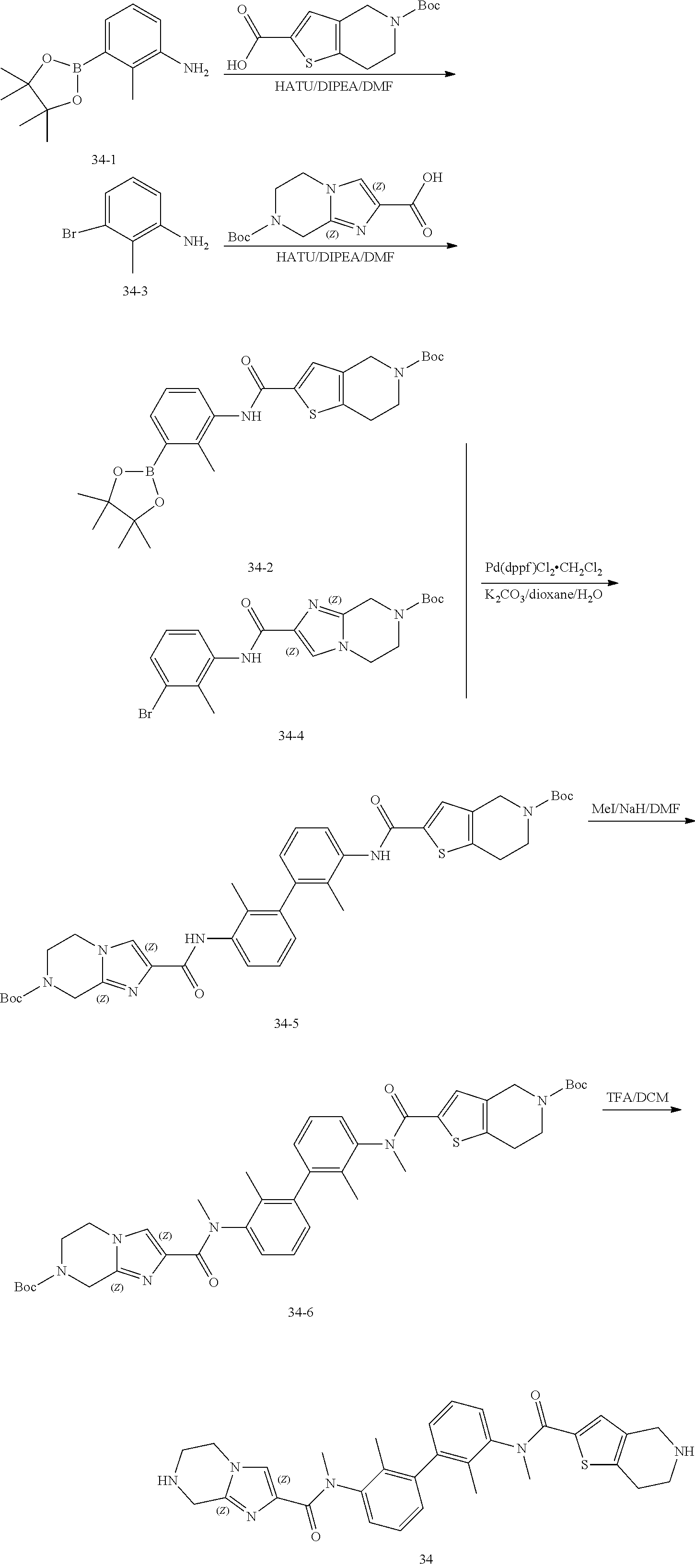

### Step 1: Preparation of tert-butyl 2-((2-methyl-3-(4,4,5,5-tetramethyl-1,3,2-dioxaborolan-2-yl)phenyl)carbamoyl)-6,7-dihydrothieno[3,2-c]pyridine-5(4H)-carboxylat (34-2)

In a 100 ml round bottom flask, was placed compound 1 (197 mg) and 5-(tert-butoxycarbonyl)-4,5,6,7-tetrahydrothieno[3,2-c]pyridine-2-carboxylic acid (200 mg), HATU (322 mg), DMF (5 mL), DIPEA (274 mg), the reaction was stirred for 2 hrs at r.t.

To the above mixture, H₂O (10 mL) was added, EA (15 mL) extract for 3 times, the organic phase was combined, washed with saturated NaCl(aq), dried with Na₂SO₄, concentrated, purified with flash chromatographic column (hexane/EA, EA=0-15%), 287 mg product was obtained with a little yellow solid.

### Step 2: Preparation of tert-butyl 2-((3-bromo-2-methylphenyl)carbamoyl)-5,6-dihydroimidazo[1,2-a]pyrazine-7(8H)-carboxylate (34-4)

In a 100 mL round bottom flask, was placed compound 34-3 (153 mg) and 7-(tert-butoxycarbonyl)-5,6,7,8-tetrahydroimidazo[1,2-a]pyrazine-2-carboxylic acid (200 mg), HATU (341 mg), DIPEA (290 mg), DMF (5 mL), stirred overnight at 40° C. Concentrated, the result mixture was purified with a flash chromatographic column (hexane/EA, EA=−15%), 462 mg product was obtained with a yellow solid.

### Step 3: Preparation of tert-butyl 2-((3′-(7-(tert-butoxycarbonyl)-5,6,7,8-tetrahydroimidazo[1,2-a]pyrazine-2-carboxamido)-2,2′-dimethyl-[1,1′-biphenyl]-3-yl)carbamoyl)-6,7-dihydrothieno[3,2-c]pyridine-5(4H)-carboxylate (34-5)

In a 100 mL round bottom flask was placed 2 (200 mg) and 4 (134 mg), Pd(dppf)Cl.CH₂Cl₂ (25 mg), K₂CO₃ (85 mg), dioxane (10 mL), H₂O (1 mL), with a N₂ atmosphere, stirred for 2 hrs at 80° C. Cooled down, concentrated, purified with flash chromatographic column (DCM/MeOH, MeOH=0-5%), 57 mg product was obtained with a little yellow solid.

### Step 4: Preparation of tert-butyl 2-((3′-(7-(tert-butoxycarbonyl)-N-methyl-5,6,7,8-tetrahydroimidazo[1,2-a]pyrazine-2-carboxamido)-2,2′-dimethyl-[1,1′-biphenyl]-3-yl)(methyl)carbamoyl)-6,7-dihydrothieno[3,2-c]pyridine-5(4H)-carboxylate (34-6)

In a 100 mL round bottom flash was placed 5 (50 mg), THF (2 mL), was added NaH (8 mg) at 0° C., stirred for 30 min, then CH₃I (24 mg) was added, stirred for another 30 min at r.t. Quenced with H₂O (5 mL), extracted with EA (15 mL*3), the organic phase was combined, dried with Na₂SO₄, concentrated, purified with a flash chromatographic column (DCM/MeOH, MeOH=0-10%), 80 mg product was obtained with a yellow solid.

### Step 5: N-(2,2′-dimethyl-3′-(N-methyl-5,6,7,8-tetrahydroimidazo[1,2-a]pyrazine-2-carboxamido)-[1,1′-biphenyl]-3-yl)-N-methyl-4,5,6,7-tetrahydrothieno[3,2-c]pyridine-2-carboxamide (34)

In a 50 mL round bottom flask was placed 6 (80 mg), TFA (3 mL) and DCM (9 mL), stirred for 1 h at r.t. Concentrated, diluted with H₂O (0 mL), adjusted pH=7-8 with NaHCO₃(aq), extracted with DCM (15 mL*3), the organic phase was combined and washed with saturated NaCl(aq), dried with Na₂SO₄, concentrated, the result product was washed with Et₂O (10 mL), 18 mg pure product was obtained with a red solid.

In some Examples, some of examples are used as a starting material, which undergoes the corresponding reaction with other material, such as methyl 2-bromoacetate. For example, preparing the following Example 102 and 103 using Example 33 as a starting material, which is describing for Example 102 and 103.

### Example 102 and 103 Synthesis of Compound 102 and 103

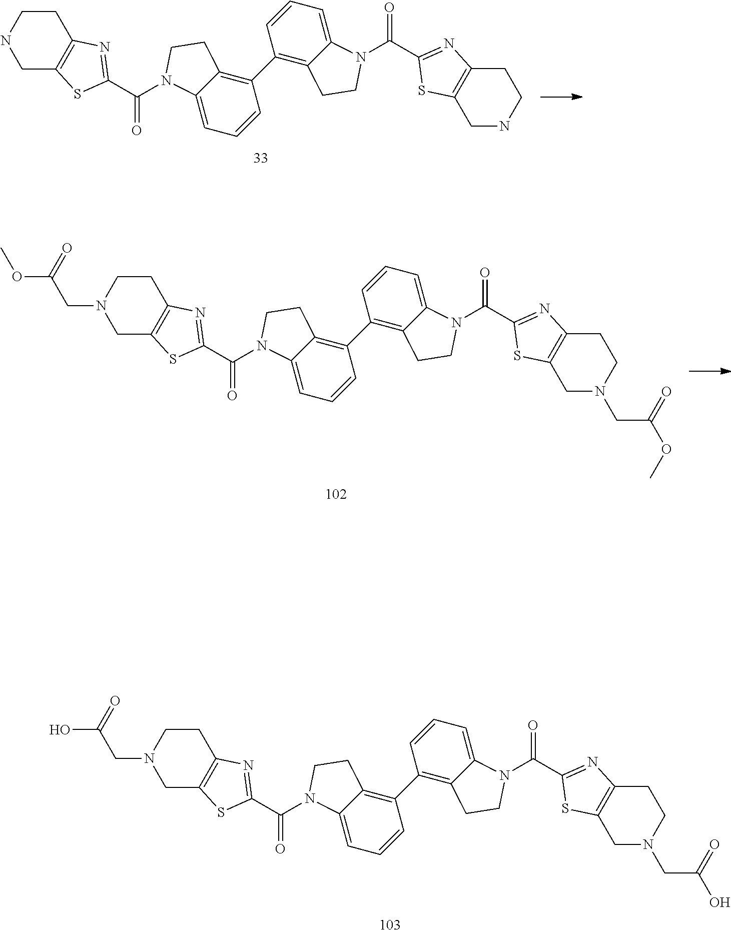 [SMILES: O=C(c1nc2c(s1)CNCC2)N1CCc2c(-c3cccc4c3CCN4C(=O)c3nc4c(s3)CNCC4)cccc21 ; COC(=O)CN1CCc2nc(C(=O)N3CCc4c(-c5cccc6c5CCN6C(=O)c5nc6c(s5)CN(CC(=O)OC)CC6)cccc43)sc2C1 ; "102" ; O=C(O)CN1CCc2nc(C(=O)N3CCc4c(-c5cccc6c5CCN6C(=O)c5nc6c(s5)CN(CC(=O)O)CC6)cccc43)sc2C1 ; "103"]

### Step 1: dimethyl 2,2′-(([4,4′-biindoline]-1,1′-dicarbonyl)bis(6,7-dihydrothiazolo[5,4-c]pyridine-2,5(4H)-diyl))diacetate (102)

To a solution of [4,4′-biindoline]-1,1′-diylbis((6,7-dihydro-4H-512-thiazolo[5,4-c]pyridin-2-yl)methanone) (50 mg) in DMF (2 mL) was added K₂CO₃ (60 mg), methyl 2-chloroacetate (50 mg), potassium iodide (5 mg), and stirred at 80° C. for 10 hrs. Reaction was add H₂O (20 mL) and extracted by EtOAc for 3 times. The combined organic phase was washed with water and brine (30 mL*5) then dried over Na₂SO₄. The resulting solution was concentrated, the resulted solid was purified by Column chromatography to get the dimethyl 2,2′-(([4,4′-biindoline]-1,1′-dicarbonyl)bis(6,7-dihydrothiazolo[5,4-c]pyridine-2,5(4H)-diyl))diacetate (compound 102) as a light yellow solid (35 mg).

### Step 2: 2,2′-(([4,4′-biindoline]-1,1′-dicarbonyl)bis(6,7-dihydrothiazolo[5,4-c]pyridine-2,5(4H)-diyl))diacetic acid (103)

To a solution of dimethyl 2,2′-(([4,4′-biindoline]-1,1′-dicarbonyl)bis(6,7-dihydrothiazolo[5,4-c]pyridine-2,5(4H)-diyl))diacetate (compound 2-6) (30 mg) in THF:CH₃OH=1:1 (15 mL) was added LiOH (10 mg) and H₂O (2 mL), and stirred at 25° C. for 2 hrs. The reaction was quenched by 1M HCl and the PH value was adjusted to 4-5. Water and THF was evaporated out. The resulted solid was purified by RP-column (mobile phase: MeCN:water using a gradient from 10:90 to 30:70) to afford 2,2′-(([4,4′-biindoline]-1,1′-dicarbonyl)bis(6,7-dihydrothiazolo[5,4-c]pyridine-2,5(4H)-diyl))diacetic acid (compound 110)(20 mg).

Prepare the following examples (shown in Table 2) essentially as described for Example 1, 33, 34, 102 or 103 using the corresponding starting materials. For example, prepare the following Example 44 (shown in Table 2) essentially as described for Example 1 using

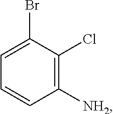

instead of

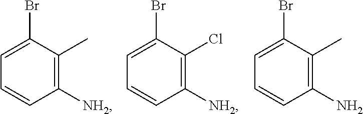

and other starting materials are either commercially available or made by known procedures in the reported literature or as illustrated.

TABLE 2

| | | | Physical |
|---|---|---|---|
| | | | Data |
| | | | (MS) |
| NO. | Chemical Name | Structure | (M + H)⁺ |
| 33 | [4,4′-biindoline]-1,1′-diylbis((6,7- dihydro-4H-5l2-thiazolo[5,4-c]pyridin- 2-yl)methanone) | 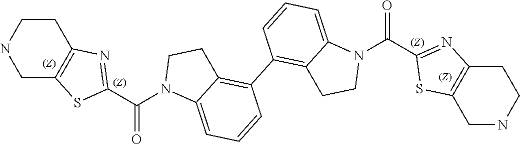 [SMILES: O=C(c1nc2c(s1)CNCC2)N1CCc2c(-c3cccc4c3CCN4C(=O)c3nc4c(s3)CNCC4)cccc21] | 567.2 |
| 34 | N-(2,2′-dimethyl-3′-(N-methyl- 5,6,7,8-tetrahydroimidazo[1,2- a]pyrazine-2-carboxamido)-[1,1′- biphenyl]-3-yl)-N-methyl-4,5,6,7- tetrahydrothieno[3,2-c]pyridine-2- carboxamide | 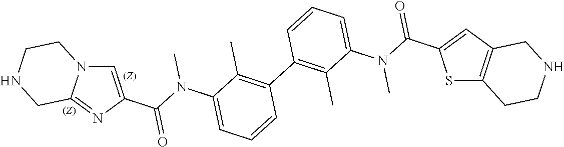 [SMILES: Cc1c(-c2cccc(N(C)C(=O)c3cc4c(s3)CCNC4)c2C)cccc1N(C)C(=O)c1cn2c(n1)CNCC2] | 555.3 |
| 35 | N-(2,2′-dimethyl-3′-(5,6,7,8- tetrahydroimidazo[1,2-a]pyrazine-2- carboxamido)-[1,1′-biphenyl]-3-yl)- 4,5,6,7-tetrahydrothieno[3,2- c]pyridine-2-carboxamide | 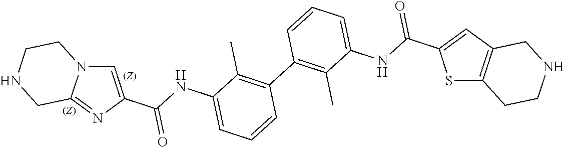 [SMILES: Cc1c(NC(=O)c2cn3c(n2)CNCC3)cccc1-c1cccc(NC(=O)c2cc3c(s2)CCNC3)c1C] | 526.7 |
| 36 | N,N′-(2,2′-dimethyl-[1,1′- biphenyl]-3,3′-diyl)bis(7-(1- hydroxypropan-2-yl)-5,6,7,8- tetrahydroimidazo[1,2-a]pyrazine-2- carboxamide) | 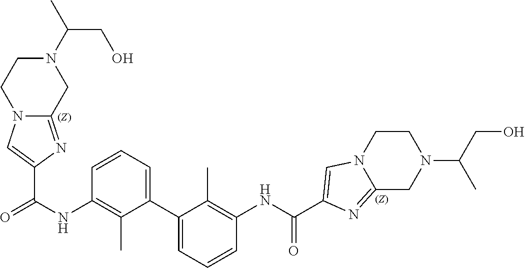 [SMILES: Cc1c(NC(=O)c2cn3c(n2)CN(C(C)CO)CC3)cccc1-c1cccc(NC(=O)c2cn3c(n2)CN(C(C)CO)CC3)c1C] | 627.3 |
| 37 | N,N′-(2,2′-dimethyl-[1,1′- biphenyl]-3,3′-diyl)bis(5- (guanidinomethyl)-1,3,4-thiadiazole-2- carboxamide) | 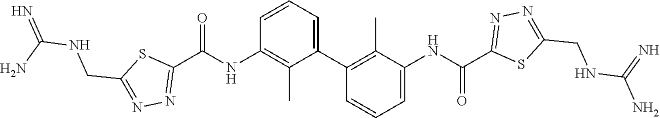 [SMILES: Cc1c(NC(=O)c2nnc(CNC(=N)N)s2)cccc1-c1cccc(NC(=O)c2nnc(CNC(=N)N)s2)c1C] | 579.2 |
| 38 | N,N′-(2,2′-dimethyl-[1,1′- biphenyl]-3,3′-diyl)bis(5-(1- guanidinocyclobutyl)-1,3,4-thiadiazole- 2-carboxamide) | 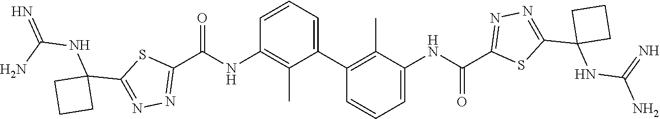 [SMILES: Cc1c(NC(=O)c2nnc(C3(NC(=N)N)CCC3)s2)cccc1-c1cccc(NC(=O)c2nnc(C3(NC(=N)N)CCC3)s2)c1C] | 659.2 |
| 39 | N,N′-(2,2′-dimethyl-[1,1′- biphenyl]-3,3′-diyl)bis(5,6,7,8- tetrahydro-[1,2,4]triazolo[4,3- a]pyrazine-3-carboxamide) | 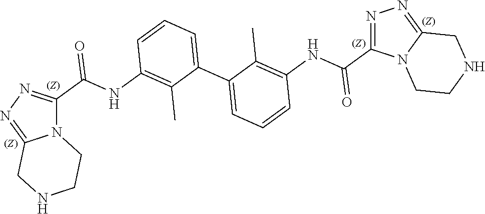 [SMILES: Cc1c(NC(=O)c2nnc3n2CCNC3)cccc1-c1cccc(NC(=O)c2nnc3n2CCNC3)c1C] | 513.2 |
| 40 | N,N′-(2,2′-dicyano-[1,1′- biphenyl]-3,3′-diyl)bis(5,6,7,8- tetrahydroimidazo[1,2-a]pyrazine-2- carboxamide) | 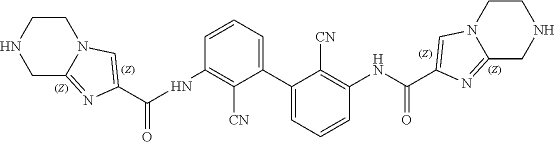 [SMILES: N#Cc1c(NC(=O)c2cn3c(n2)CNCC3)cccc1-c1cccc(NC(=O)c2cn3c(n2)CNCC3)c1C#N] | 533.2 |
| 41 | N,N′-(2,2-dimethyl-[1,1′- biphenyl]-3,3′-diyl)bis(5-(pyrrolidin-2- yl)-1,3,4-oxadiazole-2-carboxamide) | 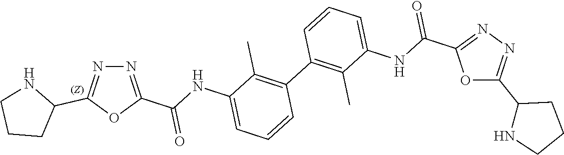 [SMILES: Cc1c(NC(=O)c2nnc(C3CCCN3)o2)cccc1-c1cccc(NC(=O)c2nnc(C3CCCN3)o2)c1C] | 543.2 |
| 42 | N-(2,2′-dimethyl-3′-(5,6,7,8- tetrahydroimidazo[1,2-a]pyrazine-2- carboxamido)-[1,1′-biphenyl]-3-yl)- 4,5,6,7-tetrahydrothieno[2,3- c]pyridine-2-carboxamide | 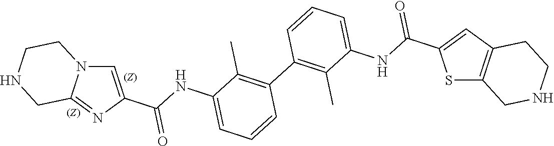 [SMILES: Cc1c(NC(=O)c2cn3c(n2)CNCC3)cccc1-c1cccc(NC(=O)c2cc3c(s2)CNCC3)c1C] | 527.2 |
| 43 | N,N′-(2,2′-dichloro-[1,1′- biphenyl]-3,3′-diyl)bis(4,5,6,7- tetrahydrothiazolo[5,4-c]pyridine-2- carboxamide) | 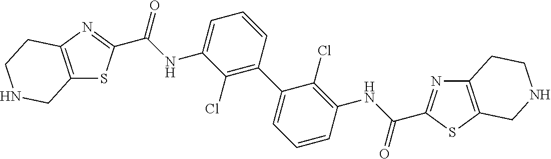 [SMILES: O=C(Nc1cccc(-c2cccc(NC(=O)c3nc4c(s3)CNCC4)c2Cl)c1Cl)c1nc2c(s1)CNCC2] | 586.1 |
| 44 | N,N′-(2,2′-dichloro-[1,1′- biphenyl]-3,3′-diyl)bis(5-methyl- 4,5,6,7-tetrahydrothiazolo[5,4- c]pyridine-2-carboxamide) | 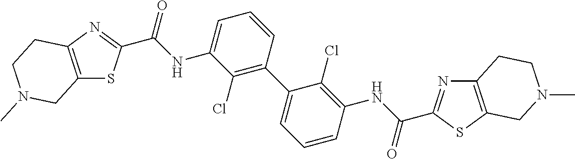 | 614.1 |
| 45 | N,N′-(2,2′-dimethyl-[1,1′- biphenyl]-3,3′-diyl)bis(5-(1- hydroxypropan-2-yl)-4,5,6,7- tetrahydrothiazolo[5,4-c]pyridine-2- carboxamide) | 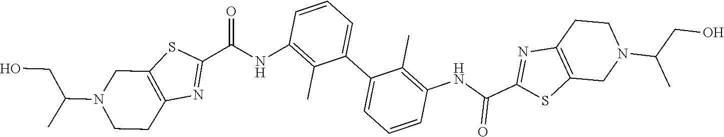 [SMILES: Cc1c(NC(=O)c2nc3c(s2)CN(C(C)CO)CC3)cccc1-c1cccc(NC(=O)c2nc3c(s2)CN(C(C)CO)CC3)c1C] | 661.3 |
| 46 | 1,1′-bis((6,7-dihydro-4H-5l2- thiazolo[5,4-c]pyridin-2-yl)methyl)- 4,4'-biindoline | 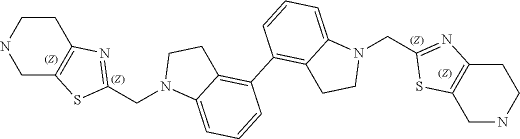 [SMILES: c1cc(-c2cccc3c2CCN3Cc2nc3c(s2)CNCC3)c2c(c1)N(Cc1nc3c(s1)CNCC3)CC2] | 539.2 |
| 47 | 1,1′-bis(1-(5-methyl-4,5,6,7- tetrahydrothiazolo[5,4-c]pyridin-2- yl)ethyl)-4,4′-biindoline | 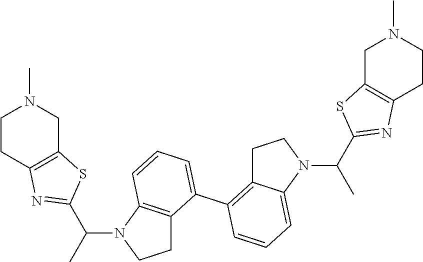 [SMILES: CC(c1nc2c(s1)CN(C)CC2)N1CCc2c(-c3cccc4c3CCN4C(C)c3nc4c(s3)CN(C)CC4)cccc21] | 597.3 |
| 48 | N,N′-(2-chloro-2′-methylbiphenyl- 3,3′-diyl)bis(4,5,6,7- tetrahydrothiazolo[5,4-c]pyridine-2- carboxamide) | 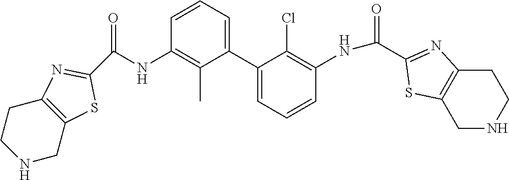 [SMILES: Cc1c(NC(=O)c2nc3c(s2)CNCC3)cccc1-c1cccc(NC(=O)c2nc3c(s2)CNCC3)c1Cl] | 565.1 |
| 49 | N,N′-(2-chloro-2′-methylbiphenyl- 3,3′-diyl)bis(5-methyl-4,5,6,7- tetrahydrothiazolo[5,4-c]pyridine-2- carboxamide) | 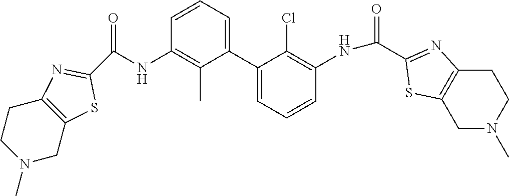 [SMILES: Cc1c(NC(=O)c2nc3c(s2)CN(C)CC3)cccc1-c1cccc(NC(=O)c2nc3c(s2)CN(C)CC3)c1Cl] | 593.2 |
| 50 | N-(2-chloro-2′-methyl-3′-(4,5,6,7- tetrahydrothiazolo[5,4-c]pyridine-2- carboxamido)biphenyl-3-yl)-5-methyl- 4,5,6,7-tetrahydrothiazolo[5,4- c]pyridine-2-carboxamide | 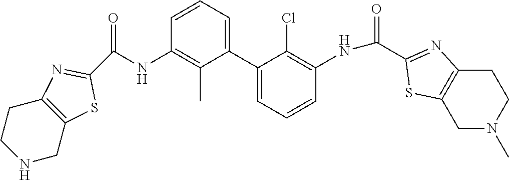 [SMILES: Cc1c(NC(=O)c2nc3c(s2)CNCC3)cccc1-c1cccc(NC(=O)c2nc3c(s2)CN(C)CC3)c1Cl] | 579.1 |
| 51 | N,N′-(2,2′-dimethylbiphenyl-3,3′- diyl)bis(5-((2- hydroxyethylamino)methyl)-4- methylthiazole-2-carboxamide) | 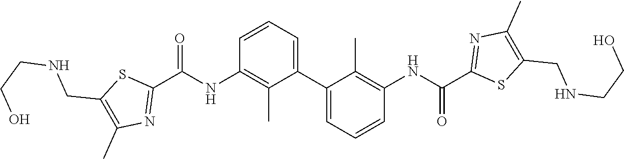 [SMILES: Cc1nc(C(=O)Nc2cccc(-c3cccc(NC(=O)c4nc(C)c(CNCCO)s4)c3C)c2C)sc1CNCCO] | 609.2 |
| 52 | (2S,2′S)-1,1′-(2,2′-(2,2′- dimethylbiphenyl-3,3′- diyl)bis(azanediyl)bis(oxomethylene) bis(4-methylthiazole-5,2- diyl))bis(methylene)dipiperidine-2- carboxylic acid | 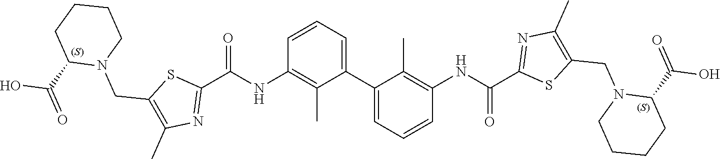 | 745.3 |
| 53 | (2S,2′S)-2,2′-(2,2′-(2,2′- dimethylbiphenyl-3,3′- diyl)bis(azanediyl)bis(oxomethylene) bis(4-methylthiazole-5,2- diyl))bis(methylene)bis(azanediyl) bis(2-(tetrahydro-2H-pyran-4- yl)acetic acid) | 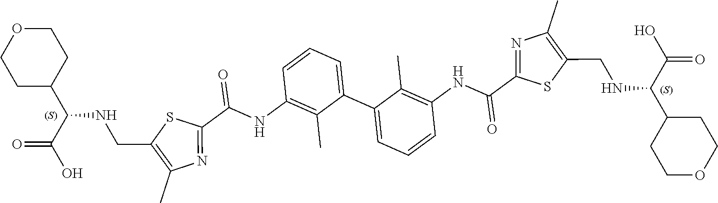 | 805.3 |
| 54 | (2S,2′S)-1,1′-(2,2′-(2-chloro-2′- methylbiphenyl-3,3′- diyl)bis(azanediyl)bis(oxomethylene) bis(4-methylthiazole-5,2- diyl))bis(methylene)dipiperidine-2- carboxylic acid | 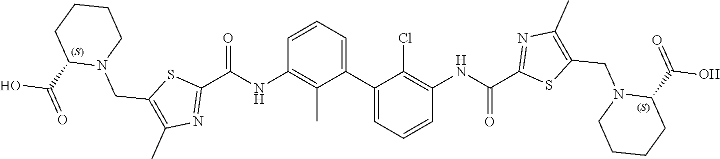 | 765.2 |
| 55 | (2S,2′S)-2,2′-(2,2′-(2-chloro-2′- methylbiphenyl-3,3′- diyl)bis(azanediyl)bis(oxomethylene) bis(4-methylthiazole-5,2- diyl))bis(methylene)bis(azanediyl) bis(2-(tetrahydro-2H-pyran-4- yl)acetic acid) | 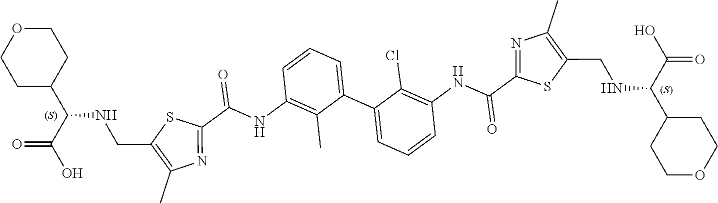 | 825.2 |
| 56 | (2S,2′S)-1,1′-(2,2′-(4,4′-biindoline- 1,1′-diylbis(oxomethylene))bis(4- methylthiazole-5,2- diyl))bis(methylene)dipiperidine-2- carboxylic acid | 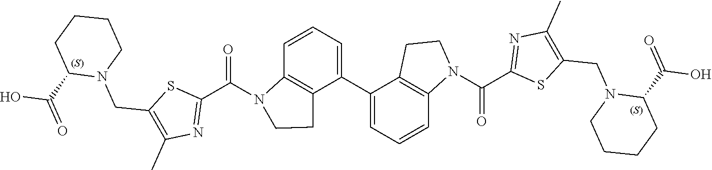 | 769.3 |
| 57 | (2S,2′S)-2,2′-(2,2′-(4,4′-biindoline- 1,1′-diylbis(oxomethylene))bis(4- methylthiazole-5,2- diyl))bis(methylene)bis(azanediyl) bis(2-(tetrahydro-2H-pyran-4- yl)acetic acid) | 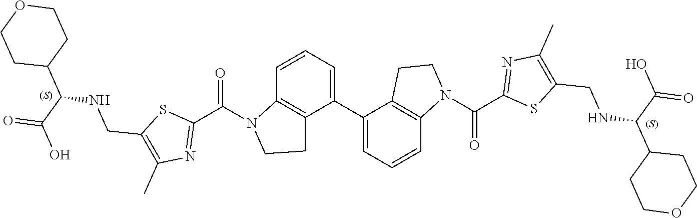 | 829.30 |
| 58 | (S)-1-((2-(3-(1-(5-(((S)-2- carboxypiperidin-1-yl)methyl)-4- methylthiazole-2-carbonyl)indolin-4- yl)-2-methylphenylcarbamoyl)-4- methylthiazol-5-yl)methyl)piperidine- 2-carboxylic acid | 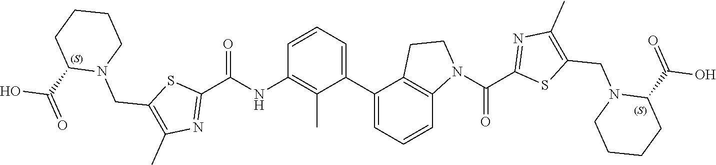 | 757.3 |
| 59 | (S)-2-((2-(3-(1-(5-(((S)- carboxy(tetrahydro-2H-pyran-4- yl)methylamino)methyl)-4- methylthiazole-2-carbonyl)indolin-4- yl)-2-methylphenylcarbamoyl)-4- methylthiazol-5-yl)methylamino)-2- (tetrahydro-2H-pyran-4-yl)acetic acid | 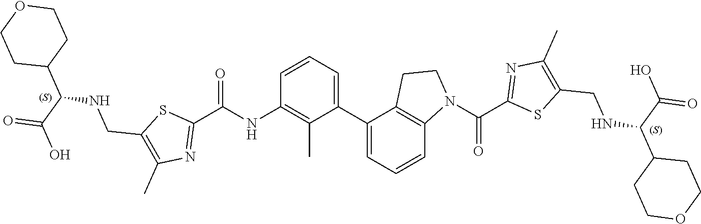 | 817.3 |
| 60 | (S)-1-((2-(3-(1-(5-(((S)-2- carboxypiperidin-1-yl)methyl)-4- methylthiazole-2-carbonyl)indolin-4- yl)-2-chlorophenylcarbamoyl)-4- methylthiazol-5-yl)methyl)piperidine- 2-carboxylic acid | 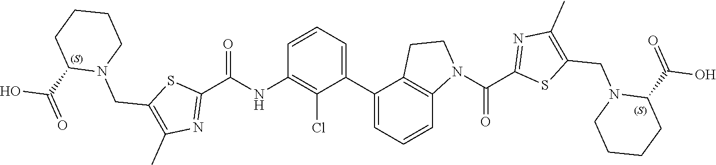 | 777.2 |
| 61 | (S)-2-((2-(3-(1-(5-(((S)- carboxy(tetrahydro-2H-pyran-4- yl)methylamino)methyl)-4- methylthiazole-2-carbonyl)indolin-4- yl)-2-chlorophenylcarbamoyl)-4- methylthiazol-5-yl)methylamino)-2- (tetrahydro-2H-pyran-4-yl)acetic acid | 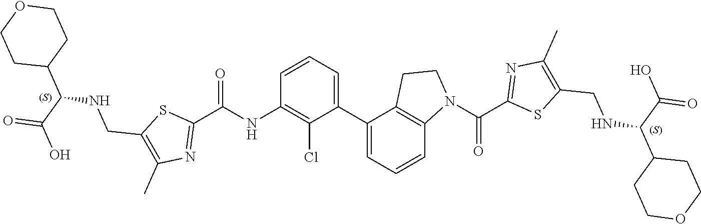 | 837.2 |
| 62 | (S)-1-((2-(3-(1-(5-(((S)-2- carboxypiperidin-1-yl)methyl)-4- methylthiazole-2-carbonyl)indolin-4- yl)phenylcarbamoyl)-4-methylthiazol- 5-yl)methyl)piperidine-2-carboxylic acid | 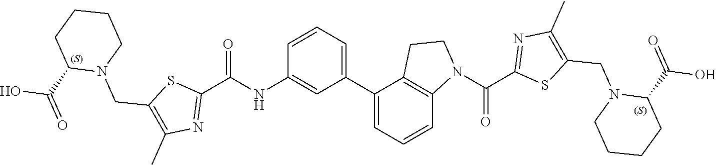 | 743.3 |
| 63 | (S)-2-((2-(3-(1-(5-(((S)- carboxy(tetrahydro-2H-pyran-4- yl)methylamino)methyl)-4- methylthiazole-2-carbonyl)indolin-4- yl)phenylcarbamoyl)-4-methylthiazol- 5-yl)methylamino)-2-(tetrahydro-2H- pyran-4-yl)acetic acid | 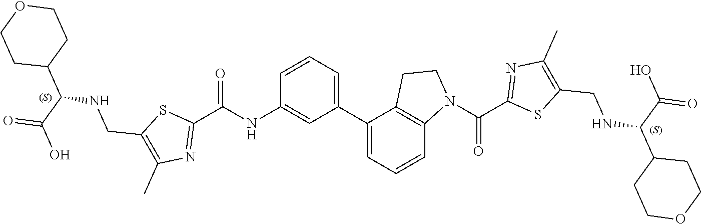 | 803.3 |
| 64 | N,N′-(2,2′-dimethylbiphenyl-3,3′- diyl)bis(3-methyl-4,5,6,7-tetrahydro- 3H-imidazo[4,5-c]pyridine-2- carboxamide) | 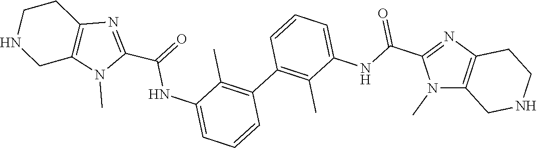 [SMILES: Cc1c(NC(=O)c2nc3c(n2C)CNCC3)cccc1-c1cccc(NC(=O)c2nc3c(n2C)CNCC3)c1C] | 539.3 |
| 65 | 2,2′-((((2,2′-dichloro-[1,1′- biphenyl]-3,3′-diyl)bis (azanediyl))bis(carbonyl))bis(6,7- dihydrothiazolo[5,4-c]pyridine- 2,5(4H)-diyl))diacetic acid | 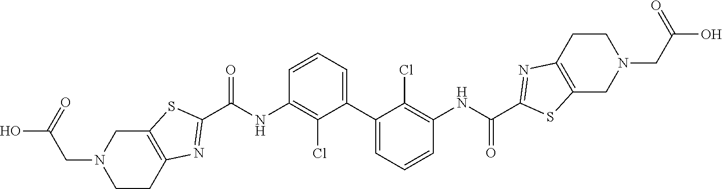 [SMILES: O=C(O)CN1CCc2nc(C(=O)Nc3cccc(-c4cccc(NC(=O)c5nc6c(s5)CN(CC(=O)O)CC6)c4Cl)c3Cl)sc2C1] | 702.1 |
| 66 | N,N′-(2,2′-dichloro-[1,1′- biphenyl]-3,3′-diyl)bis(5-(((2- hydroxyethyl)amino)methyl) thiazole-2-carboxamide) | 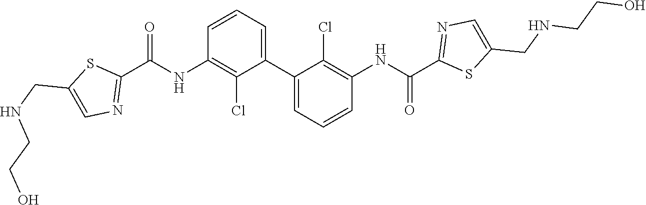 [SMILES: O=C(Nc1cccc(-c2cccc(NC(=O)c3ncc(CNCCO)s3)c2Cl)c1Cl)c1ncc(CNCCO)s1] | 622.1 |
| 67 | (2S,2′S,4R,4′R)-1,1′-(((((2,2′- dichloro-[1,1′-biphenyl]-3,3′- diyl)bis(azanediyl))bis(carbonyl))bis (thiazole-2,5-diyl))bis(methylene)) bis(4-hydroxypyrrolidine-2- carboxylic acid) | 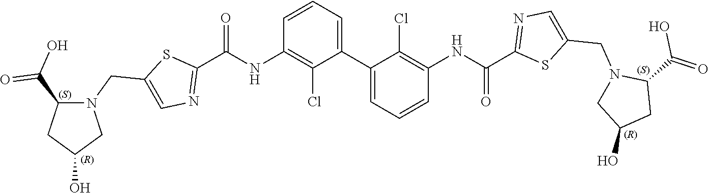 | 762.1 |
| 68 | N,N′-(2,2′-dichloro-[1,1′- biphenyl]-3,3′-diyl)bis(5-(((2,2,2- trifluoroethyl)amino)methyl)thiazole-2- carboxamide) | 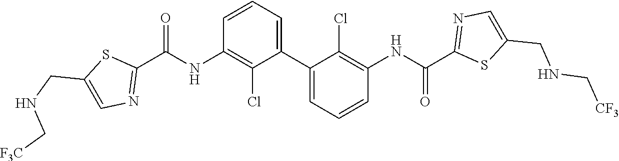 [SMILES: O=C(Nc1cccc(-c2cccc(NC(=O)c3ncc(CNCC(F)(F)F)s3)c2Cl)c1Cl)c1ncc(CNCC(F)(F)F)s1] | 698.0 |
| 69 | ((2-((3′-(5-((((S)-1-carboxy-2- methylpropyl)amino)methyl)thiazole-2- carboxamido)-2,2′-dichloro-[1,1′- biphenyl]-3-yl)carbamoyl)thiazol-5- yl)methyl)-L-valine | 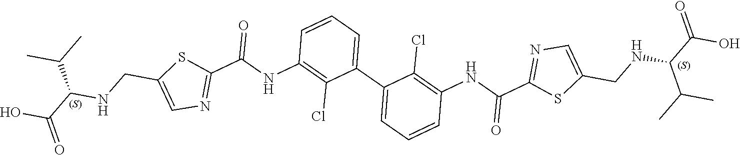 | 734.1 |
| 70 | [4,4′-biindoline]-1,1′-diylbis((5- (((2- hydroxyethyl)amino)methyl)thiazol-2- yl)methanone) | 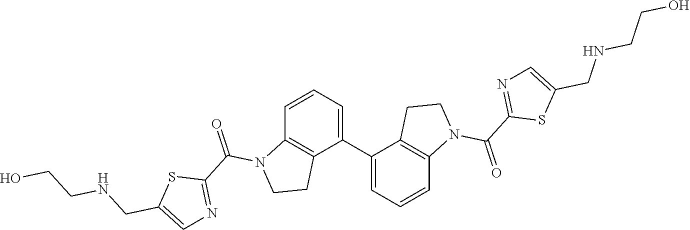 [SMILES: O=C(c1ncc(CNCCO)s1)N1CCc2c(-c3cccc4c3CCN4C(=O)c3ncc(CNCCO)s3)cccc21] | 605.2 |
| 71 | (2S,2′S)-2,2′-((((((2,2′-dichloro- [1,1′-biphenyl]-3,3′- diyl)bis(azanediyl))bis(carbonyl)) bis(thiazole-2,5- diyl))bis(methylene))bis(azanediyl)) bis(2-(tetrahydro-2H-pyran-4- yl)acetic acid) | 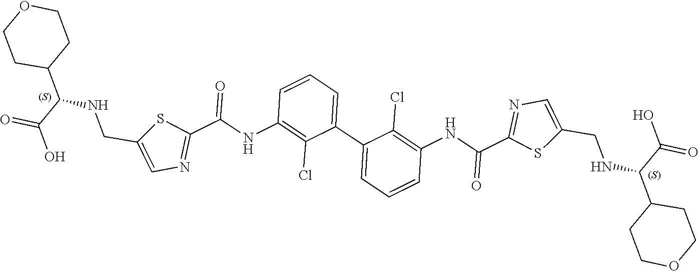 | 818.2 |
| 72 | (S)-1-((2-((3-(1-(5-(((S)-2- carboxypiperidin-1-yl)methyl)thiazole- 2-carbonyl)indolin-4-yl)-2- methylphenyl)carbamoyl)thiazol-5- yl)methyl)piperidine-2-carboxylic acid | 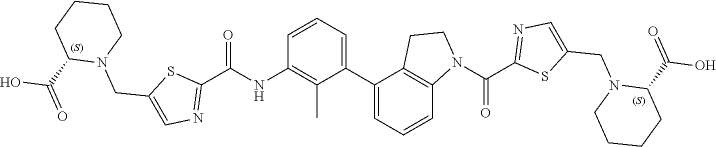 | 729.3 |
| 73 | N,N′-(2,2′-dimethyl-[1,1′-biphenyl]- 3,3′-diyl)bis(5-(((2- hydroxyethyl)amino)methyl)-1,3,4- thiadiazole-2-carboxamide) | 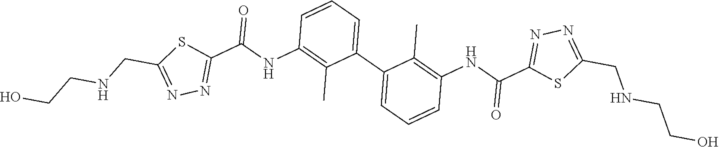 [SMILES: Cc1c(NC(=O)c2nnc(CNCCO)s2)cccc1-c1cccc(NC(=O)c2nnc(CNCCO)s2)c1C] | 583.2 |
| 74 | N,N′-(2,2′-dicyano-[1,1′-biphenyl]-3,3′- diyl)bis(4,5,6,7-tetrahydrothiazolo[5,4- c]pyridine-2-carboxamide) | 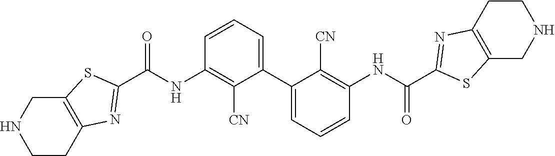 [SMILES: N#Cc1c(NC(=O)c2nc3c(s2)CNCC3)cccc1-c1cccc(NC(=O)c2nc3c(s2)CNCC3)c1C#N] | 567.1 |
| 75 | N,N′-(2,2′-dimethyl-[1,1′-biphenyl]- 3,3′-diyl)bis(5-(3,4-dichlorobenzyl)- 4,5,6,7-tetrahydrothiazolo[5,4- c]pyridine-2-carboxamide) | 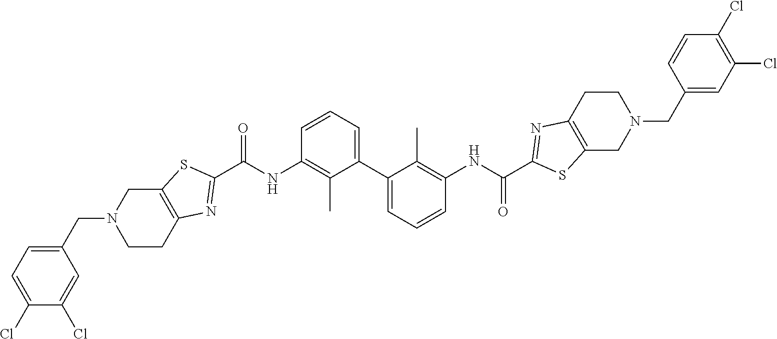 [SMILES: Cc1c(NC(=O)c2nc3c(s2)CN(Cc2ccc(Cl)c(Cl)c2)CC3)cccc1-c1cccc(NC(=O)c2nc3c(s2)CN(Cc2ccc(Cl)c(Cl)c2)CC3)c1C] | 860.1 |
| 76 | N,N′-(2,2′-dimethyl-[1,1′-biphenyl]- 3,3′-diyl)bis(5-(3-hydroxy-2,2- dimethylpropyl)-4,5,6,7- tetrahydrothiazolo[5,4-c]pyridine-2- carboxamide) | 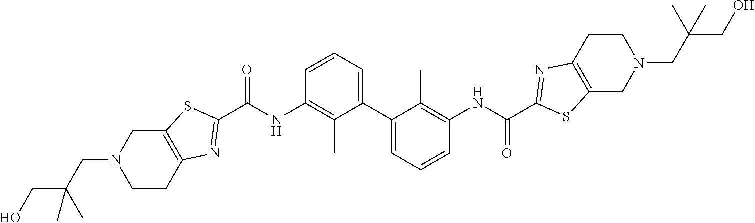 [SMILES: Cc1c(NC(=O)c2nc3c(s2)CN(CC(C)(C)CO)CC3)cccc1-c1cccc(NC(=O)c2nc3c(s2)CN(CC(C)(C)CO)CC3)c1C] | 717.3 |
| 77 | N,N′-(2,2′-dimethyl-[1,1′-biphenyl]- 3,3′-diyl)bis(5-isopropyl-4,5,6,7- tetrahydrothiazolo[5,4-c]pyridine-2- carboxamide) | 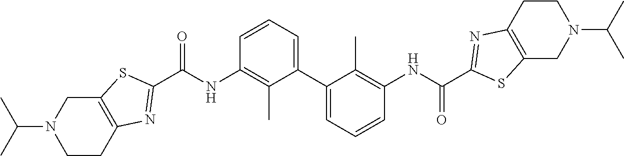 [SMILES: Cc1c(NC(=O)c2nc3c(s2)CN(C(C)C)CC3)cccc1-c1cccc(NC(=O)c2nc3c(s2)CN(C(C)C)CC3)c1C] | 629.3 |
| 78 | 4,4′-((((2,2′-dimethyl-[1,1′- biphenyl]-3,3′- diyl)bis(azanediyl))bis(carbonyl)) bis(6,7-dihydrothiazolo[5,4- c]pyridine-2,5(4H)-diyl))dibutyric acid | 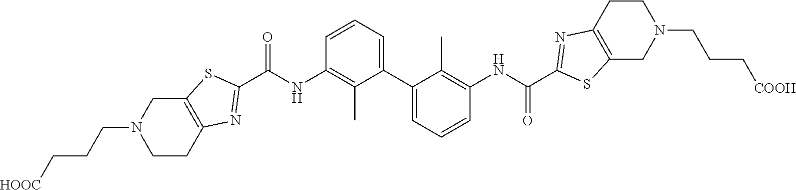 [SMILES: Cc1c(NC(=O)c2nc3c(s2)CN(CCCC(=O)O)CC3)cccc1-c1cccc(NC(=O)c2nc3c(s2)CN(CCCC(=O)O)CC3)c1C] | 717.3 |
| 79 | 2,2′-((((2,2′-dimethyl-[1,1′-biphenyl]- 3,3′- diyl)bis(azanediyl))bis(carbonyl))bis(6, 7-dihydrothiazolo[5,4-c]pyridine- 2,5(4H)-diyl))dipropionic acid | 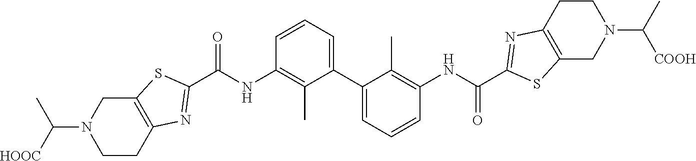 [SMILES: Cc1c(NC(=O)c2nc3c(s2)CN(C(C)C(=O)O)CC3)cccc1-c1cccc(NC(=O)c2nc3c(s2)CN(C(C)C(=O)O)CC3)c1C] | 689.2 |
| 80 | N,N′-(2,2′-dimethyl-[1,1′-biphenyl]- 3,3′-diyl)bis(5-(cyanomethyl)-4,5,6,7- tetrahydrothiazolo[5,4-c]pyridine-2- carboxamide) | 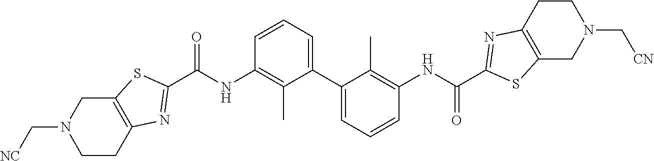 [SMILES: Cc1c(NC(=O)c2nc3c(s2)CN(CC#N)CC3)cccc1-c1cccc(NC(=O)c2nc3c(s2)CN(CC#N)CC3)c1C] | 623.2 |
| 81 | N,N′-(2,2′-dimethyl-[1,1′-biphenyl]- 3,3′-diyl)bis(5-(3-morpholinopropyl)- 4,5,6,7-tetrahydrothiazolo[5,4- c]pyridine-2-carboxamide) | 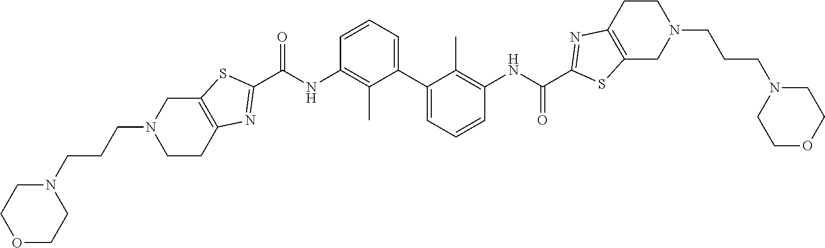 [SMILES: Cc1c(NC(=O)c2nc3c(s2)CN(CCCN2CCOCC2)CC3)cccc1-c1cccc(NC(=O)c2nc3c(s2)CN(CCCN2CCOCC2)CC3)c1C] | 799.4 |
| 82 | N,N′-(2,2′-dimethyl-[1,1′-biphenyl]- 3,3′-diyl)bis(5-(2-hydroxyethyl)- 4,5,6,7-tetrahydrothiazolo[5,4- c]pyridine-2-carboxamide) | 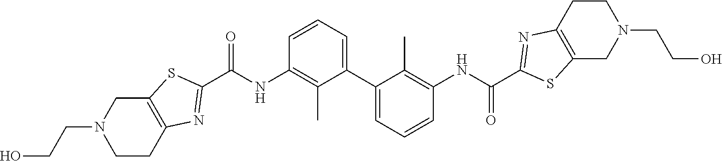 [SMILES: Cc1c(NC(=O)c2nc3c(s2)CN(CCO)CC3)cccc1-c1cccc(NC(=O)c2nc3c(s2)CN(CCO)CC3)c1C] | 633.2 |
| 83 | [4,4′-biindoline]-1,1′-diylbis((5- methyl-4,5,6,7- tetrahydrothiazolo[5,4- c]pyridin-2-yl)methanone) | 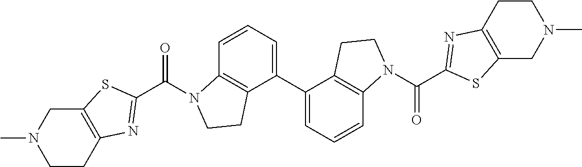 | 597.2 |
| 84 | [4,4′-biindoline]-1,1′-diylbis((5- (2,2,2-trifluoroethyl)-4,5,6,7- tetrahydrothiazolo[5,4-c]pyridin-2- yl)methanone) | 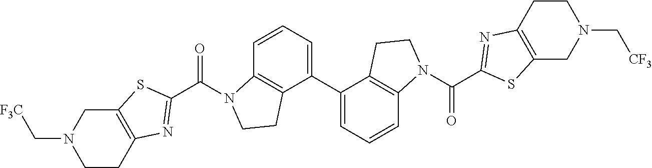 [SMILES: O=C(c1nc2c(s1)CN(CC(F)(F)F)CC2)N1CCc2c(-c3cccc4c3CCN4C(=O)c3nc4c(s3)CN(CC(F)(F)F)CC4)cccc21] | 733.2 |
| 85 | [4,4′-biindoline]-1,1′-diylbis((5-(2,2- difluoroethyl)-4,5,6,7- tetrahydrothiazolo[5,4-c]pyridin-2- yl)methanone) | 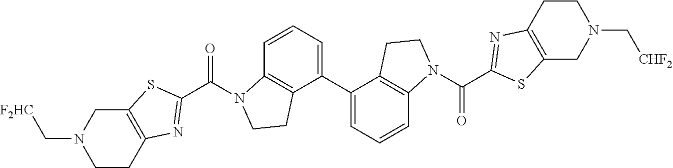 [SMILES: O=C(c1nc2c(s1)CN(CC(F)F)CC2)N1CCc2c(-c3cccc4c3CCN4C(=O)c3nc4c(s3)CN(CC(F)F)CC4)cccc21] | 697.2 |
| 86 | 2,2′-(([4,4′-biindoline]-1,1′- dicarbonyl)bis(6,7- dihydrothiazolo[5,4-c]pyridine- 2,5(4H)-diyl))diacetamide | 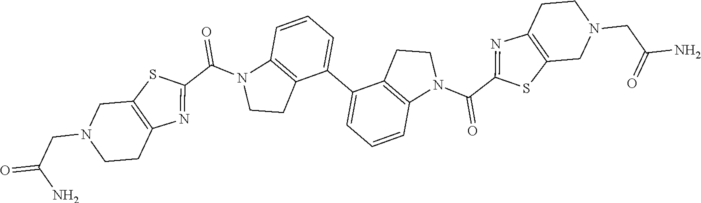 [SMILES: NC(=O)CN1CCc2nc(C(=O)N3CCc4c(-c5cccc6c5CCN6C(=O)c5nc6c(s5)CN(CC(N)=O)CC6)cccc43)sc2C1] | 683.2 |
| 87 | [4,4′-biindoline]-1,1′-diylbis((5-(2- fluoroethyl)-4,5,6,7- tetrahydrothiazolo[5,4-c]pyridin-2- yl)methanone) | 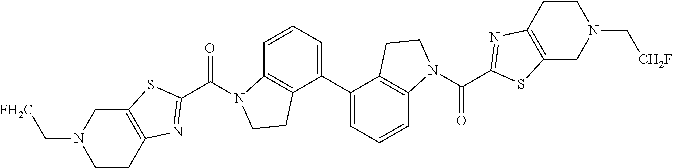 | 661.2 |
| 88 | [4,4′-biindoline]-1,1′-diylbis((5- (oxetan-3-yl)-4,5,6,7- tetrahydrothiazolo[5,4-c]pyridin-2- yl)methanone) | 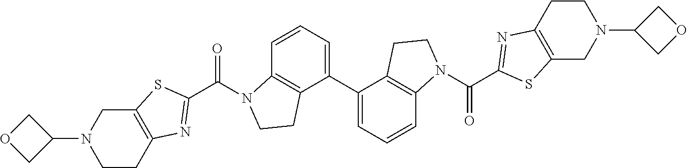 [SMILES: O=C(c1nc2c(s1)CN(C1COC1)CC2)N1CCc2c(-c3cccc4c3CCN4C(=O)c3nc4c(s3)CN(C3COC3)CC4)cccc21] | 681.2 |
| 89 | N,N′-(2,2′-dichloro-[1,1′-biphenyl]-3,3′- diyl)bis(4,5,6,7-tetrahydrothiazolo[5,4- c]pyridine-2-carboxamide) | 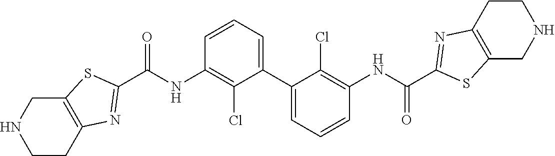 [SMILES: O=C(Nc1cccc(-c2cccc(NC(=O)c3nc4c(s3)CNCC4)c2Cl)c1Cl)c1nc2c(s1)CNCC2] | 586.1 |
| 90 | N,N′-(2,2′-dichloro-[1,1′-biphenyl]-3,3′- diyl)bis(5-(2-amino-2-oxoethyl)- 4,5,6,7-tetrahydrothiazolo[5,4- c]pyridine-2-carboxamide) | 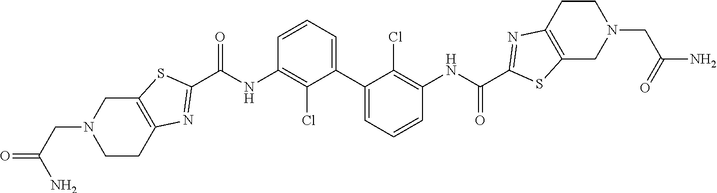 [SMILES: NC(=O)CN1CCc2nc(C(=O)Nc3cccc(-c4cccc(NC(=O)c5nc6c(s5)CN(CC(N)=O)CC6)c4Cl)c3Cl)sc2C1] | 700.1 |
| 91 | N,N′-(2,2′-dichloro-[1,1′-biphenyl]-3,3′- diyl)bis(5-(2-hydroxyethyl)-4,5,6,7- tetrahydrothiazolo[5,4-c]pyridine-2- carboxamide) | 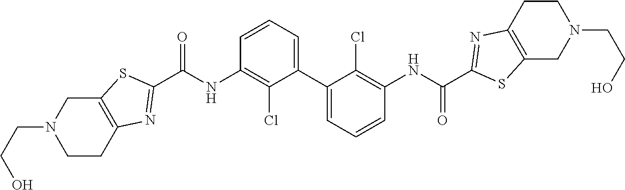 [SMILES: O=C(Nc1cccc(-c2cccc(NC(=O)c3nc4c(s3)CN(CCO)CC4)c2Cl)c1Cl)c1nc2c(s1)CN(CCO)CC2] | 674.1 |
| 92 | N,N′-(2,2′-dichloro-[1,1′-biphenyl]-3,3′- diyl)bis(5-(oxetan-3-yl)-4,5,6,7- tetrahydrothiazolo[5,4-c]pyridine-2- carboxamide) | 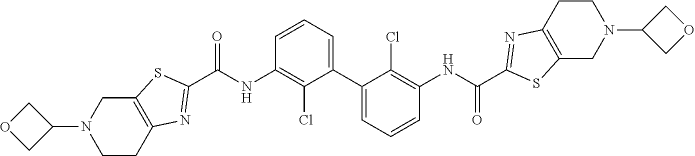 [SMILES: O=C(Nc1cccc(-c2cccc(NC(=O)c3nc4c(s3)CN(C3COC3)CC4)c2Cl)c1Cl)c1nc2c(s1)CN(C1COC1)CC2] | 698.1 |
| 93 | N,N′-(5,5′-dichloro-2,2′-dimethyl-[1,1′- biphenyl]-3,3′-diyl)bis(4,5,6,7- tetrahydrothiazolo[5,4-c]pyridine-2- carboxamide) | 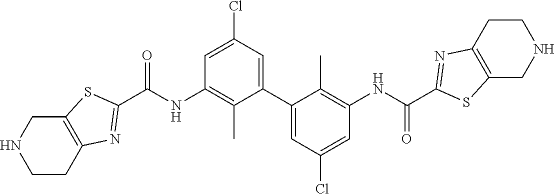 [SMILES: Cc1c(NC(=O)c2nc3c(s2)CNCC3)cc(Cl)cc1-c1cc(Cl)cc(NC(=O)c2nc3c(s2)CNCC3)c1C] | 614.1 |
| 94 | N,N′-(2,2′,4,4′-tetramethyl-[1,1′- biphenyl]-3,3′-diyl)bis(4,5,6,7- tetrahydrothiazolo[5,4-c]pyridine-2- carboxamide) | 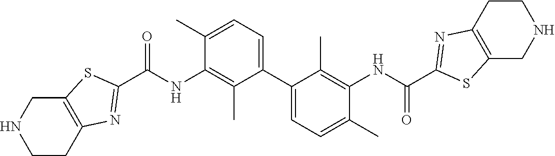 [SMILES: Cc1ccc(-c2ccc(C)c(NC(=O)c3nc4c(s3)CNCC4)c2C)c(C)c1NC(=O)c1nc2c(s1)CNCC2] | 573.2 |
| 95 | N-(5-fluoro-2-methyl-3-(1-(4,5,6,7- tetrahydrothiazolo[5,4-c]pyridine-2- carbonyl)indolin-4-yl)phenyl)-4,5,6,7- tetrahydrothiazolo[5,4-c]pyridine-2- carboxamide | 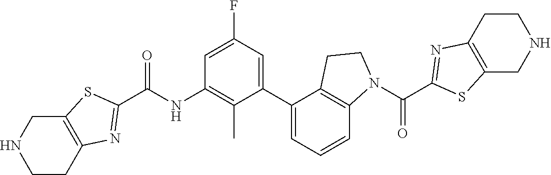 [SMILES: Cc1c(NC(=O)c2nc3c(s2)CNCC3)cc(F)cc1-c1cccc2c1CCN2C(=O)c1nc2c(s1)CNCC2] | 575.2 |
| 96 | N,N′-(5,5′-difluoro-2,2′-dimethyl-[1,1′- biphenyl]-3,3′-diyl)bis(4,5,6,7- tetrahydrothiazolo[5,4-c]pyridine-2- carboxamide) | 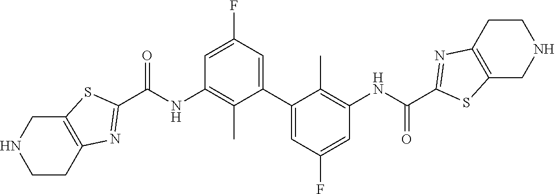 [SMILES: Cc1c(NC(=O)c2nc3c(s2)CNCC3)cc(F)cc1-c1cc(F)cc(NC(=O)c2nc3c(s2)CNCC3)c1C] | 581.2 |
| 97 | N-(2,2′-dichloro-3′-(5-(((2- hydroxyethyl)amino)methyl)thiazole-2- carboxamido)-[1,1′-biphenyl]-3-yl)- 4,5,6,7-tetrahydrothiazolo[5,4- c]pyridine-2-carboxamide | 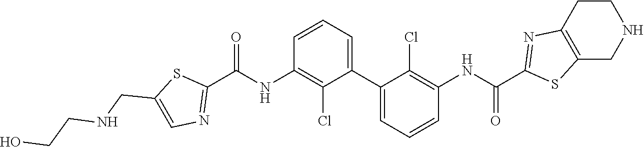 [SMILES: O=C(Nc1cccc(-c2cccc(NC(=O)c3nc4c(s3)CNCC4)c2Cl)c1Cl)c1ncc(CNCCO)s1] | 604.1 |
| 98 | N-(2,2′-dichloro-3′-((3-(((2- hydroxyethyl)amino)methyl)-1,7- naphthyridin-8-yl)amino)-[1,1′- biphenyl]-3-yl)-5-(((2- hydroxyethyl)amino)methyl)thiazole-2- carboxamide | 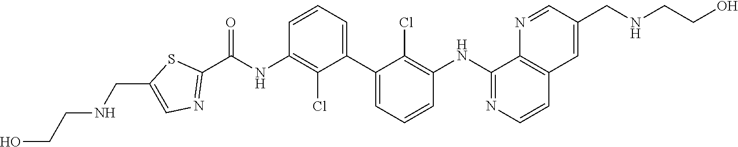 [SMILES: O=C(Nc1cccc(-c2cccc(Nc3nccc4cc(CNCCO)cnc34)c2Cl)c1Cl)c1ncc(CNCCO)s1] | 639.1 |
| 99 | (5-(((2- hydroxyethyl)amino)methyl)thiazol-2- yl)(1′-(4,5,6,7-tetrahydrothiazolo[5,4- c]pyridine-2-carbonyl)-[4,4′-biindolin]- 1-yl)methanone | 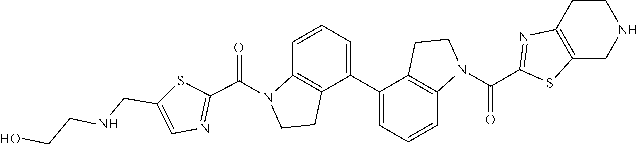 [SMILES: O=C(c1ncc(CNCCO)s1)N1CCc2c(-c3cccc4c3CCN4C(=O)c3nc4c(s3)CNCC4)cccc21] | 587.2 |
| 100 | (1′-(3-(((2- hydroxyethyl)amino)methyl)-1,7- naphthyridin-8-yl)-[4,4′-biindolin]-1- yl)(4,5,6,7-tetrahydrothiazolo[5,4- c]pyridin-2-yl)methanone | 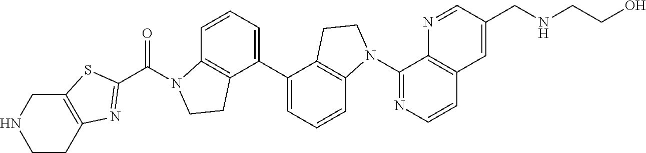 [SMILES: O=C(c1nc2c(s1)CNCC2)N1CCc2c(-c3cccc4c3CCN4c3nccc4cc(CNCCO)cnc34)cccc21] | 604.2 |
| 101 | N-(2-chloro-3-(1-(4,5,6,7- tetrahydrothiazolo[5,4-c]pyridine-2- carbonyl)indolin-4-yl)phenyl)-5-(((2- hydroxyethyl)amino)methyl)thiazole-2- carboxamide | 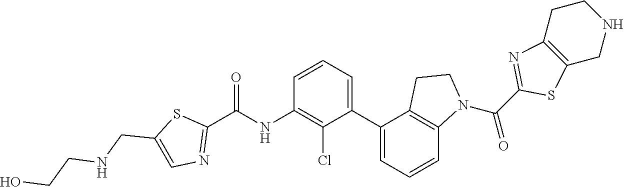 [SMILES: O=C(Nc1cccc(-c2cccc3c2CCN3C(=O)c2nc3c(s2)CNCC3)c1Cl)c1ncc(CNCCO)s1] | 596.1 |
| 102 | dimethyl 2,2′-(([4,4′-biindoline]-1,1′- dicarbonyl)bis(6,7-dihydrothiazolo[5,4- c]pyridine-2,5(4H)-diyl))diacetate | 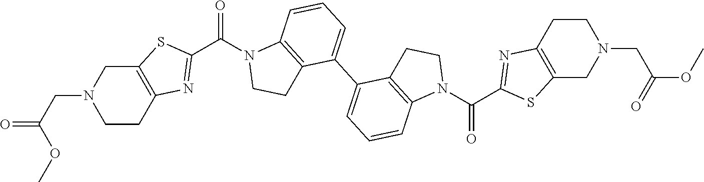 [SMILES: COC(=O)CN1CCc2nc(C(=O)N3CCc4c(-c5cccc6c5CCN6C(=O)c5nc6c(s5)CN(CC(=O)OC)CC6)cccc43)sc2C1] | 713.2 |
| 103 | 2,2′-(([4,4′-biindoline]-1,1′- dicarbonyl)bis(6,7-dihydrothiazolo[5,4- c]pyridine-2,5(4H)-diyl))diacetic acid | 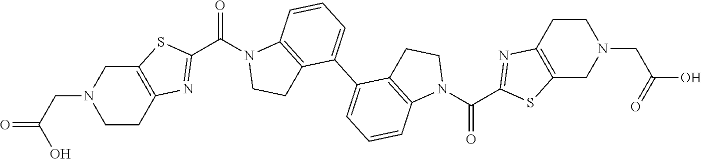 [SMILES: O=C(O)CN1CCc2nc(C(=O)N3CCc4c(-c5cccc6c5CCN6C(=O)c5nc6c(s5)CN(CC(=O)O)CC6)cccc43)sc2C1] | 685.2 |
| 104 | 2,2′-(([4,4′-biindoline]-1,1′- dicarbonyl)bis(6,7-dihydrothiazolo[5,4- c]pyridine-2,5(4H)-diyl))diacetamide | 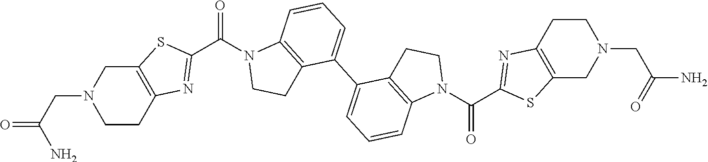 [SMILES: NC(=O)CN1CCc2nc(C(=O)N3CCc4c(-c5cccc6c5CCN6C(=O)c5nc6c(s5)CN(CC(N)=O)CC6)cccc43)sc2C1] | 683.2 |
| 105 | 2,2′-(([4,4′-biindoline]-1,1′- dicarbonyl)bis(6,7-dihydrothiazolo[5,4- c]pyridine-2,5(4H)-diyl))bis(N- methylacetamide) | 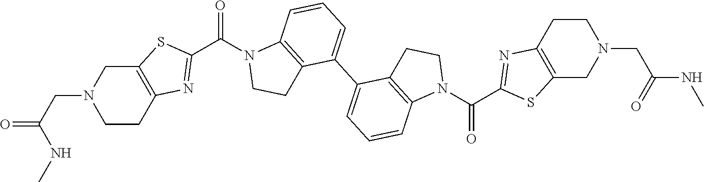 [SMILES: CNC(=O)CN1CCc2nc(C(=O)N3CCc4c(-c5cccc6c5CCN6C(=O)c5nc6c(s5)CN(CC(=O)NC)CC6)cccc43)sc2C1] | 711.3 |
| 106 | 2,2′-(([4,4′-biindoline]-1,1′- dicarbonyl)bis(6,7-dihydrothiazolo[5,4- c]pyridine-2,5(4H)-diyl))bis(N,N- dimethylacetamide) | 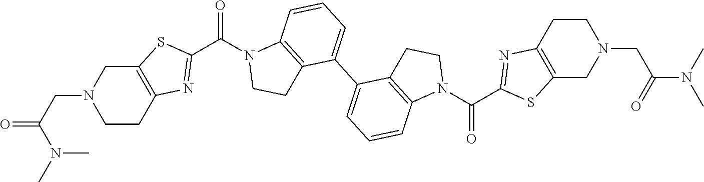 [SMILES: CN(C)C(=O)CN1CCc2nc(C(=O)N3CCc4c(-c5cccc6c5CCN6C(=O)c5nc6c(s5)CN(CC(=O)N(C)C)CC6)cccc43)sc2C1] | 739.3 |
| 107 | 2-(2-(1′-(5-(2-amino-2-oxoethyl)- 4,5,6,7-tetrahydrothiazolo[5,4- c]pyridine-2-carbonyl)-[4,4′- biindoline]-1-carbonyl)-6,7- dihydrothiazolo[5,4-c]pyridin-5(4H)- yl)acetic acid | 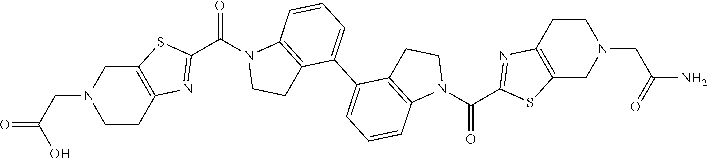 [SMILES: NC(=O)CN1CCc2nc(C(=O)N3CCc4c(-c5cccc6c5CCN6C(=O)c5nc6c(s5)CN(CC(=O)O)CC6)cccc43)sc2C1] | 684.2 |
| 108 | 2-(2-(1′-(5-(2-(methylamino)-2- oxoethyl)-4,5,6,7- tetrahydrothiazolo[5,4-c]pyridine-2- carbonyl)-[4,4′-biindoline]-1-carbonyl)- 6,7-dihydrothiazolo[5,4-c]pyridin- 5(4H)-yl)acetic acid | 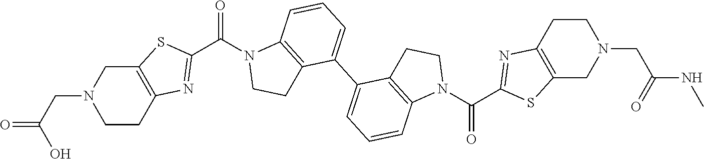 [SMILES: CNC(=O)CN1CCc2nc(C(=O)N3CCc4c(-c5cccc6c5CCN6C(=O)c5nc6c(s5)CN(CC(=O)O)CC6)cccc43)sc2C1] | 698.2 |
| 109 | 2-(2-(1′-(5-(2-(dimethylamino)-2- oxoethyl)-4,5,6,7- tetrahydrothiazolo[5,4-c]pyridine-2- carbonyl)-[4,4′-biindoline]-1-carbonyl)- 6,7-dihydrothiazolo[5,4-c]pyridin- 5(4H)-yl)acetic acid | 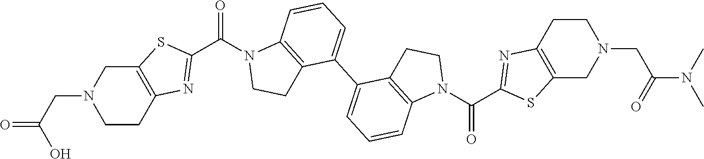 [SMILES: CN(C)C(=O)CN1CCc2nc(C(=O)N3CCc4c(-c5cccc6c5CCN6C(=O)c5nc6c(s5)CN(CC(=O)O)CC6)cccc43)sc2C1] | 712.2 |
| 110 | 2,2′-(([4,4′-biindoline]-1,1′- dicarbonyl)bis(5,6,7,8-tetrahydro-4H- 5,8-epiminocyclohepta[d]thiazole-2,9- diyl))diacetic acid | 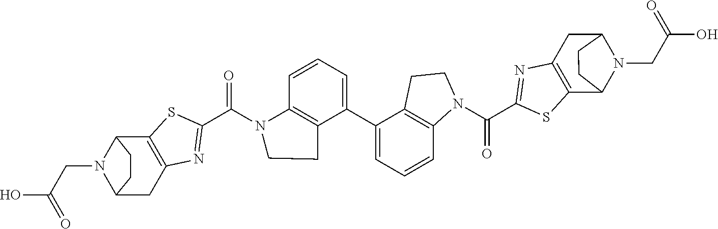 [SMILES: O=C(O)CN1C2CCC1c1sc(C(=O)N3CCc4c(-c5cccc6c5CCN6C(=O)c5nc6c(s5)C5CCC(C6)N5CC(=O)O)cccc43)nc1C2] | 737.2 |
| 111 | 2-(2-(1′-(9-(2-ammo-2-oxoethyl)- 5,6,7,8-tetrahydro-4H-5,8- epiminocyclohepta[d]thiazole-2- carbonyl)-[4,4′-biindoline]-1-carbonyl)- 5,6,7,8-tetrahydro-4H-5,8- epiminocyclohepta[d]thiazol-9- yl)acetic acid | 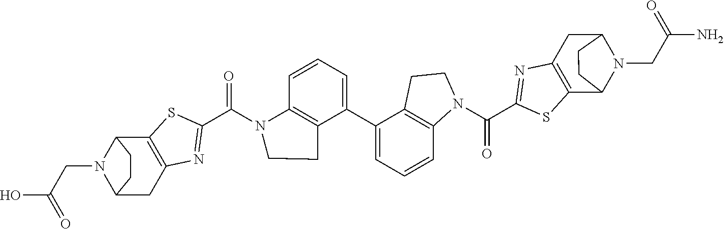 [SMILES: NC(=O)CN1C2CCC1c1sc(C(=O)N3CCc4c(-c5cccc6c5CCN6C(=O)c5nc6c(s5)C5CCC(C6)N5CC(=O)O)cccc43)nc1C2] | 736.2 |
| 112 | [4,4′-biindoline]-1,1′-diylbis((5- ((dimethylamino)methyl)thiazol-2- yl)methanone) | 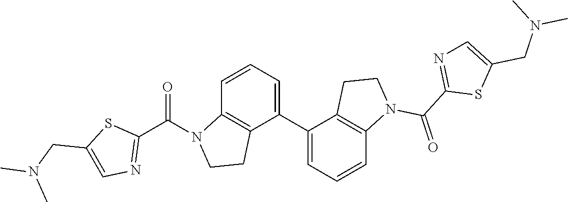 | 573.2 |
| 113 | [4,4′-biindoline]-1,1′-diylbis((5- ((methylamino)methyl)thiazol-2- yl)methanone) | 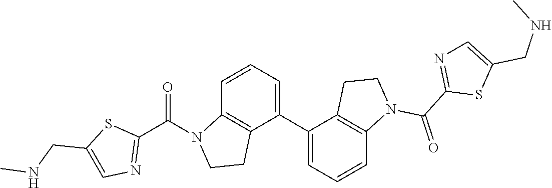 [SMILES: CNCc1cnc(C(=O)N2CCc3c(-c4cccc5c4CCN5C(=O)c4ncc(CNC)s4)cccc32)s1] | 545.2 |
| 114 | N-(3-(1-(5-((dimethylamino)methyl)-4- methylthiazole-2-carbonyl)indolin-4- y])phenyl)-5-(((2- hydroxyethyl)(methyl)amino)methyl) thiazole-2-carboxamide | 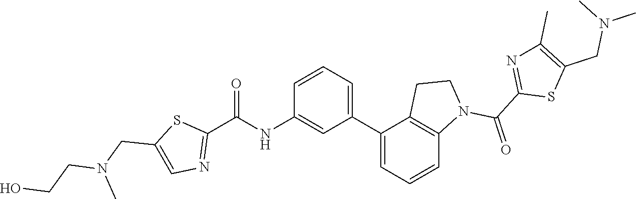 [SMILES: Cc1nc(C(=O)N2CCc3c(-c4cccc(NC(=O)c5ncc(CN(C)CCO)s5)c4)cccc32)sc1CN(C)C] | 591.2 |
| 115 | (5-((dimethylamino)methyl)-4- methylthiazol-2-yl)(1′-(5-(((2- hydroxyethyl)(methyl)amino)methyl) thiazole-2-carbonyl)-[4,4′-biindolin]- 1-yl)methanone | 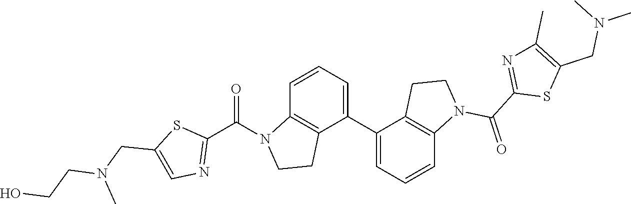 [SMILES: Cc1nc(C(=O)N2CCc3c(-c4cccc5c4CCN5C(=O)c4ncc(CN(C)CCO)s4)cccc32)sc1CN(C)C] | 617.2 |
| 116 | [4,4′-biindoline]-1,1′-diylbis((5-(((2- hydroxyethyl)(methyl)amino)methyl) thiazol-2-yl)methanone) | 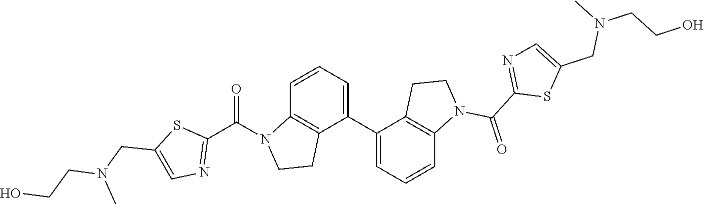 [SMILES: CN(CCO)Cc1cnc(C(=O)N2CCc3c(-c4cccc5c4CCN5C(=O)c4ncc(CN(C)CCO)s4)cccc32)s1] | 633.2 |
| 117 | N,N′-((([4,4′-biindoline]-1,1′- dicarbonyl)bis(6,7-dihydrothiazolo[5,4- c]pyridine-2,5(4H)-diyl))bis(ethane- 2,1-diyl))dimethanesulfonamide | 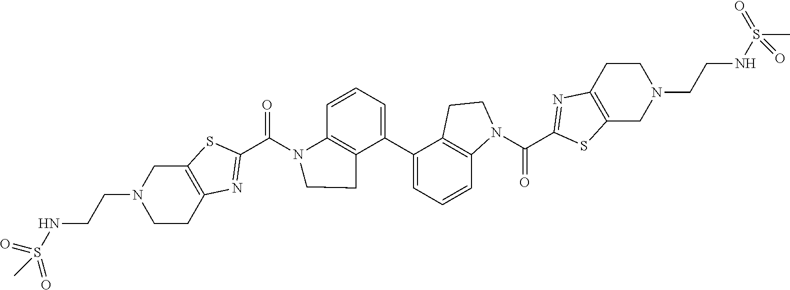 [SMILES: CS(=O)(=O)NCCN1CCc2nc(C(=O)N3CCc4c(-c5cccc6c5CCN6C(=O)c5nc6c(s5)CN(CCNS(C)(=O)=O)CC6)cccc43)sc2C1] | 811.2 |
| 118 | N,N′-((([4,4′-biindoline]-1,1′- dicarbonyl)bis(6,7-dihydrothiazolo[5,4- c]pyridine-2,5(4H)-diyl))bis(ethane- 2,1-diyl))bis(N- methylmethanesulfonamide) | 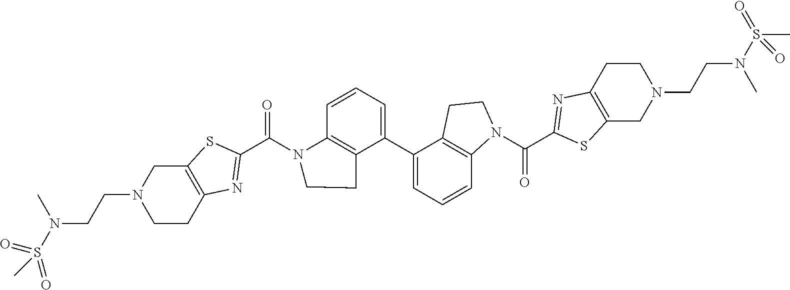 [SMILES: CN(CCN1CCc2nc(C(=O)N3CCc4c(-c5cccc6c5CCN6C(=O)c5nc6c(s5)CN(CCN(C)S(C)(=O)=O)CC6)cccc43)sc2C1)S(C)(=O)=O] | 839.3 |
| 119 | 2,2′-(([4,4′-biindoline]-1,1′- dicarbonyl)bis(6,7-dihydrothiazolo[4,5- c]pyridine-2,5(4H)-diyl))diacetic acid | 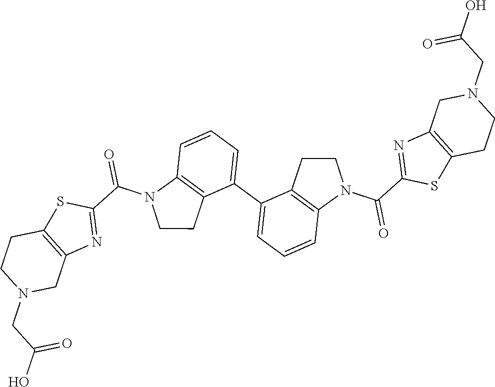 [SMILES: O=C(O)CN1CCc2sc(C(=O)N3CCc4c(-c5cccc6c5CCN6C(=O)c5nc6c(s5)CCN(CC(=O)O)C6)cccc43)nc2C1] | 685.2 |
| 120 | [4,4′-biindoline]-1,1′-diylbis((5-(2-(2- hydroxyethoxy)ethyl)-4,5,6,7- tetrahydrothiazolo[5,4-c]pyridin-2- yl)methanone) | 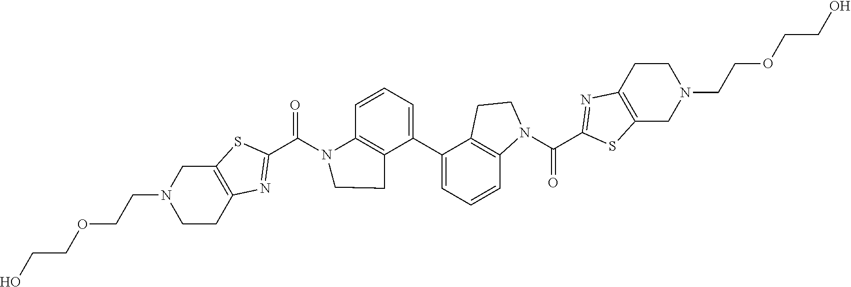 [SMILES: O=C(c1nc2c(s1)CN(CCOCCO)CC2)N1CCc2c(-c3cccc4c3CCN4C(=O)c3nc4c(s3)CN(CCOCCO)CC4)cccc21] | 745.3 |
| 121 | ((([4,4′-biindoline]-1,1′- dicarbonyl)bis(6,7-dihydrothiazolo[5,4- c]pyridine-2,5(4H)-diyl))bis(propane- 3,1-diyl))diboronic acid | 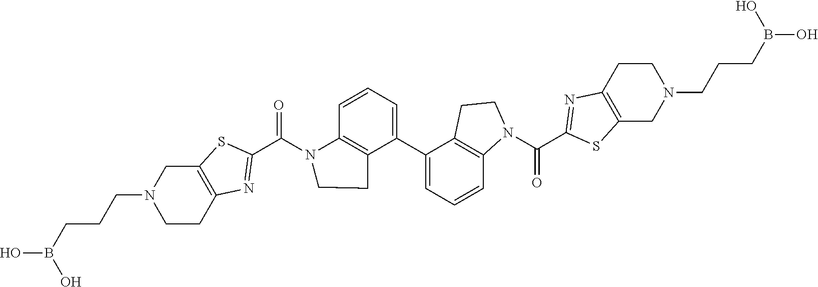 [SMILES: O=C(c1nc2c(s1)CN(CCCB(O)O)CC2)N1CCc2c(-c3cccc4c3CCN4C(=O)c3nc4c(s3)CN(CCCB(O)O)CC4)cccc21] | 739.3 |
| 122 | tetramethyl ((([4,4′-biindoline]-1,1′- dicarbonyl)bis(6,7-dihydrothiazolo[5,4- c]pyridine-2,5(4H)-diyl))bis(propane- 3,1-diyl))diboronate | 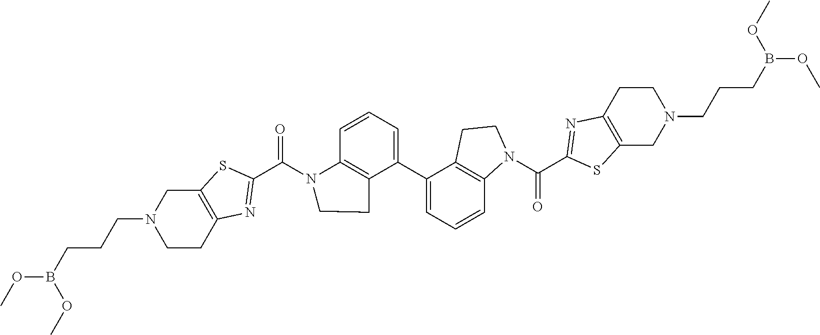 | 795.4 |
| 123 | dimethyl ((2-(1′-(5- ((dimethoxyphosphoryl)methyl)- 4,5,6,7-tetrahydrothiazolo[5,4- c]pyridine-2-carbonyl)-[4,4′- biindoline]-1-carbonyl)-6,7- dihydrothiazolo[5,4-c]pyridin-5(4H)- yl)methyl)phosphonate | 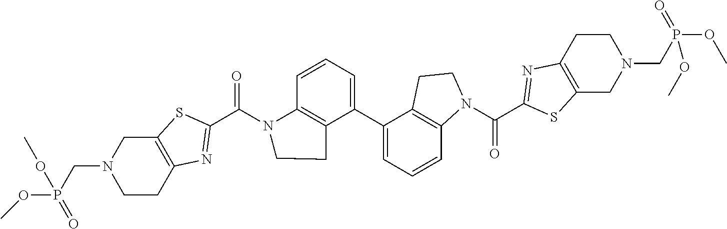 | 813.2 |
| 124 | dimethyl (2-(2-(1′-(5-(2- (dimethoxyphosphoryl)ethyl)-4,5,6,7- tetrahydrothiazolo[5,4-c]pyridine-2- carbonyl)-[4,4′-biindoline]-1-carbonyl)- 6,7-dihydrothiazolo[5,4-c]pyridin- 5(4H)-yl)ethyl)phosphonate | 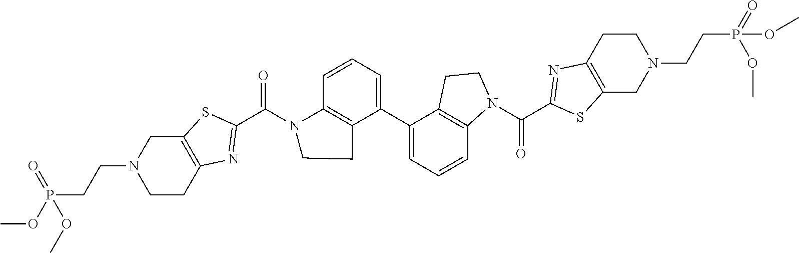 | 841.2 |
| 125 | [4,4′-biindoline]-1,1′-diylbis((5-((2H- tetrazol-5-yl)methyl)-4,5,6,7- tetrahydrothiazolo[5,4-c]pyridin-2- yl)methanone) | 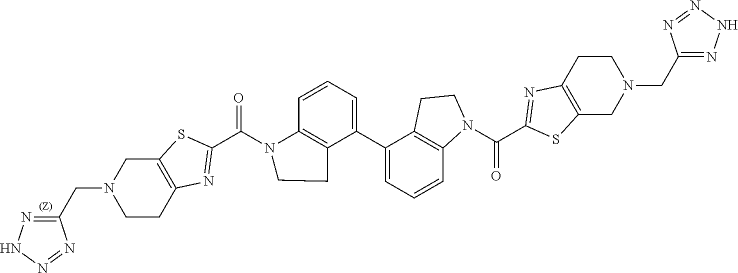 [SMILES: O=C(c1nc2c(s1)CN(Cc1nn[nH]n1)CC2)N1CCc2c(-c3cccc4c3CCN4C(=O)c3nc4c(s3)CN(Cc3nn[nH]n3)CC4)cccc21] | 733.2 |
| 126 | N,N′-((([4,4′-biindoline]-1,1′- dicarbonyl)bis(5,6,7,8-tetrahydro-4H- 5,8-epiminocyclohepta[d]thiazole-2,9- diyl))bis(ethane-2,1- diyl))dimethanesulfonamide | 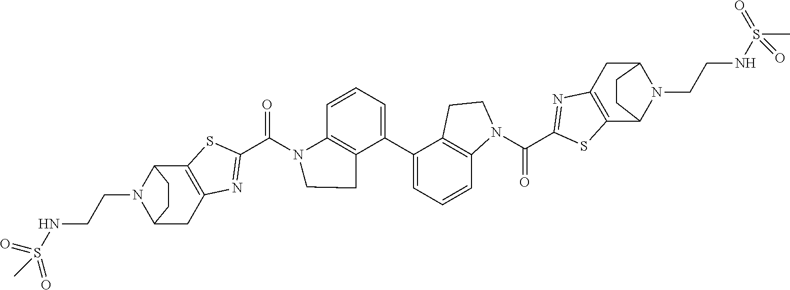 [SMILES: CS(=O)(=O)NCCN1C2CCC1c1sc(C(=O)N3CCc4c(-c5cccc6c5CCN6C(=O)c5nc6c(s5)C5CCC(C6)N5CCNS(C)(=O)=O)cccc43)nc1C2] | 863.3 |
| 127 | 2,2′-(([4,4′-biindoline]-1,1′- dicarbonyl)bis(6,7-dihydrothiazolo[5,4- c]pyridine-2,5(4H)-diyl))bis(N- (methylsulfonyl)propanamide) | 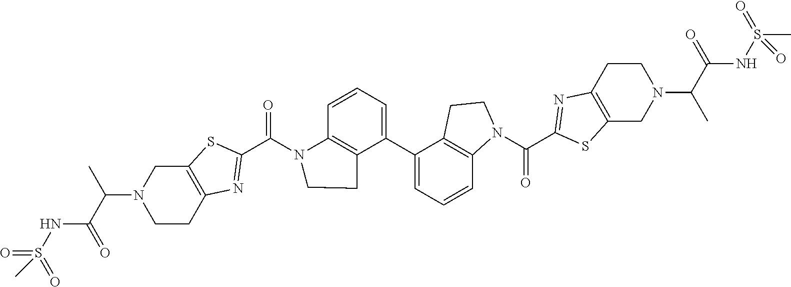 [SMILES: CC(C(=O)NS(C)(=O)=O)N1CCc2nc(C(=O)N3CCc4c(-c5cccc6c5CCN6C(=O)c5nc6c(s5)CN(C(C)C(=O)NS(C)(=O)=O)CC6)cccc43)sc2C1] | 867.2 |
| 128 | 2,2′-(([4,4′-biindoline]-1,1′- dicarbonyl)bis(6,7-dihydrothiazolo[5,4- c]pyridine-2,5(4H)-diyl))bis(2-methyl- N-(methylsulfonyl)propanamide) | 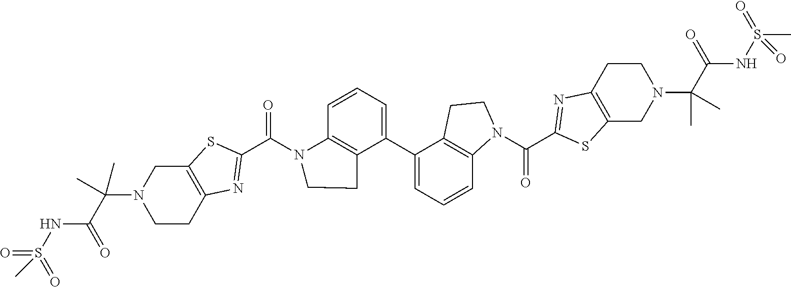 [SMILES: CC(C)(C(=O)NS(C)(=O)=O)N1CCc2nc(C(=O)N3CCc4c(-c5cccc6c5CCN6C(=O)c5nc6c(s5)CN(C(C)(C)C(=O)NS(C)(=O)=O)CC6)cccc43)sc2C1] | 895.2 |
| 129 | 1,1′-(([4,4′-biindoline]-1,1′- dicarbonyl)bis(6,7-dihydrothiazolo[5,4- c]pyridine-2,5(4H)-diyl))bis(N- (methylsulfonyl)cyclopropane-1- carboxamide) | 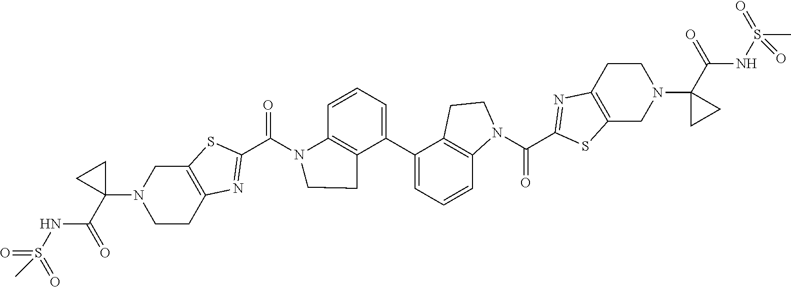 [SMILES: CS(=O)(=O)NC(=O)C1(N2CCc3nc(C(=O)N4CCc5c(-c6cccc7c6CCN7C(=O)c6nc7c(s6)CN(C6(C(=O)NS(C)(=O)=O)CC6)CC7)cccc54)sc3C2)CC1] | 891.2 |
| 130 | dimethyl ((([4,4′-biindoline]-1,1′- dicarbonyl)bis(6,7-dihydrothiazolo[5,4- c]pyridine-2,5(4H)-diyl))bis(ethane- 2,1-diyl))dicarbamate | 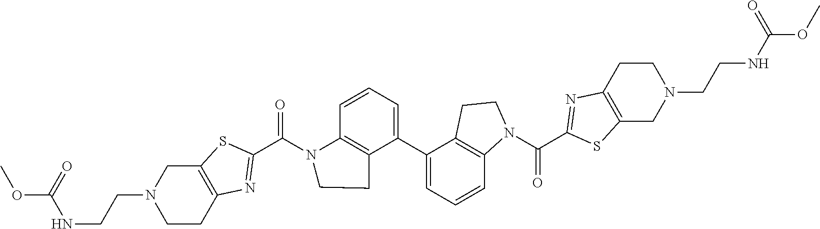 | 771.3 |
| 131 | dimethyl (((([4,4′-biindoline]-1,1′- dicarbonyl)bis(6,7-dihydrothiazolo[5,4- c]pyridine-2,5(4H)- diyl))bis(cyclopropane-1,1- diyl))bis(methylene))dicarbamate | 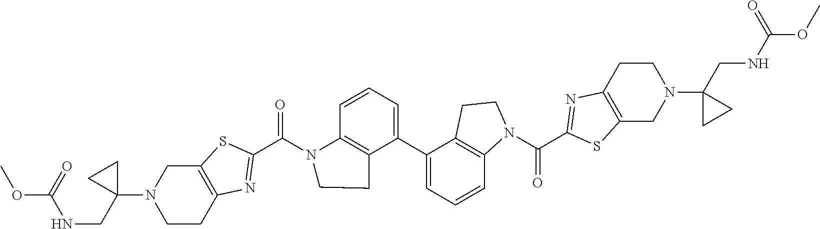 | 823.3 |
| 132 | dimethyl (((([4,4′-biindoline]-1,1′- dicarbonyl)bis(6,7-dihydrothiazolo[5,4- c]pyridine-2,5(4H)- diyl))bis(methylene))bis(cyclopropane- 1,1-diyl))dicarbamate | 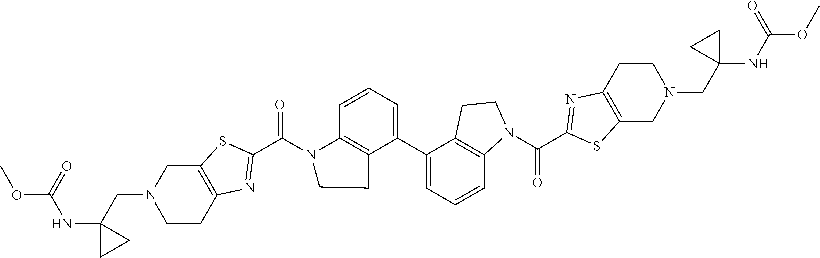 | 823.3 |
| 133 | 1,1′-((([4,4′-biindoline]-1,1′- dicarbonyl)bis(6,7-dihydrothiazolo[5,4- c]pyridine2,5(4H)-diyl))bis(ethane-2,1- diyl))bis(thiourea) | 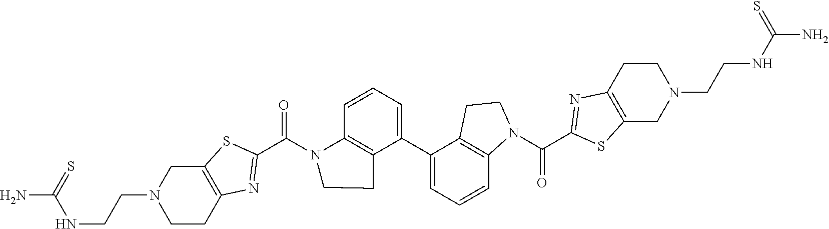 [SMILES: NC(=S)NCCN1CCc2nc(C(=O)N3CCc4c(-c5cccc6c5CCN6C(=O)c5nc6c(s5)CN(CCNC(N)=S)CC6)cccc43)sc2C1] | 773.2 |
| 134 | 1,1′-((([4,4′-biindoline]-1,1′- dicarbonyl)bis(6,7-dihydrothiazolo[5,4- c]pyridine-2,5(4H)-diyl))bis(ethane- 2,1-diyl))diurea | 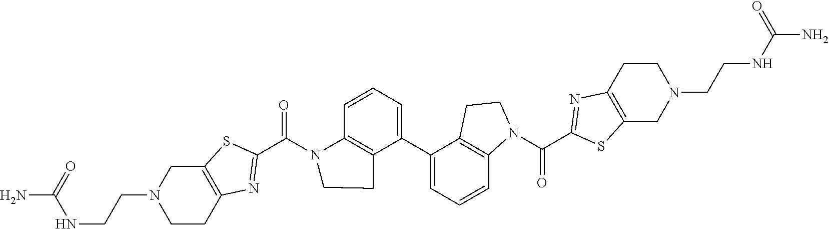 [SMILES: NC(=O)NCCN1CCc2nc(C(=O)N3CCc4c(-c5cccc6c5CCN6C(=O)c5nc6c(s5)CN(CCNC(N)=O)CC6)cccc43)sc2C1] | 741.2 |
| 135 | dimethyl 2,2′-(((([4,4′-biindoline]-1,1′- dicarbonyl)bis(6,7-dihydrothiazolo[5,4- c]pyridine-2,5(4H)- diyl))bis(methylene))bis(1H-1,2,3- triazole-4,1-diyl))diacetate | 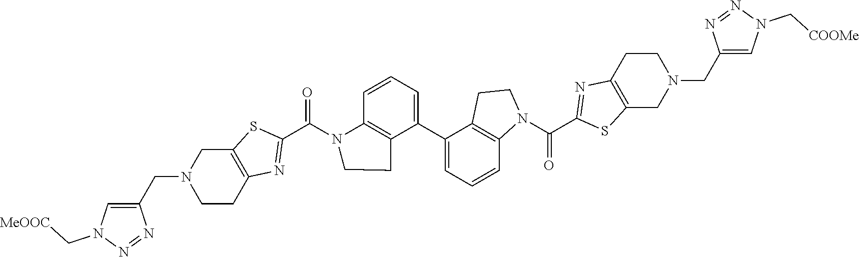 [SMILES: COC(=O)Cn1cc(CN2CCc3nc(C(=O)N4CCc5c(-c6cccc7c6CCN7C(=O)c6nc7c(s6)CN(Cc6cn(CC(=O)OC)nn6)CC7)cccc54)sc3C2)nn1] | 875.3 |
| 136 | [4,4′-biindoline]-1,1′-diylbis((5,6,7,8- tetrahydro-[1,2,4]triazolo[1,5- a]pyrazin-2-yl)methanone) | 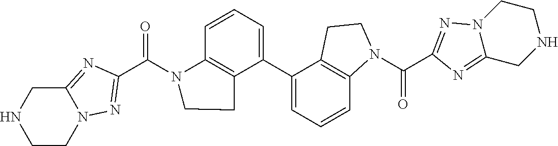 [SMILES: O=C(c1nc2n(n1)CCNC2)N1CCc2c(-c3cccc4c3CCN4C(=O)c3nc4n(n3)CCNC4)cccc21] | 537.2 |
| 137 | (5-((l2-azanyl)methyl)-1H-1,2,4- triazol-3-yl)(1′-(5-(aminomethyl)-1H- 1,2,4-triazole-3-carbonyl)-[4,4′- biindolin]-1-yl)methanone | 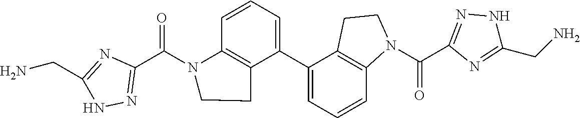 [SMILES: NCc1nc(C(=O)N2CCc3c(-c4cccc5c4CCN5C(=O)c4n[nH]c(CN)n4)cccc32)n[nH]1] | 485.2 |

The present invention also exemplarily provides a method of preparing other compounds, for example, compound 6.

### Example 6 Synthesis of Compound 6

### Step 1: Preparing of 8-chloro-3-vinyl-1,7-naphthyridine

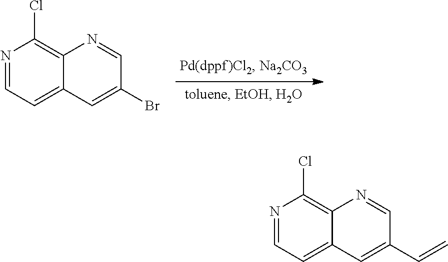

To a solution of 3-bromo-8-chloro-1,7-naphthyridine (2.43 g) in toluene (30 mL), EtOH (10 mL), and 10% Na₂CO₃ aq. (10 mL) Pd(dppf)Cl₂.DCM (420 mg) was added. 4,4,5,5-tetramethyl-2-vinyl-1,3,2-dioxaborolane (3.1 g) was added dropwise under N₂ protection. The mixture was allowed to stir at 100° C. for 16 h. The reaction was quenched by H₂O (50 mL) and extracted by EtOAc for 3 times. Organic layer was combined and washed with brine. The resulting solution was concentrated and purified by silicagel (eluting with hexane-EtOAc using a gradient from 8:1 to 5:1) to afford 8-chloro-3-vinyl-1,7-naphthyridine (1.1 g) as a brown solid.

### Step 2: Preparing of (8-chloro-1,7-naphthyridin-3-yl)methanol

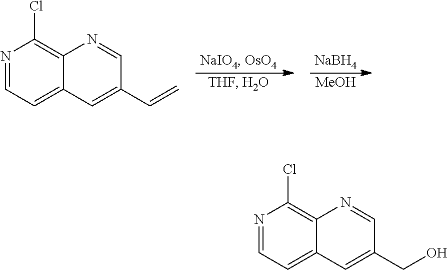

To a solution of 8-chloro-3-vinyl-1,7-naphthyridine (380 mg) in 1,4-dioxane (20 mL) and water (20 mL) Os₄ (0.9 mL, 4% in water) was added and stirred for 30 min at room temperature. NaIO₄ (4.0 g) was added in small portions at the same temperature. After stirring for 3 h, the reaction was quenched with saturated Na₂S₂O₃ solution. The mixture was extracted with DCM (40 mL) for 3 times. Organic layer was combined and dried over Na₂SO₄. The resulting solution was concentrated to afford 8-chloro-1,7-naphthyridine-3-carbaldehyde as a crude product which can be used directly in next step.

The above aldehyde was dissolved in 20 mL MeOH. NaBH₄ (400 mg) was added in one portion. The resulting mixture was stirred for 2 h at room temperature then quenched by water (30 mL). The mixture was extracted with DCM (20 mL) for 3 times and the organic phase was dried over Na₂SO₄. The resulting solution was concentrated and purified by silicagel (eluting with hexane-EtOAc using a gradient from 4:1 to 2:1) to afford (8-chloro-1,7-naphthyridin-3-yl)methanol (50 mg) as a brown solid.

### Step 3: Preparing of (8-((3-bromo-2-methylphenyl)amino)-1,7-naphthyridin-3-yl)methanol

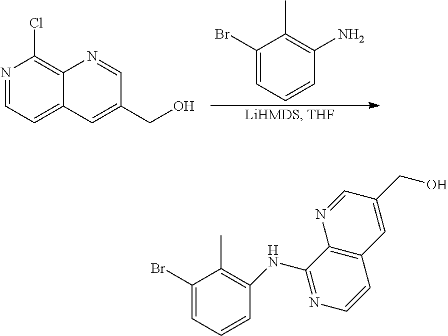

To a microwave reaction vial were added 3-bromo-2-methylaniline (370 mg), (8-chloro-1,7-naphthyridin-3-yl)methanol (98 mg), LiHMDS (1.0 M in THF, 4.0 mL) and THF (3.5 mL). The vial was capped and the reaction mixture was heated at 60° C. for 4 h. It was diluted with 20 mL of water and then extracted with DCM (20 mL*2). The combined organic extracts were washed with brine, dried over MgSO₄ and concentrated in vacuo. The residue was purified directly by RP-column (mobile phase: MeCN:water=30:70) to afford (8-((3-bromo-2-methylphenyl)amino)-1,7-naphthyridin-3-yl)methanol (73 mg) as a black solid.

### Step 4: Preparing of (8-((2-methyl-3-(4,4,5,5-tetramethyl-1,3,2-dioxaborolan-2-yl)phenyl)amino)-1,7-naphthyridin-3-yl)methanol

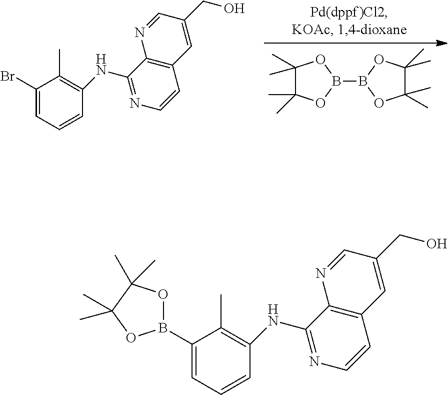

To a microwave reaction vial were added (8-((3-bromo-2-methylphenyl)amino)-1,7-naphthyridin-3-yl)methanol (68 mg), Bis(pinacolato)diboron (120 mg), Pd(dppf)Cl₂.DCM (10 mg), KOAc (102 mg), and 1,4-dioxane (3.0 mL). The vial was capped and the reaction mixture was heated at 100° C. for 2 h. It was diluted with 20 mL of water and then extracted with DCM (20 mL*2). The combined organic extracts were washed with brine, dried over MgSO₄ and concentrated in vacuo. The residue was purified silicagel (eluting with hexane-EtOAc using a gradient from 4:1 to 2:1) to afford (8-((2-methyl-3-(4,4,5,5-tetramethyl-1,3,2-dioxaborolan-2-yl)phenyl)amino)-1,7-naphthyridin-3-yl)methanol (50 mg) as a brown solid.

### Step 5: Preparing of (((2,2′-dimethyl-[1,1′-biphenyl]-3,3′-diyl)bis(azanediyl))bis(1,7-naphthyridine-8,3-diyl))dimethanol

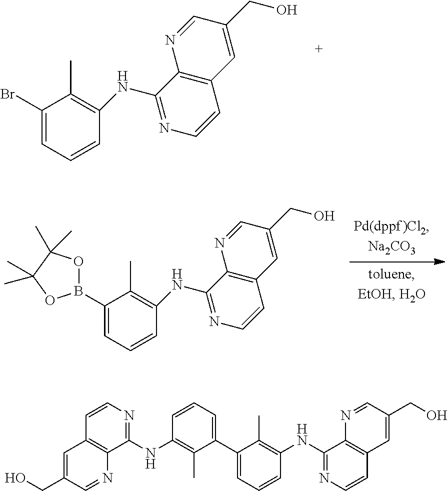 [SMILES: Cc1c(Br)cccc1Nc1nccc2cc(CO)cnc12 ; Cc1c(Nc2nccc3cc(CO)cnc23)cccc1B1OC(C)(C)C(C)(C)O1 ; Cc1c(Nc2nccc3cc(CO)cnc23)cccc1-c1cccc(Nc2nccc3cc(CO)cnc23)c1C]

To a solution of (8-((3-bromo-2-methylphenyl)amino)-1,7-naphthyridin-3-yl)methanol (50 mg), (8-((2-methyl-3-(4,4,5,5-tetramethyl-1,3,2-dioxaborolan-2-yl)phenyl)amino)-1,7-naphthyridin-3-yl)methanol (50 mg) in toluene (3 mL), EtOH (1 mL), and 10% Na₂CO₃ aq. (1 mL), Pd(dppf)Cl₂.DCM (10 mg) was added under N₂ protection. The mixture was allowed to stir at 100° C. for 16 h. The reaction was quenched by H₂O (20 mL) and extracted by DCM for 3 times. Organic layer was combined and washed with brine. The resulting solution was concentrated and purified by silicagel (eluting with DCM-MeOH using a gradient from 15:1 to 8:1) to afford (((2,2′-dimethyl-[1,1′-biphenyl]-3,3′-diyl)bis(azanediyl))bis(1,7-naphthyridine-8,3-diyl))dimethanol (40 mg) as a brown semi-solid.

### Step 6: Preparing of 2-(((8-(3′-((3-(2-hydroxyethyl)amino)-1,7-naphthyridin-8-yl)amino)-2,2′-dimethyl-[1,1′-biphenyl]-3-yl)amino)-1,7-naphthyridin-3-yl)methyl)amino)ethan-1-ol

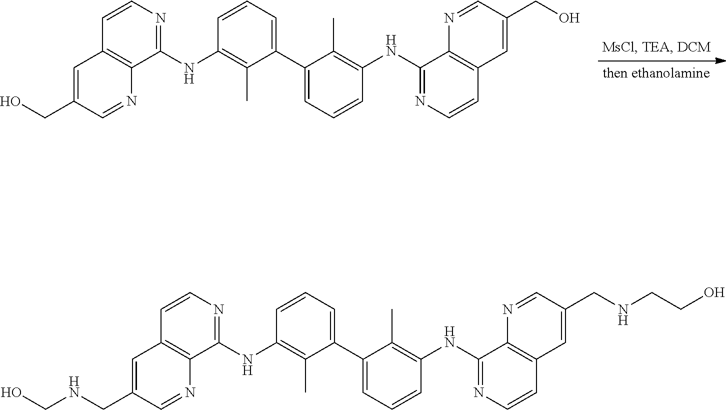 [SMILES: Cc1c(Nc2nccc3cc(CO)cnc23)cccc1-c1cccc(Nc2nccc3cc(CO)cnc23)c1C ; Cc1c(Nc2nccc3cc(CNCO)cnc23)cccc1-c1cccc(Nc2nccc3cc(CNCCO)cnc23)c1C]

To a solution of (((2,2′-dimethyl-[1,1′-biphenyl]-3,3′-diyl)bis(azanediyl))bis(1,7-naphthyridine-8,3-diyl))dimethanol, TEA (100 mg) in DCM (5.0 mL). MsCl (80 mg) was added dropwise at 0° C. The reaction was allowed to stir at room temperature for 90 min. The resulting mixture was concentrated under vacuo and redissolved by THF (3 mL). Ethanolamine was added then the reaction was continued to stir at room temperature for another 2 h unstill above methanesulfonate was consumed. The residue was concentrated and purified directly by RP-column (mobile phase: MeCN:water=10:90 with 0.1% HCl) to afford 2-(((8-((3′-((3-((2-hydroxyethyl)amino)-1,7-naphthyridin-8-yl)amino)-2,2′-dimethyl-[1,1′-biphenyl]-3-yl)amino)-1,7-naphthyridin-3-yl)methyl)amino)ethan-1-ol (17 mg) as an off-white solid.

### Example 167 Synthesis of Compound 167

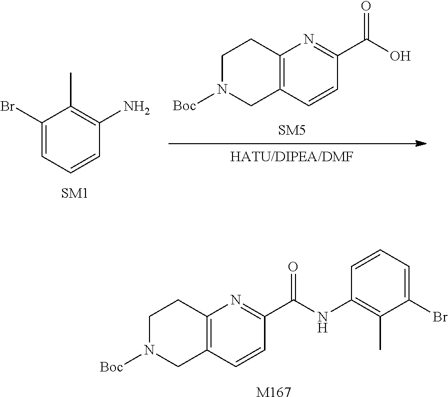

SM1(1.00 g), SM5(1.79 g), HATU (4.1 g), DMF (15 mL), and DIPEA (2.08 g) was placed in a 100 mL round bottom flask and stirred for 2 h at r.t. Monitored by TLC till the SM1 consumed. Ice water (25 mL) was added, and extracted with EA (20 mL*3), the organic phase was washed by saturated NaCl(aq) (20 mL*3) and dried with anhydrous Na₂SO₄, and concentrated. Purified with silica column (hexane/EA=5/1) to give the desired product with off-white solid.

The compound 167 was synthesized with M167 according to the method described above.

Prepare the following examples (shown in Table 3) essentially as described for Example 6 using the corresponding starting materials.

TABLE 3

| | | | Physical |
|---|---|---|---|
| | | | Data |
| | | | (MS) |
| NO. | Chemical Name | Structure | (M + H)+ |
| 138 | 2,2′-(((((2,2′-dimethyl-[1,1′-biphenyl]- 3,3′-diyl)bis(azanediyl))bis(1,7- naphthyridine-8,3- diyl))bis(methylene))bis(azanediyl)) diacetic acid | 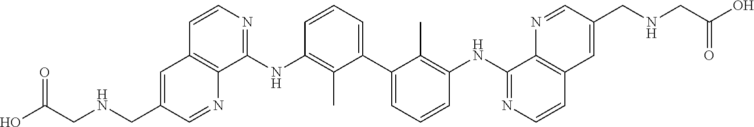 [SMILES: Cc1c(Nc2nccc3cc(CNCC(=O)O)cnc23)cccc1-c1cccc(Nc2nccc3cc(CNCC(=O)O)cnc23)c1C] | 643.3 |
| 139 | ((8-((3′-((3-(((2- hydroxyethyl)amino)methyl)-1,7- naphthyridin-8-yl)amino)-2,2′-dimethyl- [1,1′-biphenyl]-3-yl)amino)-1,7- naphthyridin-3-yl)methyl)glycine | 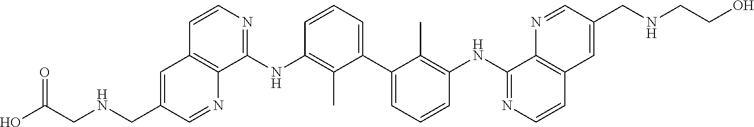 [SMILES: Cc1c(Nc2nccc3cc(CNCCO)cnc23)cccc1-c1cccc(Nc2nccc3cc(CNCC(=O)O)cnc23)c1C] | 629.3 |
| 140 | 2,2′-(((((2,2′-dimethyl-[1,1′-biphenyl]- 3,3′-diyl)bis(azanediyl))bis(1,7- naphthyridine-8,3- diyl))bis(methylene))bis(azanediyl)) dipropionic acid | 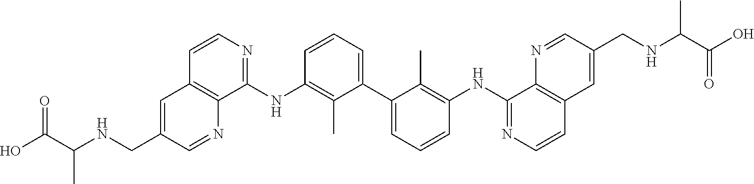 [SMILES: Cc1c(Nc2nccc3cc(CNC(C)C(=O)O)cnc23)cccc1-c1cccc(Nc2nccc3cc(CNC(C)C(=O)O)cnc23)c1C] | 671.3 |
| 141 | (S)-4-(((8-((3′-((3- (((carboxymethyl)amino)methyl)-1,7- naphthyridin-8-yl)amino)-2,2′-dimethyl- [1,1′-biphenyl]-3-yl)amino)-1,7- naphthyridin-3-yl)methyl)amino)-3- hydroxybutanoic acid | 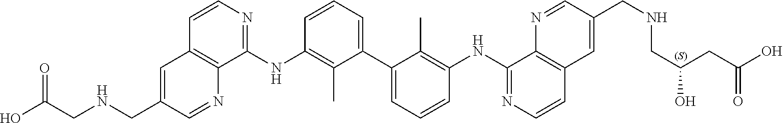 | 687.3 |
| 142 | 2,2′-(((((2,2′-dimethyl-[1,1′-biphenyl]- 3,3′-diyl)bis(azanediyl))bis(1,7- naphthyridine-8,3- diyl))bis(methylene))bis(azanediyl)) bis(3-methylbutanoic acid) | 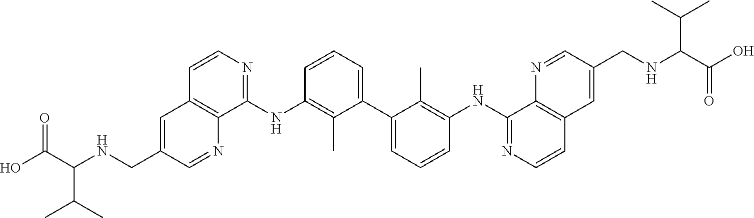 [SMILES: Cc1c(Nc2nccc3cc(CNC(C(=O)O)C(C)C)cnc23)cccc1-c1cccc(Nc2nccc3cc(CNC(C(=O)O)C(C)C)cnc23)c1C] | 727.4 |
| 143 | (3S,3′S)-4,4′-(((((2,2′-dimethyl-[1,1′- biphenyl]-3,3′- diyl)bis(azanediyl))bis(1,7- naphthyridine-8,3- diyl))bis(methylene))bis(azanediyl)) bis(3-hydroxybutanoic acid) | 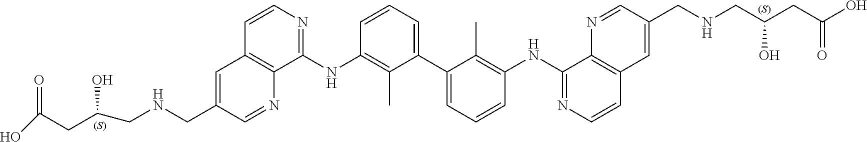 | 731.3 |
| 144 | 2,2′-(((((2,2′-dimethyl-[1,1′-biphenyl]- 3,3′-diyl)bis(azanediyl))bis(1,7- naphthyridine-8,3- diyl))bis(methylene))bis(azanediyl)) diacetamide | 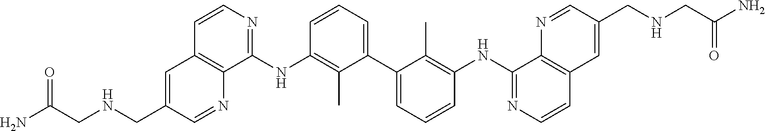 [SMILES: Cc1c(Nc2nccc3cc(CNCC(N)=O)cnc23)cccc1-c1cccc(Nc2nccc3cc(CNCC(N)=O)cnc23)c1C] | 641.3 |
| 145 | 2,2′-(((((2,2′-dimethyl-[1,1′-biphenyl]- 3,3′-diyl)bis(azanediyl))bis(1,7- naphthyridine-8,3- diyl))bis(methylene))bis(azanediyl)) bis(N,N-dimethylacetamide) | 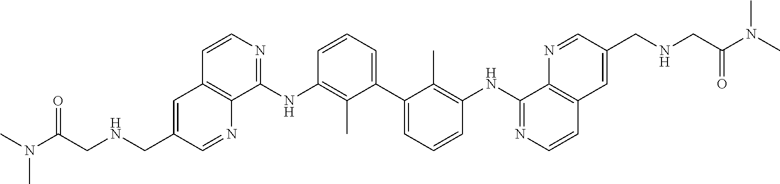 | 697.4 |
| 146 | 2-(((8-((2,2′-dimethyl-3′-((3-(((2,2,2- trifluoroethyl)amino)methyl)-1,7- naphthyridin-8-yl)amino)-[1,1′- biphenyl]-3-yl)amino)-1,7-naphthyridin- 3-yl)methyl)amino)-N,N- dimethylacetamide | 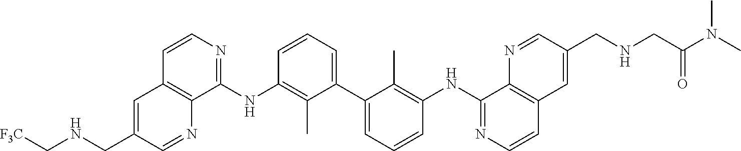 [SMILES: Cc1c(Nc2nccc3cc(CNCC(=O)N(C)C)cnc23)cccc1-c1cccc(Nc2nccc3cc(CNCC(F)(F)F)cnc23)c1C] | 694.3 |
| 147 | 2-(((8-((2,2′-dimethyl-3′-((3-(((2- (methylamino)-2- oxoethyl)amino)methyl)-1,7- naphthyridin-8-yl)amino)-[1,1′- biphenyl]-3-yl)amino)-1,7-naphthyridin- 3-yl)methyl)amino)-N-(2-hydroxyethyl)- N-methylacetamide | 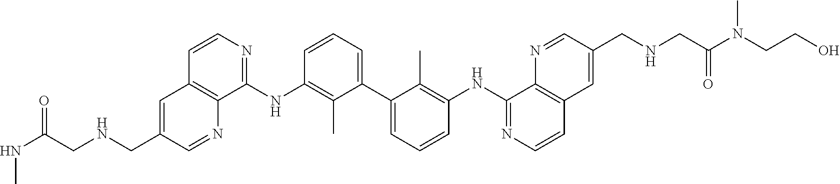 [SMILES: CNC(=O)CNCc1cnc2c(Nc3cccc(-c4cccc(Nc5nccc6cc(CNCC(=O)N(C)CCO)cnc56)c4C)c3C)nccc2c1] | 713.4 |
| 148 | ((8-((3′-((3-(((2-(dimethylamino)-2- oxoethyl)amino)methyl)-1,7- naphthyridin-8-yl)amino)-2,2′-dimethyl- [1,1′-biphenyl]-3-yl)amino)-1,7- naphthyridin-3-yl)methyl)glycine | 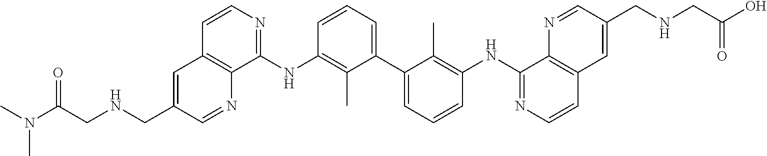 [SMILES: Cc1c(Nc2nccc3cc(CNCC(=O)O)cnc23)cccc1-c1cccc(Nc2nccc3cc(CNCC(=O)N(C)C)cnc23)c1C] | 670.3 |
| 149 | ((8-((3′-((3-(azetidin-1-ylmethyl)-1,7- naphthyridin-8-yl)amino)-2,2′-dimethyl- [1,1′-biphenyl]-3-yl)amino)-1,7- naphthyridin-3-yl)methyl)glycine | 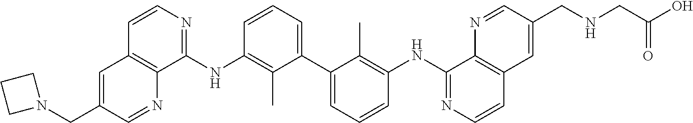 [SMILES: Cc1c(Nc2nccc3cc(CNCC(=O)O)cnc23)cccc1-c1cccc(Nc2nccc3cc(CN4CCC4)cnc23)c1C] | 625.3 |
| 150 | 1,1′-((((2,2′-dimethyl-[1,1′-biphenyl]- 3,3′-diyl)bis(azanediyl))bis(1,7- naphthyridine-8,3- diyl))bis(methylene))bis(azetidin-3-ol) | 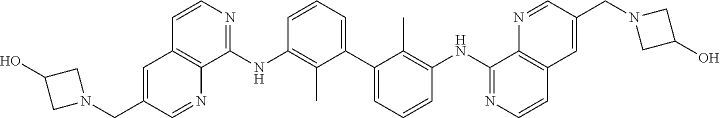 [SMILES: Cc1c(Nc2nccc3cc(CN4CC(O)C4)cnc23)cccc1-c1cccc(Nc2nccc3cc(CN4CC(O)C4)cnc23)c1C] | 639.3 |
| 151 | 2,2′-dimethyl-N3,N3′-bis(3- (morpholinomethyl)-1,7-naphthyridin-8- yl)-[1,1′-biphenyl]-3,3′-diamine | 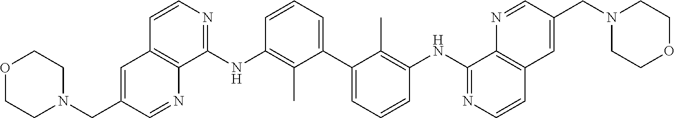 [SMILES: Cc1c(Nc2nccc3cc(CN4CCOCC4)cnc23)cccc1-c1cccc(Nc2nccc3cc(CN4CCOCC4)cnc23)c1C] | 667.4 |
| 152 | N,N′-((((((2,2′-dimethyl-[1,1′-biphenyl]- 3,3′-diyl)bis(azanediyl))bis(1,7- naphthyridine-8,3- diyl))bis(methylene))bis(azanediyl)) bis(ethane-2,1-diyl))diacetamide | 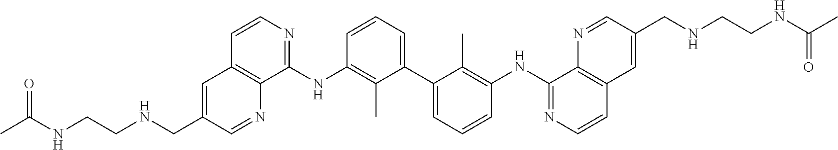 [SMILES: CC(=O)NCCNCc1cnc2c(Nc3cccc(-c4cccc(Nc5nccc6cc(CNCCNC(C)=O)cnc56)c4C)c3C)nccc2c1] | 697.4 |
| 153 | N3,N3′-bis(3-(((S)-3- (dimethylamino)pyrrolidin-1-yl)methyl)- 1,7-naphthyridin-8-yl)-2,2′-dimethyl- [1,1′-biphenyl]-3,3′-diamine | 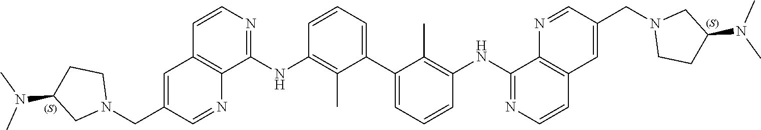 | 721.4 |
| 154 | (S)-((8-((3′-((3-((3- (dimethylamino)pyrrolidin-1-yl)methyl)- 1,7-naphthyridin-8-yl)amino)-2,2′- dimethyl-[1,1′-biphenyl]-3-yl)amino)- 1,7-naphthyridin-3-yl)methyl)glycine | 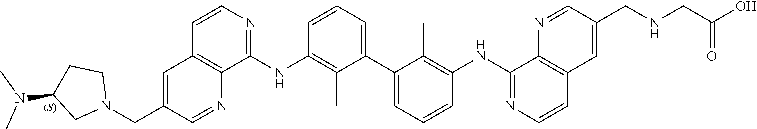 | 682.4 |
| 155 | 1,1′-((((2,2′-dimethyl-[1,1′-biphenyl]- 3,3′-diyl)bis(azanediyl))bis(1,7- naphthyridine-8,3- diyl))bis(methylene))bis(piperidine-2- carboxylic acid) | 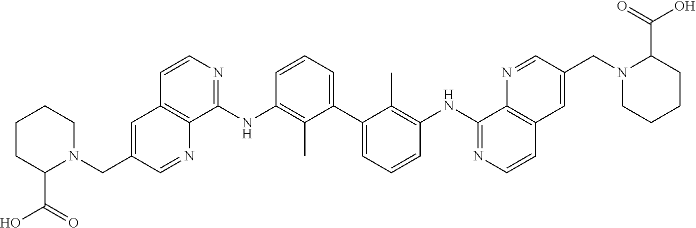 [SMILES: Cc1c(Nc2nccc3cc(CN4CCCCC4C(=O)O)cnc23)cccc1-c1cccc(Nc2nccc3cc(CN4CCCCC4C(=O)O)cnc23)c1C] | 751.4 |
| 156 | 2-(((8-((2,2′-dimethyl-3′-((3-(((2,2,2- trifluoroethyl)amino)methyl)-1,7- naphthyridin-8-yl)amino)-[1,1′, biphenyl]-3-yl)amino)-1,7-naphthyridin- 3-yl)methyl)amino)ethan-1-ol | 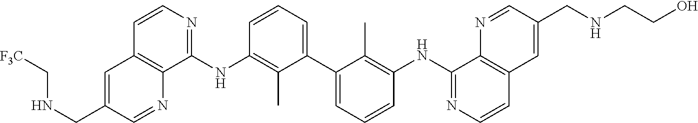 [SMILES: Cc1c(Nc2nccc3cc(CNCCO)cnc23)cccc1-c1cccc(Nc2nccc3cc(CNCC(F)(F)F)cnc23)c1C] | 653.3 |
| 157 | (1S,1′S)-1,1′-((((((2,2′-dimethyl-[1,1′- biphenyl]-3,3′- diyl)bis(azanediyl))bis(1,7- naphthyridine-8,3-diyl)) bis(methylene))bis(azanediyl))bis (cyclopropane-1,1-diyl))bis(ethan- 1-ol) | 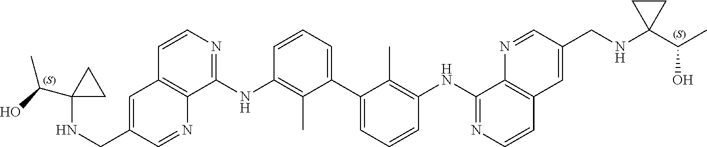 | 695.4 |
| 158 | dimethyl ((((((2,2′-dimethyl-[1,1′- biphenyl]-3,3′- diyl)bis(azanediyl))bis(1,7- naphthyridine-8,3- diyl))bis(methylene))bis(azanediyl)) bis(ethane-2,1-diyl))dicarbamate | 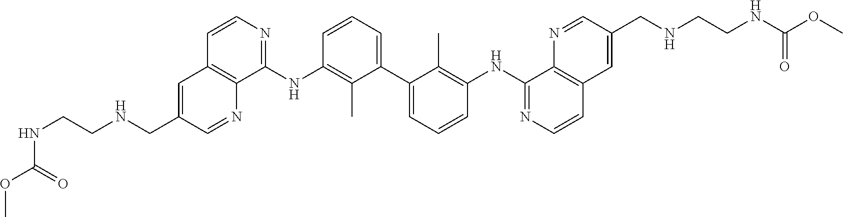 [SMILES: COC(=O)NCCNCc1cnc2c(Nc3cccc(-c4cccc(Nc5nccc6cc(CNCCNC(=O)OC)cnc56)c4C)c3C)nccc2c1] | 729.4 |
| 159 | 2-(2-(4-(3-((3-(((2- ((methoxycarbonyl)amino)ethyl)amino) methyl)-1,7-naphthyridin-8-yl)amino)-2- methylphenyl)indoline-1-carbonyl)- 3a,6,7,7a-tetrahydrothiazolo[5,4- c]pyridin-5(4H)-yl)acetic acid | 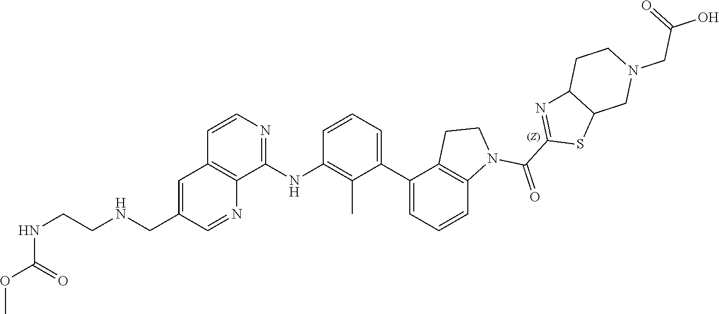 [SMILES: COC(=O)NCCNCc1cnc2c(Nc3cccc(-c4cccc5c4CCN5C(=O)C4=NC5CCN(CC(=O)O)CC5S4)c3C)nccc2c1] | 709.3 |
| 160 | ((8-((3-(1-(5-(carboxymethyl)- 3a,4,5,6,7,7a-hexahydrothiazolo[5,4- c]pyridine-2-carbonyl)indolin-4-yl)-2- methylphenyl)amino)-1,7-naphthyridin- 3-yl)methyl)glycine | 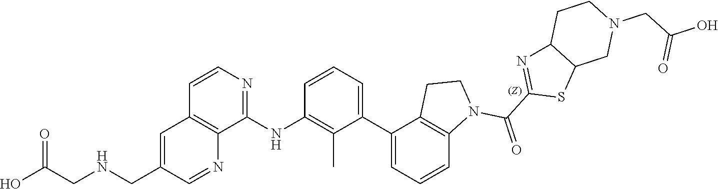 [SMILES: Cc1c(Nc2nccc3cc(CNCC(=O)O)cnc23)cccc1-c1cccc2c1CCN2C(=O)C1=NC2CCN(CC(=O)O)CC2S1] | 666.3 |
| 161 | 3-(2-(4-(3-((3- (((carboxymethyl)amino)methyl)-1,7- naphthyridin-8-yl)amino)-2- methylphenyl)indoline-1-carbonyl)- 3a,6,7,7a-tetrahydrothiazolo[5,4- c]pyridin-5(4H)-yl)propanoic acid | 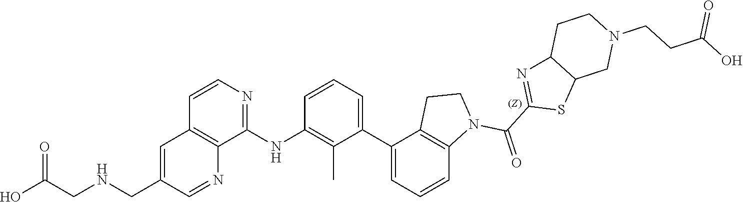 [SMILES: Cc1c(Nc2nccc3cc(CNCC(=O)O)cnc23)cccc1-c1cccc2c1CCN2C(=O)C1=NC2CCN(CCC(=O)O)CC2S1] | 680.3 |
| 162 | 3-(2-(4-(3-((3-(((2- hydroxyethyl)amino)methyl)-1,7- naphthyridin-8-yl)amino)-2- methylphenyl)indoline-1-carbonyl)- 3a,6,7,7a-tetrahydrothiazolo[5,4- c]pyridin-5(4H)-yl)propanoic acid | 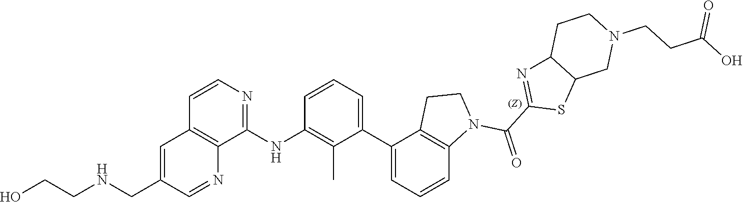 [SMILES: Cc1c(Nc2nccc3cc(CNCCO)cnc23)cccc1-c1cccc2c1CCN2C(=O)C1=NC2CCN(CCC(=O)O)CC2S1] | 666.3 |
| 163 | ((8-((3-(1-(5-(carboxymethyl)- 3a,4,5,6,7,7a-hexahydrothiazolo[5,4- c]pyridine-2-carbonyl)indolin-4-yl)-2- methylphenyl)amino)-1,7-naphthyridin- 3-yl)methyl)alanine | 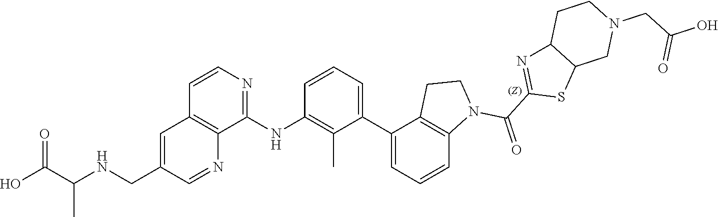 [SMILES: Cc1c(Nc2nccc3cc(CNC(C)C(=O)O)cnc23)cccc1-c1cccc2c1CCN2C(=O)C1=NC2CCN(CC(=O)O)CC2S1] | 680.3 |
| 164 | 2-(2-(4-(3-((3-(((2-amino-2- oxoethyl)amino)methyl)-1,7 - naphthyridin-8-yl)amino)-2- methylphenyl)indoline-1-carbonyl)- 3a,6,7,7a-tetrahydrothiazolo[5,4- c]pyridin-5(4H)-yl)acetic acid | 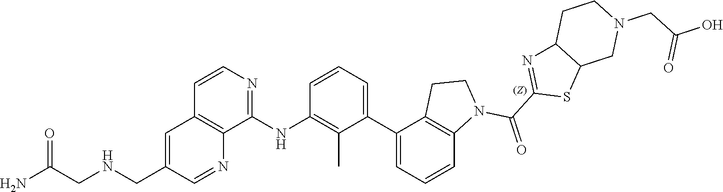 [SMILES: Cc1c(Nc2nccc3cc(CNCC(N)=O)cnc23)cccc1-c1cccc2c1CCN2C(=O)C1=NC2CCN(CC(=O)O)CC2S1] | 665.3 |
| 165 | 2-(2-(4-(3-((3-(((2-(dimethylamino)-2- oxoethyl)amino)methyl)-1,7- naphthyridin-8-yl)amino)-2- methylphenyl)indoline-1-carbonyl)- 3a,6,7,7a-tetrahydrothiazolo[5,4- c]pyridin-5(4H)-yl)acetic acid | 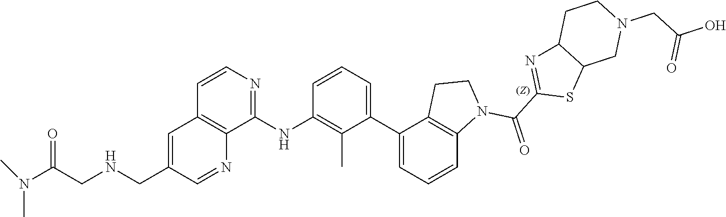 [SMILES: Cc1c(Nc2nccc3cc(CNCC(=O)N(C)C)cnc23)cccc1-c1cccc2c1CCN2C(=O)C1=NC2CCN(CC(=O)O)CC2S1] | 693.3 |
| 166 | 2-(2-(4-(3-((3-(((2-((2- hydroxyethyl)(methyl)amino)-2- oxoethyl)amino)methyl)-1,7- naphthyridin-8-yl)amino)-2- methylphenyl)indoline-1-carbonyl)- 3a,6,7,7a-tetrahydrothiazolo[5,4- c]pyridin-5(4H)-yl)acetic acid | 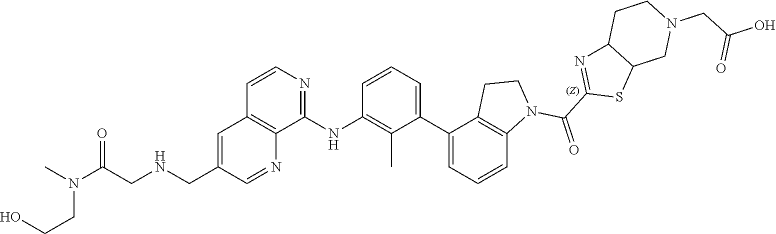 [SMILES: Cc1c(Nc2nccc3cc(CNCC(=O)N(C)CCO)cnc23)cccc1-c1cccc2c1CCN2C(=O)C1=NC2CCN(CC(=O)O)CC2S1] | 723.3 |
| 167 | N,N′-(2,2′-dimethyl-[1,1′-biphenyl]-3,3′- diyl)bis(5,6,7,8-tetrahydro-1,6- naphthyridine-2-carboxamide) | 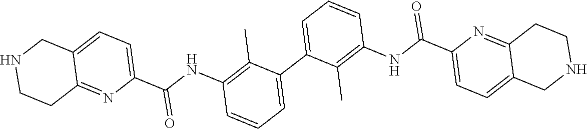 [SMILES: Cc1c(NC(=O)c2ccc3c(n2)CCNC3)cccc1-c1cccc(NC(=O)c2ccc3c(n2)CCNC3)c1C] | 533.3 |
| 168 | dimethyl ((2S,2′S)-(([4,4′-biindoline]- 1,1′-dicarbonyl)bis(7,8-dihydro-1,6- naphthyridine-2,6(5H)-diyl))bis(3- methyl-1-oxobutane-1,2- diyl))dicarbamate | 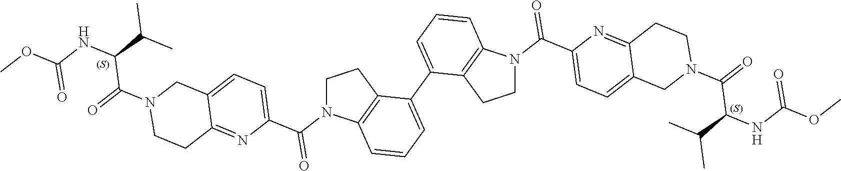 | 871.4 |
| 169 | dimethyl ((2S,2′S)-((((2,2′-dimethyl- [1,1′-biphenyl]-3,3′-diyl) bis(azanediyl))bis(carbonyl))bis(7,8- dihydro-1,6-naphthyridine-2,6(5H)- diyl))bis(3-methyl-1-oxobutane-1,2- diyl))dicarbamate | 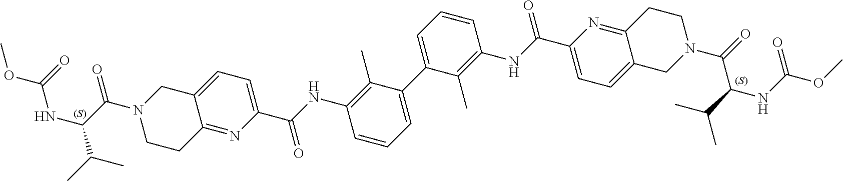 | 847.4 |
| 170 | ethyl 2-(2-((2,2′-dimethyl-3′-(5,6,7,8- tetrahydro-1,6-naphthyridine-2- carboxamido)-[1,1′-biphenyl]-3- yl)carbamoyl)-7,8-dihydro-1,6- naphthyridin-6(5H)-yl)acetate | 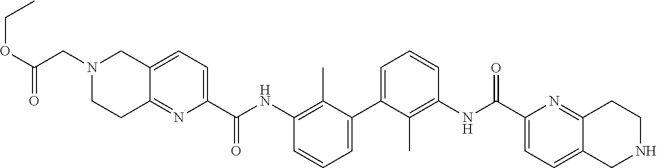 [SMILES: CCOC(=O)CN1CCc2nc(C(=O)Nc3cccc(-c4cccc(NC(=O)c5ccc6c(n5)CCNC6)c4C)c3C)ccc2C1] | 619.3 |
| 171 | 2-((3′-(6,6-dimethyl-5,6,7,8-tetrahydro- 6l4-1,6-naphthyridin-7-ylium-2- carboxamido)-2,2′-dimethyl-[1,1′- biphenyl]-3-yl)carbamoyl)-6,6-dimethyl- 5,6,7,8-tetrahydro-1,6-naphthyridin-6- ium chloride | 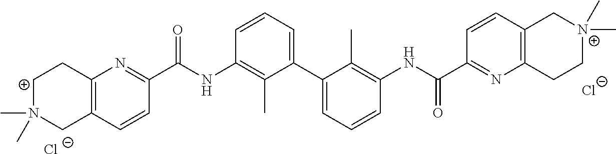 | 661.26 |
| 172 | di-tert-butyl 2,2′-((((2,2′-dimethyl-[1,1′- biphenyl]-3,3′-diyl) bis(azanediyl))bis(carbonyl))bis(7,8- dihydro-1,6-naphthyridine-2,6(5H)- diyl))diacetate | 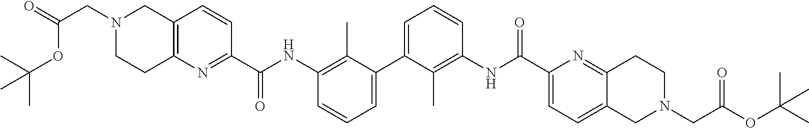 [SMILES: Cc1c(NC(=O)c2ccc3c(n2)CCN(CC(=O)OC(C)(C)C)C3)cccc1-c1cccc(NC(=O)c2ccc3c(n2)CCN(CC(=O)OC(C)(C)C)C3)c1C] | 761.4 |
| 173 | 2,2′-((((2,2′-dimethyl-[1,1′-biphenyl]- 3,3′-diyl) bis(azanediyl))bis(carbonyl))bis(7,8- dihydro-1,6-naphthyridine-2,6(5H)- diyl))diacetic acid | 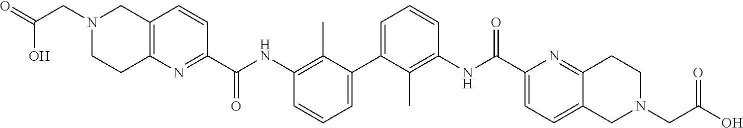 [SMILES: Cc1c(NC(=O)c2ccc3c(n2)CCN(CC(=O)O)C3)cccc1-c1cccc(NC(=O)c2ccc3c(n2)CCN(CC(=O)O)C3)c1C] | 649.3 |
| 174 | N,N′-(2,2′-dimethyl-[1,1′-biphenyl]-3,3′- diyl)bis(6-(2-hydroxyethyl)-5,6,7,8- tetrahydro-1,6-naphthyridine-2- carboxamide) | 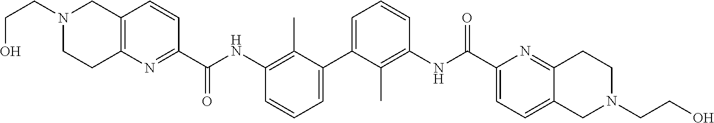 [SMILES: Cc1c(NC(=O)c2ccc3c(n2)CCN(CCO)C3)cccc1-c1cccc(NC(=O)c2ccc3c(n2)CCN(CCO)C3)c1C] | 621.3 |
| 175 | N,N′-(2,2′-dimethyl-[1,1′-biphenyl]-3,3′- diyl)bis(6-methyl-5,6,7,8-tetrahydro-1,6- naphthyridine-2-carboxamide) | 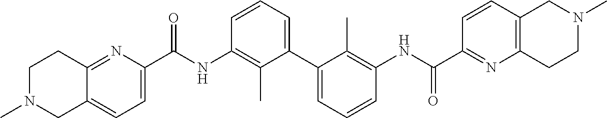 | 561.3 |
| 176 | N,N′-(2,2′-dimethyl-[1,1′-biphenyl]-3,3′- diyl)bis(6-(2-morpholinoethyl)-5,6,7,8- tetrahydro-1,6-naphthyridine-2- carboxamide) | 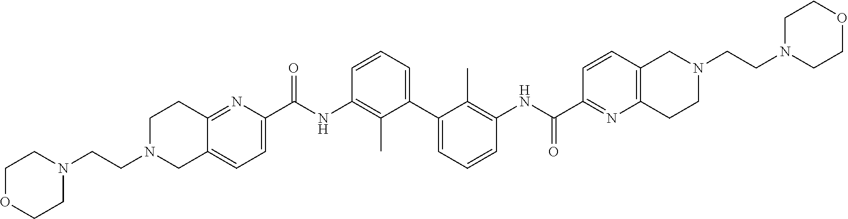 [SMILES: Cc1c(NC(=O)c2ccc3c(n2)CCN(CCN2CCOCC2)C3)cccc1-c1cccc(NC(=O)c2ccc3c(n2)CCN(CCN2CCOCC2)C3)c1C] | 759.4 |
| 177 | diethyl (2-(2-((3′-(((6-(2- (diethoxyphosphoryl)ethyl)-5,6,7,8- tetrahydro-1,6-naphthyridin-2- yl)methyl)amino)-2,2′-dimethyl-[1,1′- biphenyl]-3-yl)carbamoyl)-7,8-dihydro- 1,6-naphthyridin-6(5H)- yl)ethyl)phosphonate | 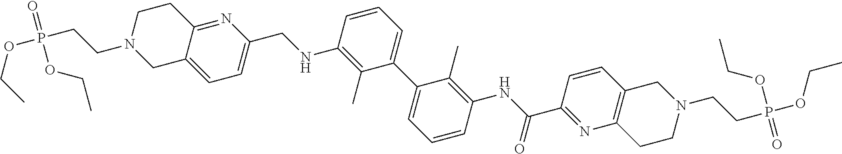 [SMILES: CCOP(=O)(CCN1CCc2nc(CNc3cccc(-c4cccc(NC(=O)c5ccc6c(n5)CCN(CCP(=O)(OCC)OCC)C6)c4C)c3C)ccc2C1)OCC] | 847.4 |
| 178 | 2-(2-((2,2′-dimethyl-3′-(5,6,7,8- tetrahydro-1,6-naphthyridine-2- carboxamido)-[1,1′-biphenyl]-3- yl)carbamoyl)-7,8-dihydro-1,6- naphthyridin-6(5H)-yl)acetic acid | 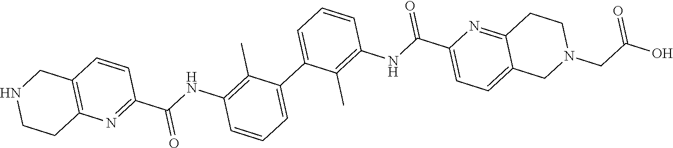 [SMILES: Cc1c(NC(=O)c2ccc3c(n2)CCNC3)cccc1-c1cccc(NC(=O)c2ccc3c(n2)CCN(CC(=O)O)C3)c1C] | 591.3 |
| 179 | diethyl 2,2′-((((((2,2′-dimethyl-[1,1′- biphenyl]-3,3′-diyl)bis(azanediyl)) bis(carbonyl))bis(pyrazine- 5,2-diyl))bis(ethane-1,1- diyl))bis(azanediyl))diacetate | 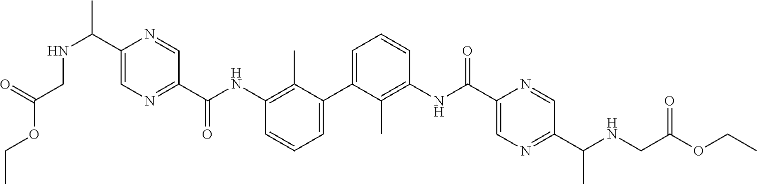 [SMILES: CCOC(=O)CNC(C)c1cnc(C(=O)Nc2cccc(-c3cccc(NC(=O)c4cnc(C(C)NCC(=O)OCC)cn4)c3C)c2C)cn1] | 683.3 |
| 180 | N,N′-(2,2′-dimethyl-[1,1′-biphenyl]-3,3′- diyl)bis(5-(1- (methylamino)ethyl)pyrazine-2- carboxamide) | 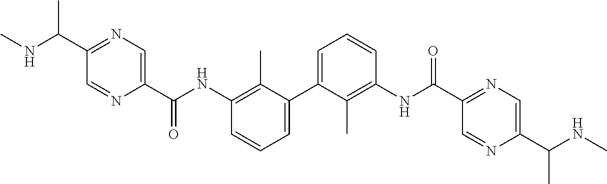 | 539.3 |
| 181 | N,N′-(2,2′-dimethyl-[1,1′-biphenyl]-3,3′- diyl)bis(5-(1-((2- hydroxyethyl)amino)ethyl)pyrazine-2- carboxamide) | 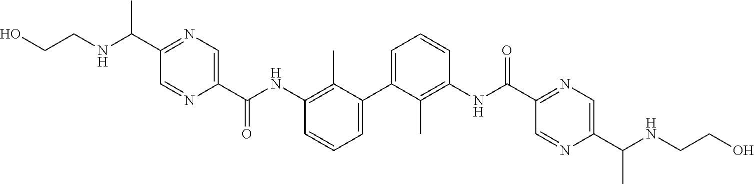 [SMILES: Cc1c(NC(=O)c2cnc(C(C)NCCO)cn2)cccc1-c1cccc(NC(=O)c2cnc(C(C)NCCO)cn2)c1C] | 599.3 |
| 182 | 6-acetyl-N-(2,2′-dimethyl-3′-(5,6,7,8- tetrahydro-1,6-naphthyridine-2- carboxamido)-[1,1′-biphenyl]-3-yl)- 5,6,7,8-tetrahydro-1,6-naphthyridine-2- carboxamide | 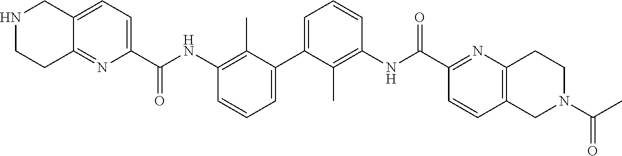 [SMILES: CC(=O)N1CCc2nc(C(=O)Nc3cccc(-c4cccc(NC(=O)c5ccc6c(n5)CCNC6)c4C)c3C)ccc2C1] | 575.3 |
| 183 | N-(3′-(5-acetyl-1-methyl-4,5,6,7- tetrahydro-1H-imidazo[4,5-c]pyridine-2- carboxamido)-2,2′-dimethyl-[1,1′- biphenyl]-3-yl)-5,6,7,8-tetrahydro-1,6- naphthyridine-2-carboxamide | 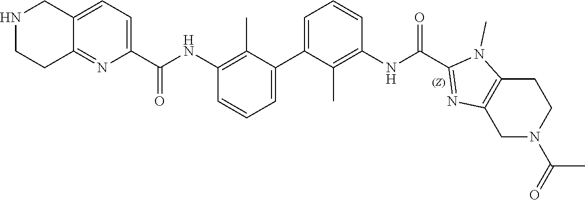 [SMILES: CC(=O)N1CCc2c(nc(C(=O)Nc3cccc(-c4cccc(NC(=O)c5ccc6c(n5)CCNC6)c4C)c3C)n2C)C1] | 578.3 |
| 184 | bis((5-methyl-2-oxo-1,3-dioxol-4- yl)methyl) 2,2′-(((2,2′-dimethyl-[1,1′- biphenyl]-3,3′-diyl) bis(azanediyl))bis(carbonyl))bis(7,8- dihydro-1,6-naphthyridine-6(5H)- carboxylate) | 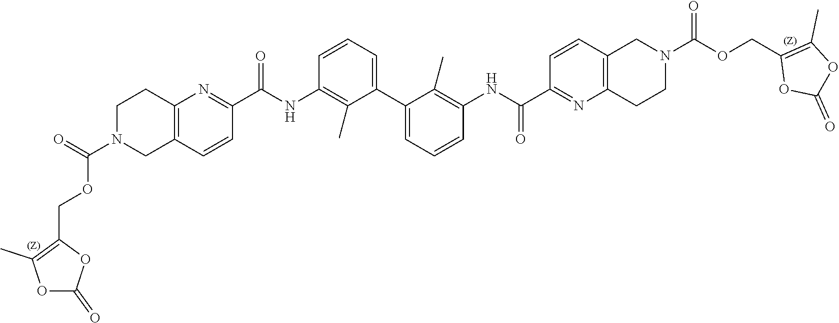 [SMILES: Cc1oc(=O)oc1COC(=O)N1CCc2nc(C(=O)Nc3cccc(-c4cccc(NC(=O)c5ccc6c(n5)CCN(C(=O)OCc5oc(=O)oc5C)C6)c4C)c3C)ccc2C1] | 845.3 |
| 185 | N-(2,2′-dimethyl-3′-(5,6,7,8-tetrahydro- 1,6-naphthyridine-2-carboxamido)-[1,1′- biphenyl]-3-yl)-6-methyl-5,6,7,8- tetrahydro-1,6-naphthyridine-2- carboxamide | 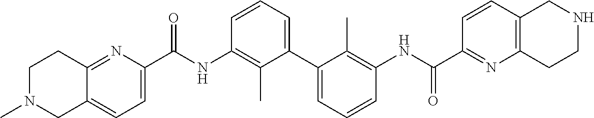 [SMILES: Cc1c(NC(=O)c2ccc3c(n2)CCNC3)cccc1-c1cccc(NC(=O)c2ccc3c(n2)CCN(C)C3)c1C] | 547.3 |
| 186 | N-(2,2′-dimethyl-3′-(5,6,7,8-tetrahydro- 1,6-naphthyridine-2-carboxamido)-[1,1′- biphenyl]-3-yl)-6-(2-hydroxyethyl)- 5,6,7,8-tetrahydro-1,6-naphthyridine-2- carboxamide | 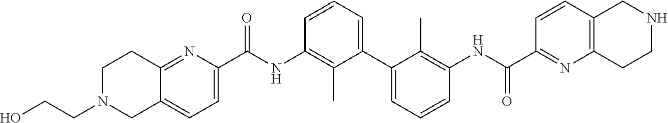 [SMILES: Cc1c(NC(=O)c2ccc3c(n2)CCNC3)cccc1-c1cccc(NC(=O)c2ccc3c(n2)CCN(CCO)C3)c1C] | 577.3 |
| 187 | N,N′-(2,2′-dimethyl-[1,1′-biphenyl]-3,3′- diyl)bis(6-(2,2-difluoroethyl)-5,6,7,8- tetrahydro-1,6-naphthyridine-2- carboxamide) | 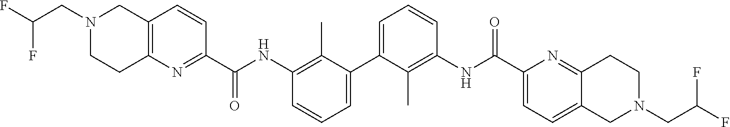 [SMILES: Cc1c(NC(=O)c2ccc3c(n2)CCN(CC(F)F)C3)cccc1-c1cccc(NC(=O)c2ccc3c(n2)CCN(CC(F)F)C3)c1C] | 661.3 |
| 188 | N,N′-(2,2′-dimethyl-[1,1′-biphenyl]-3,3′- diyl)bis(5,6,7,8-tetrahydropyrido[4,3- d]pyrimidine-2-carboxamide) | 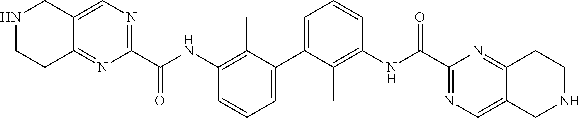 [SMILES: Cc1c(NC(=O)c2ncc3c(n2)CCNC3)cccc1-c1cccc(NC(=O)c2ncc3c(n2)CCNC3)c1C] | 535.3 |
| 189 | [4,4′-biindoline]-1,1′-diylbis((5,6,7,8- tetrahydropyrido[4,3-d]pyrimidin-2- yl)methanone) | 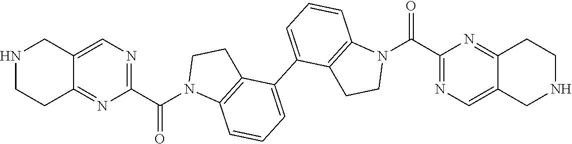 [SMILES: O=C(c1ncc2c(n1)CCNC2)N1CCc2c(-c3cccc4c3CCN4C(=O)c3ncc4c(n3)CCNC4)cccc21] | 559.3 |
| 190 | 2,2′-((((2,2′-dimethyl-[1,1′-biphenyl]- 3,3′-diyl) bis(azanediyl))bis(carbonyl))bis(7,8- dihydropyrido[4,3-d]pyrimidine- 2,6(5H)-diyl))diacetic acid | 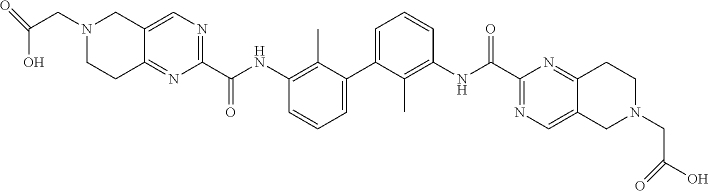 [SMILES: Cc1c(NC(=O)c2ncc3c(n2)CCN(CC(=O)O)C3)cccc1-c1cccc(NC(=O)c2ncc3c(n2)CCN(CC(=O)O)C3)c1C] | 651.3 |

### Example 191 Synthesis of Comparative Example 1

### N-(2-methyl-[1,1′-biphenyl]-3-yl)-4,5,6,7-tetrahydrothiazolo[5,4-c]pyridine-2-carboxamide

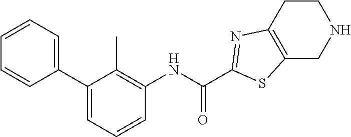 [SMILES: Cc1c(NC(=O)c2nc3c(s2)CNCC3)cccc1-c1ccccc1]

### Step 1: Preparation of 2-methyl-[1,1′-biphenyl]-3-amine (M11)

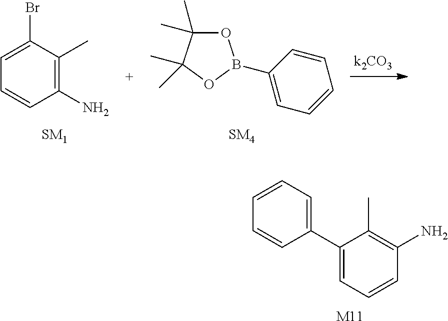

A mixture of 3-bromo-2-methylaniline (1.000 g), 4,4,5,5-tetramethyl-2-phenyl-1,3,2-dioxaborolane (1.320 g) and Potassium carbonate (1.480 g) in 1,4-dioxane (14.8 mL) and water (1.0 mL) was purged with nitrogen for 10 min. [1,1′-Bis(diphenylphosphino)ferrocene]-dichloropalladium DCM adduct (27 mg) was added, the mixture was purged for another 5 min then was heated at reflux for 1.5 hrs. The mixture was cooled and filtered through Celite. The solids were washed with EtOAc, and the combined filtrates were washed with water and brine, and dried and concentrated. The residue was purified by column chromatography (eluting with hexane-EtOAc using a gradient from 20:1 to 25:1). 2-methyl-[1,1′-biphenyl]-3-amine (compound M1) as a light yellow waxy solid (950 mg).

### Step 2: Preparation of tert-butyl 2-((2-methyl-[1,1′-biphenyl]-3-yl)carbamoyl)-6,7-dihydrothiazolo[5,4-c]pyridine-5(4H)-carboxylate (M22)

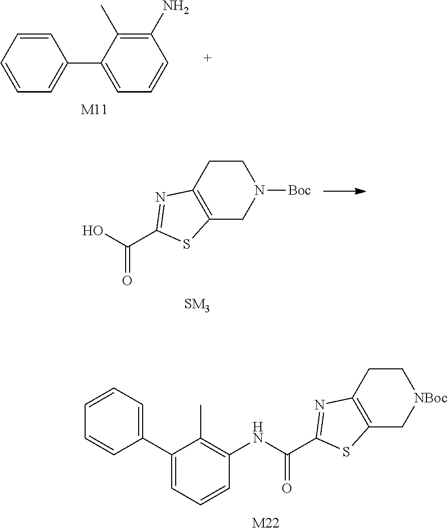

To a solution of 2-methyl-[1,1′-biphenyl]-3-amine (850 mg) in dry dicloromethane was added HATU (2.180 g) and DIEA (1.750 g). A solution of 6-(tert-butoxycarbonyl)-4,5,6,7-tetrahydrothieno[2,3-c]pyridine-2-carboxylic acid (1.450 g) in dicloromethane was added slowly and stirred at 40° C. for 4 hrs and then at room temperature overnight. Reaction mass was then concentrated and purified by column chromatography to afford the tert-butyl 2-((2-methyl-[1,1′-biphenyl]-3-yl)carbamoyl)-4,7-dihydrothieno[2,3-c]pyridine-6(5H)-carboxylate (compound M22) as a light yellow solid (1.450 g).

### Step 3: Preparation of N-(2-methyl-[1,1′-biphenyl]-3-yl)-4,5,6,7-tetrahydrothiazolo[5,4-c]pyridine-2-carboxamide (Comparative Example 1)

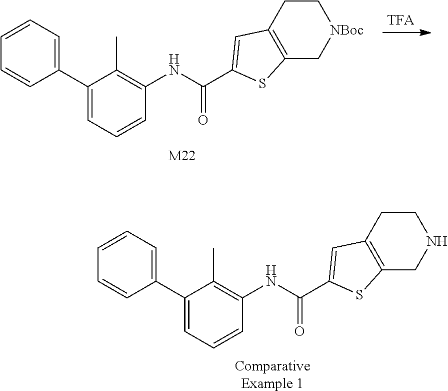

To a solution of tert-butyl 2-((2-methyl-[1,1′-biphenyl]-3-yl)carbamoyl)-4,7-dihydrothieno[2,3-c]pyridine-6(5H)-carboxylate (800 mg) in dicloromethane was added TFA (5 mL), and stirred at 40° C. for hrs. Reaction mass was then concentrated and washed by n-hexane to afford the N-(2-methyl-[1,1′-biphenyl]-3-yl)-4,5,6,7-tetrahydrothieno[2,3-c]pyridine-2-carboxamide (Comparative Example 1) as a light yellow solid (540 mg).

### Example 192 Synthesis of Comparative Example 2

### N-(2-methyl-[1,1′-biphenyl]-3-yl)-4,5,6,7-tetrahydrothiazolo[5,4-c]pyridine-2-carboxamide

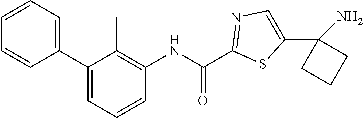 [SMILES: Cc1c(NC(=O)c2ncc(C3(N)CCC3)s2)cccc1-c1ccccc1]

Prepare the above comparative example essentially as described for Example 34 using the corresponding intermediates.

Resolved Fluorescence (HTRF) Binding Assay

The assays were conducted in a standard black 384-well polystyrene plate with a final volume of 20 μL. Inhibitors were first serially diluted in DMSO and then added to the plate wells before the addition of other reaction components. The final concentration of DMSO in the assay was 1%. The assays were carried out at 25° C. in the PBS buffer (pH 7.4) with 0.05% Tween-20 and 0.1% BSA. Recombinant human PD-L1 protein (19-238) with a His-tag at the C-terminus was purchased from AcroBiosy stems (PD1-H5229). Recombinant human PD-1 protein (25-167) with Fc tag at the C-terminus was also purchased from AcroBiosystems (PD1-H5257). PD-L1 and PD-1 proteins were diluted in the assay buffer and 10 μL was added to the plate well. Plates were centrifuged and proteins were preincubated with inhibitors for 40 min. The incubation was followed by the addition of 0 μL of HTRF detection buffer supplemented with Europium cryptate-labeled anti-human IgG (PerkinElmer-AD0212) specific for Fc and anti-His antibody conjugated to SureLight®-Allophycocyanin (APC, PerkinElmer-AD0059H). After centrifugation, the plate was incubated at 25° C. for 60 min. Before reading on a PHERAstar FS plate reader (665 nm/620 nm ratio). Final concentrations in the assay were −3 nM PD1, 10 nM PD-L1, 1 nM europium anti-human IgG and 20 nM anti-His-Allophycocyanin. IC₅₀ determination was performed by fitting the curve of percent control activity versus the log of the inhibitor concentration using the GraphPad Prism 5.0 software.

Compounds of the present disclosure, as exemplified in the Examples, showed IC₅₀ values in the following ranges: “*” stands for “IC₅₀-25 nM”; “**” stands for “25 nM<IC₅₀≤100 nM”; “***” stands for “100 nM<IC₅₀≤200 nM”; “****” stands for “IC₅₀>200 nM”.

Data obtained for the Example compounds using the PD-1/PD-L1 homogenous time-resolved fluorescence (HTRF) binding assay described in Example A is provided in Table 4.

TABLE 4

| EX No. | IC₅₀ | EX No. | IC₅₀ |
|---|---|---|---|
| Com. EX. No. 1 | 212 | Com. EX. No. 2 | 130.7 |
| 1 | 0.48 | 84 | ** |
| 2 | 0.74 | 85 | ** |
| 3 | * | 86 | * |
| 4 | * | 87 | * |
| 5 | *** | 88 | * |
| 6 | * | 89 | * |
| 7 | * | 90 | * |
| 8 | * | 91 | * |
| 9 | * | 92 | * |
| 10 | * | 93 | * |
| 11 | *** | 94 | * |
| 12 | ** | 95 | * |
| 13 | ** | 96 | * |
| 14 | * | 97 | * |
| 15 | * | 98 | * |
| 16 | * | 99 | * |
| 17 | * | 100 | * |
| 18 | ** | 101 | * |
| 19 | ** | 102 | * |
| 20 | * | 103 | 0.37 |
| 21 | *** | 104 | * |
| 22 | **** | 105 | * |
| 23 | *** | 106 | * |
| 24 | *** | 107 | * |
| 25 | ** | 108 | * |
| 26 | ** | 109 | * |
| 27 | 3.13 | 110 | * |
| 28 | * | 111 | * |
| 29 | * | 112 | * |
| 30 | * | 113 | * |
| 31 | * | 114 | * |
| 32 | 18.1 | 115 | * |
| 33 | 0.21 | 116 | * |
| 34 | * | 117 | * |
| 35 | * | 118 | * |
| 36 | * | 119 | * |
| 37 | * | 120 | * |
| 38 | * | 121 | * |
| 39 | * | 122 | * |
| 40 | * | 123 | * |
| 41 | * | 124 | * |
| 42 | * | 125 | * |
| 43 | * | 126 | * |
| 44 | 0.62 | 127 | * |
| 45 | * | 128 | ** |
| 46 | * | 129 | ** |
| 47 | * | 130 | * |
| 48 | * | 131 | ** |
| 49 | * | 132 | ** |
| 50 | * | 133 | * |
| 51 | * | 134 | * |
| 52 | * | 135 | * |
| 53 | * | 136 | *** |
| 54 | * | 137 | *** |
| 55 | * | 138 | * |
| 56 | * | 139 | * |
| 57 | * | 140 | * |
| 58 | * | 141 | * |
| 59 | * | 142 | * |
| 60 | * | 143 | * |
| 61 | * | 144 | * |
| 62 | * | 145 | * |
| 63 | * | 146 | * |
| 64 | * | 147 | * |
| 65 | * | 148 | * |
| 66 | * | 149 | * |
| 67 | * | 150 | * |
| 68 | * | 151 | * |
| 69 | * | 152 | * |
| 70 | * | 153 | * |
| 71 | * | 154 | * |
| 72 | * | 155 | * |
| 73 | * | 156 | * |
| 74 | * | 157 | * |
| 75 | *** | 158 | * |
| 76 | * | 159 | * |
| 77 | ** | 160 | * |
| 78 | * | 161 | * |
| 79 | * | 162 | * |
| 80 | * | 163 | * |
| 81 | * | 164 | * |
| 82 | * | 165 | * |
| 83 | * | 166 | * |
| 167 | * | 179 | ** |
| 168 | * | 180 | * |
| 169 | * | 181 | * |
| 170 | * | 182 | * |
| 171 | * | 183 | * |
| 172 | *** | 184 | **** |
| 173 | * | 185 | * |
| 174 | * | 186 | * |
| 175 | * | 187 | ** |
| 176 | ** | 188 | * |

1. A compound of Formula I, or a stereoisomer, tautomer, pharmaceutically acceptable salt, prodrug, chelate, non-covalent complex, or solvate thereof, 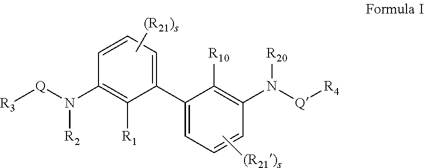
wherein,
Q and Q′ are each independently selected from absent, C(O) or C(R₂)₂;
R₁ and R₁₀ are each independently selected from H, halogen, CN, or C₁₋₈alkyl;
R₂ and R₂₀ are each independently selected from H, or C₁₋₈alkyl; or
R₁ and R₂ together with the atoms to which they are attached form a 5- to 6-member heterocyclic ring; or
R₁₀ and R₂₀ together with the atoms to which they are attached form a 5- to 6-member heterocyclic ring;
R₃ and R₄ are each independently selected from heterocyclic ring or heteroaryl ring optionally comprising 1, 2 or 3 hetero atoms independently selected from N, S, or O, wherein the heterocyclic ring or heteroaryl ring is monocyclic or bicyclic, optionally substituted with C₁₋₈alkyl, —(CH₂)ₚ—COOH, —(CH₂)ₚ—CONR₃₀R₄₀, —(CH₂)ₚ—OH, —(CH₂)ₚ-heterocyclyl, —C₃₋₇cycloalkyl, —C₃₋₇heterocyclyl, or —(CH₂)ₚ—NR₃₀R₄₀, wherein C₁₋₈alkyl, —(CH₂)ₚ—COOH, —(CH₂)ₚ—CONR₃₀R₄₀, —(CH₂)ₚ—OH, —(CH₂)ₚ-heterocyclyl, —C₃₋₇cycloalkyl, —C₃₋₇heterocyclyl, or —(CH₂)ₚ—NR₃₀R₄₀ is optionally substituted with C₁₋₈alkyl, halogen, OH, CN, COOH, or NR₃₀R₄₀;
R₃₀ and R₄₀ are each independently selected from H, C₁₋₈alkyl, —C₃₋₇cycloalkyl, or —C₃₋₇heterocyclyl; or
R₃₀ and R₄₀ together with the atoms to which they are attached form a 5- to 6-member heterocyclic ring;
R₂₁ and R₂₁′ are each independently selected from halogen, CN, OH, COOH, or C₁₋₈alkyl;
s and p are each independently selected from 0, 1, 2 or 3.

2. The compound of claim 1, wherein Q and Q′ are each independently selected from absent, C(O) or CH₂; or
s and p are each independently selected from 0 or 1; or
R₁ and R₁₀ are each independently selected from H, methyl, F or Cl; or
R₂ and R₂₀ are each independently selected from H or methyl; or
R₁ and R₂ together with the atoms to which they are attached form a 5-member heterocyclic ring; or
R₁₀ and R₂₀ together with the atoms to which they are attached form a 5-member heterocyclic ring; or
R₂₁ and R₂₁′ are each independently selected from —CH₃, F, Cl or CN.

3-8. (canceled)

9. The compound of claim 1, wherein R₃ and R₄ are each independently selected from 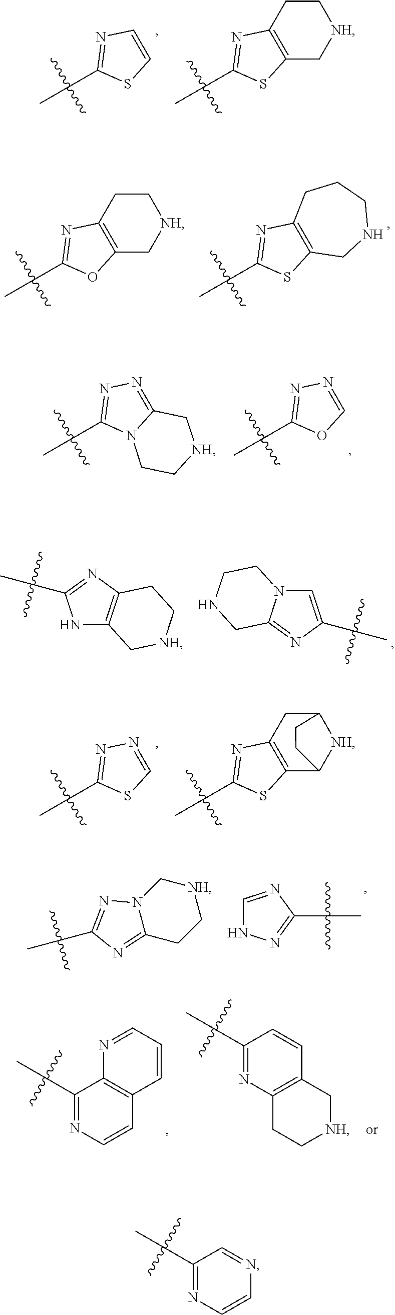
which is each unsubstituted or substituted with at least one substituent selected from C₁₋₆alkyl, —(CH₂)ₚ—COOH, —(CH₂)ₚ—CONR₃₀R₄₀, —(CH₂)ₚ—OH, —(CH₂)ₚ-heterocyclyl, —C₃₋₇cycloalkyl, —C₃₋₇heterocyclyl, or —(CH₂)ₚ—NR₃₀R₄₀, wherein C₁₋₆alkyl, —(CH₂)ₚ—COOH, —(CH₂)ₚ—NH₂, —(CH₂)ₚ—CONR₃₀R₄₀, —(CH₂)ₚ—OH, —(CH₂)ₚ-heterocyclyl, —C₃₋₇cycloalkyl, —C₃₋₇heterocyclyl, or —(CH₂)ₚ—NR₃₀R₄₀ is optionally substituted with C₁₋₈alkyl, halogen, OH, CN, COOH, or NR₃₀R₄₀;
R₃₀ and R₄₀ are each independently selected from H, C₁₋₈alkyl, —C₃₋₇cycloalkyl, or —C₃₋₇heterocyclyl; or
R₃₀ and R₄₀ together with the atoms to which they are attached form a 5- to 6-member heterocyclic ring.

10. The compound of claim 9, wherein, R₃₀ and R₄₀ are each independently selected from H, or C₁₋₃alkyl; or
R₃ and R₄ are each independently selected from

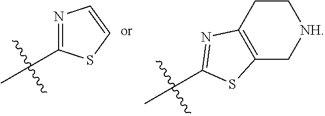 [SMILES: *c1nccs1 ; *c1nc2c(s1)CNCC2]

11. (canceled)

12. The compound of claim 1, wherein R₃ and R₄ are each independently selected from

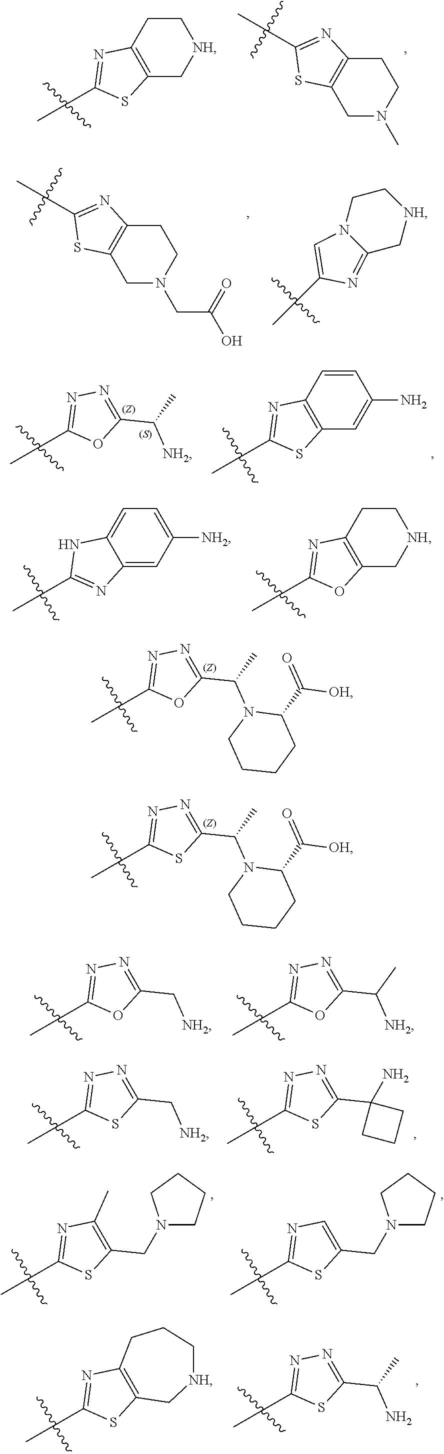

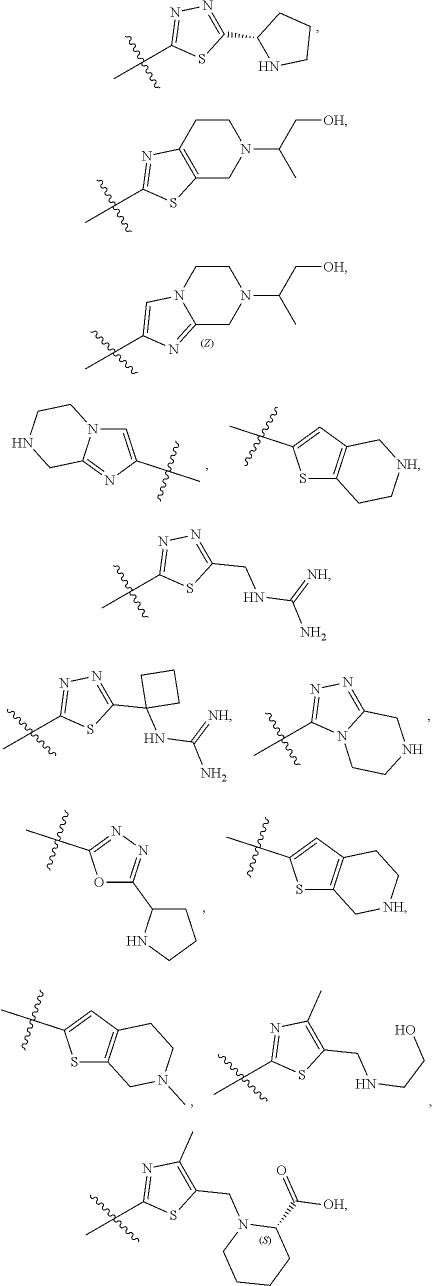

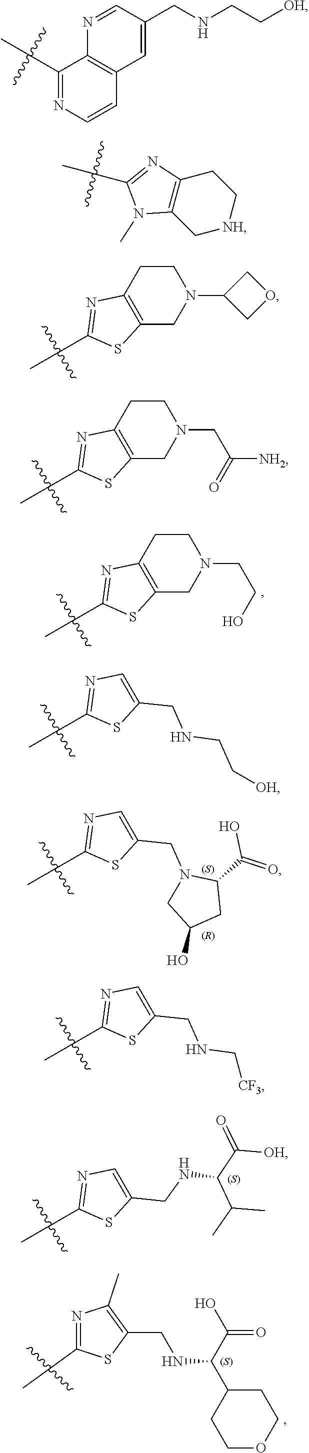

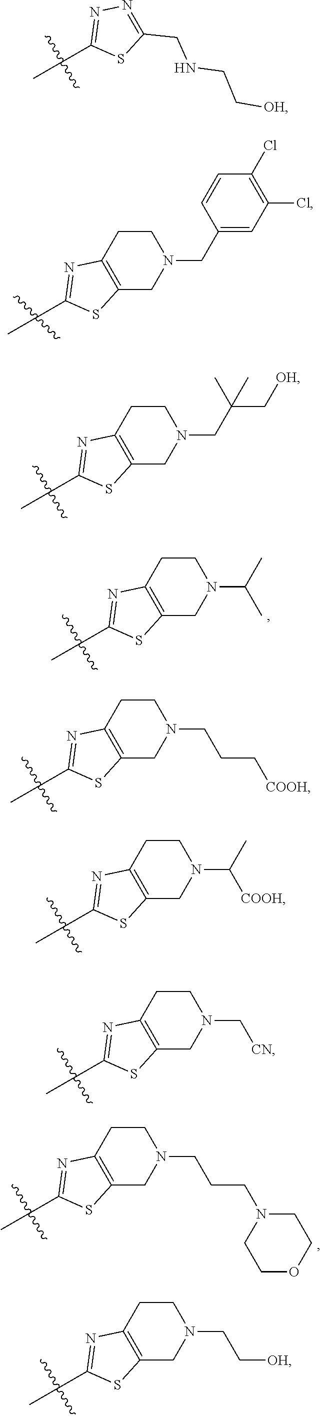

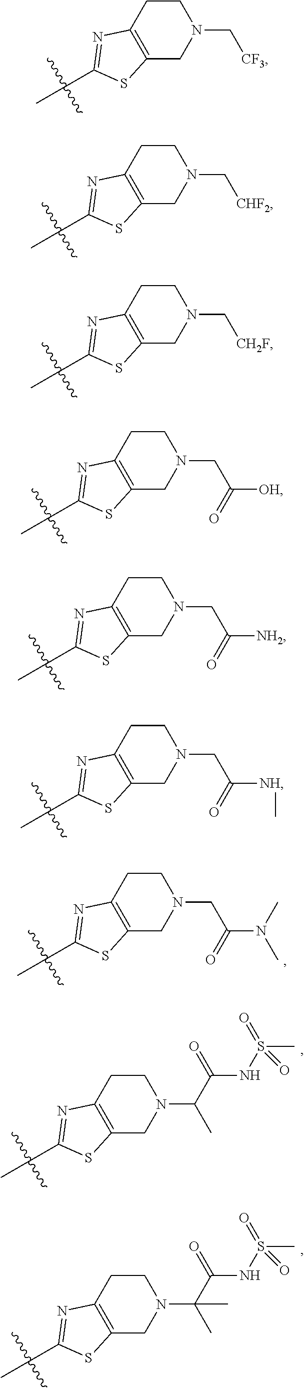

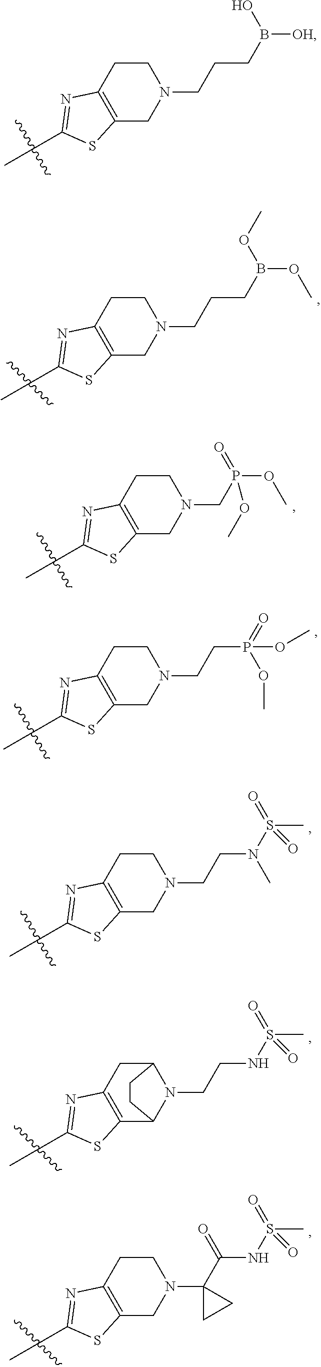

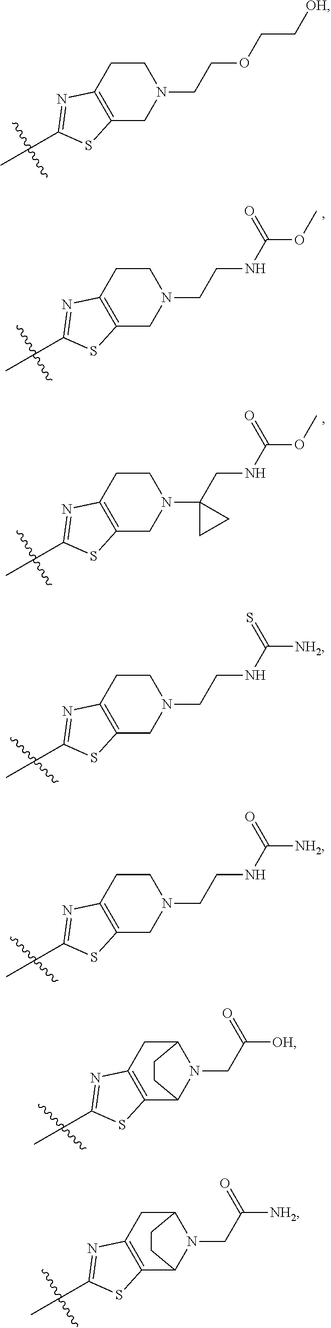

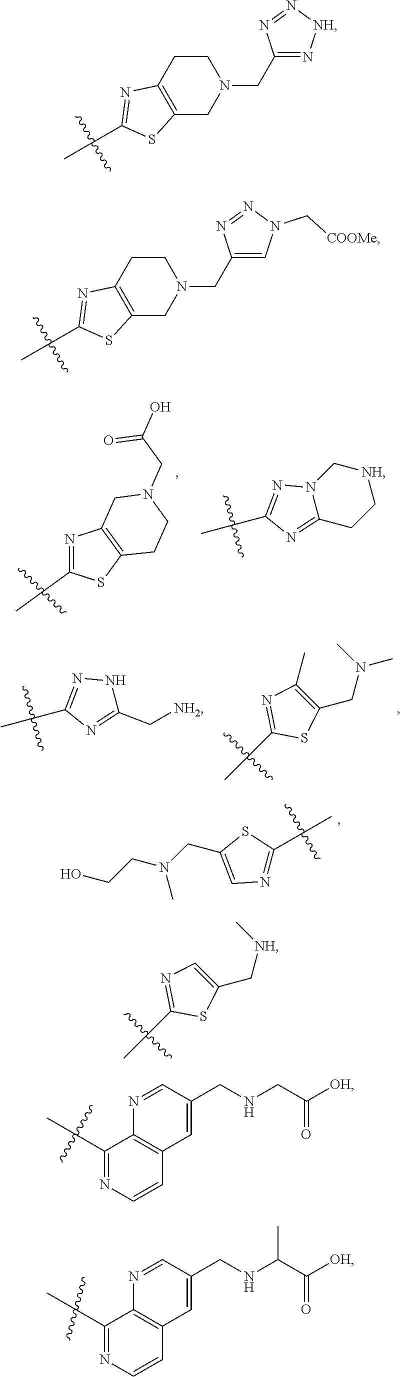

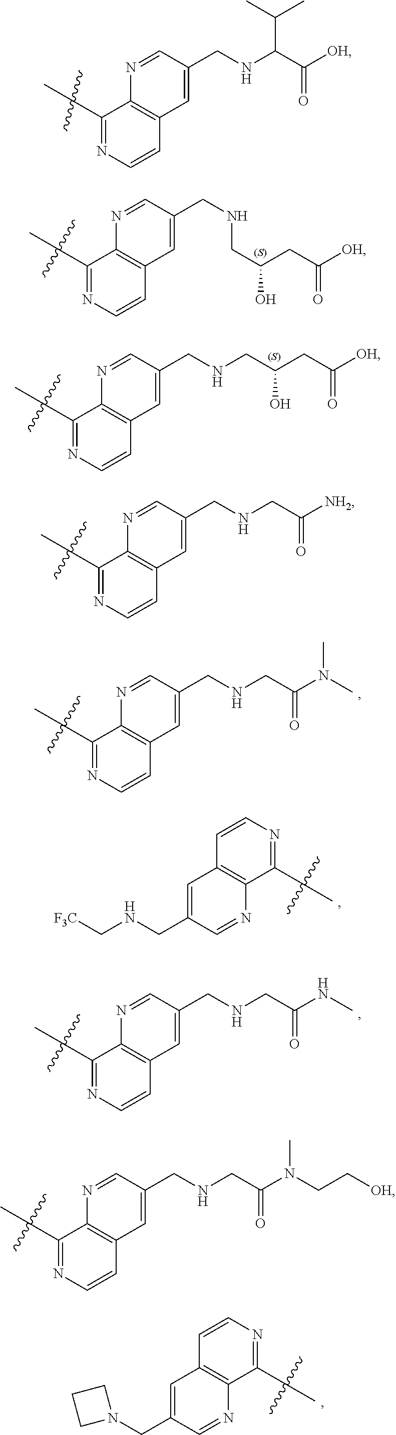

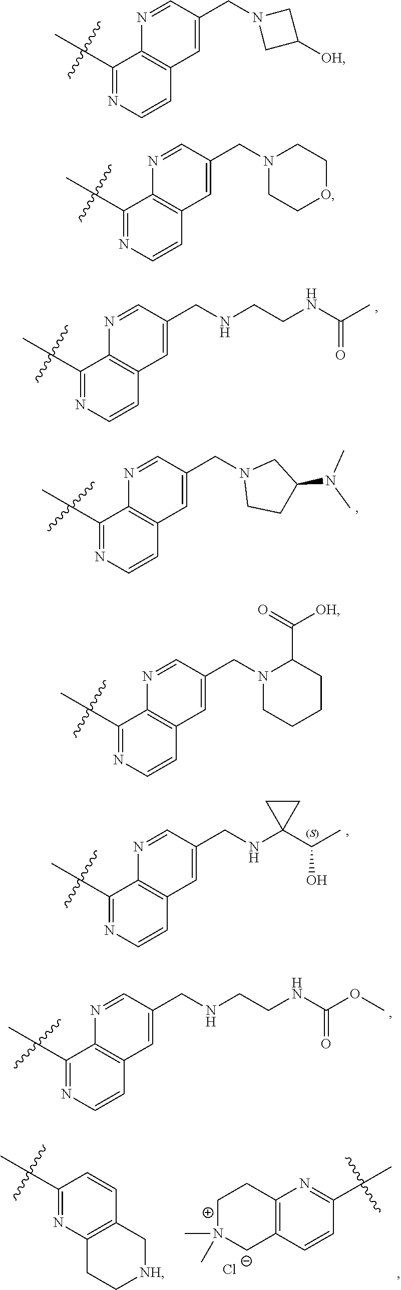

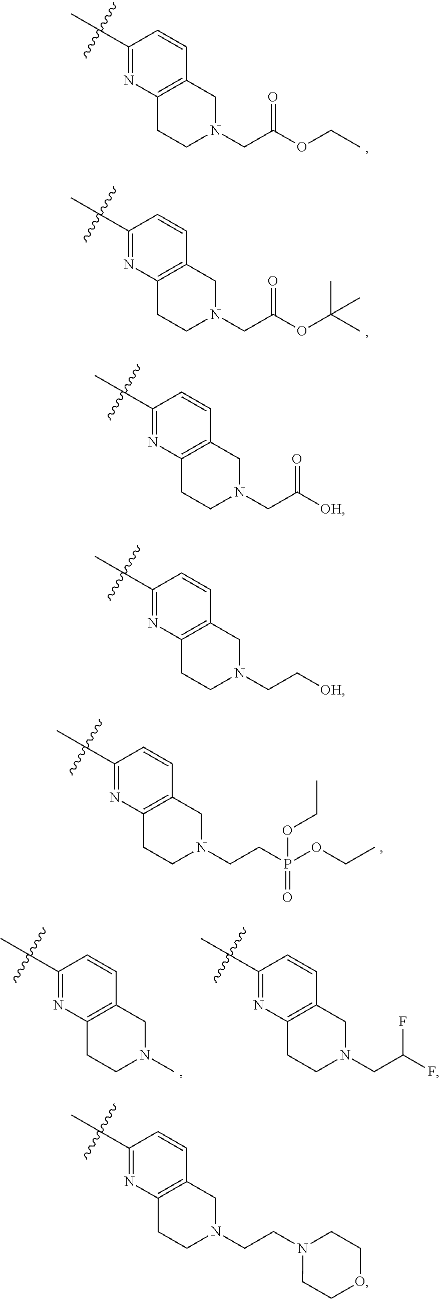

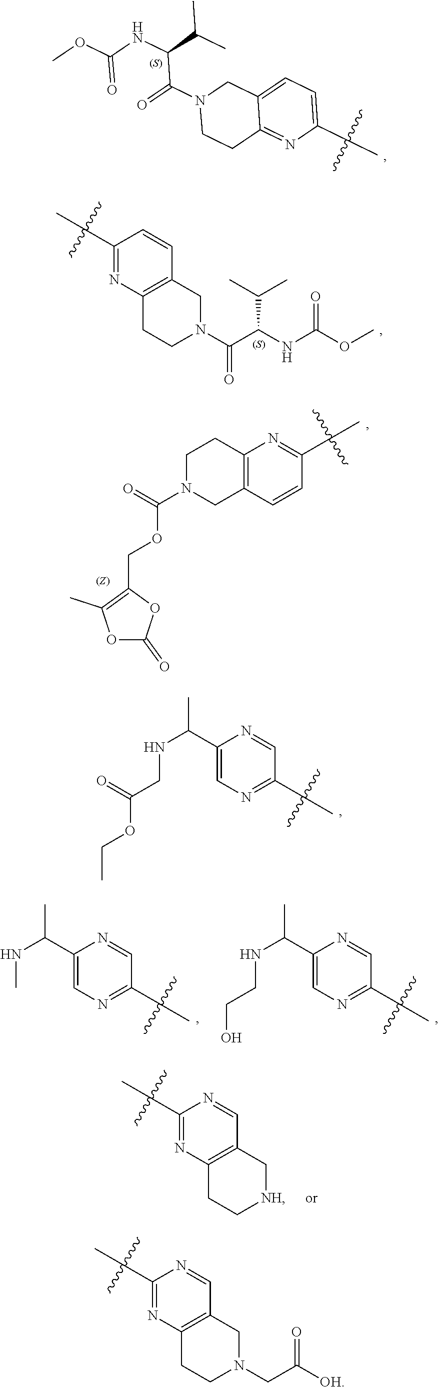

13. The compound of claim 1, wherein the compound is of Formula II: 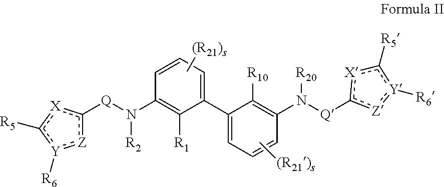
wherein,
R₁ and R₁₀ are each independently selected from H, halogen, CN, or C₁₋₈ alkyl;
R₂ and R₂₀ are each independently selected from H, or C₁₋₈ alkyl; or
R₁ and R₂ together with the atoms to which they are attached form a 5- to 6-member heterocyclic ring; or
R₁₀ and R₂₀ together with the atoms to which they are attached form a 5- to 6-member heterocyclic ring;
Q and Q′ are each independently selected from absent, C(O) or C(R₂)₂;
X, Y, Z, X′, Y′ or Z′ is independently selected from N, S, O or C;
R₅, R₆, R₅′ and R₆′ are each independently selected from (CH₂)ₚ—NR₃₀R₄₀, (CH₂)ₘ-cycloalkyl, (CH₂)ₘ-heterocyclyl, C₁₋₈alkyl, wherein (CH₂)ₚ—NR₃₀R₄₀, (CH₂)ₘ-heterocyclyl, C₁₋₈alkyl are each unsubstituted or substituted with at least one substituent selected from C₁₋₈alkyl, —COOH, —NH₂, (CH₂)ₘ-hydroxyl, or CN; or
R₅ and R₆ together with the atoms to which they are attached form a 6- to 7-member heterocyclic ring comprising 1, 2 or 3 hetero atoms independently selected from N, O or S, the heterocyclic ring being unsubstituted or substituted with at least one substituent selected from C₁₋₈alkyl, (CH₂)ₘ-carboxyl, (CH₂)ₘ-hydroxyl, (CH₂)ₘ-heterocyclyl, (CH₂)ₘ-aryl, (CH₂)ₘ-amido, (CH₂)ₘ—CN, (CH₂)ₘ—CF₃, (CH₂)ₘ—CHF₂, (CH₂)ₘCH₂F, or (CH₂)ₘ—NH₂; or
R₅′ and R₆′ together with the atoms to which they are attached form a 6- to 7-member heterocyclic ring comprising 1, 2 or 3 hetero atoms independently selected from N, O or S, the heterocyclic ring being unsubstituted or substituted with at least one substituent selected from C₁₋₈alkyl, (CH₂)ₘ-carboxyl, (CH₂)ₘ-hydroxyl, (CH₂)ₘ-heterocyclyl, (CH₂)ₘ-aryl, (CH₂)ₘ-amido, (CH₂)ₘ—CN, (CH₂)ₘ—CF₃, (CH₂)ₘ—CHF₂, (CH₂)ₘ—CH₂F, or (CH₂)ₘ—NH₂;
R₃₀ and R₄₀ are each independently selected from H, C₁₋₈alkyl, —C₃₋₇cycloalkyl, or —C₃₋₇heterocyclyl; or
R₃₀ and R₄₀ together with the atoms to which they are attached form a 5- to 6-member heterocyclic ring;
R₂₁ and R₂₁′ are each independently selected from halogen, CN, OH, COOH, or C₁₋₈alkyl;
p, s, and m are each independently selected from 0, 1, 2 or 3; [symbol]
is a single bond or a double bond.

14. The compound of claim 13, wherein R₁ and R₁₀ are independently selected from H, methyl, F or Cl; or
R₂ and R₂₀ are independently selected from H or methyl; or
R₁ and R₂ together with the atoms to which they are attached form a 5-member heterocyclic ring; or
R₁₀ and R₂₀ together with the atoms to which they are attached form a 5-member heterocyclic ring; or
R₂₁ and R₂₁′ are each independently selected from —CH₃, F, Cl or CN; or
X and X′ are each independently selected from O, S or N; or
Y and Y′ are each independently selected from C, S or N; or
Z and Z′ are each independently selected from C, or N; or
R₅ and R₅′ are each independently selected from —CH₃, 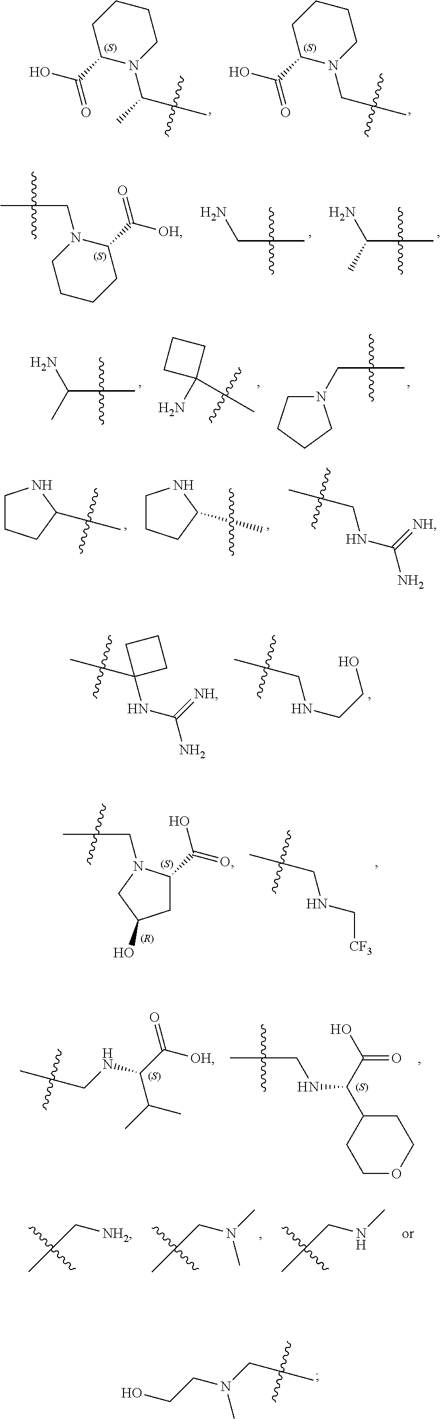
or
R₆ and R₆′ are each independently selected from absent, H or methyl.

15-23. (canceled)

24. The compound of claim 1, the compound is of Formula III: 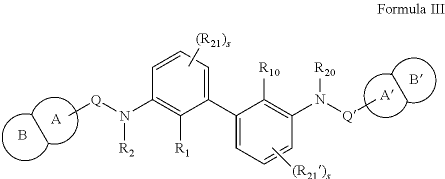
wherein,
R₁ and R₁₀ are each independently selected from H, halogen, CN, or C₁₋₈alkyl;
R₂ and R₂₀ are each independently selected from H, or C₁₋₈alkyl; or
R₁ and R₂ together with the atoms to which they are attached form a 5- to 6-member heterocyclic ring; or
R₁₀ and R₂₀ together with the atoms to which they are attached form a 5- to 6-member heterocyclic ring;
Q and Q′ are each independently selected from absent, C(O) or C(R₂)₂;
Ring A and Ring A′ are independently a 5-6 membered aromatic heterocyclic ring, which is each unsubstituted or substituted with at least one substituent selected from C₁₋₈alkyl, halogen, OH, or CN;
Ring B and Ring B′ are independently a 5-7 membered heterocyclic ring, which is each unsubstituted or substituted with at least one substituent selected from C₁₋₈alkyl, —(CH₂)ₚ—COOH, —(CH₂)ₚ—CONR₃₀R₄₀, —(CH₂)ₚ—OH, —(CH₂)ₚ-heterocyclyl, —C₃₋₇cycloalkyl, —C₃₋₇heterocyclyl, or —(CH₂)ₚ—NR₃₀R₄₀, wherein C₁₋₈alkyl, —(CH₂)ₚ—COOH, —(CH₂)ₚ—CONR₃₀R₄₀, —(CH₂)ₚ—OH, —(CH₂)ₚ-heterocyclyl, —C₃₋₇cycloalkyl, —C₃₋₇heterocyclyl, or —(CH₂)ₚ—NR₃₀R₄₀ is optionally substituted with C₁₋₈alkyl halogen, OH, CN, COOH, or NR₃₀R₄₀;
R₃₀ and R₄₀ are each independently selected from H, C₁₋₈alkyl, —C₃₋₇cycloalkyl, or —C₃₋₇heterocyclyl; or
R₃₀ and R₄₀ together with the atoms to which they are attached form a 5- to 6-member heterocyclic ring;
R₂₁ and R₂₁′ are each independently selected from halogen, CN, OH, COOH, or C₁₋₈alkyl;
p and s are each independently selected from 0, 1, 2 or 3.

25. The compound of claim 24, wherein R₁ and R₁₀ are independently selected from H, methyl, F or Cl; or
R₂ and R₂₀ are independently selected from H or methyl; or
R₁ and R₂ together with the atoms to which they are attached form a 5-membered heterocyclic ring; or
R₁₀ and R₂₀ together with the atoms to which they are attached form a 5-membered heterocyclic ring; or
R₂₁ and R₂₁′ are each independently selected from —CH₃, F, Cl or CN; or
s and p are each independently selected from 0 or 1; or 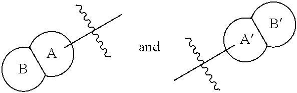
and are each independently selected from

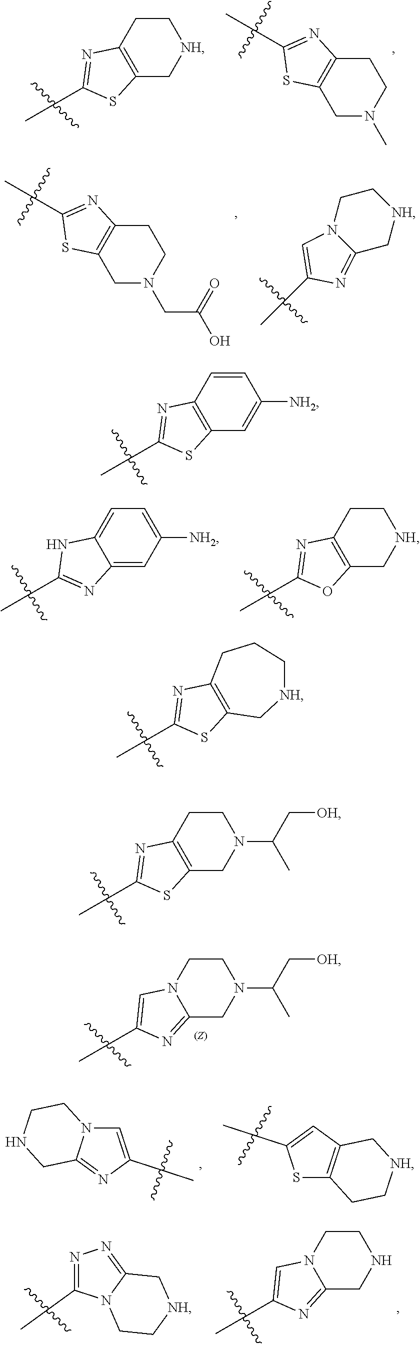

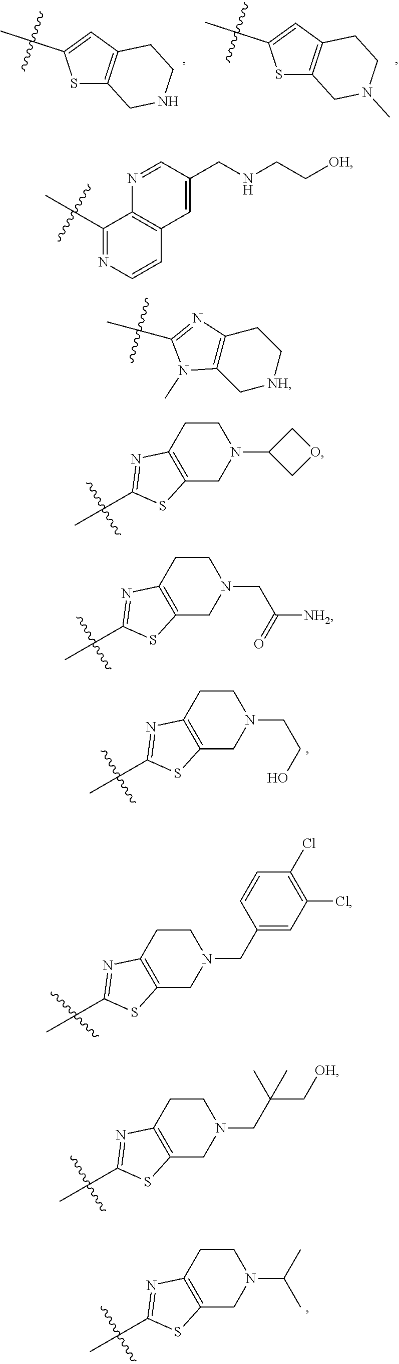

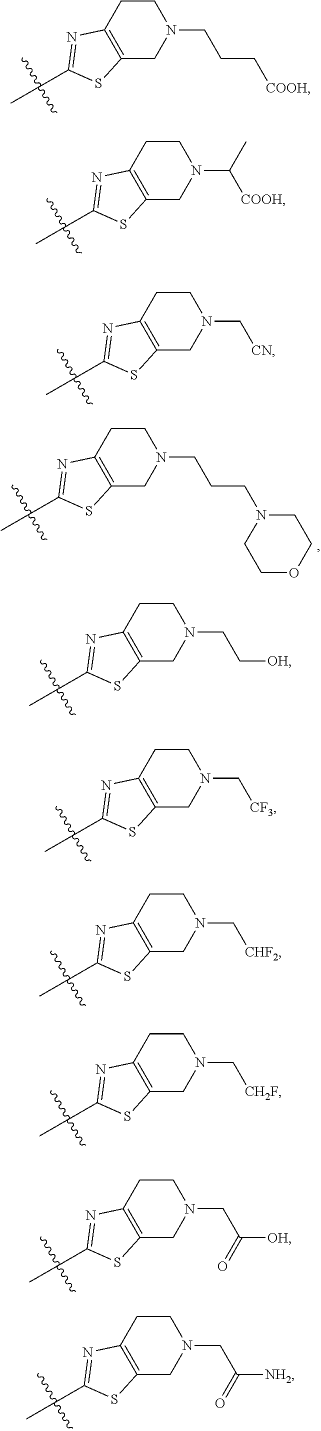

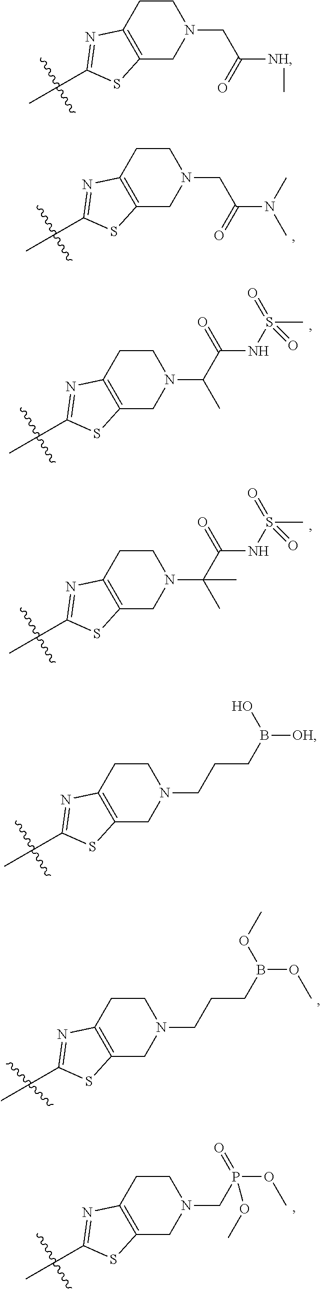

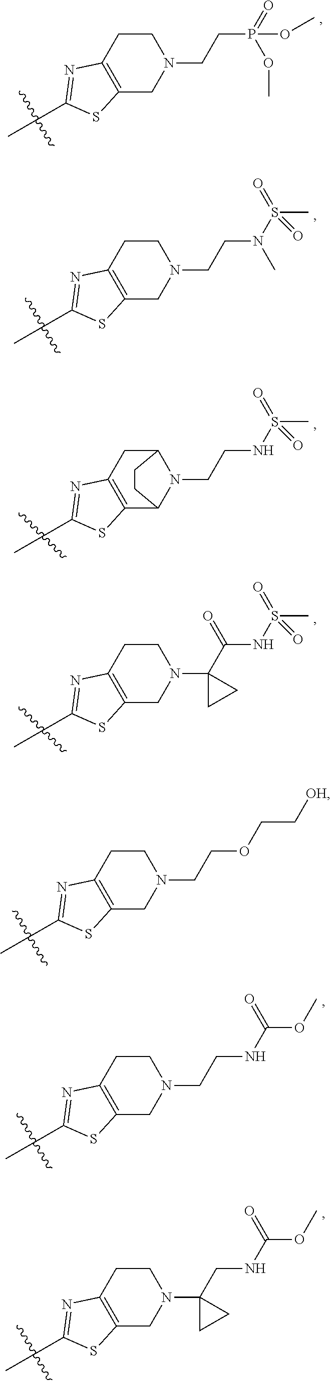

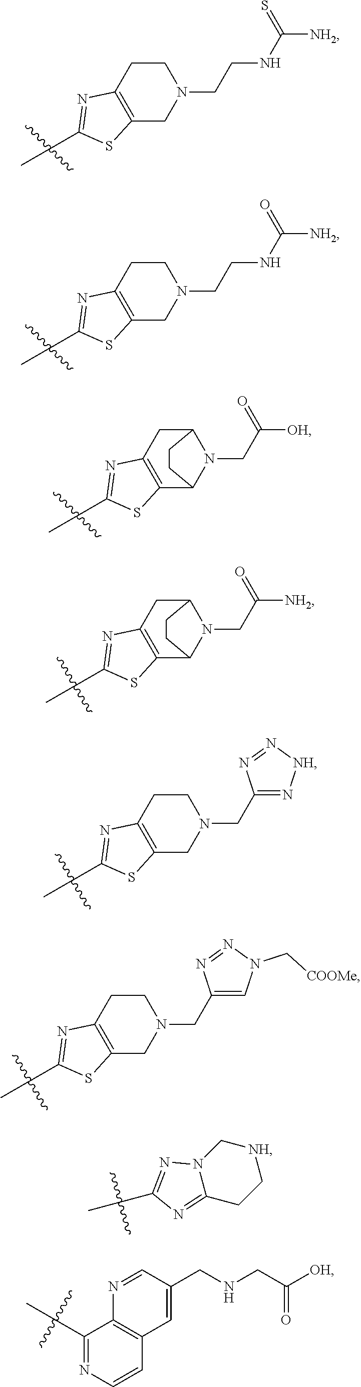

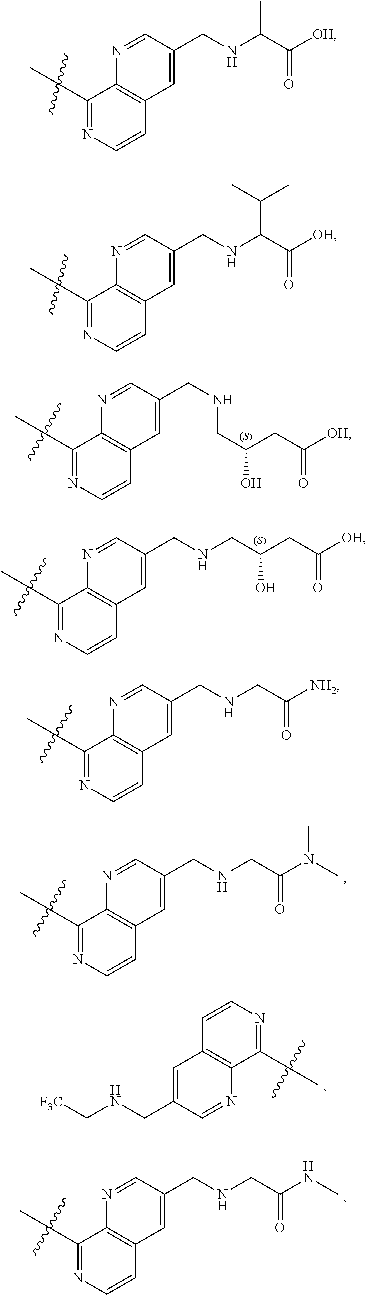

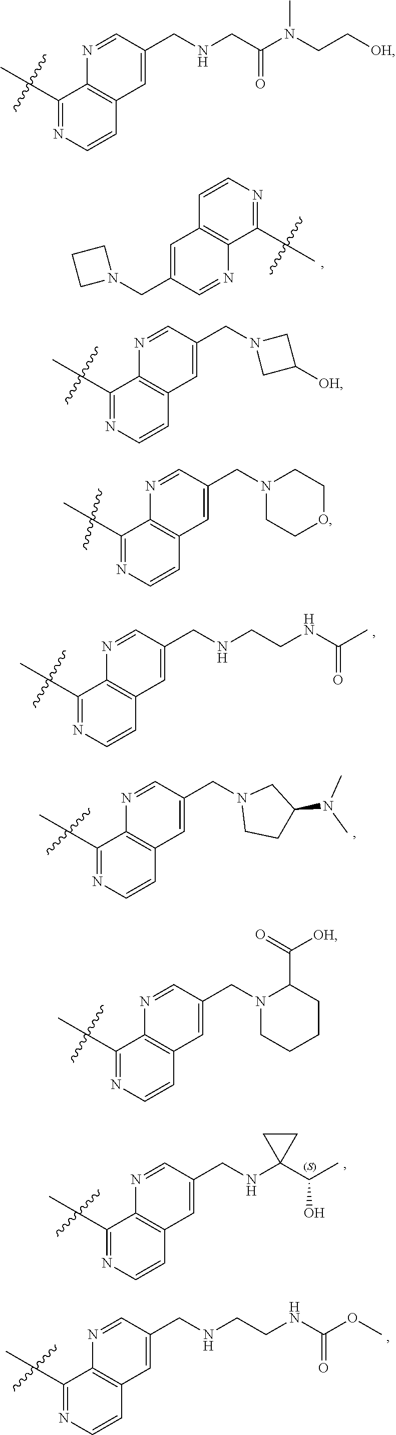

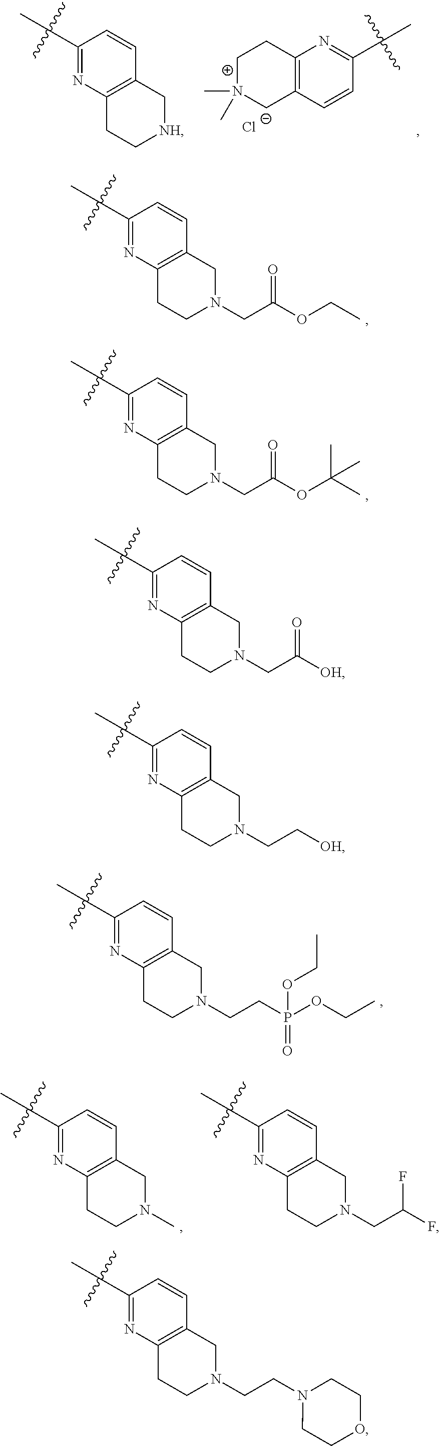

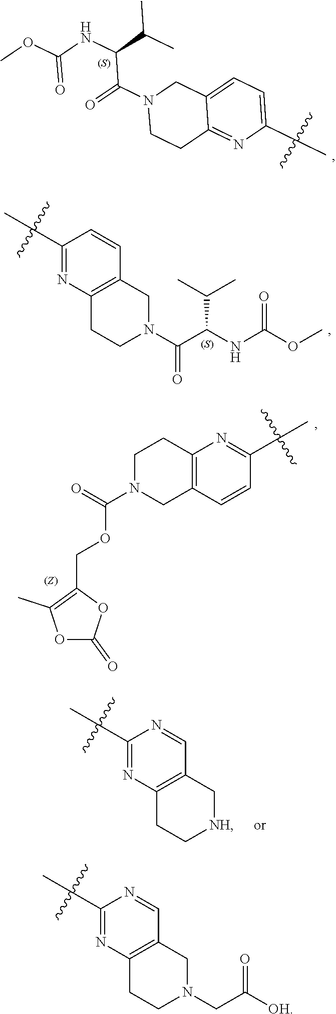

26-31. (canceled)

32. The compound of claim 1, wherein Q and Q′ are the same, R₁ and R₁₀ are the same, R₂ and R₂₀ are the same, R₃ and R₄ are the same, R₂₁ and R₂₁′ are the same.

33. The compound of claim 1, wherein the compound is
1) N,N′-(2,2′-dimethyl-[1,1′-biphenyl]-3,3′-diyl)bis(4,5,6,7-tetrahydrothiazolo[5,4-c]pyridine-2-carboxamide);
2) N,N′-(2,2′-dimethyl-[1,1′-biphenyl]-3,3′-diyl)bis(5-methyl-4,5,6,7-tetrahydrothiazolo[5,4-c]pyridine-2-carboxamide);
3) N,N′-(2,2′-dimethyl-[1,1′-biphenyl]-3,3′-diyl)bis(5-(pyrrolidin-2-yl)-1,3,4-thiadiazole-2-carboxamide);
4) 2,2′-((((2,2′-dimethyl-[1,1′-biphenyl]-3,3′-diyl)bis(azanediyl))bis(carbonyl))bis(6,7-dihydrothiazolo[5,4-c]pyridine-2,5(4H)-diyl))diacetic acid;
5) N-(2,2′-dimethyl-3′-(N-methyl-5,6,7,8-tetrahydroimidazo[1,2-a]pyrazine-2-carboxamido)-[1,1′-biphenyl]-3-yl)-N-methyl-4,5,6,7-tetrahydrothieno[2,3-c]pyridine-2-carboxamide;
6) 2-((8-((3′-((3-(((2-hydroxyethyl)amino)methyl)-1,7-naphthyridin-8-yl)amino)-2,2′-dimethyl-[1,1′-biphenyl]-3-yl)amino)quinolin-3-yl)amino)ethan-1-ol;
7) N,N′-(2,2′-dimethyl-[1,1′-biphenyl]-3,3′-diyl)bis(5-(((2-hydroxyethyl)amino)methyl)thiazole-2-carboxamide);
8) N,N′-(2,2′-dimethyl-[1,1′-biphenyl]-3,3′-diyl)bis(5,6,7,8-tetrahydroimidazo[1,2-a]pyrazine-2-carboxamide);
9) N,N′-(2,2′-dimethyl-[1,1′-biphenyl]-3,3′-diyl)bis(5-((S)-1-aminoethyl)-1,3,4-oxadiazole-2-carboxamide);
10) N-(2,2′-dimethyl-3′-(5,6,7,8-tetrahydroimidazo[1,2-a]pyrazine-2-carboxamido)-[1,1′-biphenyl]-3-yl)-4,5,6,7-tetrahydrothiazolo[5,4-c]pyridine-2-carboxamide;
11) N-(3′-(6-aminobenzo[d]thiazole-2-carboxamido)-2,2′-dimethyl-[1,1′-biphenyl]-3-yl)-4,5,6,7-tetrahydrothiazolo[5,4-c]pyridine-2-carboxamide;
12) N-(3′-(5-amino-1H-benzo[d]imidazole-2-carboxamido)-2,2′-dimethyl-[1,1′-biphenyl]-3-yl)-4,5,6,7-tetrahydrothiazolo[5,4-c]pyridine-2-carboxamide;
13) N,N′-(2,2′-dimethyl-[1,1′-biphenyl]-3,3′-diyl)bis(4,5,6,7-tetrahydrooxazolo[5,4-c]pyridine-2-carboxamide);
14) N-(2,2′-dimethyl-3′-(4,5,6,7-tetrahydrothiazolo[5,4-c]pyridine-2-carboxamido)-[1,1′-biphenyl]-3-yl)-4,5,6,7-tetrahydrooxazolo[5,4-c]pyridine-2-carboxamide;
15) N-(2,2′-dimethyl-3′-(4,5,6,7-tetrahydrothieno[3,2-c]pyridine-2-carboxamido)-[1,1′-biphenyl]-3-yl)-4,5,6,7-tetrahydrothiazolo[5,4-c]pyridine-2-carboxamide;
16)(2S,2'S)-1,1′-((1S,1'S)-((((2,2′-dimethyl-[1,1′-biphenyl]-3,3′-diyl)bis(azanediyl))bis(carbonyl))bis(1,3,4-oxadiazole-5,2-diyl))bis(ethane-1,1-diyl))bis(piperidine-2-carboxylic acid);
17)(2S,2'S)-1,1′-((1S,1'S)-((((2,2′-dimethyl-[1,1′-biphenyl]-3,3′-diyl)bis(azanediyl))bis(carbonyl))bis(1,3,4-thiadiazole-5,2-diyl))bis(ethane-1,1-diyl))bis(piperidine-2-carboxylic acid);
18) N,N′-(2,2′-dimethyl-[1,1′-biphenyl]-3,3′-diyl)bis(5-(aminomethyl)-1,3,4-oxadiazole-2-carboxamide);
19) N,N′-(2,2′-dimethyl-[1,1′-biphenyl]-3,3′-diyl)bis(5-(1-aminoethyl)-1,3,4-oxadiazole-2-carboxamide);
20) N,N′-(2,2′-dimethyl-[1,1′-biphenyl]-3,3′-diyl)bis(5-(aminomethyl)-1,3,4-thiadiazole-2-carboxamide);
21) N-(3′-(6-amino-N-methylbenzo[d]thiazole-2-carboxamido)-2,2′-dimethyl-[1,1′-biphenyl]-3-yl)-N-methyl-4,5,6,7-tetrahydrothiazolo[5,4-c]pyridine-2-carboxamide;
22) N3-((6-aminobenzo[d]thiazol-2-yl)methyl)-N3,N3′,2,2′-tetramethyl-N3′-((4,5,6,7-tetrahydrothiazolo[5,4-c]pyridin-2-yl)methyl)-[1,1′-biphenyl]-3,3′-diamine;
23) N,N′-(2,2′-dimethyl-[1,1′-biphenyl]-3,3′-diyl)bis(N-methyl-4,5,6,7-tetrahydrothiazolo[5,4-c]pyridine-2-carboxamide);
24) N3,N3′,2,2′-tetramethyl-N3,N3′-bis((4,5,6,7-tetrahydrothiazolo[5,4-c]pyridin-2-yl)methyl)-[1,1′-biphenyl]-3,3′-diamine;
25)2,2′-dimethyl-N3,N3′-bis((4,5,6,7-tetrahydrothiazolo[5,4-c]pyridin-2-yl)methyl)-[1,1′-biphenyl]-3,3′-diamine;
26) N3,N3′-bis((5-(1-aminocyclobutyl)-1,3,4-thiadiazol-2-yl)methyl)-2,2′-dimethyl-[1,1′-biphenyl]-3,3′-diamine;
27) N,N′-(2,2′-dimethyl-[1,1′-biphenyl]-3,3′-diyl)bis(5-(1-aminocyclobutyl)-1,3,4-thiadiazole-2-carboxamide);
28) N,N′-(2,2′-dimethyl-[1,1′-biphenyl]-3,3′-diyl)bis(4-methyl-5-(pyrrolidin-1-ylmethyl)thiazole-2-carboxamide);
29) N,N′-(2,2′-dimethyl-[1,1′-biphenyl]-3,3′-diyl)bis(5-(pyrrolidin-1-ylmethyl)thiazole-2-carboxamide);
30) N,N′-(2,2′-dimethyl-[1,1′-biphenyl]-3,3′-diyl)bis(5,6,7,8-tetrahydro-4H-thiazolo[5,4-c]azepine-2-carboxamide);
31) N,N′-(2,2′-dimethyl-[1,1′-biphenyl]-3,3′-diyl)bis(5-((S)-1-aminoethyl)-1,3,4-thiadiazole-2-carboxamide);
32) N,N′-(2,2′-dimethyl-[1,1′-biphenyl]-3,3′-diyl)bis(4,5,6,7-tetrahydrothieno[2,3-c]pyridine-2-carboxamide);
33)[4,4′-biindoline]-1,1′-diylbis((6,7-dihydro-4H-512-thiazolo[5,4-c]pyridin-2-yl)methanone);
34) N-(2,2′-dimethyl-3′-(N-methyl-5,6,7,8-tetrahydroimidazo[1,2-a]pyrazine-2-carboxamido)-[1,1′-biphenyl]-3-yl)-N-methyl-4,5,6,7-tetrahydrothieno[3,2-c]pyridine-2-carboxamide;
35) N-(2,2′-dimethyl-3′-(5,6,7,8-tetrahydroimidazo[1,2-a]pyrazine-2-carboxamido)-[1,1′-biphenyl]-3-yl)-4,5,6,7-tetrahydrothieno[3,2-c]pyridine-2-carboxamide;
36) N,N′-(2,2′-dimethyl-[1,1′-biphenyl]-3,3′-diyl)bis(7-(1-hydroxypropan-2-yl)-5,6,7,8-tetrahydroimidazo[1,2-a]pyrazine-2-carboxamide);
37) N,N′-(2,2′-dimethyl-[1,1′-biphenyl]-3,3′-diyl)bis(5-(guanidinomethyl)-1,3,4-thiadiazole-2-carboxamide);
38) N,N′-(2,2′-dimethyl-[1,1′-biphenyl]-3,3′-diyl)bis(5-(1-guanidinocyclobutyl)-1,3,4-thiadiazole-2-carboxamide);
39) N,N′-(2,2′-dimethyl-[1,1′-biphenyl]-3,3′-diyl)bis(5,6,7,8-tetrahydro-[1,2,4]triazolo[4,3-a]pyrazine-3-carboxamide);
40) N,N′-(2,2′-dicyano-[1,1′-biphenyl]-3,3′-diyl)bis(5,6,7,8-tetrahydroimidazo[1,2-a]pyrazine-2-carboxamide);
41) N,N′-(2,2′-dimethyl-[1,1′-biphenyl]-3,3′-diyl)bis(5-(pyrrolidin-2-yl)-1,3,4-oxadiazole-2-carboxamide);
42) N-(2,2′-dimethyl-3′-(5,6,7,8-tetrahydroimidazo[1,2-a]pyrazine-2-carboxamido)-[1,1′-biphenyl]-3-yl)-4,5,6,7-tetrahydrothieno[2,3-c]pyridine-2-carboxamide;
43) N,N′-(2,2′-dichloro-[1,1′-biphenyl]-3,3′-diyl)bis(4,5,6,7-tetrahydrothiazolo[5,4-c]pyridine-2-carboxamide);
44) N,N′-(2,2′-dichloro-[1,1′-biphenyl]-3,3′-diyl)bis(5-methyl-4,5,6,7-tetrahydrothiazolo[5,4-c]pyridine-2-carboxamide);
45) N,N′-(2,2′-dimethyl-[1,1′-biphenyl]-3,3′-diyl)bis(5-(1-hydroxypropan-2-yl)-4,5,6,7-tetrahydrothiazolo[5,4-c]pyridine-2-carboxamide);
46) 1,1′-bis((6,7-dihydro-4H-512-thiazolo[5,4-c]pyridin-2-yl)methyl)-4,4′-biindoline;
47)1,1′-bis(1-(5-methyl-4,5,6,7-tetrahydrothiazolo[5,4-c]pyridin-2-yl)ethyl)-4,4′-biindoline;
48) N,N′-(2-chloro-2′-methylbiphenyl-3,3′-diyl)bis(4,5,6,7-tetrahydrothiazolo[5,4-c]pyridine-2-carboxamide);
49) N,N′-(2-chloro-2′-methylbiphenyl-3,3′-diyl)bis(5-methyl-4,5,6,7-tetrahydrothiazolo[5,4-c]pyridine-2-carboxamide);
50) N-(2-chloro-2′-methyl-3′-(4,5,6,7-tetrahydrothiazolo[5,4-c]pyridine-2-carboxamido)biphenyl-3-yl)-5-methyl-4,5,6,7-tetrahydrothiazolo[5,4-c]pyridine-2-carboxamide;
51) N,N′-(2,2′-dimethylbiphenyl-3,3′-diyl)bis(5-((2-hydroxyethylamino)methyl)-4-methylthiazole-2-carboxamide);
52) (2S,2'S)-1,1′-(2,2′-(2,2′-dimethylbiphenyl-3,3′-diyl)bis(azanediyl)bis(oxomethylene)bis(4-methylthiazole-5,2-diyl))bis(methylene)dipiperidine-2-carboxylic acid;
53) (2S,2'S)-2,2′-(2,2′-(2,2′-dimethylbiphenyl-3,3′-diyl)bis(azanediyl)bis(oxomethylene)bis(4-methylthiazole-5,2-diyl))bis(methylene)bis(azanediyl)bis(2-(tetrahydro-2H-pyran-4-yl)acetic acid);
54) (2S,2'S)-1,1′-(2,2′-(2-chloro-2′-methylbiphenyl-3,3′-diyl)bis(azanediyl)bis(oxomethylene)bis(4-methylthiazole-5,2-diyl))bis(methylene)dipiperidine-2-carboxylic acid;
55) (2S,2'S)-2,2′-(2,2′-(2-chloro-2′-methylbiphenyl-3,3′-diyl)bis(azanediyl)bis(oxomethylene)bis(4-methylthiazole-5,2-diyl))bis(methylene)bis(azanediyl)bis(2-(tetrahydro-2H-pyran-4-yl)acetic acid);
56)(2S,2'S)-1,1′-(2,2′-(4,4′-biindoline-1,1′-diylbis(oxomethylene))bis(4-methylthiazole-5,2-diyl))bis(methylene)dipiperidine-2-carboxylic acid;
57)(2S,2'S)-2,2′-(2,2′-(4,4′-biindoline-1,1′-diylbis(oxomethylene))bis(4-methylthiazole-5,2-diyl))bis(methylene)bis(azanediyl)bis(2-(tetrahydro-2H-pyran-4-yl)acetic acid);
58)(S)-1-((2-(3-(1-(5-(((S)-2-carboxypiperidin-1-yl)methyl)-4-methylthiazole-2-carbonyl)indolin-4-yl)-2-methylphenylcarbamoyl)-4-methylthiazol-5-yl)methyl)piperidine-2-carboxylic acid;
59)(S)-2-((2-(3-(1-(5-(((S)-carboxy(tetrahydro-2H-pyran-4-yl)methylamino)methyl)-4-methylthiazole-2-carbonyl)indolin-4-yl)-2-methylphenylcarbamoyl)-4-methylthiazol-5-yl)methylamino)-2-(tetrahydro-2H-pyran-4-yl)acetic acid;
60)(S)-1-((2-(3-(1-(5-(((S)-2-carboxypiperidin-1-yl)methyl)-4-methylthiazole-2-carbonyl)indolin-4-yl)-2-chlorophenylcarbamoyl)-4-methylthiazol-5-yl)methyl)piperidine-2-carboxylic acid;
61)(S)-2-((2-(3-(1-(5-(((S)-carboxy(tetrahydro-2H-pyran-4-yl)methylamino)methyl)-4-methylthiazole-2-carbonyl)indolin-4-yl)-2-chlorophenylcarbamoyl)-4-methylthiazol-5-yl)methylamino)-2-(tetrahydro-2H-pyran-4-yl)acetic acid;
62)(S)-1-((2-(3-(1-(5-(((S)-2-carboxypiperidin-1-yl)methyl)-4-methylthiazole-2-carbonyl)indolin-4-yl)phenylcarbamoyl)-4-methylthiazol-5-yl)methyl)piperidine-2-carboxylic acid;
63)(S)-2-((2-(3-(1-(5-(((S)-carboxy(tetrahydro-2H-pyran-4-yl)methylamino)methyl)-4-methylthiazole-2-carbonyl)indolin-4-yl)phenylcarbamoyl)-4-methylthiazol-5-yl)methylamino)-2-(tetrahydro-2H-pyran-4-yl)acetic acid;
64) N,N′-(2,2′-dimethylbiphenyl-3,3′-diyl)bis(3-methyl-4,5,6,7-tetrahydro-3H-imidazo[4,5-c]pyridine-2-carboxamide);
65)2,2′-((((2,2′-dichloro-[1,1′-biphenyl]-3,3′-diyl)bis(azanediyl))bis(carbonyl))bis(6,7-dihydrothiazolo[5,4-c]pyridine-2,5(4H)-diyl))diacetic acid;
66) N,N′-(2,2′-dichloro-[1,1′-biphenyl]-3,3′-diyl)bis(5-(((2-hydroxyethyl)amino)methyl)thiazole-2-carboxamide);
67)(2S,2'S,4R,4′R)-1,1′-(((((2,2′-dichloro-[1,1′-biphenyl]-3,3′-diyl)bis(azanediyl))bis(carbonyl))bis(thiazole-2,5-diyl))bis(methylene))bis(4-hydroxypyrrolidine-2-carboxylic acid);
68) N,N′-(2,2′-dichloro-[1,1′-biphenyl]-3,3′-diyl)bis(5-(((2,2,2-trifluoroethyl)amino)methyl)thiazole-2-carboxamide);
69) ((2-((3′-(5-((((S)-1-carboxy-2-methylpropyl)amino)methyl)thiazole-2-carboxamido)-2,2′-dichloro-[1,1′-biphenyl]-3-yl)carbamoyl)thiazol-5-yl)methyl)-L-valine;
70) [4,4′-biindoline]-1,1′-diylbis((5-(((2-hydroxyethyl)amino)methyl)thiazol-2-yl)methanone);
71) (2S,2'S)-2,2′-((((((2,2′-dichloro-[1,1′-biphenyl]-3,3′-diyl)bis(azanediyl))bis(carbonyl))bis(thiazole-2,5-diyl))bis(methylene))bis(azanediyl))bis(2-(tetrahydro-2H-pyran-4-yl)acetic acid);
72) (S)-1-((2-((3-(1-(5-(((S)-2-carboxypiperidin-1-yl)methyl)thiazole-2-carbonyl)indolin-4-yl)-2-methylphenyl)carbamoyl)thiazol-5-yl)methyl)piperidine-2-carboxylic acid;
73) N,N′-(2,2′-dimethyl-[1,1′-biphenyl]-3,3′-diyl)bis(5-(((2-hydroxyethyl)amino)methyl)-1,3,4-thiadiazole-2-carboxamide);
74) N,N′-(2,2′-dicyano-[1,1′-biphenyl]-3,3′-diyl)bis(4,5,6,7-tetrahydrothiazolo[5,4-c]pyridine-2-carboxamide);
75) N,N′-(2,2′-dimethyl-[1,1′-biphenyl]-3,3′-diyl)bis(5-(3,4-dichlorobenzyl)-4,5,6,7-tetrahydrothiazolo[5,4-c]pyridine-2-carboxamide);
76) N,N′-(2,2′-dimethyl-[1,1′-biphenyl]-3,3′-diyl)bis(5-(3-hydroxy-2,2-dimethylpropyl)-4,5,6,7-tetrahydrothiazolo[5,4-c]pyridine-2-carboxamide);
77) N,N′-(2,2′-dimethyl-[1,1′-biphenyl]-3,3′-diyl)bis(5-isopropyl-4,5,6,7-tetrahydrothiazolo[5,4-c]pyridine-2-carboxamide);
78)4,4′-((((2,2′-dimethyl-[1,1′-biphenyl]-3,3′-diyl)bis(azanediyl))bis(carbonyl))bis(6,7-dihydrothiazolo[5,4-c]pyridine-2,5(4H)-diyl))dibutyric acid;
79)2,2′-((((2,2′-dimethyl-[1,1′-biphenyl]-3,3′-diyl)bis(azanediyl))bis(carbonyl))bis(6,7-dihydrothiazolo[5,4-c]pyridine-2,5(4H)-diyl))dipropionic acid;
80) N,N′-(2,2′-dimethyl-[1,1′-biphenyl]-3,3′-diyl)bis(5-(cyanomethyl)-4,5,6,7-tetrahydrothiazolo[5,4-c]pyridine-2-carboxamide);
81) N,N′-(2,2′-dimethyl-[1,1′-biphenyl]-3,3′-diyl)bis(5-(3-morpholinopropyl)-4,5,6,7-tetrahydrothiazolo[5,4-c]pyridine-2-carboxamide);
82) N,N′-(2,2′-dimethyl-[1,1′-biphenyl]-3,3′-diyl)bis(5-(2-hydroxyethyl)-4,5,6,7-tetrahydrothiazolo[5,4-c]pyridine-2-carboxamide);
83) [4,4′-biindoline]-1,1′-diylbis((5-methyl-4,5,6,7-tetrahydrothiazolo[5,4-c]pyridin-2-yl)methanone);
84) [4,4′-biindoline]-1,1′-diylbis((5-(2,2,2-trifluoroethyl)-4,5,6,7-tetrahydrothiazolo[5,4-c]pyridin-2-yl)methanone);
85) [4,4′-biindoline]-1,1′-diylbis((5-(2,2-difluoroethyl)-4,5,6,7-tetrahydrothiazolo[5,4-c]pyridin-2-yl)methanone);
86)2,2′-(([4,4′-biindoline]-1,1′-dicarbonyl)bis(6,7-dihydrothiazolo[5,4-c]pyridine-2,5(4H)-diyl))diacetamide;
87)[4,4′-biindoline]-1,1′-diylbis((5-(2-fluoroethyl)-4,5,6,7-tetrahydrothiazolo[5,4-c]pyridin-2-yl)methanone);
88)[4,4′-biindoline]-1,1′-diylbis((5-(oxetan-3-yl)-4,5,6,7-tetrahydrothiazolo[5,4-c]pyridin-2-yl)methanone);
89) N,N′-(2,2′-dichloro-[1,1′-biphenyl]-3,3′-diyl)bis(4,5,6,7-tetrahydrothiazolo[5,4-c]pyridine-2-carboxamide);
90) N,N′-(2,2′-dichloro-[1,1′-biphenyl]-3,3′-diyl)bis(5-(2-amino-2-oxoethyl)-4,5,6,7-tetrahydrothiazolo[5,4-c]pyridine-2-carboxamide);
91) N,N′-(2,2′-dichloro-[1,1′-biphenyl]-3,3′-diyl)bis(5-(2-hydroxyethyl)-4,5,6,7-tetrahydrothiazolo[5,4-c]pyridine-2-carboxamide);
92) N,N′-(2,2′-dichloro-[1,1′-biphenyl]-3,3′-diyl)bis(5-(oxetan-3-yl)-4,5,6,7-tetrahydrothiazolo[5,4-c]pyridine-2-carboxamide);
93) N,N′-(5,5′-dichloro-2,2′-dimethyl-[1,1′-biphenyl]-3,3′-diyl)bis(4,5,6,7-tetrahydrothiazolo[5,4-c]pyridine-2-carboxamide);
94) N,N′-(2,2′,4,4′-tetramethyl-[1,1′-biphenyl]-3,3′-diyl)bis(4,5,6,7-tetrahydrothiazolo[5,4-c]pyridine-2-carboxamide);
95) N-(5-fluoro-2-methyl-3-(1-(4,5,6,7-tetrahydrothiazolo[5,4-c]pyridine-2-carbonyl)indolin-4-yl)phenyl)-4,5,6,7-tetrahydrothiazolo[5,4-c]pyridine-2-carboxamide;
96) N,N′-(5,5′-difluoro-2,2′-dimethyl-[1,1′-biphenyl]-3,3′-diyl)bis(4,5,6,7-tetrahydrothiazolo[5,4-c]pyridine-2-carboxamide);
97) N-(2,2′-dichloro-3′-(5-(((2-hydroxyethyl)amino)methyl)thiazole-2-carboxamido)-[1,1′-biphenyl]-3-yl)-4,5,6,7-tetrahydrothiazolo[5,4-c]pyridine-2-carboxamide;
98) N-(2,2′-dichloro-3′-((3-(((2-hydroxyethyl)amino)methyl)-1,7-naphthyridin-8-yl)amino)-[1,1′-biphenyl]-3-yl)-5-(((2-hydroxyethyl)amino)methyl)thiazole-2-carboxamide;
99) (5-(((2-hydroxyethyl)amino)methyl)thiazol-2-yl)(1′-(4,5,6,7-tetrahydrothiazolo[5,4-c]pyridine-2-carbonyl)-[4,4′-biindolin]-1-yl)methanone;
100) (1′-(3-(((2-hydroxyethyl)amino)methyl)-1,7-naphthyridin-8-yl)-[4,4′-biindolin]-1-yl)(4,5,6,7-tetrahydrothiazolo[5,4-c]pyridin-2-yl)methanone;
101) N-(2-chloro-3-(1-(4,5,6,7-tetrahydrothiazolo[5,4-c]pyridine-2-carbonyl)indolin-4-yl)phenyl)-5-(((2-hydroxyethyl)amino)methyl)thiazole-2-carboxamide;
102) dimethyl 2,2′-(([4,4′-biindoline]-1,1′-dicarbonyl)bis(6,7-dihydrothiazolo[5,4-c]pyridine-2,5(4H)-diyl))diacetate;
103) 2,2′-(([4,4′-biindoline]-1,1′-dicarbonyl)bis(6,7-dihydrothiazolo[5,4-c]pyridine-2,5(4H)-diyl))diacetic acid;
104) 2,2′-(([4,4′-biindoline]-1,1′-dicarbonyl)bis(6,7-dihydrothiazolo[5,4-c]pyridine-2,5(4H)-diyl))diacetamide;
105) 2,2′-(([4,4′-biindoline]-1,1′-dicarbonyl)bis(6,7-dihydrothiazolo[5,4-c]pyridine-2,5(4H)-diyl))bis(N-methylacetamide);
106) 2,2′-(([4,4′-biindoline]-1,1′-dicarbonyl)bis(6,7-dihydrothiazolo[5,4-c]pyridine-2,5(4H)-diyl))bis(N,N-dimethylacetamide);
107) 2-(2-(1′-(5-(2-amino-2-oxoethyl)-4,5,6,7-tetrahydrothiazolo[5,4-c]pyridine-2-carbonyl)-[4,4′-biindoline]-1-carbonyl)-6,7-dihydrothiazolo[5,4-c]pyridin-5(4H)-yl)acetic acid;
108) 2-(2-(1′-(5-(2-(methylamino)-2-oxoethyl)-4,5,6,7-tetrahydrothiazolo[5,4-c]pyridine-2-carbonyl)-[4,4′-biindoline]-1-carbonyl)-6,7-dihydrothiazolo[5,4-c]pyridin-5(4H)-yl)acetic acid;
109) 2-(2-(1′-(5-(2-(dimethylamino)-2-oxoethyl)-4,5,6,7-tetrahydrothiazolo[5,4-c]pyridine-2-carbonyl)-[4,4′-biindoline]-1-carbonyl)-6,7-dihydrothiazolo[5,4-c]pyridin-5(4H)-yl)acetic acid;
110) 2,2′-(([4,4′-biindoline]-1,1′-dicarbonyl)bis(5,6,7,8-tetrahydro-4H-5,8-epiminocyclohepta[d]thiazole-2,9-diyl))diacetic acid;
111) 2-(2-(1′-(9-(2-amino-2-oxoethyl)-5,6,7,8-tetrahydro-4H-5,8-epiminocyclohepta[d]thiazole-2-carbonyl)-[4,4′-biindoline]-1-carbonyl)-5,6,7,8-tetrahydro-4H-5,8-epiminocyclohepta[d]thiazol-9-yl)acetic acid;
112) [4,4′-biindoline]-1,1′-diylbis((5-((dimethylamino)methyl)thiazol-2-yl)methanone);
113) [4,4′-biindoline]-1,1′-diylbis((5-((methylamino)methyl)thiazol-2-yl)methanone);
114) N-(3-(1-(5-((dimethylamino)methyl)-4-methylthiazole-2-carbonyl)indolin-4-yl)phenyl)-5-(((2-hydroxyethyl)(methyl)amino)methyl)thiazole-2-carboxamide;
115) (5-((dimethylamino)methyl)-4-methylthiazol-2-yl)(1′-(5-(((2-hydroxyethyl)(methyl)amino)methyl)thiazole-2-carbonyl)-[4,4′-biindolin]-1-yl)methanone;
116) [4,4′-biindoline]-1,1′-diylbis((5-(((2-hydroxyethyl)(methyl)amino)methyl)thiazol-2-yl)methanone);
117) N,N′-((([4,4′-biindoline]-1,1′-dicarbonyl)bis(6,7-dihydrothiazolo[5,4-c]pyridine-2,5 (4H)-diyl))bis(ethane-2,1-diyl))dimethanesulfonamide;
118) N,N′-((([4,4′-biindoline]-1,1′-dicarbonyl)bis(6,7-dihydrothiazolo[5,4-c]pyridine-2,5 (4H)-diyl))bis(ethane-2,1-diyl))bis(N-methylmethanesulfonamide);
119) 2,2′-(([4,4′-biindoline]-1,1′-dicarbonyl)bis(6,7-dihydrothiazolo[4,5-c]pyridine-2,5(4H)-diyl))diacetic acid;
120) [4,4′-biindoline]-1,1′-diylbis((5-(2-(2-hydroxyethoxy)ethyl)-4,5,6,7-tetrahydrothiazolo[5,4-c]pyridin-2-yl)methanone);
121) ((([4,4′-biindoline]-1,1′-dicarbonyl)bis(6,7-dihydrothiazolo[5,4-c]pyridine-2,5(4H)-diyl))bis(propane-3,1-diyl))diboronic acid;
122) tetramethyl ((([4,4′-biindoline]-1,1′-dicarbonyl)bis(6,7-dihydrothiazolo[5,4-c]pyridine-2,5(4H)-diyl))bis(propane-3,1-diyl))diboronate;
123) dimethyl ((2-(1′-(5-((dimethoxyphosphoryl)methyl)-4,5,6,7-tetrahydrothiazolo[5,4-c]pyridine-2-carbonyl)-[4,4′-biindoline]-1-carbonyl)-6,7-dihydrothiazolo[5,4-c]pyridin-5(4H)-yl)methyl)phosphonate;
124) dimethyl (2-(2-(1′-(5-(2-(dimethoxyphosphoryl)ethyl)-4,5,6,7-tetrahydrothiazolo[5,4-c]pyridine-2-carbonyl)-[4,4′-biindoline]-1-carbonyl)-6,7-dihydrothiazolo[5,4-c]pyridin-5(4H)-yl)ethyl)phosphonate;
125) [4,4′-biindoline]-1,1′-diylbis((5-((2H-tetrazol-5-yl)methyl)-4,5,6,7-tetrahydrothiazolo[5,4-c]pyridin-2-yl)methanone);
126) N,N′-((([4,4′-biindoline]-1,1′-dicarbonyl)bis(5,6,7,8-tetrahydro-4H-5,8-epiminocyclohepta[d]thiazole-2,9-diyl))bis(ethane-2,1-diyl))dimethanesulfonamide;
127) 2,2′-(([4,4′-biindoline]-1,1′-dicarbonyl)bis(6,7-dihydrothiazolo[5,4-c]pyridine-2,5(4H)-diyl))bis(N-(methylsulfonyl)propanamide);
128) 2,2′-(([4,4′-biindoline]-1,1′-dicarbonyl)bis(6,7-dihydrothiazolo[5,4-c]pyridine-2,5(4H)-diyl))bis(2-methyl-N-(methylsulfonyl)propanamide);
129) 1,1′-(([4,4′-biindoline]-1,1′-dicarbonyl)bis(6,7-dihydrothiazolo[5,4-c]pyridine-2,5(4H)-diyl))bis(N-(methylsulfonyl)cyclopropane-1-carboxamide);
130) dimethyl ((([4,4′-biindoline]-1,1′-dicarbonyl)bis(6,7-dihydrothiazolo[5,4-c]pyridine-2,5(4H)-diyl))bis(ethane-2,1-diyl))dicarbamate;
131) dimethyl (((([4,4′-biindoline]-1,1′-dicarbonyl)bis(6,7-dihydrothiazolo[5,4-c]pyridine-2,5(4H)-diyl))bis(cyclopropane-1,1-diyl))bis(methylene))dicarbamate;
132) dimethyl (((([4,4′-biindoline]-1,1′-dicarbonyl)bis(6,7-dihydrothiazolo[5,4-c]pyridine-2,5(4H)-diyl))bis(methylene))bis(cyclopropane-1,1-diyl))dicarbamate;
133) 1,1′-((([4,4′-biindoline]-1,1′-dicarbonyl)bis(6,7-dihydrothiazolo[5,4-c]pyridine2,5(4H)-diyl))bis(ethane-2,1-diyl))bis(thiourea);
134) 1,1′-((([4,4′-biindoline]-1,1′-dicarbonyl)bis(6,7-dihydrothiazolo[5,4-c]pyridine-2,5(4H)-diyl))bis(ethane-2,1-diyl))diurea;
135) dimethyl 2,2′-(((([4,4′-biindoline]-1,1′-dicarbonyl)bis(6,7-dihydrothiazolo[5,4-c]pyridine-2,5(4H)-diyl))bis(methylene))bis(1H-1,2,3-triazole-4,1-diyl))diacetate;
136) [4,4′-biindoline]-1,1′-diylbis((5,6,7,8-tetrahydro-[1,2,4]triazolo[1,5-a]pyrazin-2-yl) methanone);
137) (5-((12-azanyl)methyl)-1H-1,2,4-triazol-3-yl)(1′-(5-(aminomethyl)-1H-1,2,4-triazole-3-carbonyl)-[4,4′-biindolin]-1-yl)methanone;
138) 2,2′-(((((2,2′-dimethyl-[1,1′-biphenyl]-3,3′-diyl)bis(azanediyl))bis(1,7-naphthyridine-8,3-diyl))bis(methylene))bis(azanediyl))diacetic acid;
139) ((8-((3′-((3-(((2-hydroxyethyl)amino)methyl)-1,7-naphthyridin-8-yl)amino)-2,2′-di methyl-[1,1′-biphenyl]-3-yl)amino)-1,7-naphthyridin-3-yl)methyl)glycine;
140) 2,2′-(((((2,2′-dimethyl-[1,1′-biphenyl]-3,3′-diyl)bis(azanediyl))bis(1,7-naphthyridine-8,3-diyl))bis(methylene))bis(azanediyl))dipropionic acid;
141) (S)-4-(((8-((3′-((3-(((carboxymethyl)amino)methyl)-1,7-naphthyridin-8-yl)amino)-2,2′-dimethyl-[1,1′-biphenyl]-3-yl)amino)-1,7-naphthyridin-3-yl)methyl)amino)-3-hydroxybutanoic acid;
142) 2,2′-(((((2,2′-dimethyl-[1,1′-biphenyl]-3,3′-diyl)bis(azanediyl))bis(1,7-naphthyridine-8,3-diyl))bis(methylene))bis(azanediyl))bis(3-methylbutanoic acid);
143) (3S,3'S)-4,4′-(((((2,2′-dimethyl-[1,1′-biphenyl]-3,3′-diyl)bis(azanediyl))bis(1,7-naphthyridine-8,3-diyl))bis(methylene))bis(azanediyl))bis(3-hydroxybutanoic acid);
144) 2,2′-(((((2,2′-dimethyl-[1,1′-biphenyl]-3,3′-diyl)bis(azanediyl))bis(1,7-naphthyridine-8,3-diyl))bis(methylene))bis(azanediyl))diacetamide;
145) 2,2′-(((((2,2′-dimethyl-[1,1′-biphenyl]-3,3′-diyl)bis(azanediyl))bis(1,7-naphthyridine-8,3-diyl))bis(methylene))bis(azanediyl))bis(N,N-dimethylacetamide);
146) 2-(((8-((2,2′-dimethyl-3′-((3-(((2,2,2-trifluoroethyl)amino)methyl)-1,7-naphthyridin-8-yl)amino)-[1,1′-biphenyl]-3-yl)amino)-1,7-naphthyridin-3-yl)methyl)amino)-N,N-dimethylacetamide;
147) 2-(((8-((2,2′-dimethyl-3′-((3-(((2-(methylamino)-2-oxoethyl)amino)methyl)-1,7-naphthyridin-8-yl)amino)-[1,1′-biphenyl]-3-yl)amino)-1,7-naphthyridin-3-yl)methyl)amino)-N-(2-hydroxyethyl)-N-methylacetamide;
148) ((8-((3′-((3-(((2-(dimethylamino)-2-oxoethyl)amino)methyl)-1,7-naphthyridin-8-yl)amino)-2,2′-dimethyl-[1,1′-biphenyl]-3-yl)amino)-1,7-naphthyridin-3-yl)methyl)glycine;
149) ((8-((3′-((3-(azetidin-1-ylmethyl)-1,7-naphthyridin-8-yl)amino)-2,2′-dimethyl-[1,1′-biphenyl]-3-yl)amino)-1,7-naphthyridin-3-yl)methyl)glycine;
150) 1,1′-((((2,2′-dimethyl-[1,1′-biphenyl]-3,3′-diyl)bis(azanediyl))bis(1,7-naphthyridine-8,3-diyl))bis(methylene))bis(azetidin-3-ol);
151) 2,2′-dimethyl-N3,N3′-bis(3-(morpholinomethyl)-1,7-naphthyridin-8-yl)-[1,1′-biphenyl]-3,3′-diamine;
152) N,N′-((((((2,2′-dimethyl-[1,1′-biphenyl]-3,3′-diyl)bis(azanediyl))bis(1,7-naphthyridine-8,3-diyl))bis(methylene))bis(azanediyl))bis(ethane-2,1-diyl))diacetamide;
153) N3,N3′-bis(3-(((S)-3-(dimethylamino)pyrrolidin-1-yl)methyl)-1,7-naphthyridin-8-yl)-2,2′-dimethyl-[1,1′-biphenyl]-3,3′-diamine;
154) (S)-((8-((3′-((3-((3-(dimethylamino)pyrrolidin-1-yl)methyl)-1,7-naphthyridin-8-yl)amino)-2,2′-dimethyl-[1,1′-biphenyl]-3-yl)amino)-1,7-naphthyridin-3-yl)methyl)glycine;
155) 1,1′-((((2,2′-dimethyl-[1,1′-biphenyl]-3,3′-diyl)bis(azanediyl))bis(1,7-naphthyridine-8,3-diyl))bis(methylene))bis(piperidine-2-carboxylic acid);
156) 2-(((8-((2,2′-dimethyl-3′-((3-(((2,2,2-trifluoroethyl)amino)methyl)-1,7-naphthyridin-8-yl)amino)-[1,1′-biphenyl]-3-yl)amino)-1,7-naphthyridin-3-yl)methyl)amino)ethan-1-ol;
157) (1S,1'S)-1,1′-((((((2,2′-dimethyl-[1,1′-biphenyl]-3,3′-diyl)bis(azanediyl))bis(1,7-naphthyridine-8,3-diyl))bis(methylene))bis(azanediyl))bis(cyclopropane-1,1-diyl))bis(ethan-1-ol);
158) dimethyl ((((((2,2′-dimethyl-[1,1′-biphenyl]-3,3′-diyl)bis(azanediyl))bis(1,7-naphthyridine-8,3-diyl))bis(m ethylene))bis(azanediyl))bis(ethane-2,1-diyl))dicarbamate;
159) 2-(2-(4-(3-((3-(((2-((methoxycarbonyl)amino)ethyl)amino)methyl)-1,7-naphthyridin-8-yl)amino)-2-methylphenyl)indoline-1-carbonyl)-3a,6,7,7a-tetrahydrothiazolo[5,4-c]pyridin-5 (4H)-yl)acetic acid;
160) ((8-((3-(1-(5-(carboxymethyl)-3a,4,5,6,7,7a-hexahydrothiazolo[5,4-c]pyridine-2-carbonyl)indolin-4-yl)-2-methylphenyl)amino)-1,7-naphthyridin-3-yl)methyl)glycine;
161) 3-(2-(4-(3-((3-(((carboxymethyl)amino)methyl)-1,7-naphthyridin-8-yl)amino)-2-methylphenyl)indoline-1-carbonyl)-3a,6,7,7a-tetrahydrothiazolo[5,4-c]pyridin-5(4H)-yl)propanoic acid;
162) 3-(2-(4-(3-((3-(((2-hydroxyethyl)amino)methyl)-1,7-naphthyridin-8-yl)amino)-2-m ethylphenyl)indoline-1-carbonyl)-3a,6,7,7a-tetrahydrothiazolo[5,4-c]pyridin-5(4H)-yl)propanoic acid;
163) ((8-((3-(1-(5-(carboxymethyl)-3a,4,5,6,7,7a-hexahydrothiazolo[5,4-c]pyridine-2-carbonyl)indolin-4-yl)-2-methylphenyl)amino)-1,7-naphthyridin-3-yl)methyl)alanine;
164) 2-(2-(4-(3-((3-(((2-amino-2-oxoethyl)amino)methyl)-1,7-naphthyridin-8-yl)amino)-2-methylphenyl)indoline-1-carbonyl)-3a,6,7,7a-tetrahydrothiazolo[5,4-c]pyridin-5(4H)-yl)acetic acid;
165) 2-(2-(4-(3-((3-(((2-(dimethylamino)-2-oxoethyl)amino)methyl)-1,7-naphthyridin-8-yl)amino)-2-methylphenyl)indoline-1-carbonyl)-3a,6,7,7a-tetrahydrothiazolo[5,4-c]pyridin-5(4H)-yl)acetic acid;
166) 2-(2-(4-(3-((3-(((2-((2-hydroxyethyl)(methyl)amino)-2-oxoethyl)amino)methyl)-1,7-naphthyridin-8-yl)amino)-2-methylphenyl)indoline-1-carbonyl)-3a,6,7,7a-tetrahydrothiazolo[5,4-c]pyridin-5(4H)-yl)acetic acid;
167) N,N′-(2,2′-dimethyl-[1,1′-biphenyl]-3,3′-diyl)bis(5,6,7,8-tetrahydro-1,6-naphthyridine-2-carboxamide);
168) dimethyl ((2S,2'S)-(([4,4′-biindoline]-1,1′-dicarbonyl)bis(7,8-dihydro-1,6-naphthyridine-2,6(5H)-diyl))bis(3-methyl-1-oxobutane-1,2-diyl))dicarbamate;
169) dimethyl ((2S,2'S)-((((2,2′-dimethyl-[1,1′-biphenyl]-3,3′-diyl)bis(azanediyl))bis(carbonyl))bis(7,8-dihydro-1,6-naphthyridine-2,6(5H)-diyl))bis(3-methyl-1-oxobutane-1,2-diyl))dicarbamate;
170) ethyl 2-(2-((2,2′-dimethyl-3′-(5,6,7,8-tetrahydro-1,6-naphthyridine-2-carboxamido)-[1,1′-biphenyl]-3-yl)carbamoyl)-7,8-dihydro-1,6-naphthyridin-6(5H)-yl)acetate;
171) 2-((3′-(6,6-dimethyl-5,6,7,8-tetrahydro-614-1,6-naphthyridin-7-ylium-2-carboxamido)-2,2′-dimethyl-[1,1′-biphenyl]-3-yl)carbamoyl)-6,6-dimethyl-5,6,7,8-tetrahydro-1,6-naphthyridin-6-ium chloride;
172) di-tert-butyl 2,2′-((((2,2′-dimethyl-[1,1′-biphenyl]-3,3′-diyl)bis(azanediyl))bis(carbonyl))bis(7,8-dihydro-1,6-naphthyridine-2,6(5H)-diyl))diacetate;
173) 2,2′-((((2,2′-dimethyl-[1,1′-biphenyl]-3,3′-diyl)bis(azanediyl))bis(carbonyl))bis(7,8-dihydro-1,6-naphthyridine-2,6(5H)-diyl))diacetic acid;
174) N,N′-(2,2′-dimethyl-[1,1′-biphenyl]-3,3′-diyl)bis(6-(2-hydroxyethyl)-5,6,7,8-tetrahydro-1,6-naphthyridine-2-carboxamide);
175) N,N′-(2,2′-dimethyl-[1,1′-biphenyl]-3,3′-diyl)bis(6-methyl-5,6,7,8-tetrahydro-1,6-naphthyridine-2-carboxamide);
176) N,N′-(2,2′-dimethyl-[1,1′-biphenyl]-3,3′-diyl)bis(6-(2-morpholinoethyl)-5,6,7,8-tetrahydro-1,6-naphthyridine-2-carboxamide);
177) diethyl (2-(2-((3′-(((6-(2-(diethoxyphosphoryl)ethyl)-5,6,7,8-tetrahydro-1,6-naphthyridin-2-yl)methyl)amino)-2,2′-dimethyl-[1,1′-biphenyl]-3-yl)carbamoyl)-7,8-dihydro-1,6-naphthyridin-6(5H)-yl)ethyl)phosphonate;
178) 2-(2-((2,2′-dimethyl-3′-(5,6,7,8-tetrahydro-1,6-naphthyridine-2-carboxamido)-[1,1′-biphenyl]-3-yl)carbamoyl)-7,8-dihydro-1,6-naphthyridin-6(5H)-yl)acetic acid;
179) diethyl 2,2′-((((((2,2′-dimethyl-[1,1′-biphenyl]-3,3′-diyl)bis(azanediyl))bis(carbonyl))bis(pyrazine-5,2-diyl))bis(ethane-1,1-diyl))bis(azanediyl))diacetate;
180) N,N′-(2,2′-dimethyl-[1,1′-biphenyl]-3,3′-diyl)bis(5-(1-(methylamino)ethyl)pyrazine-2-carboxamide);
181) N,N′-(2,2′-dimethyl-[1,1′-biphenyl]-3,3′-diyl)bis(5-(1-((2-hydroxyethyl)amino)ethyl)pyrazine-2-carboxamide);
182) 6-acetyl-N-(2,2′-dimethyl-3′-(5,6,7,8-tetrahydro-1,6-naphthyridine-2-carboxamido)-[1,1′-biphenyl]-3-yl)-5,6,7,8-tetrahydro-1,6-naphthyridine-2-carboxamide;
183) N-(3′-(5-acetyl-1-methyl-4,5,6,7-tetrahydro-1H-imidazo[4,5-c]pyridine-2-carboxamido)-2,2′-dimethyl-[1,1′-biphenyl]-3-yl)-5,6,7,8-tetrahydro-1,6-naphthyridine-2-carboxamide;
184) bis((5-methyl-2-oxo-1,3-dioxol-4-yl)methyl) 2,2′-(((2,2′-dimethyl-[1,1′-biphenyl]-3,3′-diyl)bis(azanediyl))bis(carbonyl))bis(7,8-dihydro-1,6-naphthyridine-6(5H)-carboxylate);
185) N-(2,2′-dimethyl-3′-(5,6,7,8-tetrahydro-1,6-naphthyridine-2-carboxamido)-[1,1′-biphenyl]-3-yl)-6-methyl-5,6,7,8-tetrahydro-1,6-naphthyridine-2-carboxamide;
186) N-(2,2′-dimethyl-3′-(5,6,7,8-tetrahydro-1,6-naphthyridine-2-carboxamido)-[1,1′-biphenyl]-3-yl)-6-(2-hydroxyethyl)-5,6,7,8-tetrahydro-1,6-naphthyridine-2-carboxamide;
187) N,N′-(2,2′-dimethyl-[1,1′-biphenyl]-3,3′-diyl)bis(6-(2,2-difluoroethyl)-5,6,7,8-tetrahydro-1,6-naphthyridine-2-carboxamide);
188) N,N′-(2,2′-dimethyl-[1,1′-biphenyl]-3,3′-diyl)bis(5,6,7,8-tetrahydropyrido[4,3-d]pyrimidine-2-carboxamide);
189) [4,4′-biindoline]-1,1′-diylbis((5,6,7,8-tetrahydropyrido[4,3-d]pyrimidin-2-yl)methanone); or
190) 2,2′-((((2,2′-dimethyl-[1,1′-biphenyl]-3,3′-diyl)bis(azanediyl))bis(carbonyl))bis(7,8-dihydropyrido[4,3-d]pyrimidine-2,6(5H)-diyl))diacetic acid.

34. A pharmaceutical composition comprising a compound of claim 1, or a pharmaceutically acceptable salt or a stereoisomer thereof, and at least one pharmaceutically acceptable carrier or excipient.

35. A method of inhibiting PD-1/PD-L1 interaction, said method comprising administering to a patient a compound of claim 1, or a pharmaceutically acceptable salt or a stereoisomer thereof.

36. A method of treating a disease associated with inhibition of PD-1/PD-L1 interaction, said method comprising administering to a patient in need thereof a therapeutically effective amount of a compound of claim 1, or a pharmaceutically acceptable salt or a stereoisomer thereof.

37. The method of claim 36, wherein the disease is colon cancer, gastric cancer, thyroid cancer, lung cancer, leukemia, pancreatic cancer, melanoma, multiple melanoma, brain cancer, renal cancer, prostate cancer, ovarian cancer or breast cancer.

38. A method of enhancing, stimulating and/or increasing the immune response in a patient, said method comprising administering to the patient in need thereof a therapeutically effective amount of a compound of claim 1, or a pharmaceutically acceptable salt or a stereoisomer thereof.

39. Use of the compound of claim 1 for the preparation of a medicament.

40. The use of claim 39, wherein the medicament is used for the treatment or prevention of cancer.

41. The use of claim 40, wherein the cancer is colon cancer, gastric cancer, thyroid cancer, lung cancer, leukemia, pancreatic cancer, melanoma, multiple melanoma, brain cancer, renal cancer, prostate cancer, ovarian cancer or breast cancer; or
the medicament is used as an inhibitor of PD-1/PD-L1 interaction.

42. (canceled)

43. Use of the pharmaceutical composition of claim 12 for the preparation of a medicament.
